(19) United States
(12) Patent Application Publication
(10) Pub. No.: US 2022/0024891 A1
(43) Pub. Date: Jan. 27, 2022

(54) THERAPEUTICS TARGETING MUTANT ADENOMATOUS POLYPOSIS COLI (APC) FOR THE TREATMENT OF CANCER

(71) Applicant: The Board of Regents of the University of Texas System, Austin, TX (US)

(72) Inventors: Jef K. De Brabander, Austin, TX (US); Wentian Wang, Austin, TX (US)

(21) Appl. No.: 17/299,760

(22) PCT Filed: Dec. 4, 2019
(86) PCT No.: PCT/US2019/064529

§ 371 Date: Jun. 3, 2021

Related U.S. Application Data
(60) Provisional application No. 62/775,297, filed on Dec. 4, 2018.
(60) Provisional application No. 62/838,876, filed on Apr. 25, 2019.

(51) Int. Cl. C07D 401/04; C07D 401/14; C07D 413/14; C07D 417/14; C07D 407/14; C07D 211/96; C07D 295/04; C07D 211/98; C07D 491/107; A61P 35/00; C07D 211/10; C07D 211/14; C07D 211/58; C07D 405/04

(52) U.S. Cl. CPC C07D 401/04; C07D 401/14; C07D 413/14; C07D 417/14; C07D 407/14; C07D 211/96; C07D 405/04; C07D 211/98; C07D 491/107; A61P 35/00; C07D 211/10; C07D 211/14; C07D 211/58; C07D 295/04

(57) ABSTRACT

The present disclosure reports an extensive medicinal chemistry evaluation of a large collection of Truncating APC-Selective Inhibitor (TASIN) compounds. The compounds were evaluated for activity against a series of colon cancer cell lines with and without truncating APC-mutations, as well as in an isogenic cell line pair reporting on the status of APC-dependent selectivity. A number of very potent and selective compounds were identified, including compounds with good metabolic stability and PK properties. The small molecules reported herein thus represent a first-in-class genotype-selective series that specifically target ape mutations present in the vast majority of CRC patients, and therefore serves as a translational platform towards a potential targeted therapy for colon cancer.

### CROSS-REFERENCE TO RELATED APPLICATIONS

This application claims benefit of priority under 35 U.S.C. § 119(e) of U.S. Ser. No. 62/775,297, filed Dec. 4, 2018, and U.S. Ser. No. 62/838,876, filed Apr. 25, 2019, the entire contents of both is incorporated herein by reference in its entirety.

### STATEMENT OF GOVERNMENT SUPPORT

This disclosure was made with support by the Cancer Prevention and Research Institute of Texas (grants RP130189 and RP160180). The government has certain rights in the disclosure.

### FIELD OF THE DISCLOSURE

The disclosure relates to small molecule cancer therapeutics, specifically to colon cancer.

### BACKGROUND

The major types of lipids that circulate in plasma include cholesterol and cholesteryl esters, phospholipids and triglycerides. Cholesterol contributes an essential component of mammalian cell membranes and furnishes substrate for steroid hormones and bile acids. Many cell functions depend critically on membrane cholesterol, and cells tightly regulate cholesterol content. Most of the cholesterol in plasma circulates in the form of cholesteryl esters in the core of lipoprotein particles. The enzyme lecithin cholesterol acyl transferase (LCAT) forms cholesteryl esters in the blood compartment by transferring a fatty acyl chain from phosphatidylcholine to cholesterol.

Lipoproteins are complex macromolecular structures composed of an envelope of phospholipids and free cholesterol, a core of cholesteryl esters and triglycerides. Triglycerides consist of a three-carbon glycerol backbone covalently linked to three fatty acids. Their fatty acid composition varies in terms of chain length and degree of saturation. Triglyceride molecules are nonpolar and hydrophobic, and are transported in the core of the lipoprotein. Hydrolysis of triglycerides by lipases generates free fatty acids (FFAs) used for energy. Phospholipids, constituents of all cellular membranes, consist of a glycerol molecule linked to two fatty acids. The fatty acids differ in length and in the presence of a single or multiple double bonds. The third carbon of the glycerol moiety carries a phosphate group to which one of four molecules is linked: choline (phosphatidylcholine or lecithin), ethanolamine (phosphatidylethanolamine), serine (phosphatidylserine), or inositol (phosphatidylinositol). Phospholipids, which are polar molecules, more soluble than triglycerides or cholesterol or its esters, participate in signal transduction pathways. Hydrolysis by membrane-associated phospholipases generates second messengers such as diacyl glycerols, lysophospholipids, phoshatidic acids and free fatty acids (FFAs) such as arachidonate that can regulate many cell functions.

The apolipoproteins, which comprise the protein moiety of lipoproteins, vary in size, density in the aqueous environment of plasma, and lipid and apolipoprotein content. The classification of lipoproteins reflects their density in plasma (1.006 gm/mL) as gauged by flotation in the ultracentrifuge. For example, triglyceride-rich lipoproteins consisting of chylomicrons (meaning a class of lipoproteins that transport dietary cholesterol and triglycerides after meals from the small intestine to tissues for degradation) and very low density lipoprotein (VLDL) have a density less than 1.06 gm/mL.

Apolipoproteins have four major roles: (1) assembly and secretion of the lipoprotein (apo B100 and B48); (2) structural integrity of the lipoprotein (apo B, apo E, apo A1, apo AII); (3) coactivators or inhibitors of enzymes (apo A1, C1, CII, CIII); and (4) binding or docking to specific receptors and proteins for cellular uptake of the entire particle or selective uptake of a lipid component (apoA1, B100, E). The role of several apolipoproteins (AIV, AV, D, and J) remain incompletely understood.

Low density lipoprotein (or LDL cholesterol) particles carry cholesterol throughout the body, delivering it to different organs and tissues. The excess keeps circulating in blood. LDL particles contain predominantly cholesteryl esters packaged with the protein moiety apoB100.

High density lipoproteins (or HDL cholesterol) act as cholesterol scavengers, picking up excess cholesterol in the blood and taking it back to the liver where it is broken down. Apolipoprotein A1, the main protein of HDL, is synthesized in the intestine and liver. Lipid-free Apo A1 acquires phospholipids from cell membranes and from redundant phospholipids shed during hydrolysis of triglceride-rich lipoproteins. Lipid-free apo A1 binds to ABCA1 and promotes its phosphorylation via cAMP, which increases the net efflux of phospholipids and cholesterol onto apo A1 to form a nascent HDL particle. These nascent HDL particles will mediate further cellular cholesterol efflux.

The scavenger receptor class B (SR-B1; also named CLA-1 in humans and the adenosine triphosphate binding cassette transporter A1 (ABCA1) bind HDL particles. SR-B1, a receptor for HDL (also for LDL and VLDL, but with less affinity), mediates the selective uptake of HDL cholesteryl esters in steroidogenic tissues, hepatocytes and endothelium. ABCA1 mediates cellular phospholipid (and possibly cholesterol) efflux and is necessary and essential for HDL biogenesis.

Cellular cholesterol homeostasis is achieved via at least four major routes: (1) cholesterol de novo biosynthesis from acetyl-CoA in the endoplasmic reticulum; (2) cholesterol uptake by low density lipoprotein (LDL) receptor-mediated endocytosis of LDL-derived cholesterol from plasma; 3) cholesterol efflux mediated by ABC family transporters such as ATP-binding cassette, sub-family A (ABC1), member 1 (ABCA1)/ATP-binding cassette, sub-family G, member 1 (ABCG1), and secretion mediated by apolipoprotein B (ApoB); and (4) cholesterol esterification with fatty acids to cholesterol esters (CE) by acyl-coenzyme A:cholesterol acyltransferase (ACAT).

### Cholesterol Biosynthetic Pathways

Cholesterol synthesis takes place in four stages: (1) condensation of three acetate units to form a six-carbon intermediate, mevalonate; (2) conversion of mevalonate to activated isoprene units; (3) polymerization of six 5-carbon isoprene units to form the 30-carbon linear squalene; and (4) cyclization of squalene to form the steroid nucleus, with a further series of changes to produce cholesterol.

The mevalonate arm of the cholesterol biosynthesis pathway, which includes enzymatic activity in the mitochondria, peroxisome, cytoplasm and endoplasmic reticulum, starts with the consumption of acetyl-CoA, which occurs in parallel in three cell compartments (the mitochondria, cytoplasm, and peroxisome) and terminates with the production of squalene in the endoplasmic reticulum. The following are enzymes of the mevalonate arm:

Acetyl-CoA acetyltransferase (ACAT1; ACAT2; acetoacetyl-CoA thiolase; EC 2.3.1.9) catalyzes the reversible condensation of two molecules of acetylcoA and forms acetoacetyl-CoA.

Hydroxymethylglutaryl-CoA synthase (HMGCS1 (cytoplasmic); HMGCS2 (mitochondria and peroxisome); EC 2.3.3.10 catalyzes the formation of 3-hydroxy-3-methylglutaryl CoA (3HMG-CoA) from acetyl CoA and acetoacetyl Co A.

Hydroxymethylglutaryl-CoAlysase (mitochondrial, HMGCL; EC 4.1.3.4) transforms HMG-CoA into Acetyl-CoA and acetoacetate.

3-hydroxy-3-methylglutaryl-coenzyme A reductase (HMGCR; EC 1.1.34) catalyzes the conversion of 3HMG-CoA into mevalonic acid. This step is the committed step in cholesterol formation. HMGCR is highly regulated by signaling pathways, including the SREBP pathway.

Mevalonate kinase (MVK; ATP:mevalonate 5-phosphotransferase; EC 2.7.1.36) catalyzes conversion of mevalonate into phosphomevalonate.

Phosphomevalonate kinase (PMVK; EC 2.7.4.2) catalyzes formation of mevalonate 5-diphosphate from mevalonate 5-phosphate.

Diphosphomevalonate decarboxylase (MVD; mevalonate (diphospho) decarboxylase; EC 4.1.1.33) decarboxylates mevalonate 5-diphosphate, forming isopentenyldiphosphate while hydrolyzing ATP.

Isopentenyl-diphosphate delta-isomerases (ID11; ID12; EC 5.3.3.2) isomerize isopentenyl diphosphate into dimethylallyl diphosphate, the fundamental building blocks of isoprenoids.

Farnesyl diphosphate synthase (FDPS; EC2.5.1.10; EC 2.5.1.1; dimethylallyltranstransferase) catalyzes two reactions that lead to farnesyl diphosphate formation. In the first (EC 2.5.1.1 activity), isopentyl diphosphate and dimethylallyl diphosphate are condensed to form geranyl disphosphate. Next, geranyl diphosphate and isopentenyl diphosphate are condensed to form farnesyl diphosphate (EC 2.5.1.10 activity).

Geranylgeranyl pyrophosphate synthase (GGPS1; EC 1.5.1.29; EC 2.5.1.10; farnesyl diphosphate synthase; EC 2.5.1.1; dimethylallyltranstransferase) catalyzes the two reactions of farnesyl diphosphate formation and the addition of three molecules of isopentenyl diphosphate to dimethylallyl diphosphate to form geranylgeranyl diphosphate.

Farnesyl-diphosphate farnesyltransferase 1 (FDFT1; EC 2.5.1.21; squalene synthase) catalyzes a two-step reductive dimerization of two farnesyl diphosphate molecules (C15) to form squalene (C30). The FDFT1 expression level is regulated by cholesterol status; the human FDFT1 gene has a complex promoter with multiple binding sites for SREBP-1a and SREBP-2.

The sterols arms of the pathway start with Squalene and terminate with cholesterol production on the Bloch and Kandutsch-Russell pathways and with 24 (S),25-epoxycholesterol on the shunt pathway. The following are enzymes of the sterol arms:

Squalene epoxidase (SQLE; EC 1.14.13.132, squalene monooxygenase) catalyzes the conversion of squalene into squalene-2,3-epoxide and the conversion of squalene-2,3-epoxide (2,3-oxidosqualene) into 2,3:22,23-diepoxysqualene (2,3:22,23-dioxidosqualene). The first reaction is the first oxygenation step in the cholesterol biosynthesis pathway. The second is the first step in 24(S),25-epoxycholesterol formation from squalene 2,3-epoxide.

Lanosterol synthase (LSS; OLC; OSC; 2,3-oxidosqualene:lanosterol cyclase; EC 5.4.99.7) catalyzes cyclization of squalene-2,3-epoxide to lanosterol and 2,3:22,23-depoxysqualene to 24(S),25-epoxylanosterol.

Delta(24)-sterol reductase (DHCR24; 24-dehydrocholesterol reductase; EC 1.3.1.72) catalyzes the reduction of the delta-24 double bond of intermediate metabolites. In particular it converts lanosterol into 24, 25-dihydrolanosterol, the initial metabolite of the Kandutsch-Russel pathway and also provides the last step of the Bloch pathway converting desmosterol into cholesterol. Intermediates of the Bloch pathway are converted by DHCR24 into intermediates of the Kandutsch-Russell pathway.

Lanosterol 14-alpha demethylase (CYP51A1; cytochrome P450, family 51, subfamily A, polypeptide 1; EC 1.14.13.70) converts lanosterol into 4,4-dimethyl-5α-cholesta-8,14,24-trien-3β-ol and 24,25-dihydrolanosterol into 4,4-dimethyl-5α-cholesta-8,14-dien-3β-ol in three steps.

Delta (14)-sterol reductase (TM7F2; transmembrane 7 superfamily member 2, EC 1.3.1.70) catalyzes reactions on the three branches of the cholesterol and 24(S),25-epoxycholesterol pathways.

Methylsterol monooxygenase 1 (MSMO1; SC4MOL; C-4 methylsterol oxidase; EC 1.14.13.72) catalyzes demethylation of C4 methylsterols.

Sterol-4-alpha-carboxylate 3-dehydrogenase, decarboxylating (NSDHL; NAD(P) dependent steroid dehydrogenase-like; EC 1.1.1.170) participates in several steps of post-squalene cholesterol and 24(S),25-epoxycholeseterol synthesis.

3-keto-steroid reductase (HSD17B7; 17-beta-hydroxysteroid dehydrogenase 7; EC 1.1.1.270) converts zymosterone into zymosterol in the Bloch pathway.

3-Beta-hydroxysteroid-delta(8),delta(7)-isomerase (emopamil-binding protein EBP; EC5.3.3.5) catalyzes the conversion of delta(8)-sterols into delta(7)-sterols. Id.

Lathosterol oxidase (SC5DL; sterol-C5-desaturase (ERG3 delta-5-desaturase homolog, S. cerevisiae-like; EC 1.14.21.6) catalyzes the production of 7-dehydrocholesterol, 7-dehydrodesmosterol and 24(S),25-epoxy-7-dehydrocholesterol.

7-dehydrocholesterol reductase (DHCR7; EC 1.3.1.21) catalyzes reduction of the C7-C8 double bond of 7-dehydrocholesterol and formation of cholesterol, and produces desmosterol from 7-dehydrodesmosterol and 24(S),25-epoxycholesterol from 24(S),25-epoxy-7-dehydrocholesterol.

Cytochrome P450, family 3, subfamily A, polypeptide 4 (CYP3A4; 1,8-cineole 2-exo-monooxygenase; taurochenodeoxycholate 6α-hydroxylase; EC 1.14.13.97)) catalyzes the hydroxylation of cholesterol leading to 25-hydroxycholesterol and 40-hydroxycholesterol.

Cholesterol 25-hydroxylase (CH25H; cholesterol 25-monooxygenase; EC 1.14.99.38) uses di-iron cofactors to catalyze the hydroxylation of cholesterol to produce 25-hydroxycholesterol, and has the capacity to catalyze the transition of 24-hydroxycholesterol to 24, 25-dihydroxycholesterol.

Cytochrome P450, family 7, subfamily A, polypeptide 1 (CYP7A1; cholesterol 7-alpha-hydroxylase; EC 1.14.13.17) is responsible for introducing a hydrophilic moiety at position 7 of cholesterol to form 7α-hydroxycholesterol.

Cytochrome P450, family 27, subfamily A, polypeptide 1 (CYP27A1; Sterol 27-hydroxylase; EC 1.14.13.15) catalyzes the transition of mitochondrial cholesterol to 27-hydroxycholesterol and 25-hydroxycholesterol.

Cytochrome P450 46A1 (CYP46A1, cholesterol 24-hydroxylase, EC 1.14.13.98) catalyzes transformation of cholesterol into 24(S)-hydroxycholesterol.

### Intermediates in Cholesterol Synthesis as Physiological Regulators

Intermediates in cholesterol synthesis, mostly sterols (e.g. 7-dehydrocholesterol, which is converted to cholesterol by DHCR7 (7-dehydrocholesterol reductase), but which also is a precursor for vitamin D), have been credited with having regulatory functions distinct from those of cholesterol.

C4-methylsterols are produced by lanosterol 14α-demethylase (encoded by CYP51A1 (cytochrome P450, family 51, subfamily A, polypeptide 1) and demethylated by SC4MOL (sterol-C4-methyl oxidase like 1; methylsterol monooxygenase 1) and its partner, NSDHL (NAD(P)-dependent steroid dehydrogenase-like; sterol-4-α-carboxylate 3-dehydrogenase, decarboxylating).

24, 25-dihydrolanosterol purportedly is the primary degradation signal for 3-hydroxy-3-methylglutaryl-CoA reductase (HMGCR). The nonsterol intermediate squalene has been implicated in stimulating HMGCR degradation.

A number of cholesterol synthesis intermediates can serve as activating ligands of the nuclear liver X receptor (LXR), which up-regulates cholesterol export genes and represses inflammatory genes. These sterols include 24,25-dihydrolanosterol, meiosis-activating sterols (MASs) and desmosterol.

The oxysterol 24(S),25-epoxycholesterol (24,25-EC), a potent LXR agonist is produced in a shunt pathway in sterol synthesis, and its production is determined by the relative activities of squalene monooxygenase (SM) and lanosterol synthase (LS). Partial inhibition or knockdown of LS diverts more flux into the shunt pathway, producing more 14,15-epoxycholesterol (14,15-EC), whereas overexpression of LS abolishes 24,25-EC production. Conversely, overexpression of SM increases 24,25-EC production. The extent to which SM and LS are differentially regulated to alter 14,15-EC production is not known.

Cholesterol Uptake by Low Density Lipoprotein (LDL) Receptor-Mediated Endocytosis of LDL-Derived Cholesterol from Plasma

The LDL receptor regulates the entry of cholesterol into cells; tight control mechanisms alter its expression on the cell surface, depending on need. Other receptors for lipoproteins include several that bind VLDL, but not LDL. The LDL receptor-related peptide, which mediates the uptake of chylomicron remnants and VLDL, preferentially recognizes apolipoprotein E (apo E). The LDL receptor-related peptide interacts with hepatic lipase. A specific VLDL receptor also exists. The interaction between hepatocytes and the various lipoproteins containing apo E is complex and involves cell surface proteoglycans that provide a scaffolding for lipolytic enzymes (lipoprotein lipase and hepatic lipase) involved in remnant lipoprotein recognition.

Macrophages express receptors that bind modified (especially oxidized) lipoproteins. These scavenger lipoprotein receptors mediate the uptake of oxidized LDL into macrophages. In contrast to the regulated LDL receptor, high cellular cholesterol content does not suppress scavenger receptors, enabling the intimal macrophages to accumulate abundant cholesterol, become foam cells, and form fatty streaks. Endothelial cells also can take up modified lipoproteins through a specific receptor, such as Lox-1.

Cholesterol efflux is mediated by ABC family transporters such as ATP-binding Cassette, Sub-Family a (ABC1), Member 1 (ABCA1)/ATP-Binding Cassette, Sub-Family G, Member 1 (ABCG1), and Secretion Mediated by Apolipoprotein B (ApoB)

Because most cells in the body do not express pathways for catabolizing cholesterol, efflux of cholesterol is critical for maintaining homeostasis. High density lipoprotein (HDL) comprises a heterogeneous population of microemulsion particles 7-12 nm in diameter containing a core of cholesterol ester (CE) and triglyceride (TG) molecules stabilized by a monomolecular layer of phospholipid (PL) and apolipoprotein (apo), of which apol is the principal component. The presence of PL in the particles enables HDL to solubilize and transport unesterified (free) cholesterol (FC) released from cells, thereby mediating removal of cholesterol from cholesterol-loaded arterial macrophages and transport to the liver for catabolism and elimination from the body (“reverse cholesterol transport”).

The first step in reverse cholesterol transport is efflux of FC from the cell plasma membrane to HDL. In the case of macrophages, four efflux pathways have been identified: the aqueous diffusion efflux pathway, the scavenger receptor class B, type 1 (SR-B1) pathway; the ATP binding cassette transporter G1 (ABCG1) pathway and the ATP-binding cassette transporter A1 (ABCA1) pathway. The first two processes, which are passive, involve simple diffusion (aqueous diffusion pathway) and facilitated diffusion (SR-B1-mediated pathway). The two active processes involve members of the ATP-binding cassette (ABC) family of transmembrane transporters, namely ABCA1 and ABCG1. The efficiency of an individual serum sample in accepting cellular cholesterol depends upon both the distribution of HDL particles present and the levels of cholesterol transporters expressed in the donor cells.

### Aqueous Diffusion Efflux Pathway

HDL is the component of serum responsible for mediating FC efflux from monolayers of mouse L-cell fibroblasts. Transfer occurs by an aqueous phase intermediate where monomeric FC molecules desorb from a donor particle and diffuse until they are absorbed by an acceptor particle. The rate of transfer of the highly hydrophobic cholesterol molecule from donor to acceptor is limited by the rate of desorption into the aqueous phase, which is sensitive to the physical state of the phospholipid (PL) milieu in which the transferring FC molecules are located. The net mass FC efflux from cells to HDL in the extracellular medium is promoted by metabolic trapping in which return of released FC to the cell is prevented by esterification, when lecithin-cholesterol aceyltransferase acts on HDL.

### SR-B1 Efflux Pathway

SR-B1 is a member of the CD36 superfamily of scavenger receptor proteins that also includes lysosomal integral membrane protein-2 (LIMP-2). The receptor is most abundantly expressed in liver, where it functions in the reverse cholesterol transport pathway and in steroidogenic tissue, where it mediates cholesterol delivery. It is a homo-oligomeric glycoprotein located in the plasma membrane with two N- and C-terminal transmembrane domains and a large central extracellular domain. In 1996, it was established that SR-B1 is an HDL receptor that mediates cholesterol uptake into cells. This process involves selective transfer of the cholesterol ester (CE) in an HDL particle into the cell without endocytic uptake and degradation of the HDL particle itself. In addition to promoting delivery of HDL cholesterol to cells, SR-B1 also enhances efflux of cellular cholesterol to HDL with the two processes being related. For CE selective uptake via SR-B1, HDL binding and CE uptake are tightly coupled. The mechanism for CE uptake from HDL involves a two-step process in which HDL first binds to the receptor and then CE molecules transfer from the bound HDL particle into the cell plasma membrane, with enhanced binding of larger HDL particles to SR-B1 increasing the selective delivery of CE. The binding of HDL to the extracellular domain of SR-B1 involves direct protein-protein contact with a recognition motif being the amphipathic α helix characteristic of HDL apolipoproteins. Consistent with CE selective uptake being a passive process, the rate of uptake is proportional to the amount of CE initially present in the HDL particles.

FC efflux and HDL binding are not completely coupled, and the FC efflux mechanism proceeds by different pathways at low and high extracellular HDL concentrations. At low HDL concentrations, binding of HDL to SR-B1 is critical, allowing bidirectional FC transit through the hydrophobic tunnel present in the extracellular domain of the receptor. Because the FC concentration gradient between the bound HDL particle and the cell plasma membrane is opposite to that of CE, the relatively high FC/PL ratio in the plasma membrane causes the direction of net mass FC transport to be out of the cell. Consistent with this concept, enhancing the PL content of HDL promotes FC efflux from cells. Larger HDL particles promote more FC efflux than smaller HDL, because they bind better to SR-B1. At higher HDL concentrations where binding to the receptor is saturated, FC efflux still increases with increasing HDL concentration, because SR-B1 induces reorganization of the FC in the cell plasma membrane.

### ABCG1 Effiux Pathway

ABCG1 functions as a homodimer, and is expressed in several types, where it mediates cholesterol transport through its ability to translocate cholesterol and oxysterols across membranes. Expression of ABCG1 enhances FC and PL efflux to HDL, but not to lipid-free apoA-1. The presence of the transporter induces reorganization of plasma membrane cholesterol so that it becomes accessible to cholesterol oxidase, creating an activated pool of plasma membrane FC, and desorption of FC molecules from this environment into the extracellular medium is facilitated. Increased expression of ABCG1 enhances FC efflux to HDL2 and HDL3 similarly, but has no effect on the influx of FC from these lipoprotein particles.

### ABCA1 Efflux Pathway

ABCA1 is a full transporter whose expression is up-regulated by cholesterol loading, which leads to enhanced FC efflux. Binding and hydrolysis of ATP by the two cytoplasmic, nucleotide-binding domains control the conformation of the transmembrane domains so that the extrusion pocket is available to translocate substrate from the cytoplasmic leaflet to the exofacial leaflet of the bilayer membrane. ABCA1 actively transports phosphatidylcholine, phosphatidylserine, and sphingomyelin with a preference for phosphatidylcholine. This PL translocase activity leads to the simultaneous efflux of PL and FC. The cellular FC released to apoA-1 originates from both the plasma membrane and the endosomal compartment.

The PL translocase activity of ABCA1 induces reorganization of lipid domains in the plasma membrane. ABCA1 exports PL and FC to various plasma apolipoproteins. Besides FC efflux, intracellular signaling pathways are activated by the interaction of apoA-1 with ABCA1.

It is well established that the activity of ABCA1 in the plasma membrane enhances binding of apoA-1 to the cell surface, but there has been controversy about the role of this binding in the acquisition of membrane PL by apo-A1. It has been proposed that apoA-1 acquires PL either directly from ABCA1 while it is bound to the transporter, or indirectly at a membrane lipid-binding site created by ABCA1 activity.

The ABCA1-mediated assembly of nascent HDL particles occurs primarily at the cell surface, where extracellular apoA-1 for HDL particle formation is available. The FC/PL ratio in nascent HDL particles created by ABCA1 activity is dependent upon the cell type and metabolic status of the cell, but the population of larger particles is always relatively FC-rich as compared with the smaller particles.

Regulation of cholesterol efflux depends in part on the ABCA1 pathway, controlled in turn by hydroxysterols, especially 24 and 27-OH cholesterol, which act as ligands for the liver-specific receptor (LXR) family of transcriptional regulatory factors.

Cholesterol Esterification with Fatty Acids to Cholesterol Esters (CE) by Acyl-Coenzyme A:Cholesterol Acyltransferase (ACAT)

Cholesterol content in membranes regulates the cholesterol acyltransferase (CAT) pathway at the level of protein regulation. Humans express two separate forms of ACAT (ACT1 and ACAT2), which derive from different genes and mediate cholesterol esterification in cytoplasm and in the endoplasmic reticulum lumen for lipoprotein assembly and secretion.

### Regulation of Cholesterol Content

Under conditions of cell cholesterol sufficiency, the cell can decrease its input of cholesterol by decreasing the de novo synthesis of cholesterol. The cell can also decrease the amount of cholesterol that enters the cell via the LDL-R, increase the amount stored as cholesteryl esters, and promote the removal of cholesterol by increasing its movement to the plasma membrane for efflux.

The regulation of HMG CoA reductase, the rate limiting step in cholesterol biosynthesis, has been investigated in detail. However, this enzyme acts very early in the cholesterol synthesis pathway. There is accumulating evidence that enzymes beyond HMG CoA reductase serve as flux controlling points, and that regulation of cholesterol synthesis can occur at multiple levels throughout the pathway.

### Transcriptional Regulation: Sterol Regulatory Element-Binding Proteins (SREBPs)

SREBPs, membrane bound transcription factors that coordinate the synthesis of fatty acids and cholesterol, the two major building blocks of membranes, belong to the basic helix-loop-helix-leucine zipper (bHLH-Zip) family of transcription factors. There are three SREBP proteins (SREB-1a, SREBP-1c, and SREBP-2) from two srebp genes designated srebp1 and srebp2. The SREBP2 isoform plays a major role in regulating cholesterol synthetic genes. Nearly all of the genes encoding cholesterol synthesis enzymes are SREBP targets.

SREBPs coordinately regulate the cholesterol biosynthetic pathway and receptor-mediated endocytosis of LDL at the level of gene transcription. In the cholesterol biosynthetic pathway, SREBPs regulate transcription of HMG CoA reductase as well as transcription of genes encoding many other enzymes in the cholesterol biosynthetic pathway, including HMG CoA synthase, farnesyl diphosphate synthase and squalene synthase. Studies investigating regulation of the DHCR24 promoter provided evidence of binding sites for SREBP-2. The SREBPs also regulate the LDL receptor, which supplies cholesterol through receptor mediated endocytosis, and modulate transcription of genes encoding enzymes of fatty acid synthesis and uptake, including acetyl CoA carboxylase, fatty acid synthase, stearoyl CoA desaturase-1 and lipoprotein lipase.

Nascent SREBPs are targeted to the endoplasmic reticulum (ER) membrane without any transcription activity, because they are not available for their target genes, which are located in the nucleus. To enhance transcription when cellular sterol levels are low, the active NH2-terminal domains of SREBPs are released from endoplasmic reticulum membranes by two sequential cleavages that must occur in the proper order. The first is catalyzed by Site-1 protease (S1P), a membrane bound subtilisin-related serine protease that cleaves the hydrophilic loop of SREBP that projects into the endoplasmic reticulum lumen. The second cleavage, at Site-2, requires the action of S2P, a hydrophobic protein that appears to be a zinc metalloprotease, and takes place within a membrane-spanning domain of SREBP. Sterols block SREBP processing by inhibiting S1P. Sterols block the proteolytic release process by selectively inhibiting cleavage by S1P; S2P is regulated indirectly because it cannot act until SREBP has been processed by S1P.

SREBP cleavage-activating protein (SCAP), an integral ER membrane regulatory protein, is required for cleavage at Site 1 and is the target for sterol suppression of this cleavage, i.e., SCAP loses its activity when sterols overaccumulate in cells. Within cells, SCAP is found in a tight complex with SREBPs. SCAP contains two distinct domains: a hydrophobic N-terminal domain that spans the membrane eight times and a hydrophilic C-terminal domain that projects into the cytosol. A 160 amino acid segment of the membrane domain of SCAP has been termed the sterol-sensing domain. The C-terminal domain of SCAP mediates a constitutive association with SREBPs, which is required for SCAP-dependent translocation of SREBPs from the ER to Golgi in sterol-deprived cells. The NH2-terminal bHL-Zip domain with full transcription activity is released from the membrane to reach the nucleus and act as a transcription factor to activate genes responsible for cholesterol and fatty acid biosynthesis and LDL uptake.

When sterols build up within cells, the proteolytic release of SREBPs from ER membranes is blocked, the NH2-terminal domains that have already entered the nucleus are rapidly degraded, and, as a result, transcription of all of the target genes declines. This decline is complete for the cholesterol biosynthetic enzymes whose transcription is entirely dependent on SREBPs, but less complete for the fatty acid biosynthetic enzymes whose basal transcription can be maintained by other factors.

### Other Factors

Besides SREBP, numerous other transcription factors have been implicated in the transcriptional control of the various enzymes in cholesterol biosynthesis.

### Liver X Receptors (LXRs)

Liver X receptors (LXRs) are ligand-activated transcription factors of the nuclear receptor superfamily. There are two LXR isoforms (termed alpha and beta), which, upon activation, form heterodimers with retinoid X receptor and bind to LXR response elements found in the promoter region of the target genes. High expression levels of LXRα in metabolically active tissues fit with a central role of the receptor in lipid metabolism, while LXRβ is more ubiquitously expressed. Both LXRs are found in various cells of the immune system, such as macrophages, dendritic cells and lymphocytes. In macrophages, the accumulation of excess lipoprotein-derived cholesterol activates LXR and triggers the induction of a transcriptional program for cholesterol efflux, such as ATP-binding cassette transporter (ABC) A1 (ABCA1) and ABCG1, while in parallel the receptor transrepresses inflammatory genes, such as inducible nitric oxide synthase, interleukin 10, and monocyte chemotactic protein-1. LXR has been reported to regulate cholesterol biosynthesis by directly silencing the gene expression of two cholesterogenic enzymes (FDFT1 and CYP51A1).

Endogenous agonists of the LXRs include oxysterols, which are oxidized cholesterol derivatives. LXRs have been characterized as key transcriptional regulators of lipid and carbohydrate metabolism, and were shown to function as sterol sensors protecting the cells from cholesterol overload by stimulating reverse cholesterol transport and activating its conversion to bile acids in the liver. This finding led to identification of LXR agonists as potent anti-atherogenic agents in rodent models of atherosclerosis. However, first-generation LXR activators were also shown to stimulate lipogenesis via SREBP1c leading to liver steatosis and hypertriglyceridemia.

Despite their lipogenic action, LXR agonists possess antidiabetic properties. Id. LXR activation normalizes glycemia and improves insulin sensitivity in rodent models of type 2 diabetes and insulin resistance. Although antidiabetic action of LXR agonists is thought to result predominantly from suppression of hepatic gluconeogenesis, some studies suggest that LXR activation may also enhance peripheral glucose uptake.

Published reports of anti-proliferative effects of synthetic LXR ligands on breast, prostate, ovarian, lung, skin, and colorectal cancer cells suggest that LXRs are potential targets in cancer prevention and treatment. Cell line-specific transcriptional responses and a set of common responsive genes were shown by microarray analysis of gene expression in four breast cell lines [MCF-7 (ER+), T-47D (ER+), SK-BR-3 (ER−), and MDA-MB-231] following treatment with the synthetic LXR ligand GW3965. In the common responsive gene set, upregulated genes tend to function in the known metabolic effects of LXR ligands and LXRs whereas the downregulated genes mostly include those which function in cell cycle regulation, DNA replication, and other cell proliferation-related processes. Transcription factor binding site analysis of the downregulated genes revealed an enrichment of E2F binding site sequence motifs. Correspondingly, E2F2 transcript levels are downregulated following LXR ligand treatment. Knockdown of E2F2 expression, similar to LXR ligand treatment, resulted in a significant disruption of estrogen receptor positive breast cancer cell proliferation. Ligand treatment also decreased E2F2 binding to cis-regulatory regions of target genes.

Expression of activated LXRα blocks proliferation of human colorectal cancer cells and slows the growth of xenograft tumors in mice, and reduces intestinal tumor formation after administration of chemical carcinogens in Apc(min/+) mice. A link of LXRs to apoptosis has been reported.

### MicroRNAs and Alternative Splicing

Relatively little has been reported on miRNAs in the context of cholesterol synthesis. Alternative splicing of HMGCR is regulated by sterols, with proportionally less of an unproductive transcript present when sterol levels are low and more when sterol levels are higher.

### Post-Translational Regulation

Because transcriptional down-regulation via the SREBP pathway is relatively slow, with mRNA of target genes decreasing only after several hours, rapid shutdown of cholesterol synthesis requires post-transcriptional control. Turnover of 3-hydroxy-3-methylglutaryl-CoA reductase (HMGCR) is accelerated by non-sterol and sterol products of the mevalonate pathways, with physiological sterol degradation signals, such as 24,25-dihydrolanosterol, and side chain oxysterols, such as 24,25-EC and 27-hydroxycholeseterol (generated from cholesterol itself). The regulated turnover is proteosomal, and requires the Insig proteins, which also act to suppress SREBP activation.

Regulated ER-associated degradation also occurs for a later step in cholesterol synthesis, catalyzed by squalene monooxygenase (SM), albeit by a mechanism distinct from HMGCR. Squalene monooxygenase has been proposed as a second rate-limiting enzyme in cholesterol synthesis. Cholesterol itself accelerates SM degradation, an example of end product inhibition, and unlike HMGCR, SM turnover does not require the Insig proteins.

### Feedback Regulation of Cholesterol Synthesis

Cholesterol accumulation lowers the activity of HMG CoA reductase and several other enzymes in the cholesterol biosynthetic pathway, thereby limiting the production of cholesterol.

HMG CoA reductase, an early and rate-limiting enzyme in cholesterol synthesis, and the target of statins, is subject to feedback control through multiple mechanisms that are mediated by sterol and nonsterol end-products of mevalonate metabolism such that essential nonsterol isoprenoids can be constantly supplied without risking the potentially toxic overproduction of cholesterol or one of its sterol precursors. For example, treatment of cultured cells with the statin Compactin, a competitive inhibitor of HMG-CoA reductase, blocks production of mevalonate, thereby reducing levels of sterol and nonsterol isoprenoids that normally govern this feedback regulation. Cells respond to the inhibition of HMG-CoA reductase with a compensatory increase in the reductase due to the combined effects of enhanced transcription of the reductase gene, efficient translation of mRNA, and extended half-life of reductase protein. Complete reversal of this compensatory increase in reductase requires regulatory actions of both sterol and nonsterol end-products of mevalonate metabolism.

Sterols inhibit the activity of sterol regulatory element-binding proteins (SREBPs) and the low density lipoprotein (LDL)-receptor. A nonsterol mevalonate-derived product(s) control(s) the translational effects through a poorly understood mechanism that may be mediated by the complex 5′-untranslated region of the reductase mRNA. Both sterol and nonsterol end-products of mevalonate metabolism combine to accelerate degradation of reductase protein through a mechanism mediated by the ubiquitin-proteosome pathway.

Inhibition of ER to Golgi transport of SREBPs results from sterol-induced binding of SCAP to ER retention proteins called insulin-induced gene 1 and 2 proteins (Insig-1 and Insig-2). Insig binding occludes a cytosolic binding site in SCAP recognized by COPII proteins, which incorporate cargo molecules into vesicles that deliver ER-derived proteins to the Golgi. SCAP-Insig binding is mediated by a segment of SCAP's membrane domain that includes transmembrane helices 2-6, since a similar stretch of transmembrane helices is found in at least four other polytopic proteins, including the Niemann Pick C1 protein (part of an intestinal cholesterol transporter complex), Patched, Dispatched and reductase) that have been postulated to interact with sterols. Point mutations within this region disrupt Insig binding, which relieves sterol-mediated retention of mutant SCAP-SREBP complexes in the ER.

The following observations suggest that Insigs may play a role in degradation of HMG CoA reductase. First, when Insigs are overexpressed by transfection in Chinese hamster ovary (CHO) cells, HMG CoA reductase cannot be degraded when the cells are treated with sterols. Co-expression of Insig-1 restores sterol-accelerated degradation of HMG CoA reductase, suggesting the saturation of endogenous Insigs by the overexpressed reductase. Second, reduction of both Insig-1 and Insig-2 by RNA interference (RNAi) abolishes sterol-accelerated degradation of endogenous HMG CoA reductase. Third, mutant CHO cells lacking both Insigs are impervious to sterol-stimulated degradation of HMG CoA reductase as well as sterol-mediated inhibition of SREBP processing.

Degradation of HMG CoA reductase coincides with sterol-induced binding of its membrane domain to Insigs, an action that requires a tetrapeptide sequence (YIYF) located in the second transmembrane segment of HMG CoA reductase. A mutant form of HMG CoA reductase in which the YIYF sequence is mutated to alanine residues no longer binds to Insigs, and the enzyme is not subject to rapid degradation. The YIYF sequence is also present in the second transmembrane domain of SCAP, where it mediates sterol-dependent formation of SCAP-Insig complexes. Overexpressing the sterol-sensing domain of SCAP in cells blocks Insig-mediated, sterol-accelerated degradation of HMG CoA reductase; mutation of the YIYF sequence in the SCAP sterol-sensing domain ablates this inhibition, suggesting that SCAP and HMG CoA reductase bind to the same site on Insigs and that the two proteins compete for limiting amounts of Insigs when intracellular sterol levels rise.

Glycoprotein 78 (Gp78), an E3 ubiquitin ligase, mediates ubiquitination of ApoB-100, Insig 1 and 2 proteins, and HMG-CoA reductase. High concentration of sterol (lanosterol) promote the NH2-terminal transmembrane domain of 3-hydroxy-3-methylglutaryl CoA reductase to interact with Insigs, and sterol-dependent Insig binding results in recruitment of ubiquitin ligase.

Gp78 binds Insig-1 constituitively in the ER membrane. When the cellular sterol level is high, the insig-1/gp78 complex binds the transmembrane domain of 3-hydroxy-3-methylglutaryl CoA reductase. With the assistance of at least two proteins associated with gp78, p97NCP and Aup1, the ubiquitinated reductase is translocated to lipid droplet-associated ER membrane and dislocated from membrane into cytosol for proteosomal degradation. This post-ubiquitination process can be promoted by geranylgeraniol or its metabolically active geranyl-geranyl-pyrophosphate.

In short, the ubiquitination of Insig-1 is mediated by gp78 and regulated by sterols. Insig-1 is modified by gp78 under low sterol conditions. High sterol promotes SCAP to bind Insig and gp78 is competed off, thereby stabilizing Insig-1.

Gp78-mediated ubiquitination and degradation of Insig-1 provides a mechanism for convergent feedback inhibition, whereby inhibition of SREBP processing requires convergence of newly synthesized Insig-1 and newly acquired sterols. In sterol-depleted cells, SCAP-SREBP complexes no longer bind Insig-1, which in turn becomes ubiquitinated and degraded. These SCAP-SREBP complexes are free to exit the ER and translocate to the Golgi, where the SREBPs are processed to the nuclear form that stimulates transcription of target genes, including the Insig-1 gene. Increased transcription of the Insig-1 gene leads to increased synthesis of Insig-1 protein, but the protein is ubiquitinated and degraded until sterols build up to levels sufficient to trigger SCAP binding.

Insig-2 has been defined as a membrane-bound oxysterol binding protein with binding specificity that correlates with the ability of oxysterols to inhibit SREBP processing. Oxysterols, cholesterol derivatives that contain hydroxyl groups at various positions in the iso-octyl side chain (e.g., 24-hydroxycholesterol, 25-hydroxycholesterol, 27-hydroxycholesterol), are synthesized in many tissues by specific hydrolases; oxysterols play key roles in cholesterol export, and are intermediates in the synthesis of bile acids. Oxysterols, which are significantly more soluble than cholesterol in aqueous solution, can readily pass across the plasma membrane and enter cells, and are extremely potent in inhibiting cholesterol synthesis by stimulating binding of both HMG Co A reductase and SCAP to Insigs. Thus, formation of the SCAP-Insig complex can be initiated by either binding of cholesterol to the membrane domain of SCAP or by binding of oxysterols to Insigs, both of which prevent incorporation of SCAP-SREBP into vesicles that bud from the ER en route to the Golgi.

Insig-mediated regulation of HMG Co A reductase is controlled by three classes of sterols: oxysterols, cholesterol, and methylated sterols (e.g., lanosterol and 24, 25-dihydrolanosterol). Oxysterols both accelerate degradation of HMG Co A reductase and block ER to Golgi transport of SCAP-SREBP through their direct binding to Insigs. Cholesterol does not regulate HMG Co A reductase stability directly, but binds to SCAP and triggers Insig binding, thereby preventing escape of SCAP-SREBP from the ER. Lanosterol selectively accelerates degradation of HMG Co A reductase without an effect on ER to Golgi transport of SCAP-SREBP. However, the demethylation of lanosterol has been implicated as a rate-limiting step in the post-squalene portion of cholesterol synthesis. The accumulation of lanosterol is avoided; its inability to block SREBP processing through SCAP assures that mRNAs encoding enzymes catalyzing reactions subsequent to lanosterol remain elevated, and lanosterol is metabolized to cholesterol.

It is a paradox that gp78 deficiency increases both the 3-hydroxy-3-methylglutaryl CoA reductase and Insig protein levels in mouse liver, because Insigs not only negatively regulate 3-hydroxy-3-methylglutaryl CoA reductase post-transcriptionally, but also inhibit SREBPs processing through binding to SCAP. These two outcomes are contradictory regarding cholesterol biosynthesis. Studies from L-gp78+ mice have shown that the biosynthesis of cholesterol and fatty acids is decreased in gp78-deficient mouse liver. This has been interpreted to mean that the Insig-SCAP-SREBP axis dominates, even though 3-hydroxy-3-methylglutaryl CoA (HMG CoA) reductase is elevated.

ApoB-100, an essential protein component of very low-density lipoproteins (VLDL) and low-density lipoproteins (LDL), which plays critical roles in plasma cholesterol transportation, is another substrate of g78. Under normal conditions, ApoB-100 is one of the committed secretory proteins. However, when the cellular lipid availability is limited (e.g., the new synthesized core lipids (triglyceride, cholesterol ester) or microsomal triglyceride transfer protein activity is decreased), the nascent ApoB-100 is subjected to ER-associated degradation mediated by gp78. When gp78 is overexpressed, ubiquitination and degradation through the 26S proteosome of apoB-100 is decreased. When gp78 is knocked down, the secretion of apoB-100 and the assembly of VLDL are increased in HepG2 cells. The retrotranslocation of ApoB-100 also requires p97NCP, similar to HMG CoA reductase.

### TRC8

Human TRC8 is a multi-pass membrane protein located in the ER membrane that binds both Insig-1 and Insig-2. It contains a conserved sterol sensing domain and C-terminal RING domain with ubiquitin ligase activity. RNAi studies in SV-589 cells showed that knockdown of TRC8 combined with gp78 can dramatically decrease the sterol-regulated ubiquitination as well as degradation of HMG CoA reductase, suggesting that both gp78 and TRC8 are involved in the sterol-accelerated ubiquitination of HMG CoA reductase in CHO-7 and SV-589 cells.

### TEB4

Human TEB4 is a 910 amino acid ER membrane-resident ubiquitin ligase. In mammalian cells, cholesterol stimulates the degradation of squalene monooxygenase (SM), the enzyme that catalyzes the first oxygenation step in cholesterol synthesis by which squalene is converted to the squalene-2,3-epoxide (37) mediated by TEB4. As one of the target genes of SREBP-2, both the transcription of SM and the stability of SM protein are regulated by sterols. SM protein level is negatively regulated by cholesterol in mammalian cells. When cholesterol, but not 24, 25-dihydrolanosterol, or side chain oxysterols, such as 27-hydroxycholesterol, is/are present, SM is ubiquitinated by TEB4.

### IDOL

The low-density lipoprotein receptor (LDL-R) gene family consists of cell surface proteins involved in receptor-mediated endocytosis of specific ligands. Low density lipoprotein (LDL) is normally bound at the cell membrane and taken into the cell, ending up in lysosomes where the protein is degraded and the cholesterol is made available for repression of microsomal enzyme HMG CoA reductase. At the same time, a reciprocal stimulation of cholesterol ester synthesis takes place.

Inducible degrader of LDL-R (IDOL) moderates the degradation of LDL-R and requires the E2 enzyme UBE2D.

Transcription of the LDL-R gene is regulated primarily by SREBP in a sterol responsive manner. The LDL-R is also regulated at the posttranscriptional level by protoprotein convertase subtilisin/kexin type 9 (PCSK9)-mediated degradation of LDLR in the lysosome. PCSK9 is synthesized as an about 74 kD soluble zymogen in the endoplasmic reticulum (ER), where it undergoes autocatalytic processing to release a processing enzyme of about 60 kDa to secrete from cells. PCSK9 binds the extracellular domain of LDLR, which leads to lysosomal degradation of LDLR.

IDOL also is a post-transcriptional regulator of LDL-R. Activation of LXR can decrease the abundance of LDLR without changing its mRNA level and subsequently inhibited uptake of LDL in different cells. IDOL can increase plasma cholesterol level by ubiquitination and degradation of LDL-R dependent on its cytosolic domain. The decrease or ablation of IDOL can elevate the LDL-R protein level and promote LDL uptake. The expression of Idol in liver is relatively low, and it is not regulated by LXR, while the LXR-IDOL pathway seems to be more active in peripheral cells, e.g., macrophages, small intestine, adrenals.

### Cholesterol Biosynthesis Pathway Inhibitors as Antitumor Agents

Statins, which were developed as lipid-lowering drugs to control hypercholesterolemia, competitively inhibit HMG-CoA reductase, and have been proposed as anticancer agents, because of their ability to trigger apoptosis in a variety of tumor cells in a manner that is sensitive and specific to the inhibition of HMG-CoA reductase. This apoptotic response is in part due to the downstream depletion of geranylgeranyl pyrophosphate (GGPP), and thus due to inhibition of protein prenylation. Protein prenylation creates a lipidated hydrophobic domain and plays a role in membrane attachment or protein-protein interactions. Prenylation occurs on many members of the Ras and Rho family of small guanosine triphosphatases (GTPases). Three enzymes (farnesyltransferase (FTase), geranylgeranyltransferase (GGTase) I and GGTase II can catalyze protein prenylation.

While statin therapy blocks the intracellular synthesis of cholesterol, it also alters the cholesterol content of tumor cell membranes, interfering with key signaling pathways.

Statins have been shown to have immunomodulatory activity, and to induce the depletion of prenyl pyrophosphates in human dendritic cells. Prenyl pyrophosphate deprivation translated into activation of caspase I, which cleaved the preforms of IL-1β and IL-18 and enabled the release of bioactive cytokines. The statin-treated dendritic cells (DCs) thus acquired the capability to potentially activate IL-2 primed natural killer (NK) cells. NK cells, which recognize and attack tumor cells that lack MHC class I molecules contribute to innate immune responses against neoplastic cells. The statin-induced response of IL-2-primed NK cells could be abolished completely when cell cultures were reconstituted with the isoprenoid pyrophosphate GGPP, which allows protein geranylgeranylation to occur despite statin-mediated inhibition of HMB-CoA reductase. Statins also acted directly on human carcinoma cells to induce apoptosis, and IFN-γ produced by NK cells cooperated with statins to enhance tumor cell death synergistically.

Mutant p53, which is present in more than half of all human cancers, can significantly upregulate mevalonate pathway activity in cancer cells, which contributes to maintenance of the malignant phenotype. Simvastatin was shown to reduce 3-dimensional growth of cancer cells expressing a single mutant p53 allele, and was able to induce extensive cancer cell death and a significant reduction of their invasive phenotype. In isoprenoid add-back experiments, supplementation with GGPP was sufficient to restore the invasive phenotype in the presence of HMG-CoA reductase inhibition, showing that upregulation of protein geranylgeranylation is an important effect of mutant p53.

Bisphosphonates, drugs that prevent bone resorption, act downstream of HMG-CoA reductase to inhibit farnesyl pyrophosphate (FPP) synthase. Both bisphosphonates and statins eventually cause FPP and GGPP deprivation and thus failure to perform farnesylation and geranylgeranylation of small GTPases of the Ras superfamily. With regard to bisphosphonates, the inhibition of Ras signaling due to the disruption of membrane anchoring of these GTPases eventually stops osteoclast-mediated bone resorption.

Suppressors of the mevalonate pathway also include the diverse isoprenoids, mevalonate-derived secondary metabolites of plants. The potencies of isoprenoids in suppressing hepatic HMG-CoA reductase activity was found to be strongly correlated to their potencies in tumor suppression. The tocotrienols, vitamin E molecules, and “mixed isoprenoids” with a farnesol side chain, down-regulate HMG-CoA reductase activity in tumors and consequently induce cell cycle arrest and apoptosis. The growth-suppressive effect of tocotrienols was attenuated by supplemental mevalonate.

Activity of azole antifungal compounds, such as ketoconazole, to block the function of several cytochrome P450 enzymes involved in cholesterol biosynthesis (e.g., CYP51A1, which catalyzes demethylation of lanosterol) and CYP17A1 (which mediates a step in the synthesis of androgens) has been utilized clinically to treat hormone refractory prostate cancer, and recently has been surpassed by abiraterone, a CYP17A1 antagonist. Itraconazole has shown activity against medulloblastoma, via its inhibitory effects on Smoothened in the hedgehog pathway, and suppression of angiogenesis via its interference with lysosomal cholesterol trafficking. The anti-angiogenic effect of itraconazole, a well-established CYP51/ERG11 antifungal antibiotic, is exerted via inhibition of endosomal cholesterol trafficking and suppression of mTOR signaling.

In tumor cells, increased signaling activity of growth factor or steroid hormone receptors via PI3K/AKT and MAPK/ERK1/2, HIF-1α, p53, and sonic hedgehog (SHH) pathways modulate and activate SREBP-1, the main regulatory component of lipogenesis. It has been reported that inhibiting mTORC1 using rapamycin has little effect on SREBP-1 nuclear localization and its abundance, but inhibiting its upstream factors, like EGFR, PI3K and Akt, significantly decreases SREBP-1 N-terminal levels and diminishes its abundance in the nucleus. mTOR kinase inhibitor Torin-1, which inhibits both mTORC1 and mTORC2 activity, significantly decreased SREBP-1 abundance in the nucleus compared to the inhibition of mTORC1 alone by rapamycin.

Overexpression of lipogenic enzymes has been observed in a number of carcinomas and has been described to correlate with disease severity, increased risk of recurrence and a lower chance of survival.

Accelerated synthesis of lipids and sterols also is an essential mechanistic component of malignant transformation. Oxidized LDL receptor 1 (OLR1) is required for Src kinase transformation of immortalized MCF10A mammary epithelial cells. OLR1 is significantly induced during transformation, and depletion of OLR1 by siRNA blocks morphological transformation and inhibits cell migration and invasion, and results in reduction of tumor growth in vivo. Conversely, overexpression of ORL1 protein in MCF10A and HCC1143 mammary epithelial cells leads to significant upregulating of BCL2, a negative regulator of apoptosis.

EBP in complex with dihydrocholesterol-7 reductase (DHCR7) catalyzes isomerization of the double-bond between C7 and C8 in the second cholesterol ring. This complex mediates the activity of cholesterol epoxide hydrolase.

There are several known inhibitors of EBP, and some have been described as anti-cancer agents. For example, a sterol conjugate of a naturally occurring steroidal alkaloid, 5alpha-hydroxy-6beta-[2-(1H-imidazol-4-yl)ethylamino]cholestan-3beta-ol (dendrogenin A) which is produced in normal, but not in cancer cells, and 5,6 alpha-epoxy-cholesterol and histamine, has been shown to suppress cancer cell growth and to induce differentiation in vitro in various tumor cell lines of different types of cancers. It also inhibited tumor growth in melanoma xenograft studies in vivo and prolonged animal survival.

SR31747A (cis-N-cyclohexyl-N-ethyl-3-(3-choloro-4-cyclohexyl-phenyl)propen-2-ylamine hydrochloride), a selective peripheral sigma binding site ligand whose biological activities include immunoregulation and inhibition of cell proliferation, binds to SR31747A-binding protein 1 (SR-BP) and EBP with nanomolar affinity. The effect of SR31747A on proliferative activity was evaluated in vitro on the following breast and prostate cancer cell lines: breast (hormone responsive MCF-7 cells from a breast adenocarcinoma pleural effusion; MCF-7AZ; Hormone independent MCF-7/LCC1 cells derived from MCF-7 cell lines; MCF-7LY2, resistant to the growth-inhibitory effects of the antiestrogen LY117018; Hormone unresponsive MDA-MB-321 and BT20 established from a metastatic human breast cancer tumor); and prostate (Hormone responsive prostate cancer cell line LNCaP; hormone-unresponsive PC3 cell line established from bone marrow metastasis; hormone-unresponsive DU145 established from brain metastasis). SR31747A induced concentration-dependent inhibition of cell proliferation, regardless of whether the cells were hormone responsive or unresponsive. The antiproliferative effect of SR31747A was partially reduced by adding cholesterol, thus suggesting the possible involvement of EBP. Sensitivity to SR31747A did not correlate with cellular levels of EBP. SR31747A also inhibited proliferation in vivo in the mouse xenograft model. Murine EBP cDNA overexpression in CHO cells increased resistance of these cells to SR31747A-induced inhibition of proliferation.

Tamoxifen, inhibited SR31747 binding in a competitive manner and induced the accumulation of Δ8-sterols, while Emopamil, a high affinity ligand of human sterol isomerase a calcium-channel blocking agent, and verapamil, another calcium channel-blocking agent, are inefficient in inhibiting SR31747 binding to its mammalian target, suggesting that their binding sites do not overlap. Some drugs, e.g., cis-flupentixol, trifluoroperazine, 7-ketocholestanol and tamoxifen, inhibit SR31747 binding only with mammalian EBP enzymes, whereas other drugs, e.g., haloperidol and fenpropimorph, are more effective with the yeast derived enzymes than with the mammalian ones.

While some cancer cell lines are highly sensitive to small molecule EBP inhibition, other cancer cell lines, as well as normal cell lines, do not respond to EBP inhibition, even when up to 10,000-fold higher concentrations of the EBP inhibitors are used. A determination of which cancer will respond to which inhibitor therefore has historically required an empirical hit or miss, impractical and expensive, approach.

The described invention establishes that EBP inhibition is only toxic to cancer cells that paradoxically respond to small molecule EBP inhibitors via downregulation of endogenous cholesterol biosynthesis, and provides a method for identifying such EBP inhibitors and for cancer cells that are sensitive to treatment with such inhibitors.

Colorectal Cancer (CRC) is the second leading cause of cancer deaths resulting in ˜600,000 deaths world-wide every year (49,700 in the U.S. and 152,000 in the E.U.). Despite the fact that the disease etiology for the majority of CRCs is fairly well understood, there are still no therapies available that specifically target oncogenotypes that drive CRC development and progression. In addition to early detection (colonoscopy and genetic testing) and surgical removal of precancerous adenomatous polyps (adenomas), current treatment options for advanced CRC include surgery, radiation therapy, and chemotherapy. A large body of studies have shown that the primary initiating event in both Familial Adenomatous Polyposis (FAP) and sporadic CRC is a loss of function of the Adenomatous Polyposis Coli (APC) tumor suppressor gene leading to aberrant crypts and early adenomas. According to the model of Fearon and Vogelstein, these early events in the adenoma to adenocarcinoma sequence cause genomic instability leading to the acquisition of additional mutations in various oncogenes such as KRAS or BRAF, SMAD4, TGF-β, and frequently in the tumor suppressor TP53. Although the full spectrum of biological pathways regulated by the large multifunctional apc protein remains a topic of debate, it is now commonly accepted that wild-type APC (APC^{wt}) is essential for intestinal cell differentiation and crypt homeostasis at least in part via regulation of the Wnt signaling pathway. It is estimated that mutations in the APC gene occur in >80% of patients diagnosed with CRC with >90% of those mutations targeting the Mutation Cluster Region (MCR) leading to defined truncated APC (APC^{TR}) gene products. While loss of tumor suppressive function of APC mutations is believed to be important for CRC tumorigenesis, increasing evidence suggests that the truncated form of the mutant APC m protein also endows these tumors with gain-of-function properties. For instance, it was recently shown that apc-truncations relieve the autoinhibition of C-terminal activation of Asef (APC-selective guanine exchange factor) leading to downstream Golgi fragmentation via activation of an Asef-ROCK-MLC2 signaling pathway. Another recent study demonstrates that introduction of APC^{WT} in colon cancer models reestablishes normal intestinal crypt homeostasis and function, even in the presence of potent oncogenic drivers such as KRAS and p53. In light of the above, small molecules that specifically target colon cancer cell lines with APC^{TR} while sparing normal cells with APC^{WT} would provide for a potential highly selective therapy for the vast majority of CRC patients.

### SUMMARY

In some embodiments, the present disclosure provides an EBP-modulating anti-cancer compound with the structure of Formula (I):

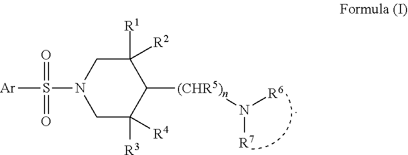

or a pharmaceutically acceptable salt or solvate, a stereoisomer, a diasteroisomer or an enantiomer thereof.

In some embodiment, R¹, R², R³, and R⁴ can be independently selected from the group consisting of H, F, CF₃, CHF₂, CH₂F, and methyl.

In some embodiment, Ar can be selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl.

In some embodiments, the optional substituent for Ar can be selected from the group consisting of F, Cl, Br, —OCF₃, —OCHF₂, —OCH₂F, —OCH₂R⁸, —OCHMeR⁸, —OCH(CF₃)R⁸, —OR⁸, —C(O)R⁸, R⁸, C1-4 alkyl, C3-5 cycloalkyl, C2-6 alkenyl, C2-6 alkynyl, —OC1-4 alkyl, —OC3-5 cycloalkyl, —OC2-6 alkenyl, and —OC2-6 alkynyl.

C1-4 alkyl or C3-5 cycloalkyl can be optionally substituted selected from the group consisting of fluorine, hydroxyl, C1-3 alkoxy group, tetrahydropyranyl optionally substituted with one or more fluorines, hydroxyl, or C1-3 alkoxy group, tetrahydrofuranyl optionally substituted with one or more fluorines, hydroxyl, or C1-3 alkoxy group, and a combination thereof.

C2-6 alkenyl or C2-6 alkynyl can be optionally substituted with fluorine, hydroxyl, C1-3 alkoxy group, or a combination thereof.

—OC1-4 alkyl or —OC3-5 cycloalkyl can be optionally substituted with fluorine, hydroxyl, C1-3 alkoxy group, or a combination thereof.

—OC2-6 alkenyl or —OC2-6 alkynyl can be optionally substituted with fluorine, hydroxyl, C1-3 alkoxy group, or a combination thereof.

In some embodiments, n can be 0 or 1. When n=1, in some embodiment, R⁵ can be selected from the group consisting of H, methyl, CF₃, CHF₂, and CH₂F.

In some embodiments, R⁶ and R⁷ can be independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F or OH; C1-3 alkoxy group; —CH₂CCH; R⁸; CH₂R⁸; OR⁸; OCH₂R⁸; OCHMe⁸.

In some embodiments, R⁶ and R⁷ can be connected to form a nitrogen-containing heterocycle, in such case, R⁶-R⁷ is to be selected from the group consisting of —(CHR¹⁰)CH₂(CHR¹⁰)O(CHR⁹)—, —(CHR⁹)O(CHR¹⁰)₂—, —CH₂(CR¹²R¹³)CH₂—, —(CH₂)₂ (CHR¹¹)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkylidenyl)-, —(CHMe)CH₂O(3,3-oxatenylidenyl)-, —(CH₂)₂(1,1-cycloalkylidenyl)CH₂—, —(CH₂)ₘ— with m=4-6 and the provision that when n=0 then m≠5 and optionally substituted with one or more substituents independently selected from the group consisting of F, OH, and R¹⁰.

In some embodiments, R⁸ can be phenyl or heteroaryl optionally substituted with one or more substituents independently selected from the group consisting of F, Cl, Br, CF₃, CHF₃, CH₂F, C1-4 alkyl, C3-5 cycloalkyl, —OC1-4 alkyl, and —OC3-5 cycloalkyl, wherein C1-4 alkyl, C3-5 cycloalkyl, —OC1-4 alkyl, or —OC3-5 cycloalkyl is optionally substituted with one or more fluorines.

In some embodiments, R⁹ can be selected from the group consisting of H, R⁸, C1-4 alkyl, —OC1-3 alkyl, and —OC3-5 cycloalkyl, wherein C1-4 alkyl, —OC1-3 alkyl, or —OC3-5 cycloalkyl can be optionally substituted substituents selected from the group consisting of F, OH, R⁸, and a combination thereof.

In some embodiments, R¹⁰ can be selected from the group consisting of H, R⁸, C1-4 alkyl, C3-6 alkenyl, C3-6 alkynyl, —OC1-3 alkyl, —OC3-5 cycloalkyl, wherein C1-4 alkyl, C3-6 alkenyl, C3-6 alkynyl, —OC1-3 alkyl, or —OC3-5 cycloalkyl is optionally substituted with substituents selected from the group consisting of F, OH, R⁸, OR, OCH₂R⁸, OCHMeR⁸, and a combination thereof.

In some embodiment, R¹¹ can be selected from the group consisting of H, CO₂H, CO₂R¹⁴, CH₂OH, CH₂OR¹⁴, C1-4 alkyl, C3-6 alkenyl, C3-6 alkynyl, and C3-5 cycloalkyl, wherein C1-4 alkyl, C3-6 alkenyl, C3-6 alkynyl, or C3-5 cycloalkyl is optionally substituted with one or more substituents selected from the group consisting of F, OH, and R⁸.

In some embodiments, R¹² and R¹³ can be independently selected from the group consisting of H, F, CF₃, CHF₂, CH₂F, CN, OH, OR⁴, NHC(O)Me, SO₂Me, OSO₂Me, CO₂H, CO₂R¹⁴, CH₂OH, CH₂OR¹⁴, R⁸ and R¹⁴. In some embodiments, R¹² and R¹³ can be optionally connected to form a cyclic structure, in such a case, R¹²-R¹³ is to be selected from the group consisting of: —CH₂OCH₂—, —(CH₂)₂O—, —(CH₂)O—, —(CH₂)₃—, —(CH₂)—, —CH₂CF₂CH₂—, —CH₂O(CHCF₃)—, —CH₂SO₂(CHCF₃)—, —CH₂(CHCO₂H)CH₂—, —CH₂(CHCO₂R¹⁴)CH₂—, —CH₂(CHCH₂OH)CH₂—, —CH₂(CHCH₂OR¹⁴)CH₂—, —(CHOH)CH₂O—, —(CHOR¹⁴)CH₂O—, —SO₂(CH₂)₂(CHOH)—, —SO₂(CH₂)₂(CHOR¹⁴)—, —SO₂(CH₂)(CHOH)CH₂—, —SO₂(CH₂)(CHOR¹⁴)CH₂—, —CH₂(CHOH)CH₂O—, —CH₂(CHOR¹⁴)CH₂O—, —(CHOH)(CH₂O—(CHOR¹⁴)(CH₂)₂O—, and —CH₂(3,3-oxetanyl)CH₂-.

In some embodiments, R¹⁴ can be selected from the group consisting of C1-4 alkyl, C3-5 cycloalkyl, C2-6 alkenyl, and C2-6 alkynyl, each of which is optionally substituted with one or more substituents selected from F, OH, and R⁸.

In some embodiment, Ar in Formula (I) can be selected from

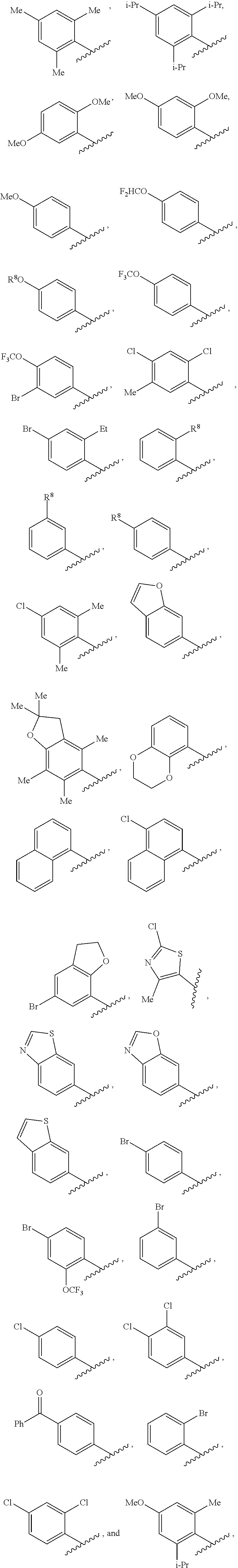

These functional groups for Ar can be optionally further substituted with one or more substituents independently selected from the group consisting of F, Cl, Br, Me, CF₃, Et, i-Pr, cyclopropyl, OMe, OEt, Oi-Pr, —Ocyclopropyl, —OCF₃, —OCHF₂, —OCH₂F, —OCH₂R⁸, —OR⁸ and R⁸.

In some embodiment, when n=0, —NR⁶R⁷ can be selected from:

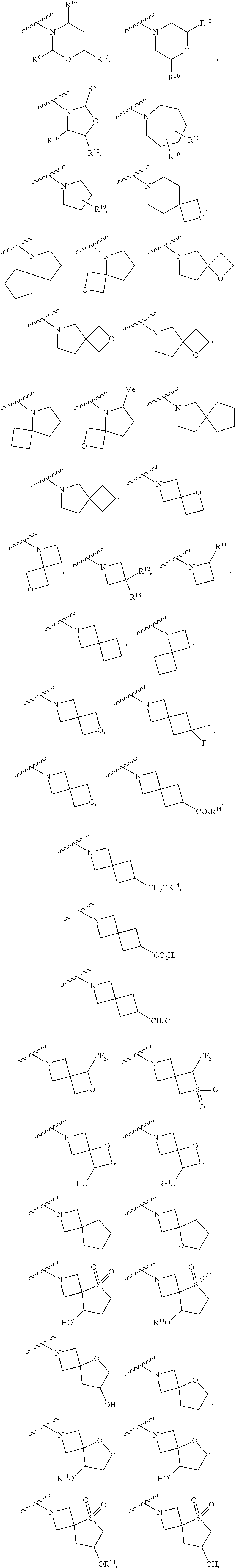

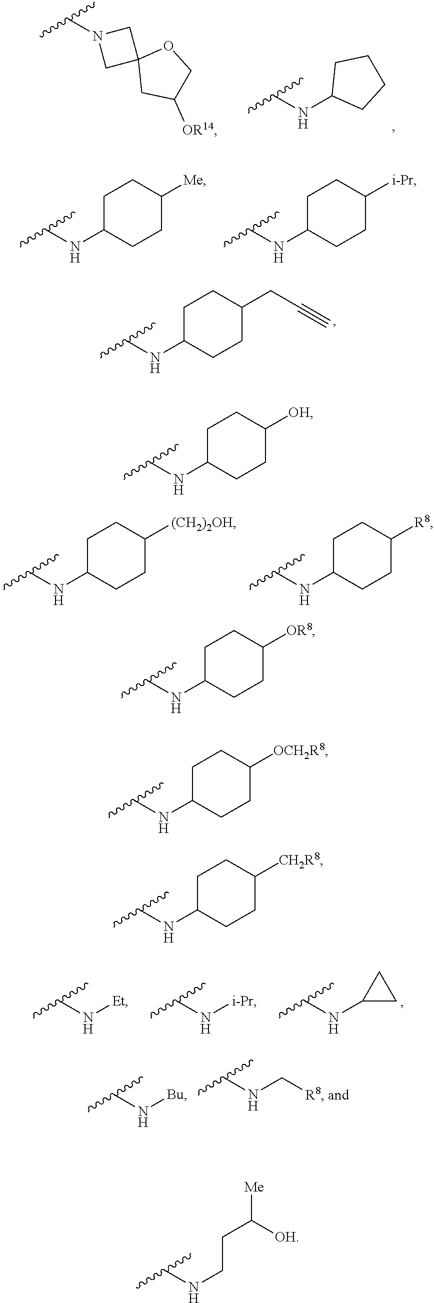

In some embodiment, when n=1, —NR⁶R⁷ can be selected from:

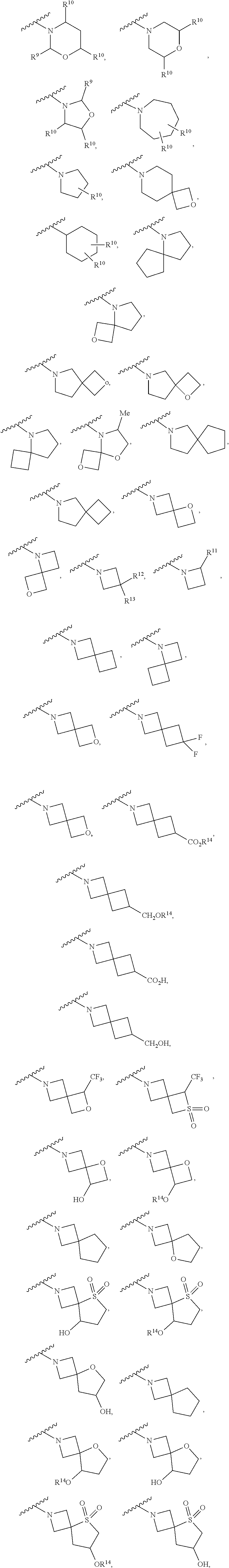

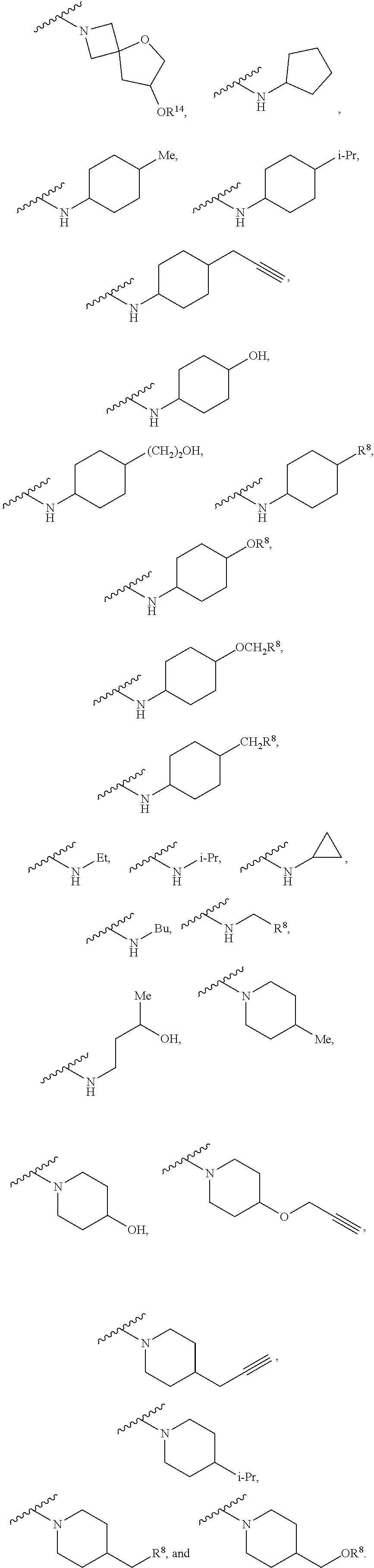

In some embodiments, the present disclosure provides a series of small molecule compounds that selectively inhibit the growth of human cancer cells that contain an APC protein. What's disclosed is a compound according to Formula (II):

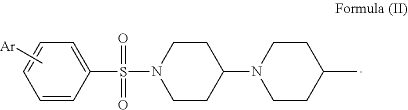

or a pharmaceutically acceptable salt or solvate, a stereoisomer, a diastereoisomer or an enantiomer thereof.

In some embodiments, Ar can be selected from the group consisting of substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl.

The optional substituents can be one or more substituents independently selected from the group consisting of F, Cl, Br, —OCF₃, —OCHF₂, —OCH₂F, —OCH₂R¹, —OCHMeR¹, —OCH(CF₃)R¹, —OR¹, —C(O)R¹, R¹, C1-4 alkyl or C3-5 cycloalkyl optionally substituted with one or more fluorines and/or hydroxy and/or C1-3 alkoxy group, tetrahydropyranyl or tetrahydrofuranyl optionally substituted with one or more fluorines and/or hydroxy and/or C1-3 alkoxy group, C2-6 alkenyl or alkynyl optionally substituted with one or more fluorines and/or hydroxy and/or C1-3 alkoxy group, —OC1-4 alkyl or —OC3-5 cycloalkyl optionally substituted with one or more fluorines and/or hydroxy and/or C1-3 alkoxy group, —OC2-6 alkenyl or alkynyl optionally substituted with one or more fluorines and/or hydroxy and/or C1-3 alkoxy group.

In some embodiments, R¹ can be phenyl or heteroaryl optionally substituted with one or more substituents independently selected from the group consisting of F, Cl, Br, CF₃, CHF₃, CH₂F, C1-4 alkyl or C3-5 cycloalkyl optionally substituted with one or more fluorines, and —OC1-4 alkyl or —OC3-5 cycloalkyl optionally substituted with one or more fluorines.

Formula (II) does not include the following compounds

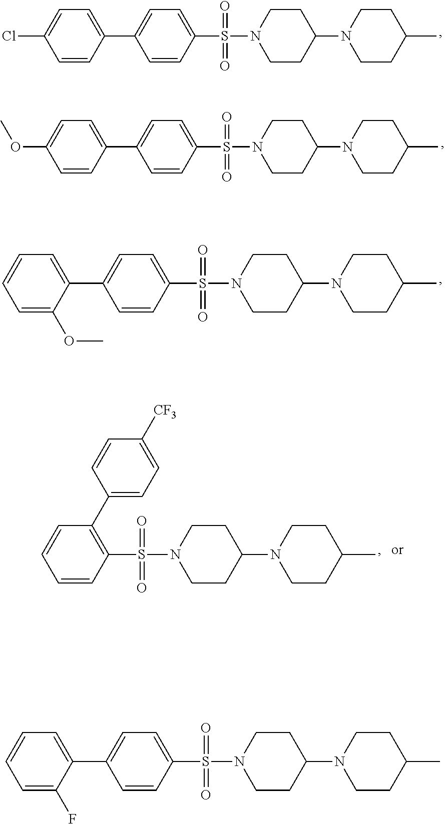

In some embodiments, the present disclosure provides an EBP-modulating anti-cancer compound with the structure of Formula (IV):

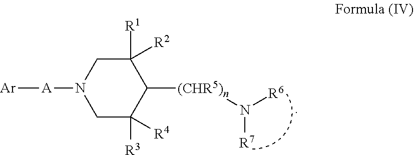

or a pharmaceutically acceptable salt or solvate, a stereoisomer, a diastereoisomer or an enantiomer thereof.

In some embodiments, A can be —NR⁸—SO₂- or —NR⁸—CO—. R¹, R², R³, and R⁴ can be independently selected from the group consisting of H, F, CF₃, CHF₂, CH₂F, and methyl. R⁸ can be selected from the group consisting of H and optionally-substituted C1-C4 alkyl.

In some embodiment, Ar can be selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl.

In some embodiments, the optional substituent for Ar can be selected from the group consisting of F, Cl, Br, —OCF₃, —OCHF₂, —OCH₂F, —OCH₂R⁹, —OCHMeR⁹, —OCH(CF₃)R⁹, —OR⁹, —C(O)R⁹, R⁹, C1-4 alkyl, C3-5 cycloalkyl, C2-6 alkenyl, C2-6 alkynyl, —OC1-4 alkyl, —OC3-5 cycloalkyl, —OC2-6 alkenyl, and —OC2-6 alkynyl.

C1-4 alkyl or C3-5 cycloalkyl can be optionally substituted selected from the group consisting of fluorine, hydroxyl, C1-3 alkoxy group, tetrahydropyranyl optionally substituted with one or more fluorines, hydroxyl, or C1-3 alkoxy group, tetrahydrofuranyl optionally substituted with one or more fluorines, hydroxyl, or C1-3 alkoxy group, and a combination thereof.

C2-6 alkenyl or C2-6 alkynyl can be optionally substituted with fluorine, hydroxyl, C1-3 alkoxy group, or a combination thereof.

—OC1-4 alkyl or —OC3-5 cycloalkyl can be optionally substituted with fluorine, hydroxyl, C1-3 alkoxy group, or a combination thereof.

—OC2-6 alkenyl or —OC2-6 alkynyl can be optionally substituted with fluorine, hydroxyl, C1-3 alkoxy group, or a combination thereof.

In some embodiments, n can be 0 or 1. When n=1, in some embodiment, R⁵ can be selected from the group consisting of H, methyl, CF₃, CHF₂, and CH₂F.

In some embodiments, R⁶ and R⁷ can be independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F or OH; C1-3 alkoxy group; —CH₂CCH; R⁹; CH₂R⁹; OR⁹; OCH₂R⁹; OCHMeR⁹.

In some embodiments, R⁶ and R⁷ can be connected to form a nitrogen-containing heterocycle, in such case, R⁶-R⁷ is to be selected from the group consisting of —(CHR¹¹)CH₂(CHR¹¹)O(CHR¹⁰)—, —(CHR¹⁰)O(CHR¹¹)₂—, —CH₂(CR¹³R¹⁴)CH₂—, —(CH₂)₂(CHR¹²)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkylidenyl)-, —(CHMe)CH₂O(3,3-oxatenylidenyl)-, —(CH₂)₂(1,1-cycloalkylidenyl)CH₂—, —(CH₂)ₘ— with m=4-6 and optionally substituted with one or more substituents independently selected from the group consisting of F, OH, and R¹¹.

In some embodiments, R⁹ can be phenyl or heteroaryl optionally substituted with one or more substituents independently selected from the group consisting of F, Cl, Br, CF₃, CHF₃, CH₂F, C1-4 alkyl, C3-5 cycloalkyl, —OC1-4 alkyl, and —OC3-5 cycloalkyl, wherein C1-4 alkyl, C3-5 cycloalkyl, —OC1-4 alkyl, or —OC3-5 cycloalkyl is optionally substituted with one or more fluorines.

In some embodiments, R¹⁰ can be selected from the group consisting of H, R⁹, C1-4 alkyl, —OC1-3 alkyl, and —OC3-5 cycloalkyl, wherein C1-4 alkyl, —OC1-3 alkyl, or —OC3-5 cycloalkyl can be optionally substituted substituents selected from the group consisting of F, OH, R⁹, and a combination thereof.

In some embodiments, R¹¹ can be selected from the group consisting of H, R⁹, C1-4 alkyl, C3-6 alkenyl, C3-6 alkynyl, —OC1-3 alkyl, —OC3-5 cycloalkyl, wherein C1-4 alkyl, C3-6 alkenyl, C3-6 alkynyl, —OC1-3 alkyl, or —OC3-5 cycloalkyl is optionally substituted with substituents selected from the group consisting of F, OH, R⁹, OR⁹, OCH₂R⁹, OCHMeR⁹, and a combination thereof.

In some embodiment, R¹² can be selected from the group consisting of H, CO₂H, CO₂R¹⁵, CH₂OH, CH₂OR¹⁵, C1-4 alkyl, C3-6 alkenyl, C3-6 alkynyl, and C3-5 cycloalkyl, wherein C1-4 alkyl, C3-6 alkenyl, C3-6 alkynyl, or C3-5 cycloalkyl is optionally substituted with one or more substituents selected from the group consisting of F, OH, and R⁹.

In some embodiments, R¹³ and R¹⁴ can be independently selected from the group consisting of H, F, CF₃, CHF₂, CH₂F, CN, OH, OR¹⁵, NHC(O)Me, SO₂Me, OSO₂Me, CO₂H, CO₂R¹⁵, CH₂OH, CH₂OR¹⁵, R⁹, and R¹⁵. In some embodiments, R¹³ and R¹⁴ can be optionally connected to form a cyclic structure, in such a case, R¹³-R¹⁴ is to be selected from the group consisting of: —CH₂OCH₂—, —(CH₂)₂O—, —(CH₂)O—, —(CH₂)₃—, —(CH₂)—, —CH₂CF₂CH₂—, —CH₂O(CHCF₃)—, —CH₂SO₂(CHCF₃)—, —CH₂(CHCO₂H)CH₂—, —CH₂(CHCO₂R¹⁵)CH₂—, —CH₂(CHCH₂OH)CH₂—, —CH₂(CHCH₂OR¹⁵)CH₂—, —(CHOH)CH₂O—, —(CHOR¹⁵)CH₂O—, —SO₂(CH₂)₂(CHOH)—, —SO₂(CH₂)₂(CHOR¹⁵)—, —SO₂(CH₂)(CHOH)CH₂—, —SO₂(CH₂)(CHOR¹⁵)CH₂—, —CH₂(CHOH)CH₂O—, —CH₂(CHOR¹⁵)CH₂O—, —(CHOH)(CH₂O—(CHOR¹⁵)(CH₂)O—, and —CH₂(3,3-oxetanyl)CH₂-.

In some embodiments, R¹⁵ can be selected from the group consisting of C1-4 alkyl, C3-5 cycloalkyl, C2-6 alkenyl, and C2-6 alkynyl, each of which is optionally substituted with one or more substituents selected from F, OH, and R⁹.

In some embodiment, Ar in Formula (IV) can be selected from:

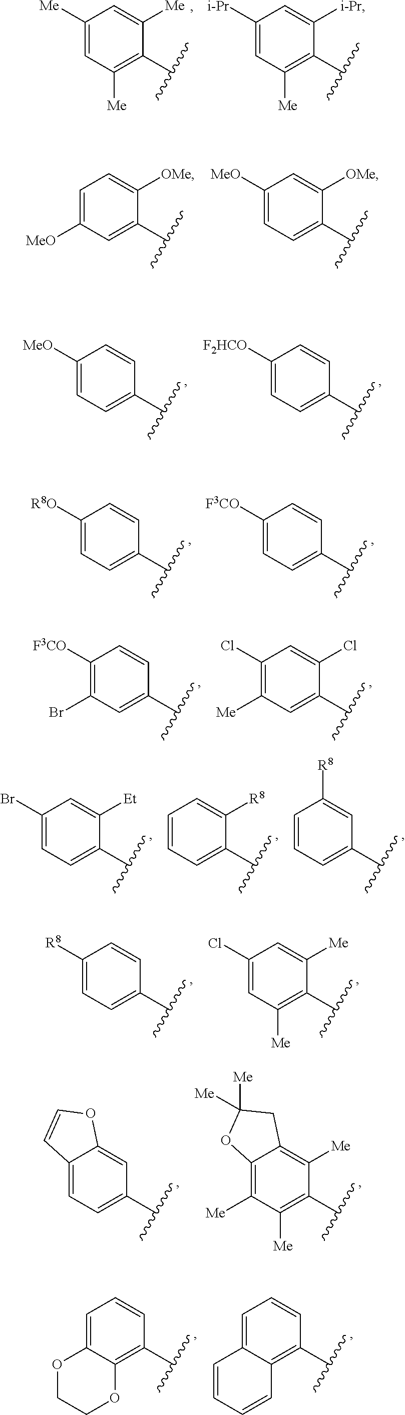

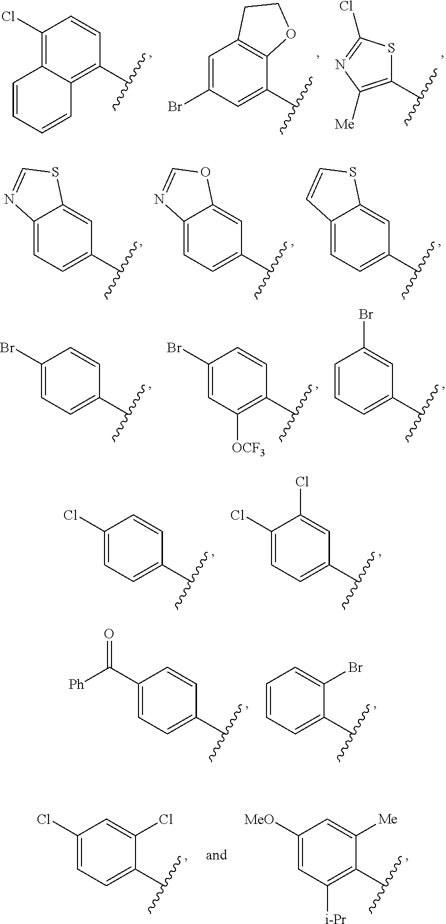

In some embodiments, these functional groups for Ar can be optionally further substituted with one or more substituents independently selected from the group consisting of F, Cl, Br, Me, CF₃, Et, i-Pr, cyclopropyl, OMe, OEt, Oi-Pr, —Ocyclopropyl, —OCF₃, —OCHF₂, —OCH₂F, —OCH₂R⁹, —OR⁹ and R⁹.

In some embodiment, when n=0, —NR⁶R⁷ can be selected from:

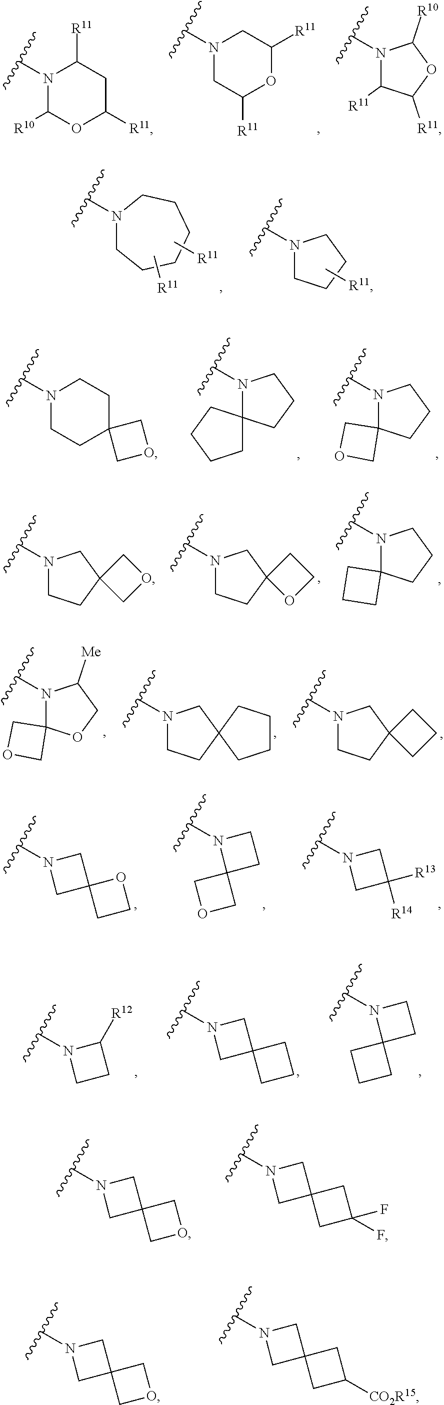

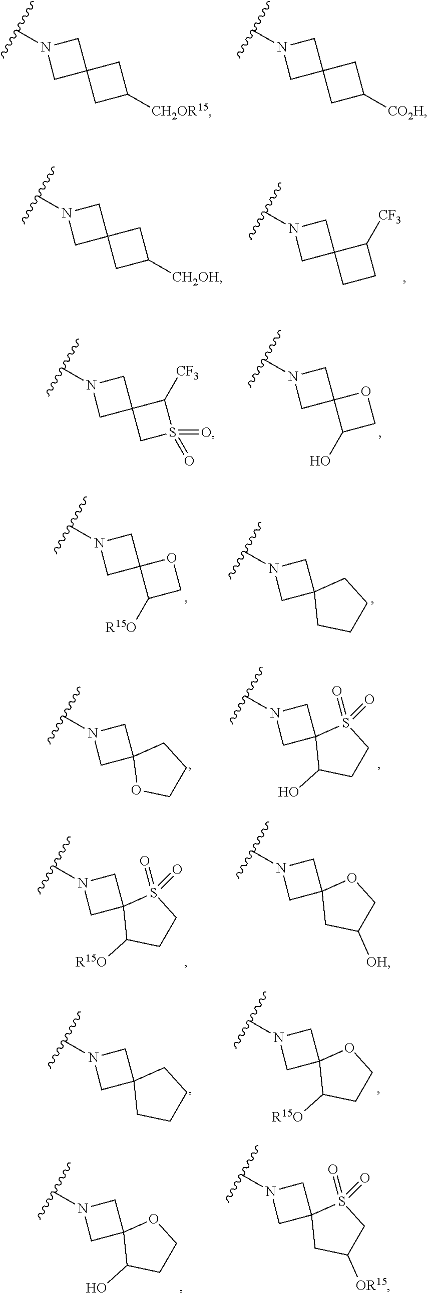

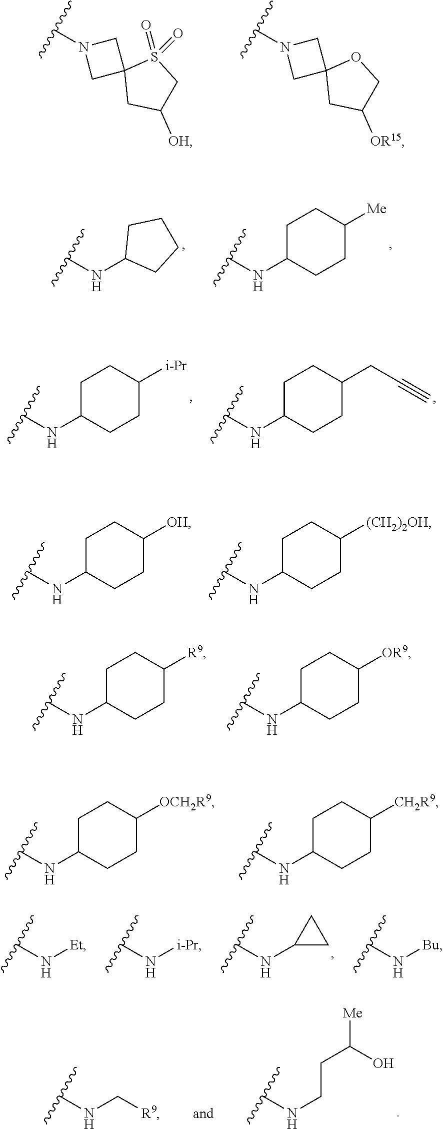

In some embodiment, when n=1, —NR⁶R⁷ can be selected from:

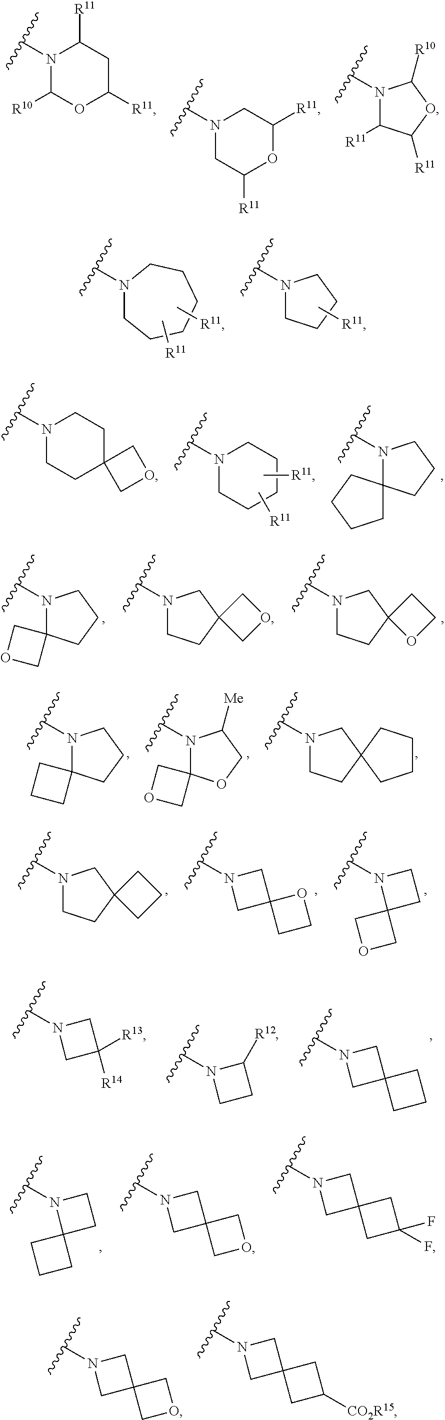

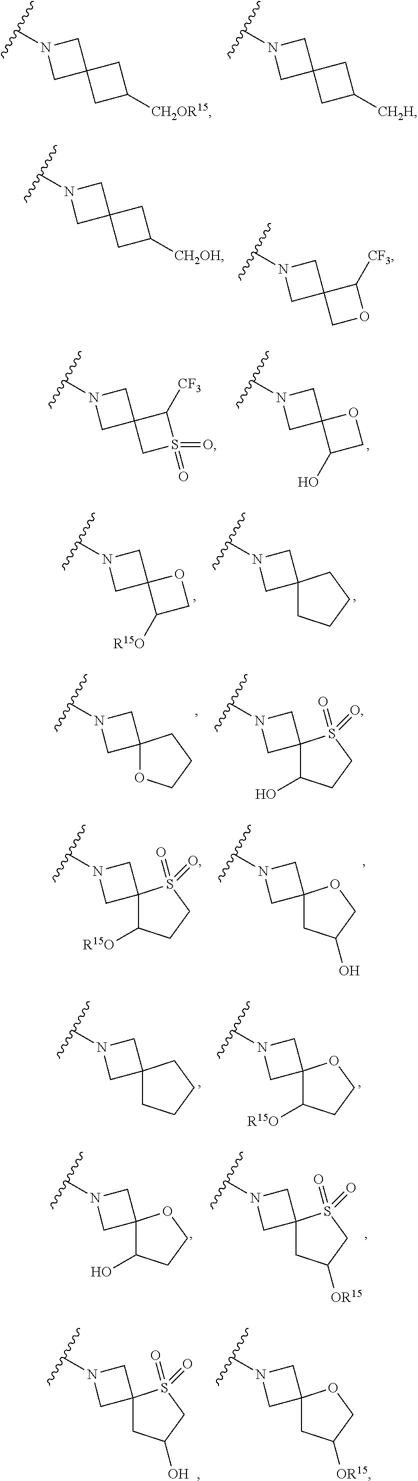

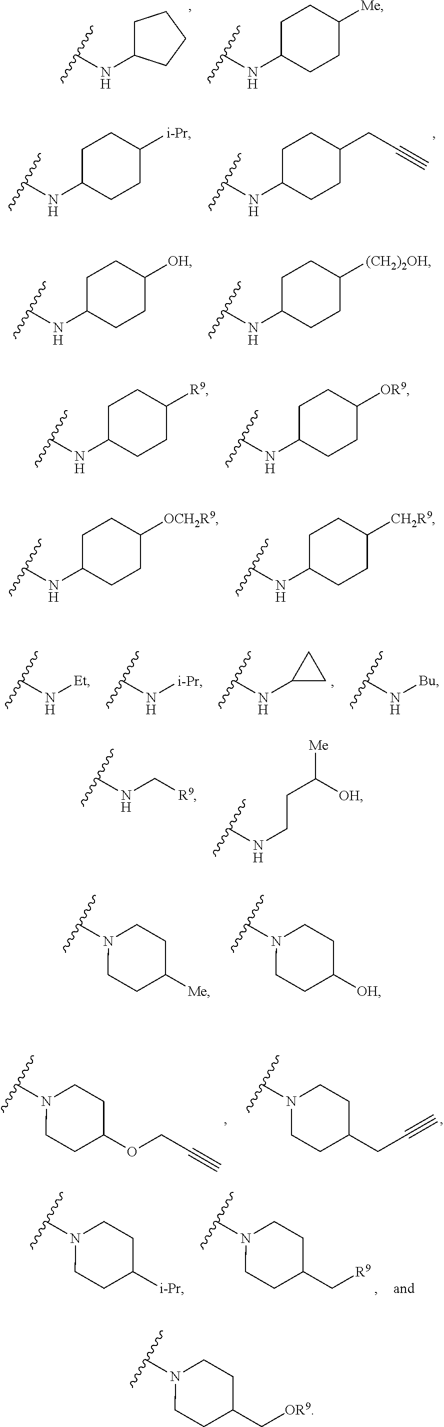

In some embodiments, disclosed herein is a compound with the structure of compound 121:

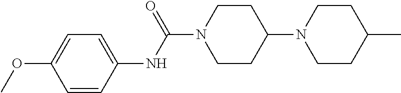

or a pharmaceutically acceptable salt or solvate, a stereoisomer, a diastereoisomer or an enantiomer thereof.

In some embodiments, the disclosed compounds can be effective to inhibit tumor growth, inhibit tumor proliferation, induce cell death or a combination thereof. In some embodiments, a therapeutic amount of the disclosed compound can be effective to inhibit Emopamil Binding Protein (EBP) or cholesterol delta8 delta7 somerase. Also disclosed is a pharmaceutical composition comprising a therapeutic amount of the disclosed compound herein and a pharmaceutically acceptable carrier.

In some embodiments, the instant disclosure provides a method for treating colorectal cancer in a subject including administering the disclosed compound. In some embodiments, the method further includes administering a chemotherapeutic agent. The disclosed compound can be administered prior to, simultaneously with or following the administration of the chemotherapeutic agent.

In some embodiment, the compound can be in form of a pharmaceutical composition comprising a therapeutic amount of the compound and a pharmaceutically acceptable carrier. In some embodiment, the compound can be administered in an therapeutic amount which is effective to inhibit tumor growth, inhibit tumor proliferation, induce cell death or a combination thereof. In some embodiment, the compound can be administered in a therapeutic amount which is effective to inhibit Emopamil Binding Protein (EBP) (also known as cholesterol delta8 delta7 isomerase.

In some embodiments, the instant disclosure provides a method for targeting Emopamil Binding Protein (EBP) for treating a subject with colorectal cancer with a pharmaceutical composition based on an Emopamil binding protein (EBP)-modulating anti-cancer compound according to Formula (I), Formula (II), Formula (III), Formula (IV), or compound 121, the method can include: (a) isolating a colorectal tumor sample comprising a population of cancer cells from the subject; (b) providing (i) an aliquot of the colorectal tumor sample in (a) as a test population of cancer cells, (ii) a known population of cancer cells sensitive to the EBP-modulating anticancer compound (positive control), and (iii) a known population of cancer cells insensitive to the EBP-modulating anticancer compound (negative control), wherein the known population of cancer cells sensitive to the EBP modulating anti-cancer compound (positive control) is a population of cancer cells selected from the group consisting of DLD1 cells, HT29 cells, SW620 cells, SE480 cells, Caco-2 cells, Lovo cells, and HCl 16 p53−/−A1309 cells, and the known population of cancer cells insensitive to the EBP-modulating anticancer compound (negative control) is a population of cancer cells selected from the group consisting of HCT116 cells and RKO cells; (c) determining whether the aliquot of the colorectal tumor sample contains a subpopulation of cancer cells sensitive to the composition comprising the EBP-modulating anti-cancer compound by (1) contacting the EBP-modulating anticancer compound to the populations of cancer cells in (b); (2) measuring EBP enzyme activity and cholesterol synthesis rate for each population of cancer cells, wherein in a cancer cell sensitive to the EBP modulating anti-cancer compound, an amount of the EBP-modulating anti-cancer compound is effective to decrease EBP enzyme activity and to decrease the rate of endogenous cholesterol synthesis, while in a cancer cell insensitive to the EBP modulating anti-cancer compound, an amount of the EBP-modulating anti-cancer compound is effective to increase EBP activity and to increase the rate of endogenous cholesterol synthesis; and (d) upon determining that the test population of colorectal cancer cells contains a population of cancer cells sensitive to the EBP modulating anti-cancer compound in (c), treating the colorectal tumor by administering to the subject the pharmaceutical composition containing a therapeutic amount of the EBP modulating anti-cancer compound.

According to one embodiment of the method, in a cancer cell sensitive to the EBP modulating anti-cancer compound, the effective amount of the EBP-modulating anti-cancer compound is effective to cause accumulation of a Δ8 sterol intermediate. According to another embodiment, the Δ8 sterol intermediate is 5α-cholest-8-(9)-en-3β-ol (Δ8-cholestenol). According to another embodiment, in the cancer cell sensitive to the EBP-modulating anticancer compound, the effective amount of the EBP modulating anti-cancer compound is effective to cause downregulation of SREBP-2. According to another embodiment, in the cancer cell sensitive to the EBP-modulating anticancer compound, the effective amount of the EBP modulating anti-cancer compound is effective to cause downregulation of SREBP-2 genes. According to another embodiment, in the cancer cell sensitive to the EBP-modulating anticancer compound, the effective amount of the EBP modulating anti-cancer compound is effective to cause downregulation of SREBP-2 and one or more SREBP-2 target genes of the cholesterol biosynthetic pathway selected from the group consisting of ACAT2; MHGCS1; HMGCR; MVK; PMVK; MVD; I 11/ID12; FDFS; GGPS1; FDFT1; SQLE; LSS; CYPS1A1; TM75F2; SCAMOL; NSDHL; HSD17B7; EBP; SC5D; DHCR7; and DHCR24. According to another embodiment, the cancer cell sensitive to the EBP-modulating anti-cancer compound comprises a truncated APC protein. According to another embodiment, the therapeutic amount of the EBP-modulating anti-cancer compound is effective to reduce proliferation of the cancer cell sensitive to the EBP modulating anti-cancer compound, to reduce invasiveness of the cancer cell sensitive to the EBP modulating anti-cancer compound, increase apoptosis of the cancer cell sensitive to the EBP modulating anti-cancer compound, reduce growth of a tumor comprising the cancer cell sensitive to the EBP modulating anti-cancer compound, reduce tumor burden, improve progression free survival, improve overall survival, achieve remission of disease, or a combination thereof. According to another embodiment, the EBP-modulating anti-cancer compound is selected from the group consisting of TASIN-1 and functional equivalents thereof, including dendrogenin A, SR31747A, tamoxifen, emopamil, verapamil, cis-flupentixol, trifluoroperazine, 7-ketocholestenol, haloperidol, and fenpropimorph.

According to another embodiment, the known population of cancer cells insensitive to the EBP-modulating anticancer compound is a population of HCT116 cells or RKO cells. According to another embodiment, the known population of cancer cells sensitive to the EBP modulating anti-cancer compound is a population of DLD1 cells, HT29 cells, SW620 cells, SE480 cells, Caco-2 cells, Lovo cells or HC116 p53−/−A1309 cells.

Also disclosed herein is a method for identifying a therapeutic EBP-modulating anticancer compound includes: (a) dividing a population of cancer cells sensitive to a known EBP-modulating anti-cancer compound into aliquoted samples of the population of cancer cells; wherein the population of cancer cells sensitive to the known EBP-modulating anti-cancer compound is a population of DLD 1 cells or HT29 cells, the known EBP-modulating anti-cancer compound is

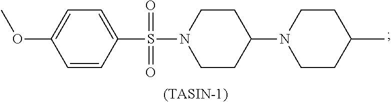

(b) contacting one sample of the population of sensitive cancer cells with a candidate EBP-modulating anti-cancer compound, contacting a second sample of the sensitive population of cancer cells with the known EBP-modulating anticancer compound (positive control), and contacting a third sample of the sensitive population of cancer cells with a compound that does not modulate EBP activity (negative control); (c) measuring EBP enzyme activity and cholesterol synthesis rate for the candidate EBP-modulating compound, the positive control and the negative control in (b), wherein an amount of the known EBP-modulating anti-cancer compound is effective to decrease EBP activity and to decrease the rate of endogenous cholesterol synthesis in a sensitive cancer cell; (d) ranking a plurality of candidate EBP-modulating anti-cancer compounds according to the measured effect on EBP activity and the parameter of endogenous cholesterol synthesis in (c); and (e) selecting a top-ranked candidate EBP-modulating anti-cancer compound in (d) from the compounds according to Formula (I) as a new EBP-modulating anti-cancer compound for treating a subject in need thereof.

According to one embodiment of the method, the population of cancer cells known to be sensitive to the EBP modulating compound is a population of DLD1 cells or HT29 cells. According to another embodiment, the EBP-modulating anti-cancer compound is selected from TASIN-1 or a functional equivalent thereof, dendrogenin A, SR31747A, tamoxifen, emopamil, verapamil, cis-flupentixol, trifluoroperazine, 7-ketocholestenol, haloperidol, and fenpropimorph. According to another embodiment, the decrease in EBP activity is measured as an accumulation of a Δ8 sterol intermediate. According to another embodiment, the Δ8 sterol intermediate is 5α-cholest-8-(9)-en-3β-ol (Δ8-cholesetenol). According to another embodiment, the effective amount of the new EBP modulating anti-cancer compound is effective to cause downregulation of SREBP-2. According to another embodiment, the effective amount of the new EBP modulating anti-cancer compound is effective to cause downregulation of one or more SREBP-2 target genes of the cholesterol biosynthetic pathway selected from the group consisting of ACAT2; MHGCS1; HMGCR; MVK; PMVK; MVD; ID11/ID12; FDFS; GGPS1; FDFT1; SQLE; LSS; CYPS1A1; TM75F2; SCAMOL; NSDHL; HSD17B7; EBP; SC5D; DHCR7; and DHCR24. According to another embodiment, the effective amount of the new EBP modulating anti-cancer compound is effective to cause downregulation of SREBP-2 and one or more SREBP-2 target genes of the cholesterol biosynthetic pathway selected from the group consisting of ACAT2; MHGCS1; HMGCR; MVK; PMVK; MVD; IDI/ID12; FDFS; GGPS1; FDFT1; SQLE; LSS; CYPS1A1; TM75F2; SCAMOL; NSDHL; HSD17B7; EBP; SC5D; DHCR7; and DHCR24.

### BRIEF DESCRIPTION OF THE DRAWINGS

In order to facilitate a full understanding of the present disclosure, reference is now made to the accompanying drawings. These drawings should not be construed as limiting the present disclosure, but are intended to be illustrative only.

FIG. 1 is an illustration of cholesterol homeostasis in a typical mammalian cell;

FIGS. 2A-2B show that DLD1 cells cultured in 0.2% serum or 2% lipoprotein deficient serum (LPPS) are sensitive to TASIN-1 (compound 6);

FIG. 3 shows that DLD1 cells adapted to 0.2% serum medium and non-adapted cells rapidly changed from 10% to low serum have similar sensitivity to TASIN-1;

FIGS. 4A-4B show that sensitivity of DLD1 cells to TASIN-1 is gradually lost by increasing serum level, but not by increasing the amount of lipoprotein poor serum;

FIGS. 5A-5C show that TASIN-1 prevents colon cancer progression, which otherwise is accelerated by a high fat diet in CPC/Apc mice;

FIG. 6 shows SDS PAGE of TASIN competitor compounds with DLD-1 cells in the presence and absence of UV light;

FIG. 7 shows that a series of UV-dependent bands are competed by active TASIN analogues but not by inactive analogues. Band p27, p22 and p18 are competiting, of which p22 is the strongest with CC002;

FIG. 8 shows a scheme for purification of p22 for mass spectrometry;

FIG. 9 shows that known EBP antagonists nafoxidine, ifenprodil, and U18666A compete with p22 (EBP);

FIGS. 10A-10B show that known EBP antagonists nafoxidine and ifenprodil recapitulate selectivity but are less potent than TASIN;

FIGS. 11A-11B show that TASIN-1 kills DLD-1 and HT29 cells in 2% Lipoprotein deficient serum (LPPS) but not in 2% FBS media;

FIGS. 12A-12B show that exemplary TASIN analogues are toxic and selective for DLD-1 in 0.2% HCEC medium;

FIGS. 13A-13B show that exogenous addition of purified lipoproteins or cholesterol to the medium decreases sensitivity of DLD1 cells to TASIN-1;

FIGS. 14A-14D show that stable knockdown of EBP, like TASIN-1, affects growth of DLD1 cells in 0.2% FBS;

FIGS. 15A-15C show that stable knockdown of EBP does not affect growth of HCT116 cells in 0.2% FBS;

FIG. 16 shows that overexpression of EBP confers resistance to TASIN-1 in DLD-1 cells;

FIG. 17 shows that APC truncation expression reduces SREBP1 & 2 cleavage in DLD-1 cells;

FIG. 18 shows that APC truncation expression down-regulates a panel of genes involved in cholesterol homeostasis;

FIG. 19 shows that knockdown of truncated APC significantly increases endogenous cholesterol biosynthesis, but reintroduction of truncated APC returns the rate of cholesterol synthesis in DLD1 cells back to DLD1 levels;

FIG. 20 shows that TASIN-1 further reduces endogenous cholesterol biosynthesis (dpm/μg protein) in cells containing truncated APC, but not in cells with wild type APC;

FIGS. 21A-21B show that simvastatin has only a slight effect on survival of DLD-1 cells (FIG. 21B), and is significantly less potent (IC50 4.5 μM) than TASIN-1 (IC50 0.063 μM, FIG. 21A);

FIG. 22 shows that 210, a biotin-labeled potent TASIN analog, interacts with EBP in DLD1 cells. DLD1 cells were incubated with 210 in the presence or absence of TASIN-land pulled down by streptavidin beads. Bound EBP was detected by Western Blot. EBP is not pulled down in DLD1shEBP cells. These results confirm the interaction between TASIN-1 and EBP in DLD-1 cells;

FIG. 23 shows that TASIN-1 decreases intracellular cholesterol level in DLD1 but not in HCT116 cells. Cells were treated with DMSO or 2.5 μM of TASIN-1 for 24 or 48 hours. Cholesterol levels were determined by Filipin III staining. Fipipin is a fluorescent chemical that specifically binds to cholesterol;

FIG. 24 shows that APC truncated protein is involved in cholesterol homeostasis. Cholesterol and fatty acid synthesis rates were measured in isogenic HCEC (1CTRPA, 1CTRPA A1309) and DLD1 cell lines (DLD1, DLD1 APC knockdown). Data represent mean±s.d., n=2. Student's t-test, *P<0.05, **P<0.01. APC truncation expression affects cholesterol and fatty acid biosynthesis rate;

FIG. 25 shows the relative SRE luciferase activity in HCT116 and DLD1 cells treated with 2.5 μM of TASIN-1 or 10 μM of Simvastatin for 24 hours. Data represent mean±s.d., n=2. Student's t-test, **P<0.01. TASIN-1 treatment increased sterol response element (SRE) luciferase activity only in HCT116 cells;

FIGS. 26A-26B show the results of Quantitative PCR analysis of the major target genes regulated by SREBP2 in HCT116 cells (FIG. 26A) or DLD1 cells (FIG. 26B) treated with 2.5 μM of TASIN-1 for 24 and 48 hours. Expression level was normalized to the control cells. Data represent mean±s.d., n=2. TASIN-1 treatment leads to up-regulation of SREBP2 target genes only in HCT116 cells;

FIG. 27 is a lipoprotein signaling PCR array (Qiagen, 90 genes) showing upregulation and downregulation of a panel of cholesterol signaling related genes in APC knockdown DLD1 cells, which are reversed by ectopic expression of APC1309. The results demonstrate gain-of-Function of APC truncation in cholesterol signaling and metabolism;

FIG. 28 shows that APC truncation affects expression of SREBP2 target genes. Quantitative PCR was performed on the isogenic DLD1 cell lines with primers against the major target genes regulated by SREBP2. Expression level was normalized to that in DLD1 cells. Data represent mean±s.d., n=2;

FIG. 29 confirms the interaction between TASIN-1 and EBP in colorectal cancer (CRC) cells. CRC cells were incubated with TASIN-1 analog #210 and labeled with Alexa532 after UV crosslinking via click reaction. Proteins were precipitated using cold acetone and resuspended in Laemmli buffer, followed by in-gel fluorescence and Western blot analysis;

FIG. 30 is a concentration-time curve in the large intestine for compound 92 administered via i.v. or i.p at a dosage of 10 mg/kg;

FIG. 31 is a concentration-time curve in the plasma and lung for compound 22 administered via i.v. at a dosage of 5 mg/kg; and

FIG. 32 depicts the pharmacological data of compound 87 in different cell lines.

### DETAILED DESCRIPTION

Despite significant advances in targeted anticancer therapies, there are still no small molecule-based therapies available that specifically target oncogenotypes that drive colorectal cancer development and progression, the second-leading cause of cancer deaths. We previously disclosed the discovery of TASIN-1, a small molecule that highly specifically targets, in vitro and in vivo, human colorectal cancer cells lines with truncating mutations in the Adenomatous Polyposis Coli (APC) tumor suppressor gene through inhibition of endogenous cholesterol biosynthesis. Here, we report an extensive medicinal chemistry evaluation of a large collection of analogs of this Truncating APC-Selective Inhibitor (TASIN). Analogs were evaluated for activity against a series of colon cancer cell lines with and without truncating APC-mutations, as well as in an isogenic cell line pair reporting on the status of APC-dependent selectivity. A number of very potent and selective analogs were identified, including compounds with good metabolic stability and PK properties. The small molecules reported herein thus represent a first-in-class genotype-selective series that specifically target apc mutations present in the vast majority of CRC patients, and therefore serves as a translational platform towards a potential targeted therapy for colon cancer.

We recently described a potent small molecule that selectively kills CRC cells with truncated apc protein termed TASIN-1 (Truncated APC-Selective Inhibitor 1) using a 250,000 compound high throughput screen (HTS) to identify small molecules with selective cytotoxic activity against an experimentally developed human colonic epithelial cell line (HCEC) with introduced oncogenes (KRAS, CDK4, TERT), coupled with loss of tumor suppressor function (p53) and expressing a mutant apc protein truncated at amino acid residue AA1309 (1CTRPA A1309). TASIN-1 was not toxic against the isogenic HCEC cell line that expressed the wild type apc protein (1CTRPA), and selectivity for apc-truncating mutations was retained in every human cell line (normal and cancer) that we tested. Based on serum and sterol rescue experiments, we postulated that TASIN-1 exerts its cytotoxic effects through inhibition of cholesterol biosynthesis. Furthermore, TASIN-1 inhibited the growth of human tumor xenografts in mice implanted with tumors derived from DLD-1 or HT29 (APC^{TR}), but not HCT116 (APC^{WT}) CRC cell lines. Also, TASIN-1 treatment significantly reduced the number of polyps and tumor size in the colons of a genetically engineered mouse apc inactivation model of colonic adenoma-carcinoma progression (CPC;APC mice). In addition, TASIN-treated mice (90-day treatment), gained weight and did not show any signs of overt toxicity (histopathology, liver function, kidney function, blood cell counts all look normal). Given these promising initial results with TASIN-1, we further characterized the TASIN chemotype and present herein our results related to an extensive medicinal chemistry program that delineates the Structure Activity Relationships (SAR) within this scaffold. A number of very potent and selective analogs were identified, including compounds with good metabolic stability against murine microsomal fractions (S9) and PK properties. The small molecules reported herein thus represent a first-in-class genotype-selective series that specifically target apc mutations present in the vast majority of CRC patients, and therefore serves as a translational platform towards a potential targeted therapy for colon cancer.

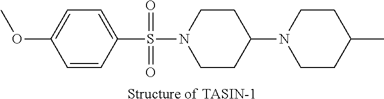

Mutations in the human APC tumor suppressor gene are linked to Familial Adenomatous Polyposis (FAP), an inherited cancer-prone condition in which numerous polyps are formed in the epithelium of the large intestine (See Kinzler et al., Science, 1991; 253:661-665; Kinzler and Vogelstein, Cell, 1996; 87:159-170; Half et al., Orphanet Journal of Rare Diseases, 2009; 4:22). The development of CRC is initiated by the aberrant outgrowth of adenomatous polyps from the colonic epithelium that ultimately evolve into aggressive carcinomas (See Kinzler and Vogelstein, Cell, 1996; 87: 159-170). About 85% of sporadic colorectal cancers have been reported to harbor APC truncating mutations (See Kinzler and Vogelstein, Cell, 1996; 87:159-170). The growth of the polyps is associated in most cases with alterations of both alleles of the Adenomatous Polyposis Coli (APC) gene. A first mutational hit occurs roughly in the middle of the open reading frame, generating a truncated APC molecule lacking the C-terminal half. Such truncation mutations are located in the so-called mutation cluster region (MCR) (See Schneikert et al., Human Molecular Genetics, 2006; 16: 199-209). The second mutational hit involves either deletion of the second allele or a mutation that leads to the synthesis of a truncated product, almost never occurring after the MCR (See Schneikert et al., Human Molecular Genetics, 2006; 16: 199-209). Thus, colon cancer cells express at least a truncated APC molecule whose length is defined by the position of the MCR and, occasionally, an additional but shorter fragment.

CRC treatment is primarily reliant upon chemotherapeutic agents that act with minimal specificity for the underlying genetic basis of disease. These chemotherapeutic agents frequently disrupt the function of normal cells while disrupting cancer cells due to shared reliance on the chemical target. Better, more precise therapeutic agents are needed to improve treatment of patients diagnosed with CRC.

### Adenomatous Polyposis Coli (APC) Gene

APC, which does not act as a classical tumor suppressor, influences Wnt signaling thereby regulating gene transcription. Wnts are a family of secreted cysteine-rich glycoproteins that have been implicated in the regulation of stem cell maintenance, proliferation, and differentiation during embryonic development. Canonical Wnt signaling increases the stability of cytoplasmic β-catenin by receptor-mediated inactivation of GSK-3 kinase activity and promotes β-catenin translocation into the nucleus. The canonical Wnt signaling pathway also functions as a stem cell mitogen via the stabilization of intracellular β-catenin and activation of the β-catenin/TCF/LEF transcription complex, resulting in activated expression of cell cycle regulatory genes, such as Myc, cyclin D1, EPhrinB (EPhB) and Msx1, which promote cell proliferation (See Cayuso and Marti, Journal of Neurobiology, 2005; 64:376-387).

APC is the negative regulator of Wnt signaling. Without this negative regulation, the Wnt pathway is more active and is important in cancer (See Polakis, Current Opinion in Genetics & Development, 2007; 17: 45-51). Studies comparing tumor cells with mutations in both APC alleles to correlate levels of Wnt signaling and severity of disease in both humans and mice have aided in establishing a model in which gene dosage effects generate a defined window of enhanced Wnt signaling, leading to polyp formation in the intestine. Combinations of ‘milder’ APC mutations, associated with weaker enhancement of Wnt signaling, give rise to tumors in extra-intestinal tissues. According to this model, the nature of the germline mutation in APC determines the type of somatic mutation that occurs in the second allele. (See Minde et al. Molecular Cancer, 2011; 10:101).

### APC Protein

The APC gene product is a 312 kDa protein consisting of multiple domains, which bind to various proteins, including beta-catenin, axin, C-terminal binding protein (CtBP), APC-stimulated guanine nucleotide exchange factors (Asefs), Ras GTPase-activating-like protein (IQGAP1), end binding-1 (EB1) and microtubules. Studies using mutant mice and cultured cells demonstrated that APC suppresses canonical Wnt signaling, which is essential for tumorigenesis, development and homeostasis of a variety of cell types, including epithelial and lymphoid cells. Further studies have suggested that the APC protein functions in several other fundamental cellular processes. These cellular processes include cell adhesion and migration, organization of actin and microtubule networks, spindle formation and chromosome segregation. Deregulation of these processes caused by mutations in APC is implicated in the initiation and expansion of colon cancer (See Aoki and Taketo, Journal of Cell Science, 2007; 120:3327-3335).

The APC protein functions as a signaling hub or scaffold, in that it physically interacts with a number of proteins relevant to carcinogenesis. Loss of APC influences cell adhesion, cell migration, the cytoskeleton, and chromosome segregation (See Aoki and Taketo, Journal of Cell Science, 2007; 120:3327-3335).

Most investigators believe that APC mutations cause a loss of function change in colon cancer. Missense mutations yield point mutations in APC, while truncation mutations cause the loss of large portions of the APC protein, including defined regulatory domains. A significant number of APC missense mutations have been reported in tumors originating from various tissues, and have been linked to worse disease outcome in invasive urothelial carcinomas (See Kastritis et al., International Journal of Cancer, 2009; 124:103-108), suggesting the functional relevance of point mutated APC protein in the development of extra-intestinal tumors. The molecular basis by which these mutations interfere with the function of APC remains unresolved.

APC mutation resulting in a change of function can influence chromosome instability in at least three manners: by diminishing kinetochore-microtubule interaction, by the loss of mitotic checkpoint function and by generating polyploid cells. For example, studies have shown that APC bound to microtubules increased microtubule stability in vivo and in vitro, suggesting a role of APC in microtubule stability (See Zumbrunn et al., Current Biology, 2001; 11:44-49). Truncated APC led to chromosomal instability in mouse embryonic stem cells (See Fodde et al., Nature Cell Biology, 2001; 3:433-438), interfered with microtubule plus-end attachments, and caused a dramatic increase in mitotic abnormalities (See Green and Kaplan, Journal of Cell Biology, 2003; 163:949-961). Studies have shown that cancer cells with APC mutations have a diminished capacity to correct erroneous kinetochore-microtubule attachments, which account for the wide-spread occurrence of chromosome instability in tumors (See Bakhoum et al., Current Biology, 2009; 19:1937-1942). In addition, abrogation of the spindle checkpoint function was reported with APC loss of function. Knockdown of APC with siRNA indicated that loss of APC causes loss of mitotic spindle checkpoint function by reducing the association between the kinetochore and checkpoint proteins Bub1 and BubR1. Thus, loss of APC reduces apoptosis and induces polyploidy (See Kaplan et al., Nature Cell Biology, 2001; 3:429-432; Dikovskaya et al., Journal of Cell Biology, 2007; 176:183-195; Rusan and Peifer, Journal of Cell Biology, 2008; 181:719-726). Polyploidy is a major source for aneuploidy since it can lead to multipolar mitosis (See Shi and King, Nature, 2005; 437:1038-1042).

While loss of function due to APC may be partially correct, there are reports showing that a large fraction of colon cancer patients have at least one APC gene product that is truncated, and that this truncated APC gene has a gain of function. Thus, truncated APC proteins may play an active role in colon cancer initiation and progression as opposed to being recessive; for example, truncated APC, but not full-length APC may activate Asef and promote cell migration.

Emopamil binding protein (EBP) in complex with dihydrocholesterol-7 reductase (DHCR7) catalyzes isomerization of the double-bond between C7 and C8 in the second cholesterol ring. (Gabitova, L. et al., “Molecular Pathways: Sterols and receptor signaling in Cancer,” Clin. Cancer Res. 19(23): 6344-50 (2013)). This complex mediates the activity of cholesterol epoxide hydrolase (Id., citing de Medina, P. et al, “Identification and pharmacological characterization of cholesterol-5,6-epoxide hydrolase as a target for tamoxifen and AEBS ligands,” Proc. Natl. Acad. Sci. USA 107: 13520-5 (2010)).

There are several known inhibitors of EBP, and some have been described as anti-cancer agents. For example, a sterol conjugate of a naturally occurring steroidal alkaloid, 5alpha-hydroxy-6beta-[2-(1H-imidazol-4-yl)ethylamino]cholestan-3beta-ol (dendrogenin A) which is produced in normal, but not in cancer cells, and 5,6 alpha-epoxy-cholesterol and histamine (Id., citing de Medina, P. et al, “Dendrogenin A arises from cholesterol and histamine metabolism and shows cell differentiation and anti-tumour properties,” Nature Communic. 4: 1840 (2013); de Medina, P. et al, “Synthesis of new alkylaminooxysterols with potent cell differentiating activities: identification of leads for the treatment of cancer and neurodegenerative diseases,” J. Med. Chem. 52: 7765-77 (2009)), has been shown to suppress cancer cell growth and to induce differentiation in vitro in various tumor cell lines of different types of cancers (Id., citing de Medina, P. et al, “Synthesis of new alkylaminooxysterols with potent cell differentiating activities: identification of leads for the treatment of cancer and neurodegenerative diseases,” J. Med. Chem. 52: 7765-77 (2009)). It also inhibited tumor growth in melanoma xenograft studies in vivo and prolonged animal survival. (Id., citing de Medina, P. et al, “Dendrogenin A arises from cholesterol and histamine metabolism and shows cell differentiation and anti-tumour properties,” Nature Comm. 4: 1840 (2013)).

SR31747A (cis-N-cyclohexyl-N-ethyl-3-(3-chloro-4-cyclohexyl-phenyl)propen-2-ylamine hydrochloride), a selective peripheral sigma binding site ligand whose biological activities include immunoregulation and inhibition of cell proliferation, binds to SR31747A-binding protein 1 (SR-BP) and EBP with nanomolar affinity. Berthois, Y. et all., “SR31747A is a sigma receptor ligand exhibiting antitumoural activity both in vitro and in vivo,” Br. J. Cancer 88: 438-46 (2003). The effect of SR31747A on proliferative activity was evaluated in vitro on the following breast and prostate cancer cell lines: breast (hormone responsive MCF-7 cells from a breast adenocarcinoma pleural effusion; MCF-7AZ; Hormone independent MCF-7/LCC1 cells derived from MCF-7 cell lines; MCF-7LY2, resistant to the growth-inhibitory effects of the antiestrogen LY117018; Hormone unresponsive MDA-MB-321 and BT20 established from a metastatic human breast cancer tumor); and prostate (Hormone responsive prostate cancer cell line LNCaP; hormone-unresponsive PC3 cell line established from bone marrow metastasis; hormone-unresponsive DU145 established from brain metastasis). Id. SR31747A induced concentration-dependent inhibition of cell proliferation, regardless of whether the cells were hormone responsive or unresponsive. Id. The antiproliferative effect of SR31747A was partially reduced by adding cholesterol (Id.; Labit-Le Bouteiller, C. et al., “Antiproliferative effects of SR31747A in animal cell lines are mediated by inhibition of cholesterol biosynthesis at the sterol isomerase step,” Eur. J. Biochem. 256: 342-49 (1998)), thus demonstrating the involvement of EBP. Sensitivity to SR31747A did not correlate with cellular levels of EBP. Berthois, Y. et all., “SR31747A is a sigma receptor ligand exhibiting antitumoural activity both in vitro and in vivo,” Br. J. Cancer 88: 438-46 (2003). SR31747A also inhibited proliferation in vivo in the mouse xenograft model. Id. Murine EBP cDNA overexpression in CHO cells increased resistance of these cells to SR31747A-induced inhibition of proliferation. Labit-Le Bouteiller, C. et al., “Antiprolifertive effects of SR31747A in animal cell lines are mediated by inhibition of cholesterol biosynthesis at the sterol isomerase step,” Eur. J. Biochem. 256: 342-49 (1998)).

Tamoxifen inhibited SR31747 binding in a competitive manner and induced the accumulation of Δ8-sterols, while Emopamil, a high affinity ligand of human sterol isomerase and a calcium channel blocker, and verapamil, another calcium channel-blocking agent, are inefficient in inhibiting SR31747 binding to its mammalian target, suggesting that their binding sites do not overlap. Paul, R. et al., “Both the immunosuppressant SR31747 and the antiestrogen tamoxifen bind to an emopamil-insensative site of Mammalian Δ8-47 sterol isomerase,” J. Pharmacol. Exptl Thera. 285(3): 1296-1302 (1998)). Some drugs, e.g., cis-flupentixol, trifluoroperazine, 7-ketocholestanol and tamoxifen, inhibit SR31747 binding only with mammalian EBP enzymes, whereas other drugs, e.g., haloperidol and fenpropimorph, are more effective with the yeast enzyme than with the mammalian ones. Id.

While some cancer cell lines are highly sensitive to small molecule EBP inhibition, other cancer cell lines, as well as normal cell lines, do not respond to EBP inhibition, even when up to 10,000-fold higher concentrations of the EBP inhibitors are used. A determination of which cancer will respond to which inhibitor therefore has required an empirical hit or miss, impractical and expensive, approach.

The instant disclosure establishes that EBP inhibition is only toxic to cancer cells that paradoxically respond to small molecule EBP inhibitors via downregulation of endogenous cholesterol biosynthesis, and provides a method for identifying such EBP inhibitors and for cancer cells that are sensitive to treatment with such inhibitors.

As such, disclosed herein are compounds 5 to 170, or a pharmaceutically acceptable salt or solvate, a stereoisomer, a diastereoisomer or an enantiomer thereof.

In some embodiments, the present disclosure provides an EBP-modulating anti-cancer compound with the structure of Formula (I):

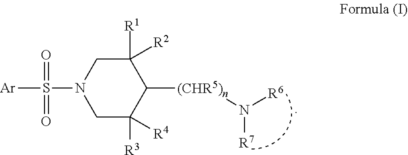

or a pharmaceutically acceptable salt or solvate, a stereoisomer, a diastereoisomer or an enantiomer thereof.

In some embodiment, R¹, R², R³, and R⁴ can be independently selected from the group consisting of H, F, CF₃, CHF₂, CH₂F, and methyl.

In some embodiment, Ar can be selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl.

In some embodiments, the optional substituent for Ar can be selected from the group consisting of F, Cl, Br, —OCF₃, —OCHF₂, —OCH₂F, —OCH₂R⁸, —OCHMeR⁸, —OCH(CF₃)R⁸, —OR⁸, —C(O)R⁸, R⁸, C1-4 alkyl, C3-5 cycloalkyl, C2-6 alkenyl, C2-6 alkynyl, —OC1-4 alkyl, —OC3-5 cycloalkyl, —OC2-6 alkenyl, and —OC2-6 alkynyl.

C1-4 alkyl or C3-5 cycloalkyl can be optionally substituted selected from the group consisting of fluorine, hydroxyl, C1-3 alkoxy group, tetrahydropyranyl optionally substituted with one or more fluorines, hydroxyl, or C1-3 alkoxy group, tetrahydrofuranyl optionally substituted with one or more fluorines, hydroxyl, or C1-3 alkoxy group, and a combination thereof.

C2-6 alkenyl or C2-6 alkynyl can be optionally substituted with fluorine, hydroxyl, C1-3 alkoxy group, or a combination thereof.

—OC1-4 alkyl or —OC3-5 cycloalkyl can be optionally substituted with fluorine, hydroxyl, C1-3 alkoxy group, or a combination thereof.

—OC2-6 alkenyl or —OC2-6 alkynyl can be optionally substituted with fluorine, hydroxyl, C1-3 alkoxy group, or a combination thereof.

In some embodiments, n can be 0 or 1. When n=1, in some embodiment, R⁵ can be selected from the group consisting of H, methyl, CF₃, CHF₂, and CH₂F.

In some embodiments, R⁶ and R⁷ can be independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F or OH; C1-3 alkoxy group; —CH₂CCH; R⁸; CH₂R⁸; OR⁸; OCH₂R₈; OCHMeR⁸.

In some embodiments, R⁶ and R⁷ can be connected to form a nitrogen-containing heterocycle, in such case, R⁶-R⁷ is to be selected from the group consisting of —(CHR¹⁰)CH₂(CHR₁₀)O(CHR⁹)—, —(CHR⁹)O(CHR¹⁰)₂—, —CH₂(CR¹²R¹³)CH₂—, —(CH₂)₂ (CHR¹¹)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkylidenyl)-, —(CHMe)CH₂O(3,3-oxatenylidenyl)-, —(CH₂)₂(1,1-cycloalkylidenyl)CH₂—, —(CH₂)ₘ— with m=4-6 and the provision that when n=0 then m≠5 and optionally substituted with one or more substituents independently selected from the group consisting of F, OH, and R¹⁰.

In some embodiments, R⁸ can be phenyl or heteroaryl optionally substituted with one or more substituents independently selected from the group consisting of F, Cl, Br, CF₃, CHF₃, CH₂F, C1-4 alkyl, C3-5 cycloalkyl, —OC1-4 alkyl, and —OC3-5 cycloalkyl, wherein C1-4 alkyl, C3-5 cycloalkyl, —OC1-4 alkyl, or —OC3-5 cycloalkyl is optionally substituted with one or more fluorines.

In some embodiments, R⁹ can be selected from the group consisting of H, R⁸, C1-4 alkyl, —OC1-3 alkyl, and —OC3-5 cycloalkyl, wherein C1-4 alkyl, —OC1-3 alkyl, or —OC3-5 cycloalkyl can be optionally substituted substituents selected from the group consisting of F, OH, R⁸, and a combination thereof.

In some embodiments, R¹⁰ can be selected from the group consisting of H, R⁸, C1-4 alkyl, C3-6 alkenyl, C3-6 alkynyl, —OC1-3 alkyl, —OC3-5 cycloalkyl, wherein C1-4 alkyl, C3-6 alkenyl, C3-6 alkynyl, —OC1-3 alkyl, or —OC3-5 cycloalkyl is optionally substituted with substituents selected from the group consisting of F, OH, R⁸, OR⁸, OCH₂R⁸, OCHMeR⁸, and a combination thereof.

In some embodiment, R¹¹ can be selected from the group consisting of H, CO₂H, CO₂R¹⁴, CH₂OH, CH₂OR¹⁴, C1-4 alkyl, C3-6 alkenyl, C3-6 alkynyl, and C3-5 cycloalkyl, wherein C1-4 alkyl, C3-6 alkenyl, C3-6 alkynyl, or C3-5 cycloalkyl is optionally substituted with one or more substituents selected from the group consisting of F, OH, and R⁸.

In some embodiments, R¹² and R¹³ can be independently selected from the group consisting of H, F, CF₃, CHF₂, CH₂F, CN, OH, OR¹⁴, NHC(O)Me, SO₂Me, OSO₂Me, CO₂H, CO₂R¹⁴, CH₂OH, CH₂OR¹⁴, R⁸, and R¹⁴. In some embodiments, R¹² and R¹³ can be optionally connected to form a cyclic structure, in such a case, R¹²-R¹³ is to be selected from the group consisting of: —CH₂OCH₂—, —(CH₂)₂O—, —(CH₂)₃O—, —(CH₂)₃—, —(CH₂)₄—, —CH₂CF₂CH₂—, —CH₂O(CHCF₃)—, —CH₂SO₂(CHCF₃)—, —CH₂(CHCO₂H)CH₂—, —CH₂(CHCO₂R¹⁴)CH₂—, —CH₂(CHCH₂OH)CH₂—, —CH₂(CHCH₂OR¹⁴)CH₂—, —(CHOH)CH₂O—, —(CHOR¹⁴)CH₂O—, —SO₂(CH₂)₂(CHOH)—, —SO₂(CH₂)₂(CHOR¹⁴)—, —SO₂(CH₂)(CHOH)CH₂—, —SO₂(CH₂)(CHOR¹⁴)CH₂—, —CH₂(CHOH)CH₂O—, —CH₂(CHOR¹⁴)CH₂O—, —(CHOH)(CH₂)₂O—(CHOR¹⁴)(CH₂)₂O—, and —CH₂(3,3-oxetanyl)CH₂-.

In some embodiments, R¹⁴ can be selected from the group consisting of C1-4 alkyl, C3-5 cycloalkyl, C2-6 alkenyl, and C2-6 alkynyl, each of which is optionally substituted with one or more substituents selected from F, OH, and R⁸.

In some embodiment, Ar in Formula (I) can be selected from:

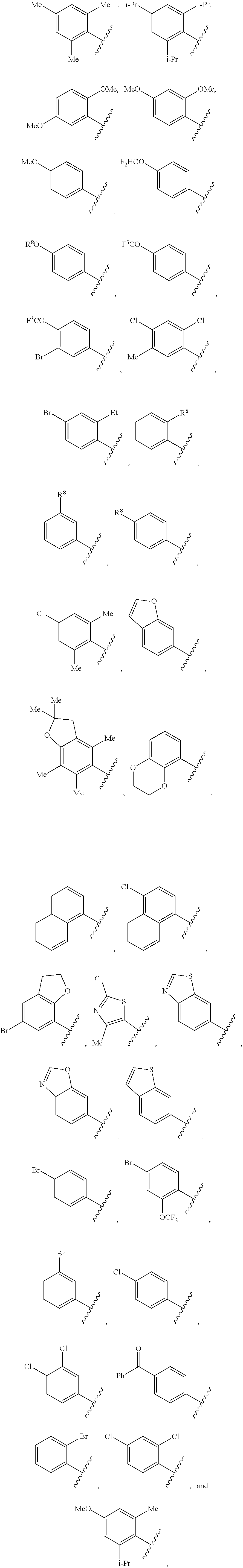

These functional groups for Ar can be optionally further substituted with one or more substituents independently selected from the group consisting of F, Cl, Br, Me, CF₃, Et, i-Pr, cyclopropyl, OMe, OEt, Oi-Pr, —Ocyclopropyl, —OCF₃, —OCHF₂, —OCH₂F, —OCH₂R⁸, —OR⁸ and R⁸.

In some embodiment, when n=0, —NR⁶R⁷ can be selected from:

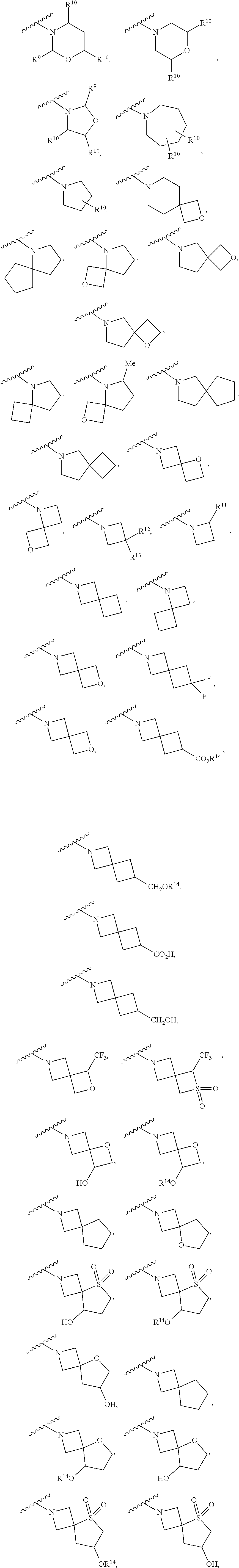

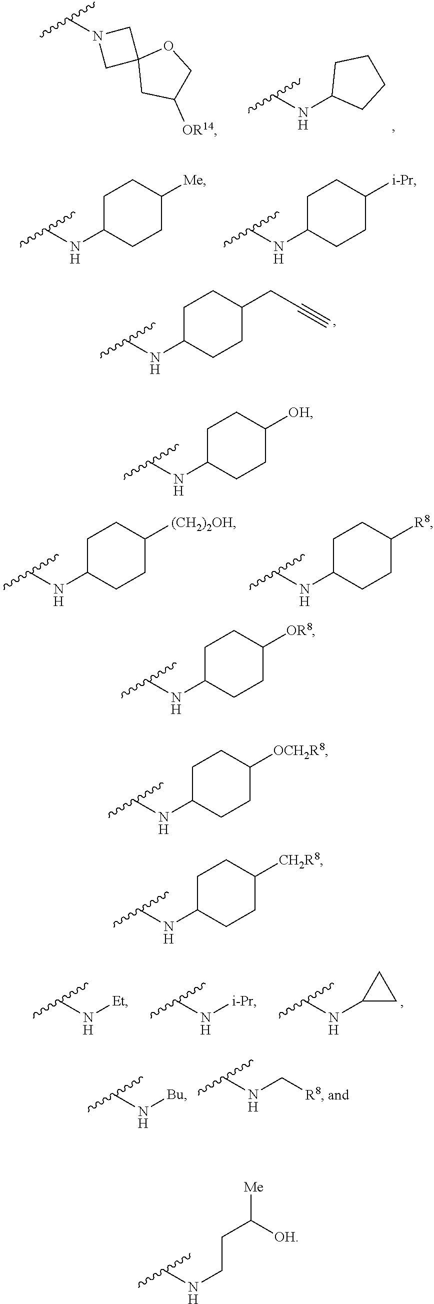

In some embodiment, when n=1, —NR⁶R⁷ can be selected from:

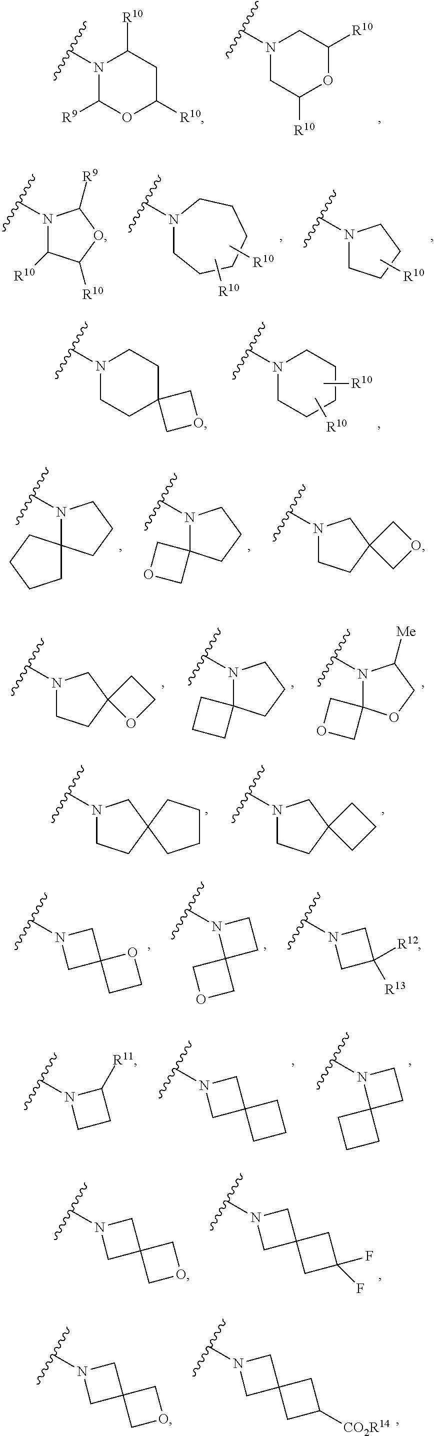

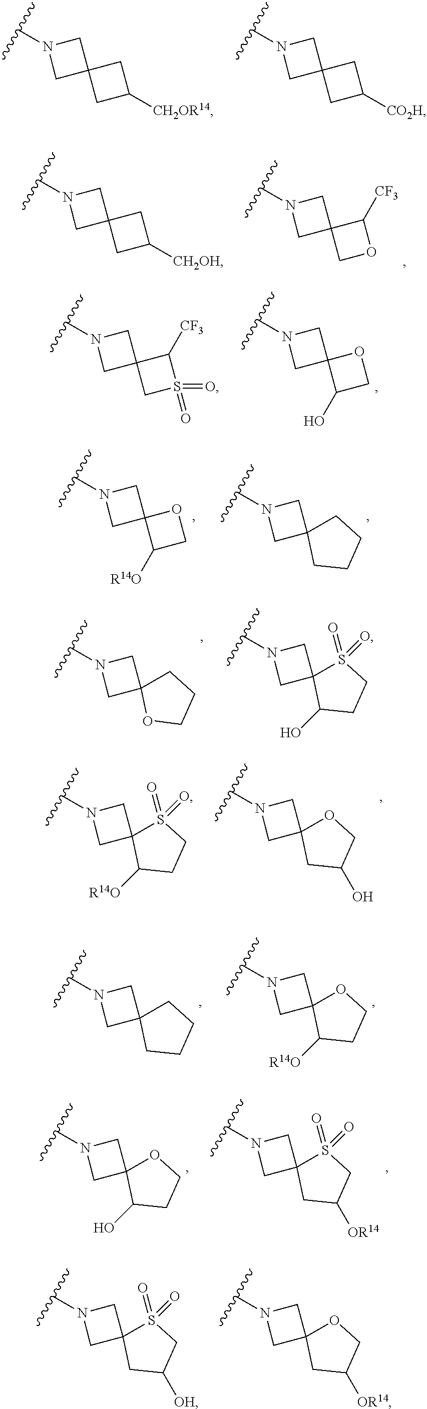

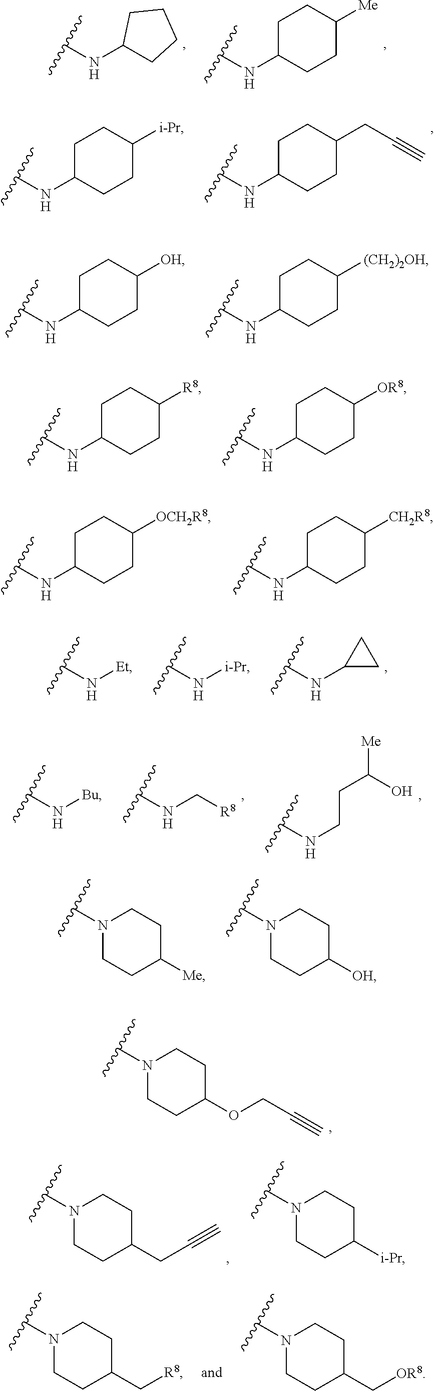

According to one aspect, the instant disclosure provides an EBP-modulating anti cancer compound of Formula (II):

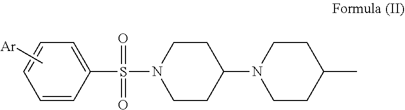

or a pharmaceutically acceptable salt or solvate, a stereoisomer, a diastereoisomer or an enantiomer thereof.

In some embodiments, Ar can be selected from the group consisting of substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl.

The optional substituents can be one or more substituents independently selected from the group consisting of F, Cl, Br, —OCF₃, —OCHF₂, —OCH₂F, —OCH₂R¹, —OCHMeR¹, —OCH(CF₃)R¹, —OR¹, —C(O)R¹, R¹, C1-4 alkyl or C3-5 cycloalkyl optionally substituted with one or more fluorines and/or hydroxy and/or C1-3 alkoxy group, tetrahydropyranyl or tetrahydrofuranyl optionally substituted with one or more fluorines and/or hydroxy and/or C1-3 alkoxy group, C2-6 alkenyl or alkynyl optionally substituted with one or more fluorines and/or hydroxy and/or C1-3 alkoxy group, —OC1-4 alkyl or —OC3-5 cycloalkyl optionally substituted with one or more fluorines and/or hydroxy and/or C1-3 alkoxy group, —OC2-6 alkenyl or alkynyl optionally substituted with one or more fluorines and/or hydroxy and/or C1-3 alkoxy group.

In some embodiments, R¹ can be phenyl or heteroaryl optionally substituted with one or more substituents independently selected from the group consisting of F, Cl, Br, CF₃, CHF₃, CH₂F, C1-4 alkyl or C3-5 cycloalkyl optionally substituted with one or more fluorines, and —OC1-4 alkyl or —OC3-5 cycloalkyl optionally substituted with one or more fluorines.

In some embodiments, Formula (II) does not include compounds with the structure

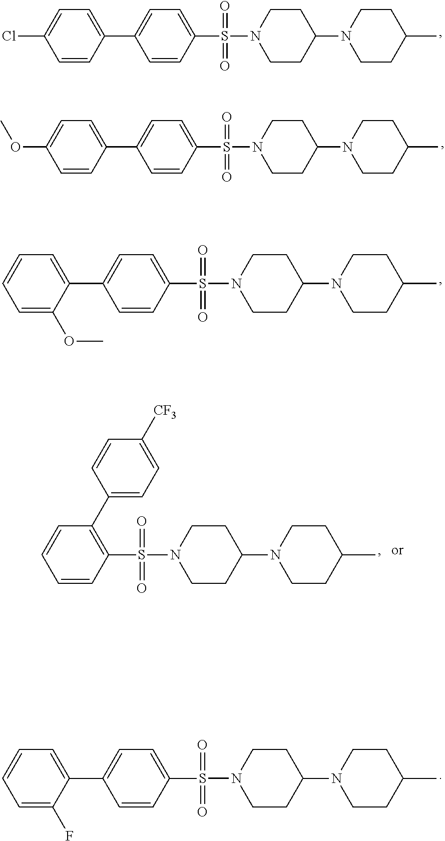

According to another aspect, the instant disclosure provides an EBP-modulating anti-cancer compound of Formula (III):

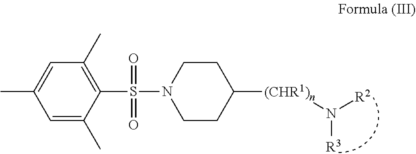

or a pharmaceutically acceptable salt or solvate, a stereoisomer, a diastereoisomer or an enantiomer thereof.

In some embodiments, n is 0 or 1. When n=1, R¹ can be selected from the group consisting of H, methyl, CF₃, CHF₂, and CH₂F.

In some embodiments, R² and R³ are independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F, OH, C1-3 alkoxy group, —CH₂CCH, R⁴, CH₂R⁴, OR⁴, OCH₂R⁴ and OCHMeR⁴. Or wherein R² and R³ are connected to form a nitrogen-containing heterocycle, in such case, R²-R³ is to be selected from the group consisting of —(CHR⁶)CH₂(CHR⁶)O(CHR⁵)—, —(CHR⁵)O(CHR⁶)₂—, —CH₂(CR⁸R⁹)CH₂—, —(CH₂)₂ (CHR⁷)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkylidenyl)-, —(CHMe)CH₂O(3,3-oxatenylidenyl)-, —(CH₂)₂(1,1-cycloalkylidenyl)CH₂—, and —(CH₂)ₘ— with m=4-6.

In some embodiments, R⁴ can be phenyl or heteroaryl optionally substituted with one or more substituents independently selected from the group consisting of F, Cl, Br, CF₃, CHF₃, CH₂F, C1-4 alkyl, C3-5 cycloalkyl optionally substituted with one or more fluorines, —OC1-4 alkyl, and —OC3-5 cycloalkyl optionally substituted with one or more fluorines.

R⁵ is selected from the group consisting of H, R⁴, C1-4 alkyl optionally substituted with one or more substituents selected from the group consisting of F, OH, R⁴, —OC1-3 alkyl, and —OC3-5 cycloalkyl optionally substituted with one or more fluorines.

R⁶ is selected from the group consisting of H, R⁴, C1-4 alkyl, C3-6 alkenyl, and C3-6 alkynyl optionally substituted with one or more substituents selected from the group consisting of F, OH, R⁴, OR⁴, OCH₂R⁴, OCHMeR⁴, —OC1-3 alkyl, and —OC3-5 cycloalkyl optionally substituted with one or more fluorines.

R⁷ can be selected from the group consisting of H, CO₂H, CO₂R¹⁰, CH₂OH, CH₂OR¹⁰, C1-4 alkyl, C3-5 cycloalkyl, C3-6 alkenyl, and C3-6 alkynyl optionally substituted with one or more substituents selected from the group consisting of F, OH, and R⁴.

R⁸ and R⁹ can be independently selected from the group consisting of H, F, CF₃, CHF₂, CH₂F, CN, OH, OR¹⁴, NHC(O)Me, SO₂Me, OSO₂Me, CO₂H, CO₂R¹⁰, CH₂OH, CH₂OR¹⁰, R⁴, and R¹⁰. Or wherein R⁸ and R⁹ are optionally connected to form a ring, in such case, R⁸-R⁹ is to be selected from the group consisting of: —CH₂OCH₂—, —(CH₂)₂O—, —(CH₂)₃O—, —(CH₂)₃—, —(CH₂)₄—, —CH₂CF₂CH₂—, —CH₂O(CHCF₃)—, —CH₂SO₂(CHCF₃)—, —CH₂(CHCO₂H)CH₂—, —CH₂(CHCO₂R¹⁰)CH₂—, —CH₂(CHCH₂OH)CH₂—, —CH₂(CHCH₂OR¹⁰)CH₂—, —(CHOH)CH₂O—, —(CHOR¹⁰)CH₂O—, —SO₂(CH₂)₂(CHOH)—, —SO₂(CH₂)₂(CHOR¹⁰)—, —SO₂(CH₂)(CHOH)CH₂—, —SO₂(CH₂)(CHOR¹⁰)CH₂—, —CH₂(CHOH)CH₂O—, —CH₂(CHOR¹⁰)CH₂O—, —(CHOH)(CH₂)₂O—(CHOR¹⁰)(CH₂)₂O—, and —CH₂(3,3-oxetanyl)CH₂-.

R¹⁰ can be selected from the group consisting of C1-4 alkyl, C3-5 cycloalkyl, C2-6 alkenyl, and C2-6 alkynyl, which are optionally substituted with one or more substituents selected from F, OH, and R⁴.

In some embodiments, the present disclosure provides an EBP-modulating anti cancer compound with the structure of Formula (IV):

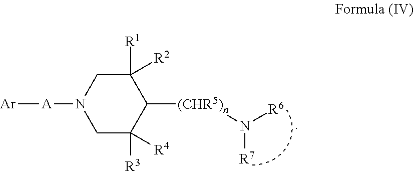

or a pharmaceutically acceptable salt or solvate, a stereoisomer, a diastereoisomer or an enantiomer thereof.

In some embodiment, A can be —NR⁸—SO₂- or —NR⁸—CO—. R¹, R², R³, and R⁴ can be independently selected from the group consisting of H, F, CF₃, CHF₂, CH₂F, and methyl. R⁸ can be selected from the group consisting of H and optionally-substituted C1-C4 alkyl.

In some embodiment, Ar can be selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl.

In some embodiments, the optional substituent for Ar can be selected from the group consisting of F, Cl, Br, —OCF₃, —OCHF₂, —OCH₂F, —OCH₂R⁹, —OCHMeR⁹, —OCH(CF₃)R⁹, —OR⁹, —C(O)R⁹, R⁹, C1-4 alkyl, C3-5 cycloalkyl, C2-6 alkenyl, C2-6 alkynyl, —OC1-4 alkyl, —OC3-5 cycloalkyl, —OC2-6 alkenyl, and —OC2-6 alkynyl.

C1-4 alkyl or C3-5 cycloalkyl can be optionally substituted selected from the group consisting of fluorine, hydroxyl, C1-3 alkoxy group, tetrahydropyranyl optionally substituted with one or more fluorines, hydroxyl, or C1-3 alkoxy group, tetrahydrofuranyl optionally substituted with one or more fluorines, hydroxyl, or C1-3 alkoxy group, and a combination thereof.

C2-6 alkenyl or C2-6 alkynyl can be optionally substituted with fluorine, hydroxyl, C1-3 alkoxy group, or a combination thereof.

—OC1-4 alkyl or —OC3-5 cycloalkyl can be optionally substituted with fluorine, hydroxyl, C1-3 alkoxy group, or a combination thereof.

—OC2-6 alkenyl or —OC2-6 alkynyl can be optionally substituted with fluorine, hydroxyl, C1-3 alkoxy group, or a combination thereof.

In some embodiments, n can be 0 or 1. When n=1, in some embodiment, R⁵ can be selected from the group consisting of H, methyl, CF₃, CHF₂, and CH₂F.

In some embodiments, R⁶ and R⁷ can be independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F or OH; C1-3 alkoxy group; —CH₂CCH; R⁹; CH₂R⁹; OR⁹; OCH₂R⁹; OCHMeR⁹.

In some embodiments, R⁶ and R⁷ can be connected to form a nitrogen-containing heterocycle, in such case, R⁶-R⁷ is to be selected from the group consisting of —(CHR¹¹)CH₂(CHR¹¹)O(CHR¹⁰)—, —(CHR¹⁰)O(CHR¹¹)₂—, —CH₂(CR¹³R¹⁴)CH₂—, —(CH₂)₂(CHR¹²)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkylidenyl)-, —(CHMe)CH₂O(3,3-oxatenylidenyl)-, —(CH₂)₂(1,1-cycloalkylidenyl)CH₂—, —(CH₂)ₘ— with m=4-6 and optionally substituted with one or more substituents independently selected from the group consisting of F, OH, and R¹¹.

In some embodiments, R⁹ can be phenyl or heteroaryl optionally substituted with one or more substituents independently selected from the group consisting of F, Cl, Br, CF₃, CHF₃, CH₂F, C1-4 alkyl, C3-5 cycloalkyl, —OC1-4 alkyl, and —OC3-5 cycloalkyl, wherein C1-4 alkyl, C3-5 cycloalkyl, —OC1-4 alkyl, or —OC3-5 cycloalkyl is optionally substituted with one or more fluorines.

In some embodiments, R¹⁰ can be selected from the group consisting of H, R⁹, C1-4 alkyl, —OC1-3 alkyl, and —OC3-5 cycloalkyl, wherein C1-4 alkyl, —OC1-3 alkyl, or —OC3-5 cycloalkyl can be optionally substituted substituents selected from the group consisting of F, OH, R⁹, and a combination thereof.

In some embodiments, R¹⁰ can be selected from the group consisting of H, R⁹, C1-4 alkyl, C3-6 alkenyl, C3-6 alkynyl, —OC1-3 alkyl, —OC3-5 cycloalkyl, wherein C1-4 alkyl, C3-6 alkenyl, C3-6 alkynyl, —OC1-3 alkyl, or —OC3-5 cycloalkyl is optionally substituted with substituents selected from the group consisting of F, OH, R⁹, OR⁹, OCH₂R⁹, OCHMeR⁹, and a combination thereof.

In some embodiment, R¹² can be selected from the group consisting of H, CO₂H, CO₂R¹⁵, CH₂OH, CH₂OR¹⁵, C1-4 alkyl, C3-6 alkenyl, C3-6 alkynyl, and C3-5 cycloalkyl, wherein C1-4 alkyl, C3-6 alkenyl, C3-6 alkynyl, or C3-5 cycloalkyl is optionally substituted with one or more substituents selected from the group consisting of F, OH, and R⁹.

In some embodiments, R¹³ and R¹⁴ can be independently selected from the group consisting of H, F, CF₃, CHF₂, CH₂F, CN, OH, OR¹⁵, NHC(O)Me, SO₂Me, OSO₂Me, CO₂H, CO₂R¹⁵, CH₂OH, CH₂OR¹⁵, R⁹, and R¹⁵. In some embodiments, R¹³ and R¹⁴ can be optionally connected to form a cyclic structure, in such a case, R¹³-R¹⁴ is to be selected from the group consisting of: —CH₂OCH₂—, —(CH₂)₂O—, —(CH₂)₃O—, —(CH₂)₃—, —(CH₂)₄—, —CH₂CF₂CH₂—, CH₂O(CHCF₃)—, —CH₂SO₂(CHCF₃)—, —CH₂(CHCO₂H)CH₂—, —CH₂(CHCO₂R¹⁵)CH₂—, CH₂(CHCH₂OH)CH₂—, —CH₂(CHCH₂OR¹⁵)CH₂—, —(CHOH)CH₂O—, —(CHOR¹⁵)CH₂O—, SO₂(CH₂)₂(CHOH)—, —SO₂(CH₂)₂(CHOR¹⁵)—, —SO₂(CH₂)(CHOH)CH₂—, —SO₂(CH₂)(CHOR¹⁵)CH₂—, —CH₂(CHOH)CH₂O—, —CH₂(CHOR¹⁵)CH₂O—, —(CHOH)(CH₂)₂O—(CHOR¹⁵)(CH₂)₂O—, and —CH₂(3,3-oxetanyl)CH₂-.

In some embodiments, R¹⁵ can be selected from the group consisting of C1-4 alkyl, C3-5 cycloalkyl, C2-6 alkenyl, and C2-6 alkynyl, each of which is optionally substituted with one or more substituents selected from F, OH, and R⁹.

In some embodiment, Ar in Formula (IV) can be selected from:

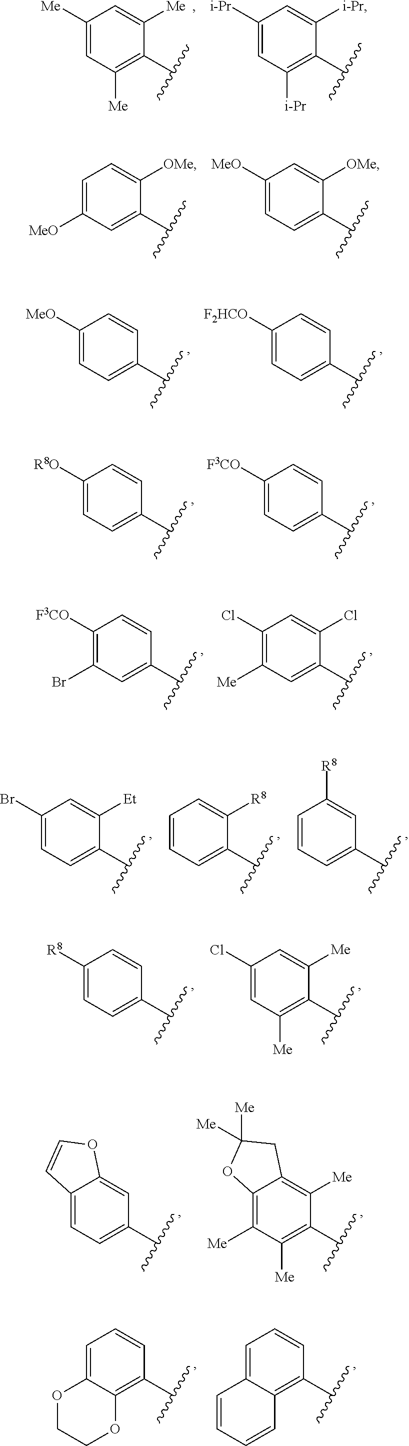

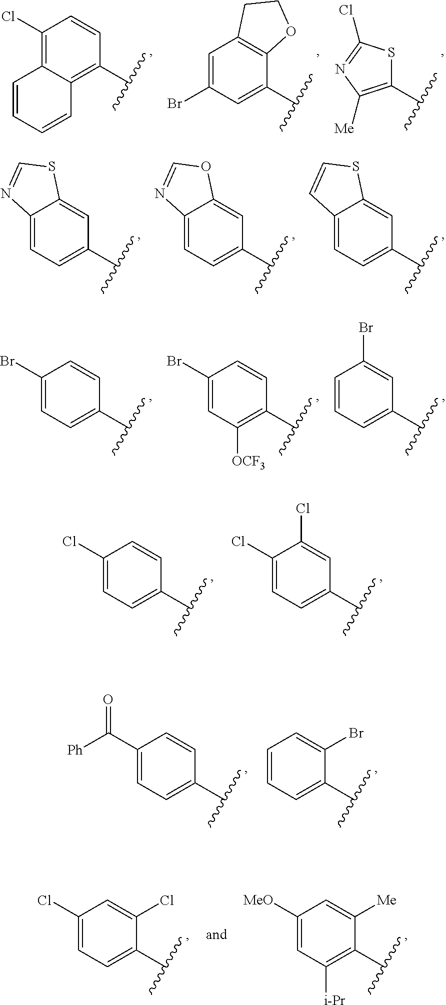

These functional groups for Ar can be optionally further substituted with one or more substituents independently selected from the group consisting of F, Cl, Br, Me, CF₃, Et, i-Pr, cyclopropyl, OMe, OEt, Oi-Pr, —Ocyclopropyl, —OCF₃, —OCHF₂, —OCH₂F, —OCH₂R⁹, —OR⁹ and R⁹.

In some embodiment, when n=0, —NR⁶R⁷ can be selected from:

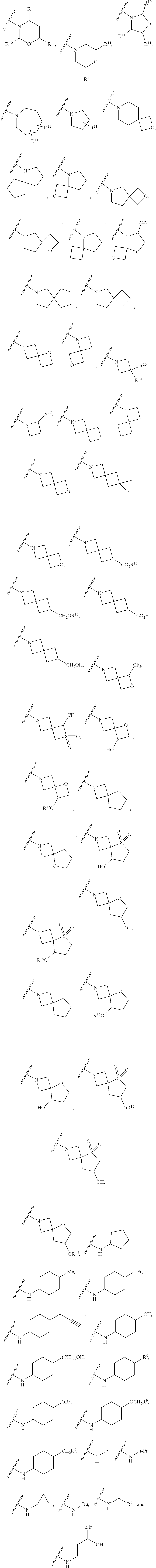

In some embodiment, when n=1, —NR⁶R⁷ can be selected from:

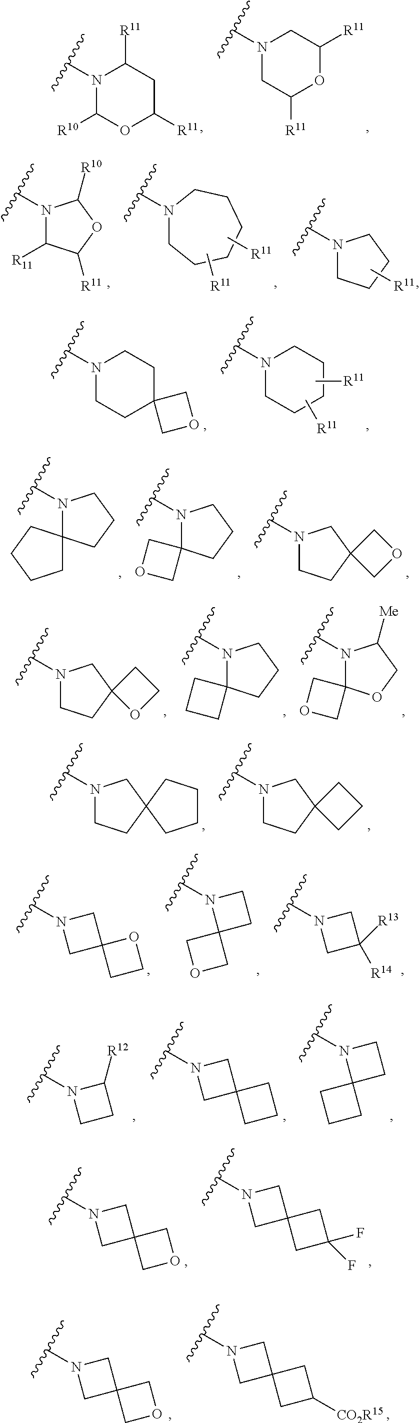

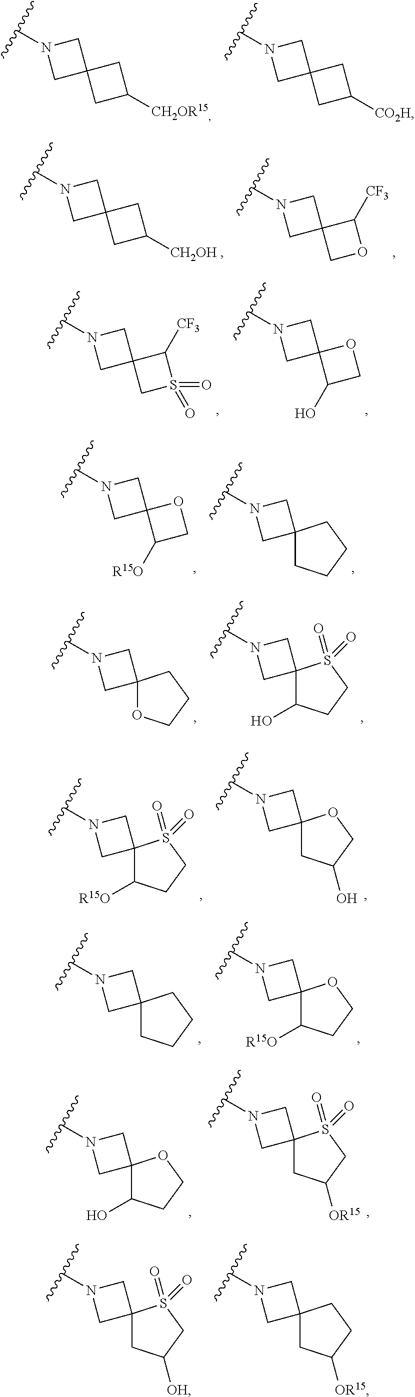

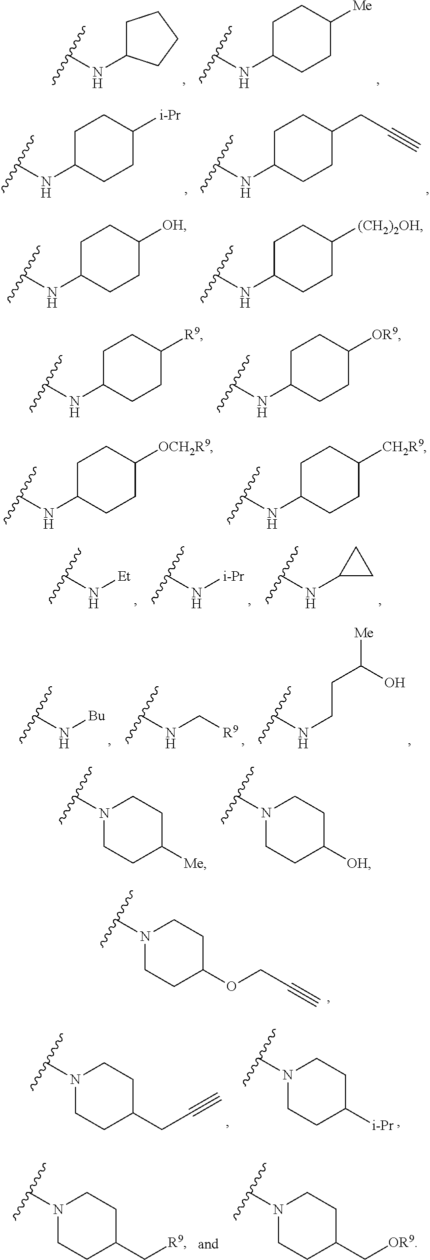

Table 1 below illustrates all the compounds as EBP-modulating anti-cancer compounds synthesized and characterized in the instant disclosure.

TABLE 1
The EBP-modulating anti-cancer compounds in the instant disclosure.

| Compound | |
|---|---|
| No. | Chemical Structure |
| 5 | 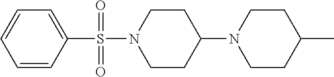 [SMILES: CC1CCN(C2CCN(S(=O)(=O)c3ccccc3)CC2)CC1] |
| 6 | 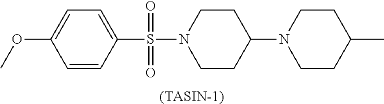 |
| 7 | 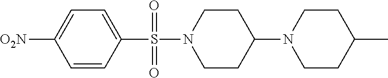 [SMILES: CC1CCN(C2CCN(S(=O)(=O)c3ccc([N+](=O)[O-])cc3)CC2)CC1] |
| 8 | 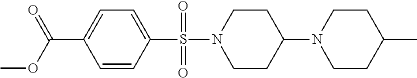 |
| 9 | 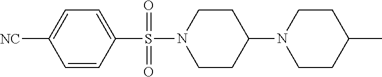 [SMILES: CC1CCN(C2CCN(S(=O)(=O)c3ccc(C#N)cc3)CC2)CC1] |
| 10 | 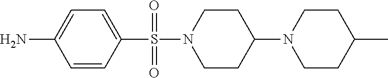 [SMILES: CC1CCN(C2CCN(S(=O)(=O)c3ccc(N)cc3)CC2)CC1] |
| 11 | 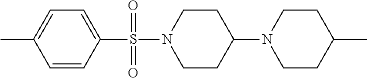 |
| 12 | 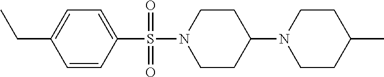 [SMILES: CCc1ccc(S(=O)(=O)N2CCC(N3CCC(C)CC3)CC2)cc1] |
| 13 | 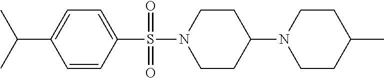 [SMILES: CC1CCN(C2CCN(S(=O)(=O)c3ccc(C(C)C)cc3)CC2)CC1] |
| 14 | 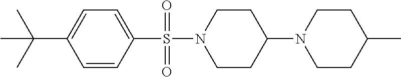 [SMILES: CC1CCN(C2CCN(S(=O)(=O)c3ccc(C(C)(C)C)cc3)CC2)CC1] |
| 15 | 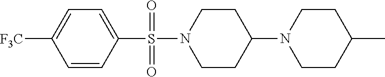 [SMILES: CC1CCN(C2CCN(S(=O)(=O)c3ccc(C(F)(F)F)cc3)CC2)CC1] |
| 16 | 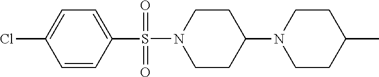 [SMILES: CC1CCN(C2CCN(S(=O)(=O)c3ccc(Cl)cc3)CC2)CC1] |
| 17 | 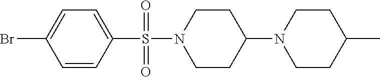 [SMILES: CC1CCN(C2CCN(S(=O)(=O)c3ccc(Br)cc3)CC2)CC1] |
| 18 | 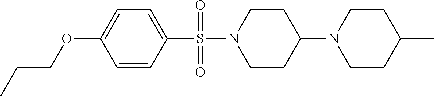 [SMILES: CCCOc1ccc(S(=O)(=O)N2CCC(N3CCC(C)CC3)CC2)cc1] |
| 19 | 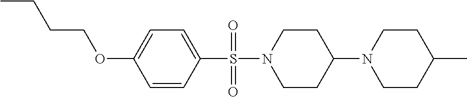 [SMILES: CCCCOc1ccc(S(=O)(=O)N2CCC(N3CCC(C)CC3)CC2)cc1] |
| 20 | 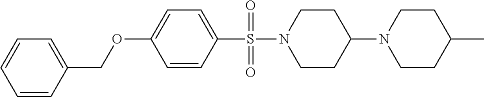 [SMILES: CC1CCN(C2CCN(S(=O)(=O)c3ccc(OCc4ccccc4)cc3)CC2)CC1] |
| 21 | 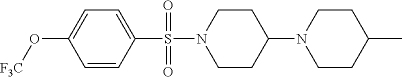 [SMILES: CC1CCN(C2CCN(S(=O)(=O)c3ccc(OC(F)(F)F)cc3)CC2)CC1] |
| 22 | 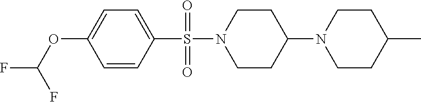 [SMILES: CC1CCN(C2CCN(S(=O)(=O)c3ccc(OC(F)F)cc3)CC2)CC1] |
| 23 | 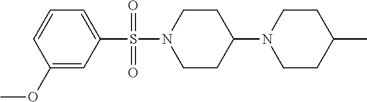 |
| 24 | 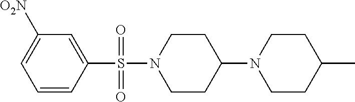 [SMILES: CC1CCN(C2CCN(S(=O)(=O)c3cccc([N+](=O)[O-])c3)CC2)CC1] |
| 25 | 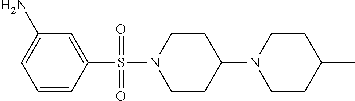 [SMILES: CC1CCN(C2CCN(S(=O)(=O)c3cccc(N)c3)CC2)CC1] |
| 26 | 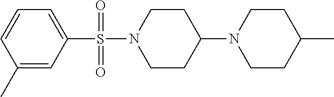 [SMILES: Cc1cccc(S(=O)(=O)N2CCC(N3CCC(C)CC3)CC2)c1] |
| 27 | 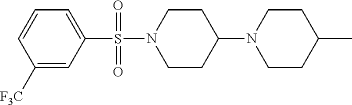 [SMILES: CC1CCN(C2CCN(S(=O)(=O)c3cccc(C(F)(F)F)c3)CC2)CC1] |
| 28 | 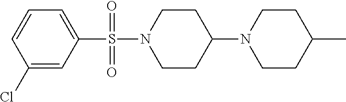 [SMILES: CC1CCN(C2CCN(S(=O)(=O)c3cccc(Cl)c3)CC2)CC1] |
| 29 | 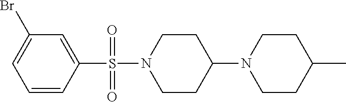 [SMILES: CC1CCN(C2CCN(S(=O)(=O)c3cccc(Br)c3)CC2)CC1] |
| 30 | 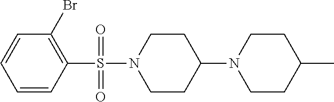 [SMILES: CC1CCN(C2CCN(S(=O)(=O)c3ccccc3Br)CC2)CC1] |
| 31 | 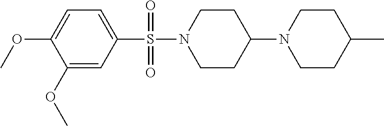 |
| 32 | 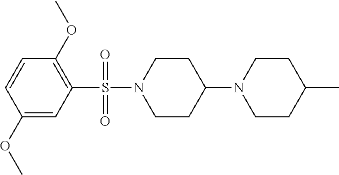 [SMILES: COc1ccc(OC)c(S(=O)(=O)N2CCC(N3CCC(C)CC3)CC2)c1] |
| 33 | 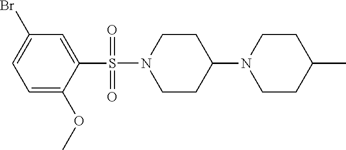 [SMILES: COc1ccc(Br)cc1S(=O)(=O)N1CCC(N2CCC(C)CC2)CC1] |
| 34 | 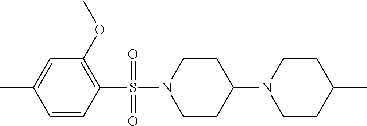 |
| 35 | 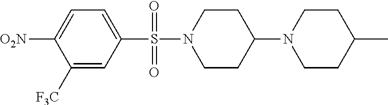 [SMILES: CC1CCN(C2CCN(S(=O)(=O)c3ccc([N+](=O)[O-])c(C(F)(F)F)c3)CC2)CC1] |
| 36 | 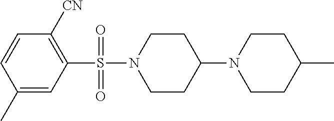 [SMILES: Cc1ccc(C#N)c(S(=O)(=O)N2CCC(N3CCC(C)CC3)CC2)c1] |
| 37 | 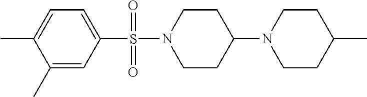 |
| 38 | 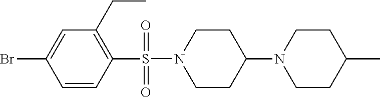 [SMILES: CCc1cc(Br)ccc1S(=O)(=O)N1CCC(N2CCC(C)CC2)CC1] |
| 39 | 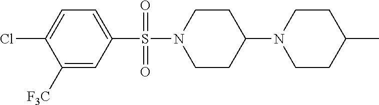 [SMILES: CC1CCN(C2CCN(S(=O)(=O)c3ccc(Cl)c(C(F)(F)F)c3)CC2)CC1] |
| 40 | 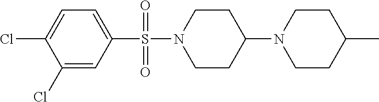 [SMILES: CC1CCN(C2CCN(S(=O)(=O)c3ccc(Cl)c(Cl)c3)CC2)CC1] |
| 41 | 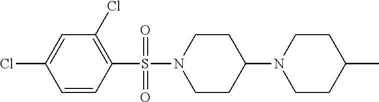 [SMILES: CC1CCN(C2CCN(S(=O)(=O)c3ccc(Cl)cc3Cl)CC2)CC1] |
| 42 | 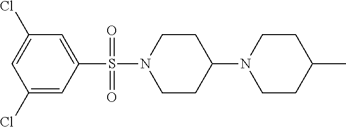 [SMILES: CC1CCN(C2CCN(S(=O)(=O)c3cc(Cl)cc(Cl)c3)CC2)CC1] |
| 43 | 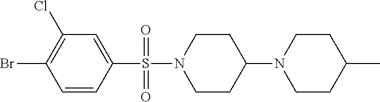 [SMILES: CC1CCN(C2CCN(S(=O)(=O)c3ccc(Br)c(Cl)c3)CC2)CC1] |
| 44 | 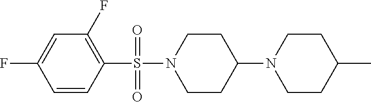 [SMILES: CC1CCN(C2CCN(S(=O)(=O)c3ccc(F)cc3F)CC2)CC1] |
| 45 | 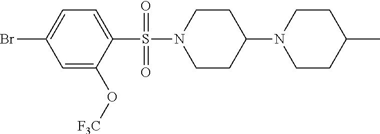 [SMILES: CC1CCN(C2CCN(S(=O)(=O)c3ccc(Br)cc3OC(F)(F)F)CC2)CC1] |
| 46 | 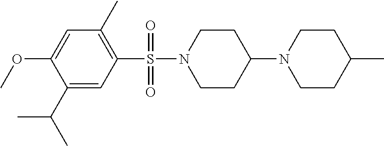 |
| 47 | 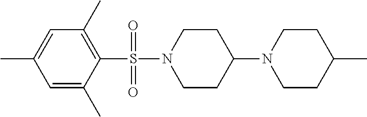 |
| 48 | 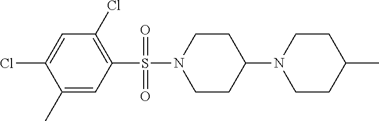 [SMILES: Cc1cc(S(=O)(=O)N2CCC(N3CCC(C)CC3)CC2)c(Cl)cc1Cl] |
| 49 | 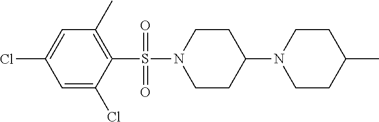 [SMILES: Cc1cc(Cl)cc(Cl)c1S(=O)(=O)N1CCC(N2CCC(C)CC2)CC1] |
| 50 | 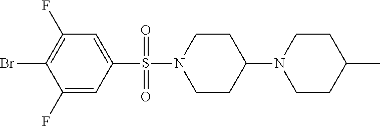 [SMILES: CC1CCN(C2CCN(S(=O)(=O)c3cc(F)c(Br)c(F)c3)CC2)CC1] |
| 51 | 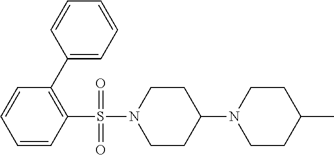 [SMILES: CC1CCN(C2CCN(S(=O)(=O)c3ccccc3-c3ccccc3)CC2)CC1] |
| 52 | 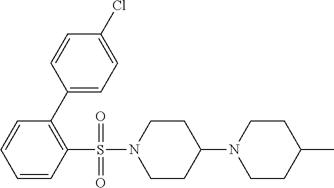 [SMILES: CC1CCN(C2CCN(S(=O)(=O)c3ccccc3-c3ccc(Cl)cc3)CC2)CC1] |
| 53 | 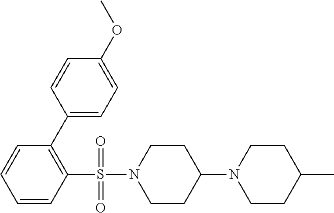 [SMILES: COc1ccc(-c2ccccc2S(=O)(=O)N2CCC(N3CCC(C)CC3)CC2)cc1] |
| 54 | 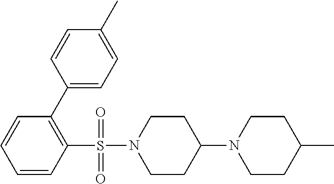 [SMILES: Cc1ccc(-c2ccccc2S(=O)(=O)N2CCC(N3CCC(C)CC3)CC2)cc1] |
| 55 | 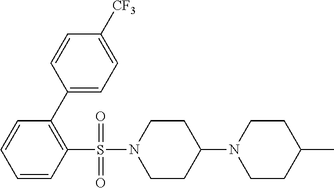 [SMILES: CC1CCN(C2CCN(S(=O)(=O)c3ccccc3-c3ccc(C(F)(F)F)cc3)CC2)CC1] |
| 56 | 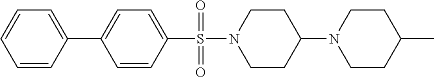 [SMILES: CC1CCN(C2CCN(S(=O)(=O)c3ccc(-c4ccccc4)cc3)CC2)CC1] |
| 57 | 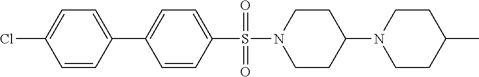 [SMILES: CC1CCN(C2CCN(S(=O)(=O)c3ccc(-c4ccc(Cl)cc4)cc3)CC2)CC1] |
| 58 | 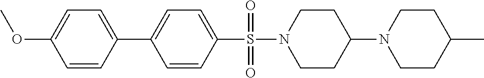 |
| 59 | 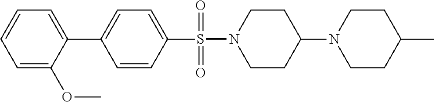 [SMILES: COc1ccccc1-c1ccc(S(=O)(=O)N2CCC(N3CCC(C)CC3)CC2)cc1] |
| 60 | 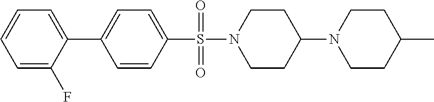 [SMILES: CC1CCN(C2CCN(S(=O)(=O)c3ccc(-c4ccccc4F)cc3)CC2)CC1] |
| 61 | 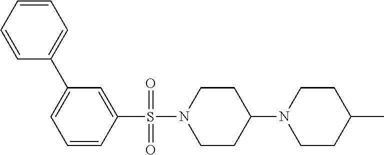 [SMILES: CC1CCN(C2CCN(S(=O)(=O)c3cccc(-c4ccccc4)c3)CC2)CC1] |
| 62 | 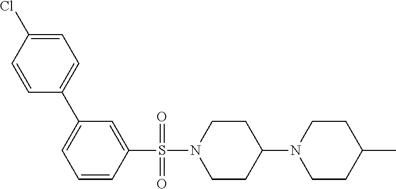 [SMILES: CC1CCN(C2CCN(S(=O)(=O)c3cccc(-c4ccc(Cl)cc4)c3)CC2)CC1] |
| 63 | 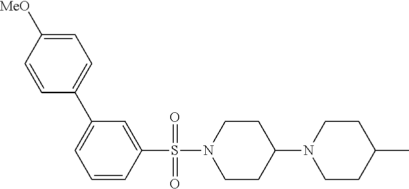 [SMILES: COc1ccc(-c2cccc(S(=O)(=O)N3CCC(N4CCC(C)CC4)CC3)c2)cc1] |
| 64 | 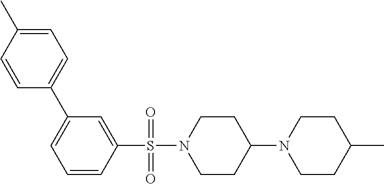 [SMILES: Cc1ccc(-c2cccc(S(=O)(=O)N3CCC(N4CCC(C)CC4)CC3)c2)cc1] |
| 65 | 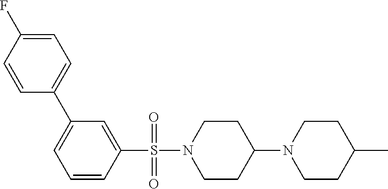 [SMILES: CC1CCN(C2CCN(S(=O)(=O)c3cccc(-c4ccc(F)cc4)c3)CC2)CC1] |
| 66 | 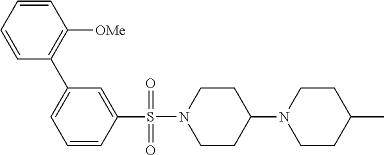 [SMILES: COc1ccccc1-c1cccc(S(=O)(=O)N2CCC(N3CCC(C)CC3)CC2)c1] |
| 67 | 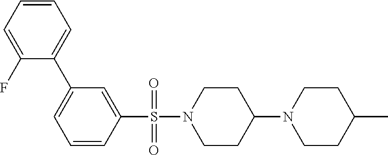 [SMILES: CC1CCN(C2CCN(S(=O)(=O)c3cccc(-c4ccccc4F)c3)CC2)CC1] |
| 68 | 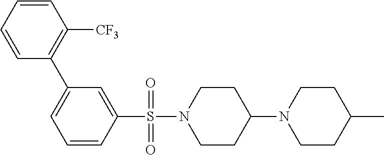 [SMILES: CC1CCN(C2CCN(S(=O)(=O)c3cccc(-c4ccccc4C(F)(F)F)c3)CC2)CC1] |
| 69 | 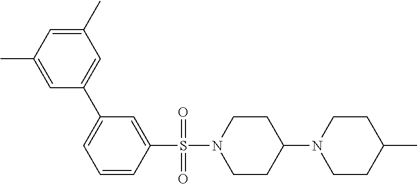 |
| 70 | 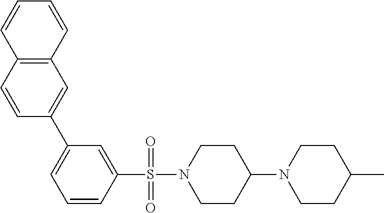 [SMILES: CC1CCN(C2CCN(S(=O)(=O)c3cccc(-c4ccc5ccccc5c4)c3)CC2)CC1] |
| 71 | 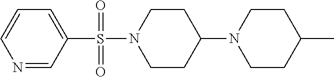 [SMILES: CC1CCN(C2CCN(S(=O)(=O)c3cccnc3)CC2)CC1] |
| 72 | 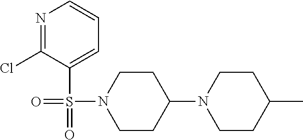 [SMILES: CC1CCN(C2CCN(S(=O)(=O)c3cccnc3Cl)CC2)CC1] |
| 73 | 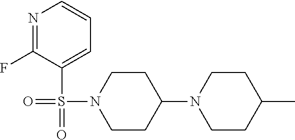 [SMILES: CC1CCN(C2CCN(S(=O)(=O)c3cccnc3F)CC2)CC1] |
| 74 | 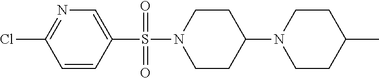 [SMILES: CC1CCN(C2CCN(S(=O)(=O)c3ccc(Cl)nc3)CC2)CC1] |
| 75 | 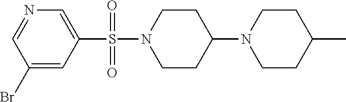 [SMILES: CC1CCN(C2CCN(S(=O)(=O)c3cncc(Br)c3)CC2)CC1] |
| 76 | 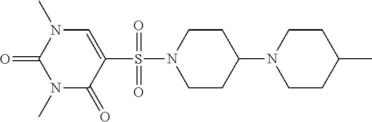 [SMILES: CC1CCN(C2CCN(S(=O)(=O)c3cn(C)c(=O)n(C)c3=O)CC2)CC1] |
| 77 | 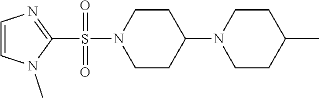 [SMILES: CC1CCN(C2CCN(S(=O)(=O)c3nccn3C)CC2)CC1] |
| 78 | 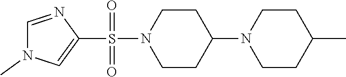 |
| 79 | 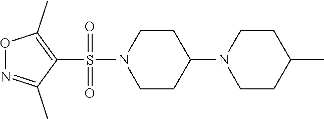 [SMILES: Cc1noc(C)c1S(=O)(=O)N1CCC(N2CCC(C)CC2)CC1] |
| 80 | 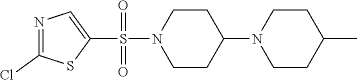 [SMILES: CC1CCN(C2CCN(S(=O)(=O)c3cnc(Cl)s3)CC2)CC1] |
| 81 | 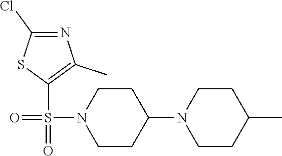 [SMILES: Cc1nc(Cl)sc1S(=O)(=O)N1CCC(N2CCC(C)CC2)CC1] |
| 82 | 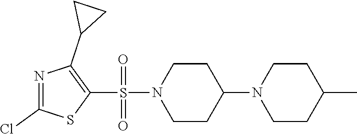 [SMILES: CC1CCN(C2CCN(S(=O)(=O)c3sc(Cl)nc3C3CC3)CC2)CC1] |
| 83 | 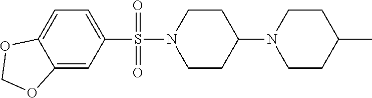 [SMILES: CC1CCN(C2CCN(S(=O)(=O)c3ccc4c(c3)OCO4)CC2)CC1] |
| 84 | 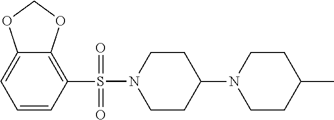 [SMILES: CC1CCN(C2CCN(S(=O)(=O)c3cccc4c3OCO4)CC2)CC1] |
| 85 | 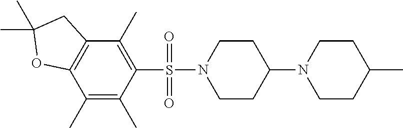 |
| 86 | 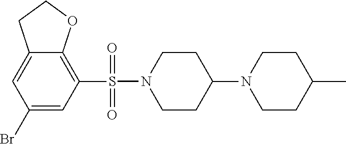 [SMILES: CC1CCN(C2CCN(S(=O)(=O)c3cc(Br)cc4c3OCC4)CC2)CC1] |
| 87 | 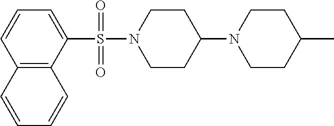 [SMILES: CC1CCN(C2CCN(S(=O)(=O)c3cccc4ccccc34)CC2)CC1] |
| 88 | 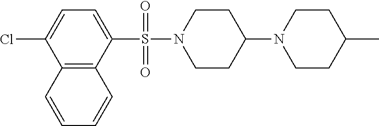 [SMILES: CC1CCN(C2CCN(S(=O)(=O)c3ccc(Cl)c4ccccc34)CC2)CC1] |
| 89 | 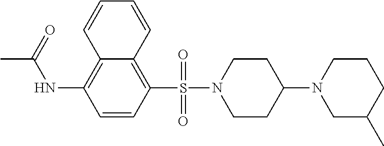 [SMILES: CC(=O)Nc1ccc(S(=O)(=O)N2CCC(N3CCCC(C)C3)CC2)c2ccccc12] |
| 90 | 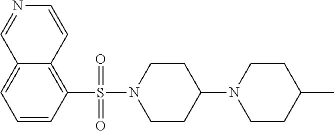 [SMILES: CC1CCN(C2CCN(S(=O)(=O)c3cccc4cnccc34)CC2)CC1] |
| 91 | 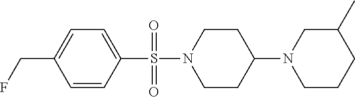 [SMILES: CC1CCCN(C2CCN(S(=O)(=O)c3ccc(CF)cc3)CC2)C1] |
| 92 | 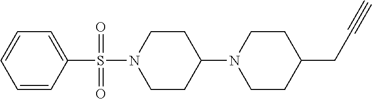 [SMILES: C#CCC1CCN(C2CCN(S(=O)(=O)c3ccccc3)CC2)CC1] |
| 93 | 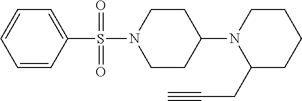 [SMILES: C#CCC1CCCCN1C1CCN(S(=O)(=O)c2ccccc2)CC1] |
| 94 | 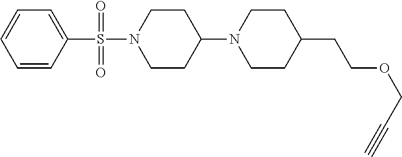 [SMILES: C#CCOCCC1CCN(C2CCN(S(=O)(=O)c3ccccc3)CC2)CC1] |
| 95 | 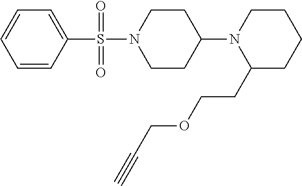 [SMILES: C#CCOCCC1CCCCN1C1CCN(S(=O)(=O)c2ccccc2)CC1] |
| 96 | 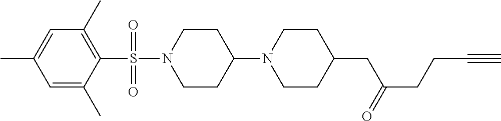 [SMILES: C#CCCC(=O)CC1CCN(C2CCN(S(=O)(=O)c3c(C)cc(C)cc3C)CC2)CC1] |
| 97 | 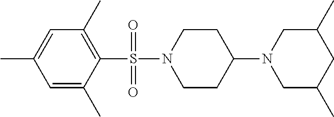 |
| 98 | 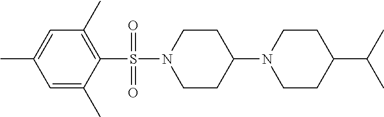 [SMILES: Cc1cc(C)c(S(=O)(=O)N2CCC(N3CCC(C(C)C)CC3)CC2)c(C)c1] |
| 99 | 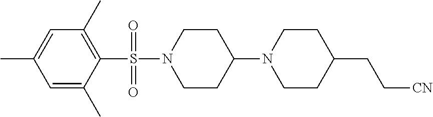 [SMILES: Cc1cc(C)c(S(=O)(=O)N2CCC(N3CCC(CCC#N)CC3)CC2)c(C)c1] |
| 100 | 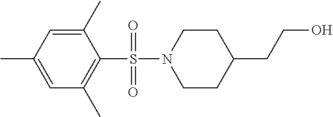 [SMILES: Cc1cc(C)c(S(=O)(=O)N2CCC(CCO)CC2)c(C)c1] |
| 101 | 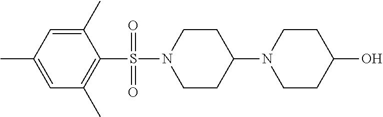 [SMILES: Cc1cc(C)c(S(=O)(=O)N2CCC(N3CCC(O)CC3)CC2)c(C)c1] |
| 102 | 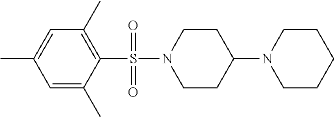 [SMILES: Cc1cc(C)c(S(=O)(=O)N2CCC(N3CCCCC3)CC2)c(C)c1] |
| 103 | 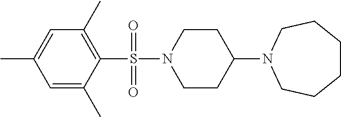 [SMILES: Cc1cc(C)c(S(=O)(=O)N2CCC(N3CCCCCC3)CC2)c(C)c1] |
| 104 | 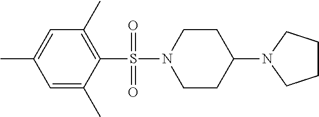 [SMILES: Cc1cc(C)c(S(=O)(=O)N2CCC(N3CCCC3)CC2)c(C)c1] |
| 105 | 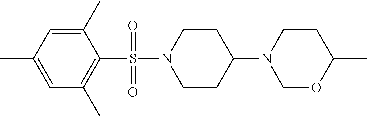 |
| 106 | 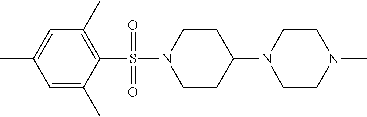 |
| 107 | 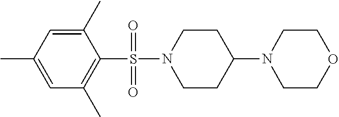 [SMILES: Cc1cc(C)c(S(=O)(=O)N2CCC(N3CCOCC3)CC2)c(C)c1] |
| 108 | 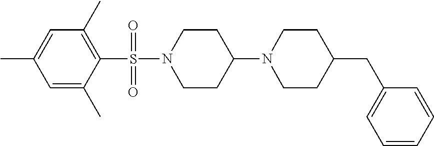 [SMILES: Cc1cc(C)c(S(=O)(=O)N2CCC(N3CCC(Cc4ccccc4)CC3)CC2)c(C)c1] |
| 109 | 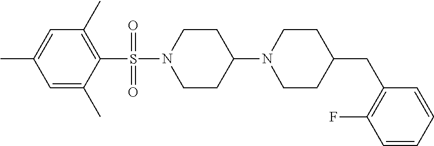 [SMILES: Cc1cc(C)c(S(=O)(=O)N2CCC(N3CCC(Cc4ccccc4F)CC3)CC2)c(C)c1] |
| 110 | 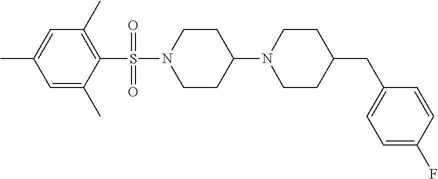 [SMILES: Cc1cc(C)c(S(=O)(=O)N2CCC(N3CCC(Cc4ccc(F)cc4)CC3)CC2)c(C)c1] |
| 111 | 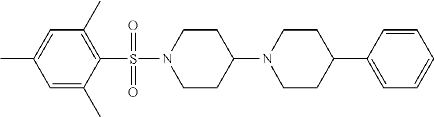 [SMILES: Cc1cc(C)c(S(=O)(=O)N2CCC(N3CCC(c4ccccc4)CC3)CC2)c(C)c1] |
| 112 | 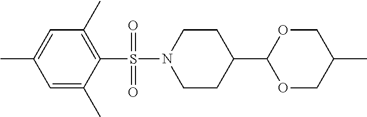 |
| 113 | 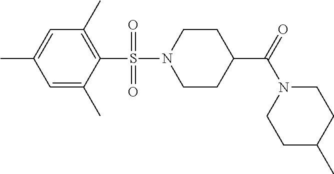 |
| 114 | 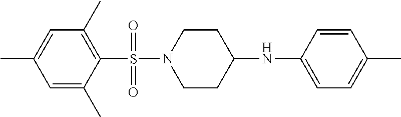 |
| 115 | 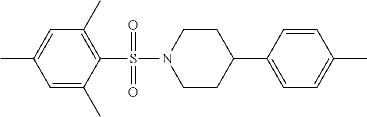 |
| 116 | 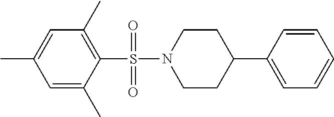 [SMILES: Cc1cc(C)c(S(=O)(=O)N2CCC(c3ccccc3)CC2)c(C)c1] |
| 117 | 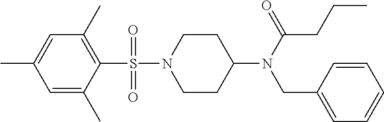 [SMILES: CCCC(=O)N(Cc1ccccc1)C1CCN(S(=O)(=O)c2c(C)cc(C)cc2C)CC1] |
| 118 | 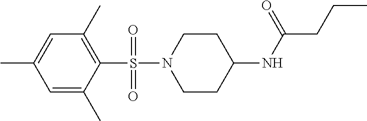 [SMILES: CCCC(=O)NC1CCN(S(=O)(=O)c2c(C)cc(C)cc2C)CC1] |
| 119 | 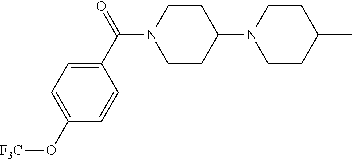 [SMILES: CC1CCN(C2CCN(C(=O)c3ccc(OC(F)(F)F)cc3)CC2)CC1] |
| 120 | 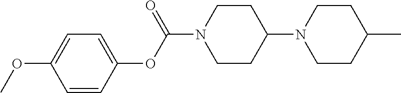 |
| 121 | 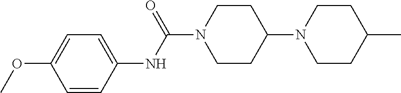 |
| 122 | 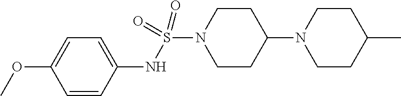 |
| 123 | 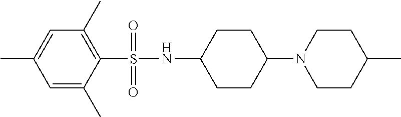 |
| 124 | 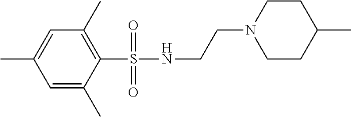 |
| 125 | 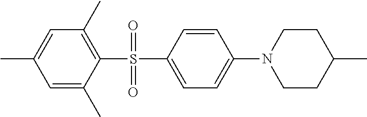 |
| 126 | 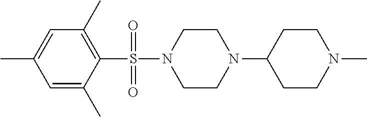 |
| 127 | 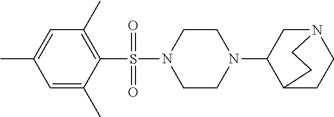 [SMILES: Cc1cc(C)c(S(=O)(=O)N2CCN(C3CN4CCC3CC4)CC2)c(C)c1] |
| 128 | 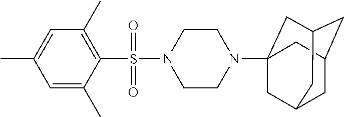 [SMILES: Cc1cc(C)c(S(=O)(=O)N2CCN(C34CC5CC(CC(C5)C3)C4)CC2)c(C)c1] |
| 129 | 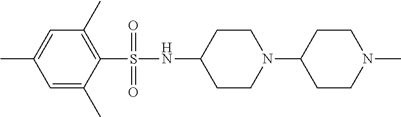 |
| 130 | 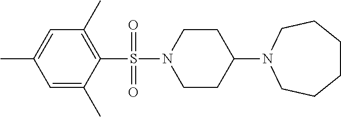 [SMILES: Cc1cc(C)c(S(=O)(=O)N2CCC(N3CCCCCC3)CC2)c(C)c1] |
| 131 | 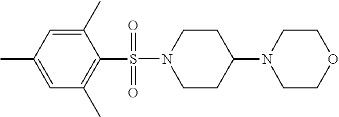 [SMILES: Cc1cc(C)c(S(=O)(=O)N2CCC(N3CCOCC3)CC2)c(C)c1] |
| 132 | 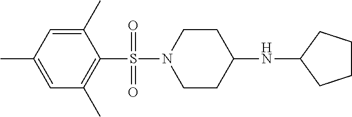 [SMILES: Cc1cc(C)c(S(=O)(=O)N2CCC(NC3CCCC3)CC2)c(C)c1] |
| 133 | 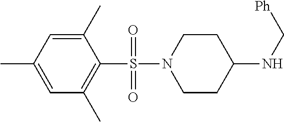 [SMILES: Cc1cc(C)c(S(=O)(=O)N2CCC(NCc3ccccc3)CC2)c(C)c1] |
| 134 | 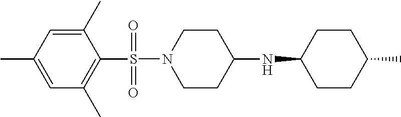 |
| 135 | 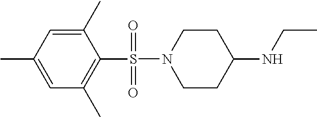 [SMILES: CCNC1CCN(S(=O)(=O)c2c(C)cc(C)cc2C)CC1] |
| 136 | 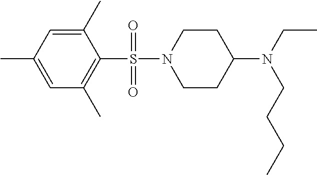 [SMILES: CCCCN(CC)C1CCN(S(=O)(=O)c2c(C)cc(C)cc2C)CC1] |
| 137 | 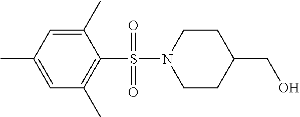 [SMILES: Cc1cc(C)c(S(=O)(=O)N2CCC(CO)CC2)c(C)c1] |
| 138 | 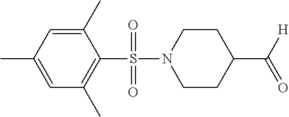 [SMILES: Cc1cc(C)c(S(=O)(=O)N2CCC(C=O)CC2)c(C)c1] |
| 139 | 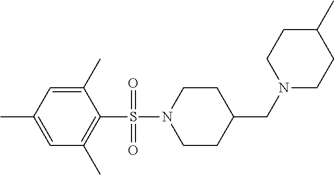 |
| 140 | 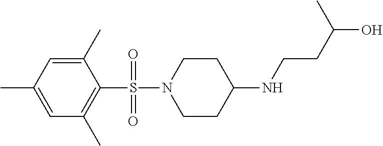 [SMILES: Cc1cc(C)c(S(=O)(=O)N2CCC(NCCC(C)O)CC2)c(C)c1] |
| 141 | 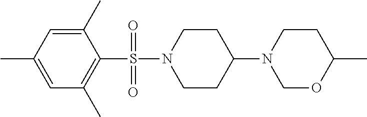 |
| 142 | 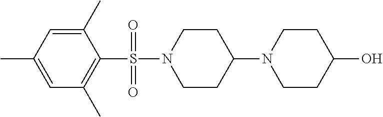 [SMILES: Cc1cc(C)c(S(=O)(=O)N2CCC(N3CCC(O)CC3)CC2)c(C)c1] |
| 143 | 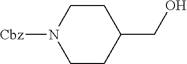 [SMILES: O=C(OCc1ccccc1)N1CCC(CO)CC1] |
| 144 | 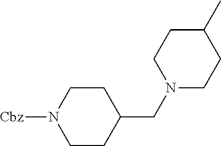 [SMILES: CC1CCN(CC2CCN(C(=O)OCc3ccccc3)CC2)CC1] |
| 145 | 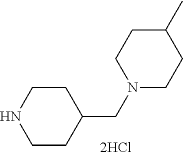 |
| 146 | 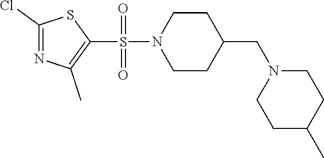 [SMILES: Cc1nc(Cl)sc1S(=O)(=O)N1CCC(CN2CCC(C)CC2)CC1] |
| 147 | 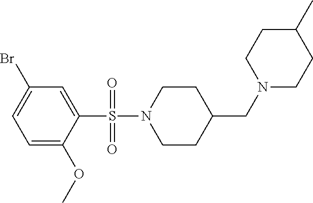 [SMILES: COc1ccc(Br)cc1S(=O)(=O)N1CCC(CN2CCC(C)CC2)CC1] |
| 148 | 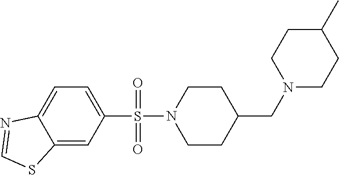 [SMILES: CC1CCN(CC2CCN(S(=O)(=O)c3ccc4ncsc4c3)CC2)CC1] |
| 149 | 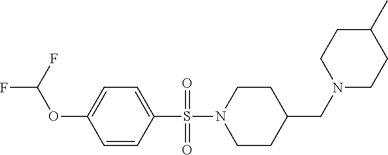 [SMILES: CC1CCN(CC2CCN(S(=O)(=O)c3ccc(OC(F)F)cc3)CC2)CC1] |
| 150 | 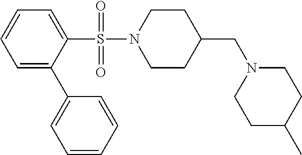 [SMILES: CC1CCN(CC2CCN(S(=O)(=O)c3ccccc3-c3ccccc3)CC2)CC1] |
| 151 | 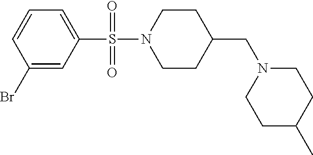 [SMILES: CC1CCN(CC2CCN(S(=O)(=O)c3cccc(Br)c3)CC2)CC1] |
| 152 | 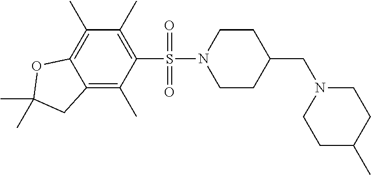 |
| 153 | 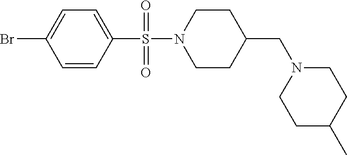 [SMILES: CC1CCN(CC2CCN(S(=O)(=O)c3ccc(Br)cc3)CC2)CC1] |
| 154 | 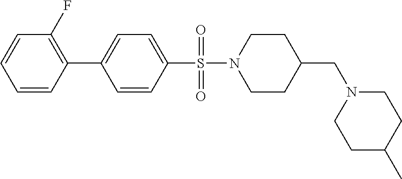 [SMILES: CC1CCN(CC2CCN(S(=O)(=O)c3ccc(-c4ccccc4F)cc3)CC2)CC1] |
| 155 | 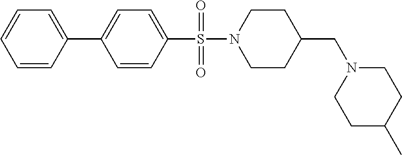 [SMILES: CC1CCN(CC2CCN(S(=O)(=O)c3ccc(-c4ccccc4)cc3)CC2)CC1] |
| 156 | 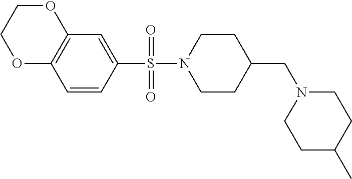 [SMILES: CC1CCN(CC2CCN(S(=O)(=O)c3ccc4c(c3)OCCO4)CC2)CC1] |
| 157 | 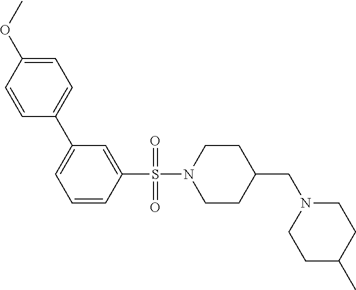 [SMILES: COc1ccc(-c2cccc(S(=O)(=O)N3CCC(CN4CCC(C)CC4)CC3)c2)cc1] |
| 158 | 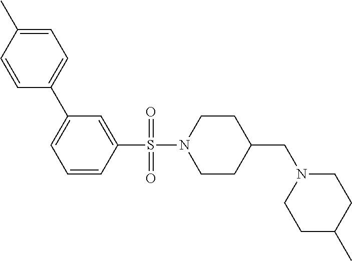 [SMILES: Cc1ccc(-c2cccc(S(=O)(=O)N3CCC(CN4CCC(C)CC4)CC3)c2)cc1] |
| 159 | 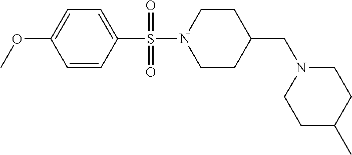 |
| 160 | 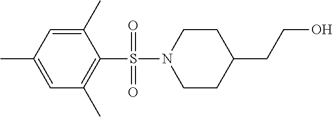 [SMILES: Cc1cc(C)c(S(=O)(=O)N2CCC(CCO)CC2)c(C)c1] |
| 161 | 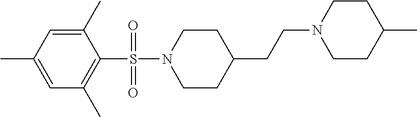 |
| 162 | 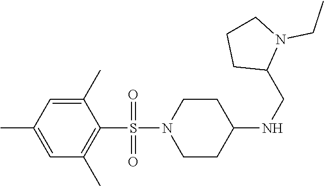 [SMILES: CCN1CCCC1CNC1CCN(S(=O)(=O)c2c(C)cc(C)cc2C)CC1] |
| 163 | 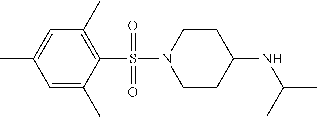 [SMILES: Cc1cc(C)c(S(=O)(=O)N2CCC(NC(C)C)CC2)c(C)c1] |
| 164 | 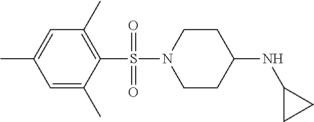 [SMILES: Cc1cc(C)c(S(=O)(=O)N2CCC(NC3CC3)CC2)c(C)c1] |
| 165 | 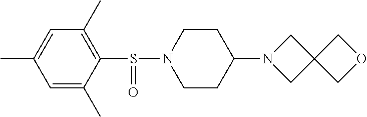 [SMILES: Cc1cc(C)c(S(=O)N2CCC(N3CC4(COC4)C3)CC2)c(C)c1] |
| 166 | 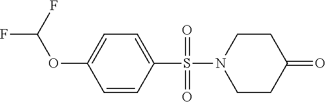 [SMILES: O=C1CCN(S(=O)(=O)c2ccc(OC(F)F)cc2)CC1] |
| 167 | 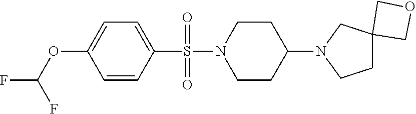 [SMILES: O=S(=O)(c1ccc(OC(F)F)cc1)N1CCC(N2CCC3(COC3)C2)CC1] |
| 168 | 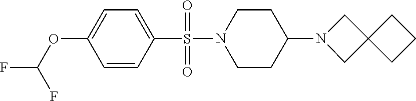 [SMILES: O=S(=O)(c1ccc(OC(F)F)cc1)N1CCC(N2CC3(CCC3)C2)CC1] |
| 169 | 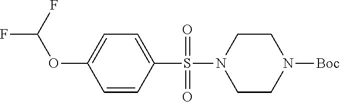 [SMILES: CC(C)(C)OC(=O)N1CCN(S(=O)(=O)c2ccc(OC(F)F)cc2)CC1] |
| 170 | 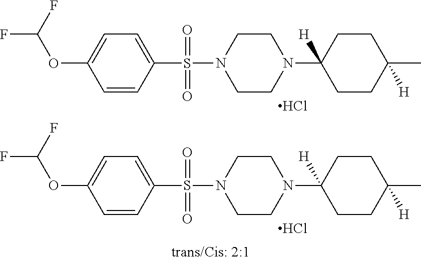 [SMILES: C[C@H]1CC[C@H](N2CCN(S(=O)(=O)c3ccc(OC(F)F)cc3)CC2)CC1 ; Cl ; C[C@H]1CC[C@@H](N2CCN(S(=O)(=O)c3ccc(OC(F)F)cc3)CC2)CC1 ; Cl] |

According to one aspect, the instant disclosure provides a method for identifying a subject who will benefit from treatment with a pharmaceutical composition comprising an EBP-modulating anti-cancer compound, the method comprising (a) isolating a tumor sample comprising a population of cancer cells from the subject; (b) providing (i) an aliquot of the tumor sample in (a) as a test population of cancer cells, (ii) a known population of cancer cells sensitive to an EBP-modulating anticancer compound (positive control), and (iii) a known population of cancer cells insensitive to an EBP-modulating anticancer compound (negative control); (c) determining whether the aliquot of the tumor sample contains a subpopulation of cancer cells sensitive to a composition comprising an EBP-modulating anti-cancer compound by (1) contacting the known EBP-modulating anticancer compound to the populations of cancer cells in (b); (2) measuring EBP enzyme activity and a parameter of endogenous cholesterol synthesis for each population of cancer cells, wherein an amount of the EBP-modulating anti-cancer compound is effective to decrease EBP enzyme activity and to decrease endogenous cholesterol synthesis in a cancer cell sensitive to the known EBP modulating anti-cancer compound, while an amount of the EBP-modulating anti-cancer compound is effective to increase EBP activity and to increase endogenous cholesterol synthesis in a cancer cell insensitive to the EBP modulating anti-cancer compound; (3) distinguishing the sensitive population of cancer cells from the insensitive population of cancer cells in the test population of cancer cells; and (d) if the test population of cancer cells contains a population of cancer cells sensitive to the EBP modulating anti-cancer compound, treating the tumor by administering to the subject a pharmaceutical composition containing a therapeutic amount of the EBP modulating anti-cancer compound. According to one embodiment of the method, in a cancer cell sensitive to the EBP modulating anti-cancer compound, the effective amount of the EBP-modulating anti-cancer compound is effective to cause accumulation of a Δ8 sterol intermediate. According to another embodiment, the Δ8 sterol intermediate is 5α-cholest-8-(9)-en-3β-ol (Δ8-cholesetenol). According to another embodiment, in the cancer cell sensitive to the EBP-modulating anticancer compound, the effective amount of the EBP modulating anti-cancer compound is effective to cause downregulation of SREBP-2. According to another embodiment, in the cancer cell sensitive to the EBP-modulating anticancer compound, the effective amount of the EBP modulating anti-cancer compound is effective to cause downregulation of SREBP-2 genes. According to another embodiment, in the cancer cell sensitive to the EBP-modulating anticancer compound, the effective amount of the EBP modulating anti-cancer compound is effective to cause downregulation of SREBP-2 and one or more SREBP-2 target genes of the cholesterol biosynthetic pathway selected from the group consisting of ACAT2; MHGCS1; HMGCR; MVK; PMVK; MVD; ID11/ID12; FDFS; GGPS1; FDFT1; SQLE; LSS; CYPS1A1; TM75F2; SCAMOL; NSDHL; HSD17B7; EBP; SC5D; DHCR7; and DHCR24. According to another embodiment, the cancer cell sensitive to the EBP-modulating anti-cancer compound comprises a truncated APC protein. According to another embodiment, the therapeutic amount of the EBP-modulating anti-cancer compound is effective to reduce proliferation of the cancer cell sensitive to the EBP modulating anti-cancer compound, to reduce invasiveness of the cancer cell sensitive to the EBP modulating anti-cancer compound, increase apoptosis of the cancer cell sensitive to the EBP modulating anti-cancer compound, reduce growth of a tumor comprising the cancer cell sensitive to the EBP modulating anti-cancer compound, reduce tumor burden, improve progression free survival, improve overall survival, achieve remission of disease, or a combination thereof. According to another embodiment, the EBP-modulating anti-cancer compound is selected from the group consisting of TASIN-1 and functional equivalents thereof, dendrogenin A, SR31747A, tamoxifen, emopamil, verapamil, cis-flupentixol, trifluoroperazine, 7-ketocholestenol, haloperidol, and fenpropimorph.

In some embodiments, the known population of cancer cells insensitive to the EBP-modulating anticancer compound is a population of HCT116 cells or RKO cells. In some embodiments, the known population of cancer cells sensitive to the EBP modulating anti-cancer compound is a population of DLD1 cells, HT29 cells, SW620 cells, SE480 cells, Caco-2 cells, Lovo cells or HC116 p53−/−A1309 cells.

In some embodiments, the instant disclosure provides a method for identifying a therapeutic EBP-modulating anticancer compound comprising (a) dividing a population of cancer cells sensitive to a known EBP-modulating anti-cancer compound into aliquoted samples of the population of cancer cells; (b) contacting one sample of the population of sensitive cancer cells with a candidate EBP-modulating anti-cancer compound, contacting a second sample of the sensitive population of cancer cells with a known EBP-modulating anticancer compound (positive control), and contacting a third sample of the sensitive population of cancer cells with a negative control; (c) measuring EBP activity and a parameter of endogenous cholesterol synthesis for the candidate EBP-modulating compound, the positive control and the negative control in (b), wherein an amount of the known EBP-modulating anti-cancer compound is effective to decrease EBP activity and to decrease endogenous cholesterol synthesis in a sensitive cancer cell, while an amount of the known EBP-modulating anti-cancer compound is effective to increase EBP activity and to increase endogenous cholesterol synthesis in a cancer cell insensitive to the known EBP modulating anti-cancer compound; (d) ranking a plurality of candidate EBP-modulating anti-cancer compounds according to the measured effect on EBP activity and the parameter of endogenous cholesterol synthesis in (c); and (e) selecting a top-ranked candidate EBP-modulating anti-cancer compound in (d) as a new EBP-modulating anti-cancer compound for treating a subject in need thereof. According to one embodiment of the method, the population of cancer cells known to be sensitive to the EBP modulating compound is a population of DLD1 cells or HT29 cells. According to another embodiment, the EBP-modulating anti-cancer compound is selected from TASIN-1 or a functional equivalent thereof, dendrogenin A, SR31747A, tamoxifen, emopamil, verapamil, cis-flupentixol, trifluoroperazine, 7-ketocholestenol, haloperidol, and fenpropimorph.

In some embodiments, the decrease in EBP activity is measured as an accumulation of a Δ8 sterol intermediate. In some embodiments, the Δ8 sterol intermediate is 5α-cholest-8-(9)-en-3β-ol (Δ8-cholesetenol). In some embodiments, the effective amount of the new EBP modulating anti-cancer compound is effective to cause downregulation of SREBP-2. In some embodiments, the effective amount of the new EBP modulating anti-cancer compound is effective to cause downregulation of one or more SREBP-2 target genes of the cholesterol biosynthetic pathway selected from the group consisting of ACAT2; MHGCS1; HMGCR; MVK; PMVK; MVD; ID11/ID12; FDFS; GGPS1; FDFT1; SQLE; LSS; CYPS1A1; TM75F2; SCAMOL; NSDHL; HSD17B7; EBP; SC5D; DHCR7; and DHCR24. In some embodiments, the effective amount of the new EBP modulating anti-cancer compound is effective to cause downregulation of SREBP-2 and one or more SREBP-2 target genes of the cholesterol biosynthetic pathway selected from the group consisting of ACAT2; MHGCS1; HMGCR; MVK; PMVK; MVD; ID11/ID12; FDFS; GGPS1; FDFT1; SQLE; LSS; CYPS1A1; TM75F2; SCAMOL; NSDHL; HSD17B7; EBP; SC5D; DHCR7; and DHCR24.

The following explanations of terms and methods are provided to better describe the present disclosure and to guide those of ordinary skill in the art in the practice of the present disclosure. The singular terms “a,” “an,” and “the” include plural referents unless context clearly indicates otherwise. Similarly, the word “or” is intended to include “and” unless the context clearly indicates otherwise. The term “comprises” means “includes.” Thus, “comprising A or B,” means “including A, B, or A and B,” without excluding additional elements. The term “about” will be understood by persons of ordinary skill in the art. Whether the term “about” is used explicitly or not, every quantity given herein refers to the actual given value, and it is also meant to refer to the approximation to such given value that would be reasonably inferred based on the ordinary skill in the art.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless indicated otherwise, the nomenclature of substituents that are not explicitly defined herein are arrived at by naming the terminal portion of the functionality followed by the adjacent functionality toward the point of attachment. A person of ordinary skill in the art would recognize that the above definitions are not intended to include impermissible substitution patterns (e.g., methyl substituted with 5 different groups, pentavalent carbon, and the like). Such impermissible substitution patterns are easily recognized by a person of ordinary skill in the art. All publications, patent applications, patents, and other references mentioned herein are incorporated by reference in their entirety. All sequences provided in the disclosed Genbank Accession numbers are incorporated herein by reference as available on Aug. 11, 2011. In case of conflict, the present specification, including explanations of terms, will control. In addition, the materials, methods, and examples are illustrative only and not intended to be limiting.

Alkyl groups refer to univalent groups derived from alkanes by removal of a hydrogen atom from any carbon atom, which include straight chain and branched chain with from 1 to 12 carbon atoms, and typically from 1 to about 10 carbons or in some embodiments, from 1 to about 6 carbon atoms, or in other embodiments having 1, 2, 3 or 4 carbon atoms. Examples of straight chain alkyl groups include, but are not limited to, methyl, ethyl, n-propyl, n-butyl, n-pentyl, and n-hexyl groups. Examples of branched chain alkyl groups include, but are not limited to isopropyl, isobutyl, sec-butyl and tert-butyl groups. Alkyl groups may be substituted or unsubstituted. Representative substituted alkyl groups may be mono-substituted or substituted more than once, such as, but not limited to, mono-, di-, or tri-substituted. As used herein, the term alkyl, unless otherwise stated, refers to both cyclic and noncyclic groups.

The terms “cyclic alkyl” or “cycloalkyl” refer to univalent groups derived from cycloalkanes by removal of a hydrogen atom from a ring carbon atom. Cycloalkyl groups are saturated or partially saturated non-aromatic structures with a single ring or multiple rings including isolated, fused, bridged, and spiro ring systems, having 3 to 14 carbon atoms, or in some embodiments, from 3 to 12, or 3 to 10, or 3 to 8, or 3, 4, 5, 6 or 7 carbon atoms. Cycloalkyl groups may be substituted or unsubstituted. Representative substituted cycloalkyl groups may be mono-substituted or substituted more than once, such as, but not limited to, mono-, di-, or tri-substituted. Examples of monocyclic cycloalkyl groups include, but are not limited to cyclopropyl, cyclobutyl, cyclopentyl, and cyclohexyl groups. Examples of multi-cyclic ring systems include, but are not limited to, bicycle[4.4.0]decane, bicycle[2.2.1]heptane, spiro[2.2]pentane, and the like. (Cycloalkyl)oxy refers to —O— cycloalkyl. (Cycloalkyl)thio refers to —S-cycloalkyl. This term also encompasses oxidized forms of sulfur, such as —S(O)-cycloalkyl, or —S(O)₂-cycloalkyl.

Alkenyl groups refer to straight and branched chain and cycloalkyl groups as defined above, with one or more double bonds between two carbon atoms. Alkenyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkenyl groups may be substituted or unsubstituted. Representative substituted alkenyl groups may be mono-substituted or substituted more than once, such as, but not limited to, mono-, di-, or tri-substituted. Examples of alkenyl groups include, but are not limited to, vinyl, allyl, —CH═CH(CH₃), —CH═C(CH₃)₂, —C(CH₃)═CH₂, cyclopentenyl, cyclohexenyl, butadienyl, pentadienyl, and hexadienyl, among others.

Alkynyl groups refer to straight and branched chain and cycloalkyl groups as defined above, with one or more triple bonds between two carbon atoms. Alkynyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkynyl groups may be substituted or unsubstituted. Representative substituted alkynyl groups may be mono-substituted or substituted more than once, such as, but not limited to, mono-, di-, or tri-substituted. Exemplary alkynyl groups include, but are not limited to, ethynyl, propargyl, and —C≡C(CH₃), among others.

Aryl groups are cyclic aromatic hydrocarbons that include single and multiple ring compounds, including multiple ring compounds that contain separate and/or fused aryl groups. Aryl groups may contain from 6 to about 18 ring carbons, or in some embodiments from 6 to 14 ring carbons or even 6 to 10 ring carbons in other embodiments. Aryl group also includes heteroaryl groups, which are aromatic ring compounds containing 5 or more ring members, one or more ring carbon atoms of which are replaced with heteroatom such as, but not limited to, N, O, and S. Aryl groups may be substituted or unsubstituted. Representative substituted aryl groups may be mono-substituted or substituted more than once, such as, but not limited to, mono-, di-, or tri-substituted. Aryl groups include, but are not limited to, phenyl, biphenylenyl, triphenylenyl, naphthyl, anthryl, and pyrenyl groups. Aryloxy refers to —O-aryl. Arylthio refers to —S-aryl, wherein aryl is as defined herein. This term also encompasses oxidized forms of sulfur, such as —S(O)-aryl, or —S(O)₂-aryl. Heteroaryloxy refers to —O-heteroaryl. Heteroarylthio refers to —S-heteroaryl. This term also encompasses oxidized forms of sulfur, such as —S(O)-heteroaryl, or —S(O)₂-heteroaryl.

Suitable heterocyclyl groups include cyclic groups with atoms of at least two different elements as members of its rings, of which one or more is a heteroatom such as, but not limited to, N, O, or S. Heterocyclyl groups may include 3 to about 20 ring members, or 3 to 18 in some embodiments, or about 3 to 15, 3 to 12, 3 to 10, or 3 to 6 ring members. The ring systems in heterocyclyl groups may be unsaturated, partially saturated, and/or saturated. Heterocyclyl groups may be substituted or unsubstituted. Representative substituted heterocyclyl groups may be mono-substituted or substituted more than once, such as, but not limited to, mono-, di-, or tri-substituted. Exemplary heterocyclyl groups include, but are not limited to, pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, aziridinyl, imidazolidinyl, pyrazolidinyl, thiazolidinyl, tetrahydrothiophenyl, tetrahydrofuranyl, dioxolyl, furanyl, thiophenyl, pyrrolyl, imidazolyl, pyrazolyl, pyrazolinyl, triazolyl, tetrazolyl, oxazolyl, isoxazolyl, thiazolyl, thiazolinyl, oxetanyl, thietanyl, homopiperidyl, oxepanyl, thiepanyl, oxazepinyl, diazepinyl, thiazepinyl, 1,2,3,6-tetrahydropyridyl, indolinyl, 2H-pyranyl, 4H-pyranyl, dioxolanyl, dioxanyl, purinyl, quinolizinyl, cinnolinyl, phthalazinyl, pteridinyl, and benzothiazolyl groups. Heterocyclyloxy refers to —O-heterocycyl. Heterocyclylthio refers to —S-heterocycyl. This term also encompasses oxidized forms of sulfur, such as —S(O)-heterocyclyl, or —S(O)₂-heterocyclyl.

Polycyclic or polycyclyl groups refer to two or more rings in which two or more carbons are common to the two adjoining rings, wherein the rings are “fused rings”; if the rings are joined by one common carbon atom, these are “spiro” ring systems. Rings that are joined through non-adjacent atoms are “bridged” rings. Polycyclic groups may be substituted or unsubstituted. Representative polycyclic groups may be substituted one or more times.

Halogen groups include F, Cl, Br, and I; nitro group refers to —NO₂; cyano group refers to —CN; isocyano group refers to —N≡C; epoxy groups encompass structures in which an oxygen atom is directly attached to two adjacent or non-adjacent carbon atoms of a carbon chain or ring system, which is essentially a cyclic ether structure. An epoxide is a cyclic ether with a three-atom ring.

An alkoxy group is a substituted or unsubstituted alkyl group, as defined above, singular bonded to oxygen. Alkoxy groups may be substituted or unsubstituted. Representative substituted alkoxy groups may be substituted one or more times. Exemplary alkoxy groups include, but are not limited to, methoxy, ethoxy, propoxy, butoxy, pentoxy, hexoxy, isopropoxy, sec-butoxy, tert-butoxy, cyclopropyloxy, cyclobutyloxy, cyclopentyloxy, and cyclohexyloxy groups.

Thiol refers to —SH. Thiocarbonyl refers to (═S). Sulfonyl refers to —SO₂-alkyl, —SO₂-substituted alkyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclyl, and —SO₂-substituted heterocyclyl. Sulfonylamino refers to —NR^{a}SO₂alkyl, —NR^{a}SO₂-substituted alkyl, —NR^{a}SO₂cycloalkyl, —NR^{a}SO₂substituted cycloalkyl, —NR^{a}SO₂aryl, —NR^{a}SO₂substituted aryl, —NR^{a}SO₂heteroaryl, —NR^{a}SO₂ substituted heteroaryl, —NR^{a}SO₂heterocyclyl, —NR^{a}SO₂ substituted heterocyclyl, wherein each R^{a} independently is as defined herein.

Carboxyl refers to —COOH or salts thereof. Carboxyester refers to —C(O)O-alkyl, —C(O)O— substituted alkyl, —C(O)O-aryl, —C(O)O-substituted aryl, —C(O)β-cycloalkyl, —C(O)O— substituted cycloalkyl, —C(O)O-heteroaryl, —C(O)O-substituted heteroaryl, —C(O)O— heterocyclyl, and —C(O)O-substituted heterocyclyl. (Carboxyester)amino refers to —NR^{a}—C(O)O-alkyl, —NR^{a}—C(O)O-substituted alkyl, —NR^{a}—C(O)O-aryl, —NR^{a}—C(O)O-substituted aryl, —NR^{a}—C(O)β-cycloalkyl, —NR^{a}—C(O)O-substituted cycloalkyl, —NR^{a}—C(O)O-heteroaryl, —NR^{a}—C(O)O-substituted heteroaryl, —NR^{a}—C(O)O-heterocyclyl, and —NR^{a}—C(O)O-substituted heterocyclyl, wherein R^{a} is as recited herein. (Carboxyester)oxy refers to —O—C(O)O-alkyl, —O—C(O)O— substituted alkyl, —O—C(O)O-aryl, —O—C(O)O-substituted aryl, —O—C(O)β-cycloalkyl, —O—C(O)O-substituted cycloalkyl, —O—C(O)O-heteroaryl, —O—C(O)O-substituted heteroaryl, —O—C(O)O-heterocyclyl, and —O—C(O)O-substituted heterocyclyl. Oxo refers to (═O).

The terms “amine” and “amino” refer to derivatives of ammonia, wherein one of more hydrogen atoms have been replaced by a substituent which include, but are not limited to alkyl, alkenyl, aryl, and heterocyclyl groups. Carbamate groups refers to —O(C═O)NR¹R₂, where R₁ and R₂ are independently hydrogen, aliphatic groups, aryl groups, or heterocyclyl groups.

Aminocarbonyl refers to —C(O)N(R^{b})₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylalkyl refers to -alkylC(O)N(R^{b})₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylamino refes to —NR^{a}C(O)N(R^{b})₂, wherein R^{a} and each R^{b} are as defined herein. Aminodicarbonylamino refers to —NR^{a}C(O)C(O)N(R^{b})₂, wherein R^{a} and each R^{b} are as defined herein. Aminocarbonyloxy refers to —O—C(O)N(R^{b})₂, wherein each R^{b} independently is as defined herein. Aminosulfonyl refers to —SO₂N(R^{b})₂, wherein each R^{b} independently is as defined herein.

Imino refers to —N═R^{c} wherein R^{c} may be selected from hydrogen, aminocarbonylalkyloxy, substituted aminocarbonylalkyloxy, aminocarbonylalkylamino, and substituted aminocarbonylalkylamino.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those needing preventive measures).

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” or the like refer to the amount of a subject compound that will elicit the biological or medical response in a tissue, system, animal or human that is being sought by administering said compound. Generally, the response is either amelioration of symptoms in a patient or a desired biological outcome. Such amount should be sufficient to inhibit MIF activity.

Also disclosed herein are pharmaceutical compositions including compounds with the structures of Formula (I). The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable carriers that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

In pharmaceutical composition comprising only the compounds described herein as the active component, methods for administering these compositions may additionally comprise the step of administering to the subject an additional agent or therapy. Such therapies include, but are not limited to, an anemia therapy, a diabetes therapy, a hypertension therapy, a cholesterol therapy, neuropharmacologic drugs, drugs modulating cardiovascular function, drugs modulating inflammation, immune function, production of blood cells; hormones and antagonists, drugs affecting gastrointestinal function, chemotherapeutics of microbial diseases, and/or chemotherapeutics of neoplastic disease. Other pharmacological therapies can include any other drug or biologic found in any drug class. For example, other drug classes can comprise allergy/cold/ENT therapies, analgesics, anesthetics, anti-inflammatories, antimicrobials, antivirals, asthma/pulmonary therapies, cardiovascular therapies, dermatology therapies, endocrine/metabolic therapies, gastrointestinal therapies, cancer therapies, immunology therapies, neurologic therapies, ophthalmic therapies, psychiatric therapies or rheumatologic therapies. Other examples of agents or therapies that can be administered with the compounds described herein include a matrix metalloprotease inhibitor, a lipoxygenase inhibitor, a cytokine antagonist, an immunosuppressant, a cytokine, a growth factor, an immunomodulator, a prostaglandin or an anti-vascular hyperproliferation compound.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology).

The compounds of this disclosure may be employed in a conventional manner for controlling the disease described herein, including, but not limited to, colorectal cancer. Such methods of treatment, their dosage levels and requirements may be selected by those of ordinary skill in the art from available methods and techniques. The compounds may be employed in such compositions either alone or together with other compounds of this disclosure in a manner consistent with the conventional utilization of such compounds in pharmaceutical compositions. For example, a compound of this disclosure may be combined with pharmaceutically acceptable adjuvants conventionally employed in vaccines and administered in prophylactically effective amounts to protect individuals over an extended period of time against the diseases described herein.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

When the compounds of this disclosure are administered in combination therapies with other agents, they may be administered sequentially or concurrently to the patient. Alternatively, pharmaceutical or prophylactic compositions according to this disclosure comprise a combination of ivermectin, or any other compound described herein, and another therapeutic or prophylactic agent. Additional therapeutic agents that are normally administered to treat a particular disease or condition may be referred to as “agents appropriate for the disease, or condition, being treated.”

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this disclosure are formulated for pharmaceutical administration to a subject or patient, e.g., a mammal, preferably a human being. Such pharmaceutical compositions are used to ameliorate, treat or prevent any of the diseases described herein in a subject.

Agents of the disclosure are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present disclosure provides pharmaceutically acceptable compositions comprising a therapeutically effective amount of one or more of a described compound, formulated together with one or more pharmaceutically acceptable carriers (additives) and/or diluents for use in treating the diseases described herein, including, but not limited to colorectal cancer. While it is possible for a described compound to be administered alone, it is preferable to administer a described compound as a pharmaceutical formulation (composition) as described herein. Described compounds may be formulated for administration in any convenient way for use in human or veterinary medicine, by analogy with other pharmaceuticals.

As described in detail, pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material, to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient. In some embodiments, this amount will range from about 5% to about 70%, from about 10% to about 50%, or from about 20% to about 40%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present disclosure. In certain embodiments, an aforementioned formulation renders orally bioavailable a described compound of the present disclosure.

Methods of preparing formulations or compositions comprising described compounds include a step of bringing into association a compound of the present disclosure with the carrier and, optionally, one or more accessory ingredients. In general, formulations may be prepared by uniformly and intimately bringing into association a compound of the present disclosure with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, compounds described herein which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

In treatment, the dose of agent optionally ranges from about 0.0001 mg/kg to 100 mg/kg, about 0.01 mg/kg to 5 mg/kg, about 0.15 mg/kg to 3 mg/kg, about 0.5 mg/kg to 2 mg/kg and about 1 mg/kg to 2 mg/kg of the subject's body weight. In other embodiments the dose ranges from about 100 mg/kg to 5 g/kg, about 500 mg/kg to 2 mg/kg and about 750 mg/kg to 1.5 g/kg of the subject's body weight. For example, depending on the type and severity of the disease, about 1 μg/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, an agent is administered to a human patient at an effective amount (or dose) of less than about 1 μg/kg, for instance, about 0.35 to 0.75 μg/kg or about 0.40 to 0.60 μg/kg. In some embodiments, the dose of an agent is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of an agent is about 2 μg/subject to about 45 μg/subject, or about 5 to about 40, or about 10 to about 30, or about 15 to about 25 μg/subject. In some embodiments, the absolute dose of an agent is about 20 μg, or about 30 μg, or about 40 μg.

In various embodiments, the dose of an agent may be determined by the human patient's body weight. For example, an absolute dose of an agent of about 2 μg for a pediatric human patient of about 0 to 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

The term “cancer” refers to a group diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS—Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland'Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In certain aspects, cancer include Lung cancer, Breast cancer, Colorectal cancer, Prostate cancer, Stomach cancer, Liver cancer, cervical cancer, Esophageal cancer, Bladder cancer, Non-Hodgkin lymphoma, Leukemia, Pancreatic cancer, Kidney cancer, endometrial cancer, Head and neck cancer, Lip cancer, oral cancer, Thyroid cancer, Brain cancer, Ovary cancer, Melanoma, Gallbladder cancer, Laryngeal cancer, Multiple myeloma, Nasopharyngeal cancer, Hodgkin lymphoma, Testis cancer and Kaposi sarcoma.

In certain aspects, the method further includes administering a chemotherapeutic agent. The compounds of the disclosure can be administered in combination with one or more additional therapeutic agents. The phrases “combination therapy”, “combined with” and the like refer to the use of more than one medication or treatment simultaneously to increase the response. The FGFR inhibitor of the present disclosure might for example be used in combination with other drugs or treatment in use to treat cancer. In various aspect, the compound is administered prior to, simultaneously with or following the administration of the chemotherapeutic agent.

The term “anti-cancer therapy” refers to any therapy or treatment that can be used for the treatment of a cancer. Anti-cancer therapies include, but are not limited to, surgery, radiotherapy, chemotherapy, immune therapy and targeted therapies.

Examples of chemotherapeutic agents or anti-cancer agents include, but are not limited to, Actinomycin, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fiuorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine, panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Raptiva (efalizumab), Tysabri (natalizumab), Zenapax (dacliximab), NeutroSpec (Technetium (99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab Gilotrif (afatinib), Lynparza (olaparib), Perjeta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib). Examples of immunotherapeutic agent include, but are not limited to, interleukins (Il-2, Il-7, Il-12), cytokines (Interferons, G-CSF, imiquimod), chemokines (CCL3, CC126, CXCL7), immunomodulatory imide drugs (thalidomide and its analogues).

The term “Adenomatous polyposis coli gene” or “APC gene” or “APC” as used herein refers to a mammalian DNA sequence coding for an APC protein. An example of a human APC gene is located at 5q21-q22 on chromosome 5, GenBank: M74088.1. Synonyms for the human APC gene include: BTPS2, DP2, DP2.5, DP3, PPP1R46 and “protein phosphatase 1, regulatory subunit 46”. An example of a mouse APC gene is located at chromosome 18 B1, MGI:88039. Synonyms for the mouse APC gene include: CC2, Min, mAPC, AAI10147805, AU020952 and AW124434.

The term “Adenomatous polyposis coli protein” or “APC protein” or “APC” as used herein refers to a mammalian protein sequence of 2843 amino acids. An example of a human APC sequence is GenBank: AAA03586. An example of a mouse APC sequence is GenBank: AAB59632.

The term “APC truncation” or “APC truncation mutant” or “APC truncation mutation” refers to a truncated protein product resulting from a mutation occurring within the APC gene. An APC truncation can be, for example, but not limited to, a 1309 amino acid product or a 1450 amino acid product.

The term “adjuvant therapy” refers to a treatment added to a primary treatment to prevent recurrence of a disease, or the additional therapy given to enhance or extend the primary therapy's effect, as in chemotherapy's addition to a surgical regimen.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “antagonist” as used herein refers to a small molecule, peptide, protein, or antibody that can bind to an enzyme, a receptor or a co-receptor, competitively or noncompetitively through a covalent bond, ionic bond, hydrogen bond, hydrophobic interaction, or a combination thereof and either directly or indirectly deactivate a related downstream signaling pathway.

The term “anti-cancer compounds” as used herein refers to small molecule compounds that selectively target cancer cells and reduce their growth, proliferation, or invasiveness, or tumor burden of a tumor containing such cancer cells.

The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be administered by means such as, but not limited to, injection, implantation, grafting, topical application, or parenterally.

The terms “analog” and “derivative” are used interchangeably to mean a compound produced from another compound of similar structure in one or more steps. A “derivative” or “analog” of a compound retains at least a degree of the desired function of the reference compound. Accordingly, an alternate term for “derivative” may be “functional derivative.” Derivatives can include chemical modifications, such as akylation, acylation, carbamylation, iodination or any modification that derivatives the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives.

The term “allosteric modulation” as used herein refers to the process of modulating a receptor by the binding of allosteric modulators at a different site (i.e., regulatory site) other than of the endogenous ligand (orthosteric ligand) of the receptor and enhancing or inhibiting the effects of the endogenous ligand. It normally acts by causing a conformational change in a receptor molecule, which results in a change in the binding affinity of the ligand. Thus, an allosteric ligand “modulates” its activation by a primary “ligand” and can adjust the intensity of the receptor's activation. Many allosteric enzymes are regulated by their substrate, such a substrate is considered a “homotropic allosteric modulator.” Non-substrate regulatory molecules are called “heterotropic allosteric modulators.”

The term “allosteric regulation” is the regulation of an enzyme or other protein by binding an effector molecule at the proteins allosteric site (meaning a site other than the protein's active site). Effectors that enhance the protein's activity are referred to as “allosteric activators”, whereas those that decrease the protein's activity are called “allosteric inhibitors.” Thus, “allosteric activation” occurs when the binding of one ligand enhances the attraction between substrate molecules and other binding sites; “allosteric inhibition” occurs when the binding of one ligand decrease the affinity for substrate at other active sites. The term “antagonist” as used herein refers to a substance that counteracts the effects of another substance.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “assay marker” or “reporter gene” (or “reporter”) refers to a gene that can be detected, or easily identified and measured. The expression of the reporter gene may be measured at either the RNA level, or at the protein level. The gene product, which may be detected in an experimental assay protocol, includes, but is not limited to, marker enzymes, antigens, amino acid sequence markers, cellular phenotypic markers, nucleic acid sequence markers, and the like. Researchers may attach a reporter gene to another gene of interest in cell culture, bacteria, animals, or plants. For example, some reporters are selectable markers, or confer characteristics upon on organisms expressing them allowing the organism to be easily identified and assayed. To introduce a reporter gene into an organism, researchers may place the reporter gene and the gene of interest in the same DNA construct to be inserted into the cell or organism. For bacteria or eukaryotic cells in culture, this may be in the form of a plasmid. Commonly used reporter genes may include, but are not limited to, fluorescent proteins, luciferase, beta-galactosidase, and selectable markers, such as chloramphenicol and kanomycin.

As used herein, the term “bioavailability” refers to the rate and extent to which the active drug ingredient or therapeutic moiety is absorbed into the systemic circulation from an administered dosage form as compared to a standard or control.

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bound” or any of its grammatical forms as used herein refers to the capacity to hold onto, attract, interact with or combine with.

The terms “cancer” or “malignancy” as used herein refer to diseases in which abnormal cells divide without control and can invade nearby tissues. Cancer cells also can spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in cells of the immune system. Central nervous system cancers are cancers that begin in the tissues of the brain and spinal cord.

The term “cell” is used herein to refer to the structural and functional unit of living organisms and is the smallest unit of an organism classified as living.

The term “cell line” as used herein refers to a population of immortalized cells, which have undergone transformation and can be passed indefinitely in culture.

The term “chemoresistance” as used herein refers to the development of a cell phenotype resistant to a variety of structurally and functionally distinct agents. Tumors can be intrinsically resistant prior to chemotherapy, or resistance may be acquired during treatment by tumors that are initially sensitive to chemotherapy. Drug resistance is a multifactorial phenomenon involving multiple interrelated or independent mechanisms. A heterogeneous expression of involved mechanisms may characterize tumors of the same type or cells of the same tumor and may at least in part reflect tumor progression. Exemplary mechanisms that can contribute to cellular resistance include: increased expression of defense factors involved in reducing intracellular drug concentration; alterations in drug-target interaction; changes in cellular response, in particular increased cell ability to repair DNA damage or tolerate stress conditions, and defects in apoptotic pathways.

The term “chemosensitive”, “chemosensitivity” or “chemosensitive tumor” as used herein refers to a tumor that is responsive to a chemotherapy or a chemotherapeutic agent. Characteristics of a chemosensitive tumor include, but are not limit to, reduced proliferation of the population of tumor cells, reduced tumor size, reduced tumor burden, tumor cell death, and slowed/inhibited progression of the population of tumor cells.

The term “chemotherapeutic agent” as used herein refers to chemicals useful in the treatment or control of a disease, e.g., cancer.

The term “chemotherapy” as used herein refers to a course of treatment with one or more chemotherapeutic agents. In the context of cancer, the goal of chemotherapy is, e.g., to kill cancer cells, reduce proliferation of cancer cells, reduce growth of a tumor containing cancer cells, reduce invasiveness of cancer cells, increase apoptosis of cancer cells.

The term “chemotherapy regimen” (“combination chemotherapy”) means chemotherapy with more than one drug in order to benefit from the dissimilar toxicities of the more than one drug. A principle of combination cancer therapy is that different drugs work through different cytotoxic mechanisms; since they have different dose-limiting adverse effects, they can be given together at full doses.

The term “compatible” as used herein means that the components of a composition are capable of being combined with each other in a manner such that there is no interaction that would substantially reduce the efficacy of the composition under ordinary use conditions.

The term “condition”, as used herein, refers to a variety of health states and is meant to include disorders or diseases caused by any underlying mechanism or injury.

The term “contact” and its various grammatical forms as used herein refers to a state or condition of touching or of immediate or local proximity. Contacting a composition to a target destination, such as, but not limited to, an organ, a tissue, a cell, or a tumor, may occur by any means of administration known to the skilled artisan.

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.” Derivatives can include chemical modifications of the peptide, such as akylation, acylation, carbamylation, iodination or any modification that derivatizes the peptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those peptides that contain one or more naturally occurring amino acid derivative of the twenty standard amino acids, for example, 4-hydroxyproline, 5-hydroxylysine, 3-methylhistidine, homoserine, omithine or carboxyglutamiate, and can include amino acids that are not linked by peptide bonds. Such peptide derivatives can be incorporated during synthesis of a peptide, or a peptide can be modified by well known chemical modification methods (see, e.g., Glazer et al., Chemical Modification of Proteins, Selected Methods and Analytical Procedures, Elsevier Biomedical Press, New York (1975)).

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like. When a nucleic acid is prepared or altered synthetically, advantage can be taken of known codon preferences of the intended host where the nucleic acid is to be expressed.

The term “detectable response” refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. The term “disease” or “disorder”, as used herein, refers to an impairment of health or a condition of abnormal functioning.

The term “DLD-1” as used herein refers to a human colon cancer cell line with a truncated APC. The term “dose” as used herein refers to the quantity of medicine prescribed to be taken at one time. The term “drug” as used herein refers to a therapeutic agent or any substance used in the prevention, diagnosis, alleviation, treatment, or cure of disease. The terms “Emopamil Binding Protein” (EBP), “Human Sterol Isomerase” (HIS) and “delta8-delta7 sterol isomerase” are used interchangeably to refer to an integral membrane protein of the endoplasmic reticulum that catalyzes the conversion of delta(8)-sterols into delta(7)-sterols.

The term “effective amount” or “amount effective” refers to the amount necessary or sufficient to realize a desired biologic effect. The term “effective dose” as used herein refers to the quantity of medicine prescribed to be taken at one time necessary or sufficient to realize a desired biologic effect.

As used herein, the term “enzymatic activity” refers to the amount of substrate consumed (or product formed) in a given time under given conditions. Enzymatic activity also may be referred to as “turnover number.”

The term “functional equivalent” or “functionally equivalent” are used interchangeably herein to refer to substances, molecules, polynucleotides, proteins, peptides, or polypeptides having similar or identical biological activity to a reference substance, molecule, polynucleotide, protein, peptide, or polypeptide. Any EBP-modulating anti-cancer compound that retains the biological activity of TASIN-1 may be used as such a functional equivalent. The term “growth” as used herein refers to a process of becoming larger, longer or more numerous, or an increase in size, number, or volume. The term “half maximal inhibitory concentration” (“IC50”) is a measure of the effectiveness of a compound in inhibiting a biological or biochemical function.

The term “HCT116” as used herein refers to a human colon cancer cell line with wild type APC. The term “HT29” as used herein refers to a human colon cancer cell line with a truncated APC.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a molecule that binds to an enzyme thereby decreasing enzyme activity. Enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injury,” as used herein, refers to damage or harm to a structure or function of the body caused by an outside agent or force, which may be physical or chemical.

The term “interfere” or “to interfere with” as used herein refers to the hampering, impeding, dampening, hindering, obstructing, blocking, reducing or preventing of an action or occurrence. By way of example, a receptor antagonist interferes with (e.g., blocks or dampens) an agonist-mediated response rather than provoking a biological response itself.

The term “invasion” or “invasiveness” as used herein refers to a process in malignant cells that includes penetration of and movement through surrounding tissues.

The term “Kaplan Meier plot” or “Kaplan Meier survival curve” as used herein refers to the plot of probability of clinical study subjects surviving in a given length of time while considering time in many small intervals. The Kaplan Meier plot assumes that: (i) at any time subjects who are censored (i.e., lost) have the same survival prospects as subjects who continue to be followed; (ii) the survival probabilities are the same for subjects recruited early and late in the study; and (iii) the event (e.g., death) happens at the time specified. Probabilities of occurrence of events are computed at a certain point of time with successive probabilities multiplied by any earlier computed probabilities to get a final estimate. The survival probability at any particular time is calculated as the number of subjects surviving divided by the number of subjects at risk. Subjects who have died, dropped out, or have been censored from the study are not counted as at risk.

The term “ligand” as used herein refers to a molecule that can bind selectively to a molecule, such that the binding interaction between the ligand and its binding partner is detectable over nonspecific interactions by a quantifiable assay. Derivatives, analogues and mimetic compounds are intended to be included within the definition of this term.

The terms “marker” and “cell surface marker” are used interchangeably herein to refer to a receptor, a combination of receptors, or an antigenic determinant or epitope found on the surface of a cell that allows a cell type to be distinguishable from other kinds of cells. Specialized protein receptors (markers) that have the capability of selectively binding or adhering to other signaling molecules coat the surface of every cell in the body. Cells use these receptors and the molecules that bind to them as a way of communicating with other cells and to carry out their proper function in the body. Cell sorting techniques are based on cellular biomarkers where a cell surface marker(s) may be used for either positive selection or negative selection, i.e., for inclusion or exclusion, from a cell population.

The term “maximum tolerated dose” (MTD) as used herein refers to the highest dose of a drug that does not produce unacceptable toxicity. The term “median survival” as used herein refers to the time after which 50% of individuals with a particular condition are still living and 50% have died. For example, a median survival of 6 months indicates that after 6 months, 50% of individuals with, e.g., colon cancer would be alive, and 50% would have passed away. Median survival is often used to describe the prognosis (i.e., chance of survival) of a condition when the average survival rate is relatively short, such as for colon cancer. Median survival is also used in clinical studies when a drug or treatment is being evaluated to determine whether or not the drug or treatment will extend life.

The term “metastasis” as used herein refers to the transference of organisms or of malignant or cancerous cells, producing disease manifestations, from one part of the body to other parts. The term “migration” as used herein refers to a movement of a population of cells from one place to another.

The term “mitotic index” as used herein refers to the ratio of the number of cells undergoing mitosis (cell division) to the number of cells not undergoing mitosis in a population of cells.

The term “modify” as used herein means to change, vary, adjust, temper, alter, affect or regulate to a certain measure or proportion in one or more particulars. The term “modifying agent” as used herein refers to a substance, composition, therapeutic component, active constituent, therapeutic agent, drug, metabolite, active agent, protein, non-therapeutic component, non-active constituent, non-therapeutic agent, or non-active agent that reduces, lessens in degree or extent, or moderates the form, symptoms, signs, qualities, character or properties of a condition, state, disorder, disease, symptom or syndrome. The term “modulate” as used herein means to regulate, alter, adapt, or adjust to a certain measure or proportion.

The term “neoplasm” as used herein refers to an abnormal proliferation of genetically altered cells. A malignant neoplasm (or malignant tumor) is synonymous with cancer. A benign neoplasm (or benign tumor) is a tumor (solid neoplasm) that stops growing by itself, does not invade other tissues and does not form metastases. The term “normal healthy control subject” as used herein refers to a subject having no symptoms or other clinical evidence of a disease. The term “normal human colonic epithelial cells” (HCECs) as used herein refers to immortalized human colonic epithelial cell (HCEC) lines generated using exogenously introduced telomerase and cdk4 (Fearon, E. R. & Vogelstein, B. A genetic model for colorectal tumorigenesis. Cell 61, 759-767 (1990)). These cells are nontransformed, karyotypically diploid and have multipotent characteristics. When placed in Matrigel® in the absence of a mesenchymal feeder layer, individual cells divide and form self-organizing, crypt-like structures with a subset of cells exhibiting markers associated with mature intestinal epithelium.

The term “outcome” as used herein refers to a specific result or effect that can be measured. Nonlimiting examples of outcomes include decreased pain, reduced tumor size, and survival (e.g., progression-free survival or overall survival).

The term “overall survival” (OS) as used herein refers to the length of time from either the date of diagnosis or the start of treatment for a disease, such as cancer, that patients diagnosed with the disease are still alive.

The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using exemplary dispersing or wetting agents and suspending agents.

The terms “primary tumor” or “primary cancer” are used interchangeably to refer to the original, or first, tumor in the body. Cancer cells from a primary cancer may spread to other parts of the body and form new, or secondary tumors. This is called metastasis. The secondary tumors are the same type of cancer as the primary cancer.

The term “progression” as used herein refers to the course of a disease as it becomes worse or spreads in the body. The term “progression-free survival” (PFS) as used herein refers to the length of time during and after the treatment of a disease that a patient lives with the disease but it does not get worse. The term “proliferation” as used herein refers to expansion of a population of cells by the continuous division of single cells into identical daughter cells, leading to a multiplying or increasing in the number of cells. The term “recurrence” as used herein refers to a disease (e.g., cancer) that has come back, usually after a period of time during which the disease could not be detected.

The term “reduce” or “reducing” as used herein refers to limit occurrence of a disorder in individuals at risk of developing the disorder. The terms “refractory” or “resistant” are used interchangeably herein refers to a disease or condition that does not respond to treatment. The disease may be resistant at the beginning of treatment or it may become resistant during treatment. The term “remission” as used herein refers to a decrease in or disappearance of signs and symptoms of a disease. In partial remission, some, but not all, signs and symptoms have disappeared. In complete remission, all signs and symptoms have disappeared although the disease may still be in the body.

The term Response Evaluation Criteria in Solid Tumors (or “RECIST”) as used herein refers to a standard way to measure how well a cancer patient responds to treatment. It is based on whether tumors shrink, stay the same, or get bigger. To use RECIST, there must be at least one tumor that can be measured on x-rays, CT scans, or MRI scans. The types of response a patient can have are a complete response (CR), a partial response (PR), progressive disease (PD), and stable disease (SD).

Rho Associated Coiled Coil Kinase (ROCK) Proteins. Cancer-associated changes in cellular behavior, such as modified cell-cell contact, increased migratory potential, and generation of cellular force, all require alteration of the cytoskeleton. ROCK proteins belong to the protein kinase A, G, and C family (AGC family) of classical serine/threonine protein kinases, a group that also includes other regulators of cell shape and motility, such as citron Rho-interacting kinase (CRIK), dystrophia myotonica protein kinase (DMPK), and the myotonic dystrophy kinase-related Cdc42-binding kinases (MRCKs). The main function of ROCK signaling is regulation of the cytoskeleton through the phosphorylation of downstream substrates, leading to increased actin filament stabilization and generation of actin-myosin contractility. (Morgan-Fisher, M. et al., 61:185-198, at 185).

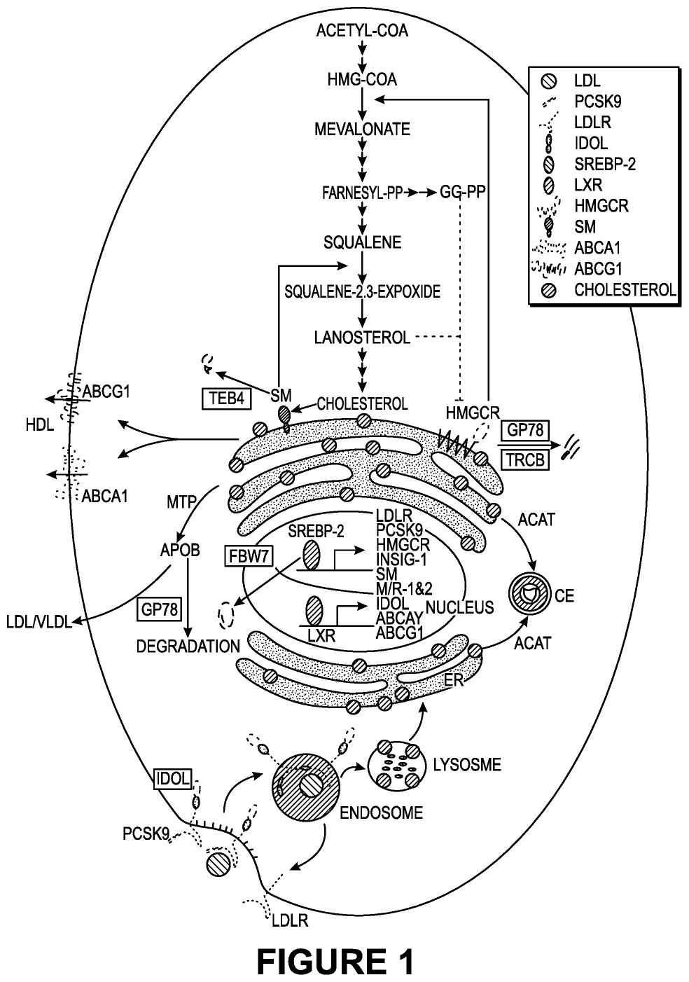

Two homologous mammalian serine/threonine kinases, Rho-associated protein kinases I and II (ROCK I and II), are key regulators of the actin cytoskeleton acting downstream of the small GTPase Rho. ROCK I (alternatively called ROK .beta.) and ROCK II (also known as Rho kinase or ROK .alpha.) are 160-kDa proteins encoded by distinct genes. The mRNA of both kinases is ubiquitously expressed, but ROCK I protein is mainly found in organs such as liver, kidney, and lung, whereas ROCK II protein is mainly expressed in muscle and brain tissue. The two kinases have the same overall domain structure and have 64% overall identity in humans, with 89% identity in the catalytic kinase domain. Both kinases contain a coiled-coil region (55% identity) containing a Rho-binding domain (RBD) and a pleckstrin homology (PH) domain split by a C1 conserved region (80% identity) (See FIG. 1). Despite a high degree of homology between the two ROCKs, as well as the fact that they share several common substrates, studies have shown that the two ROCK isoforms also have distinct and non-redundant functions. For example, ROCK I has been shown to be essential for the formation of stress fibers and focal adhesions, whereas ROCK II is required for myosin II-dependent phagocytosis.

ROCKs exist in a closed, inactive conformation under quiescent conditions, which is changed to an open, active conformation by the direct binding of guanosine triphosphate (GTP)-loaded Rho. (Id.). Rho is a small GTPase which functions as a molecular switch, cycling between guanosine diphosphate (GDP) and guanosine triphosphate (GTP) bound states under signaling through growth factors or cell adhesion receptors. (Id.). GTPases are hydrolase enzymes that bind and hydrolyze GTP. In a similar way to ATP, GTP can act as an energy carrier, but it also has an active role in signal transduction, particularly in the regulation of G protein activity. G proteins, including Rho GTPases, cycle between an inactive GDP-bound and an active GTP-bound conformation. The transition between the two conformational states occurs through two distinct mechanisms: activation by GTP loading and inactivation by GTP hydrolysis. GTP loading is a two-step process that requires the release of bound GDP and its replacement by a GTP molecule. Nucleotide release is a spontaneous but slow process that has to be catalyzed by RHO-specific guanine nucleotide exchange factors (RHOGEFs), which associate with RHO GTPases and trigger release of the nucleotide. The resulting nucleotide-free binary complex has no particular nucleotide specificity. However, the cellular concentration of GTP is markedly higher than that of GDP, which favors GTP loading, resulting in the activation of RHO GTPases.

Conversely, to turn off the switch, GTP has to be hydrolyzed. This is facilitated by RHO-specific GTPase-activating proteins (RHOGAPs), which stimulate the intrinsically slow hydrolytic activity of RHO proteins. Although guanine nucleotide exchange factors (GEFs) and GTPase-activating proteins (GAPs) are the canonical regulators of this cycle, several alternative mechanisms, such as post-translational modifications, may fine-tune the RHO switch. In addition, inactive RHO GTPases are extracted by RHO-specific guanine nucleotide dissociation inhibitors (RHOGDIs) from cell membranes to prevent their inappropriate activation and to protect them from misfolding and degradation. (Garcia-Mata, R. et al. “The ‘invisible hand’: regulation of RHO GTPases by RHOGDIs.” Nature Reviews Molecular Cell Biology. (2011) 12:493-504; at 494).

Many proteins aid in activating and inhibiting ROCK I and ROCK II. For example, small GTP-binding proteins RhoA (which controls cell adhesion and motility through organization of the actin cytoskeleton and regulation of actomyosin contractility) (Yoshioka, K. et al., “Overexpression of Small GTP-binding protein RhoA promotes Invasion of Tumor Cells,” J. Cancer Res. (1999) 59: 2004-2010), RhoB (which is localized primarily on endosomes, has been shown to regulate cytokine trafficking and cell survival) and RhoC (which may be more important in cell locomotion) (Wheeler, A. P., Ridley, A. J., “Why three Rho proteins? RhoA, RhoB, RhoC and cell motility,” Exp. Cell Res. (2004) 301(1): 43-49) associate with and activate the ROCK proteins. Other GTP binding proteins, such as RhoE, Ras associated with diabetes (Rad) and Gem (a member of the RGK family of GTP-binding proteins within the Ras superfamily possessing a ras-like core and terminal extensions whose expression inhibited ROK beta-mediated phosphorylation of myosin light chain and myosin phosphatase, but not LIM kinase, (see Ward Y., et al., J. Cell Biol. (2002) 157(2): 291-302), inhibit ROCK, binding at sites distinct from the canonical Ras binding domain (RBD). Association with the PDK1 kinase promotes ROCK I activity by blocking RhoE association.

ROCK activation leads to a concerted series of events that promote force generation and morphological changes. These events contribute directly to a number of actin-myosin mediated processes, such as cell motility, adhesion, smooth muscle contraction, neurite retraction and phagocytosis. In addition, ROCK kinases play roles in proliferation, differentiation, apoptosis and oncogenic transformation, although these responses can be cell type-dependent. (Olson, M. F. “Applications for ROCK kinase inhibition” Curr Opin Cell Biol. (2008) 20(2): 242-248, at 242-243).

ROCK I and ROCK II promote actin-myosin mediated contractile force generation through the phosphorylation of numerous downstream target proteins, including ezrin/radixin/moesin (ERM), the LIM-kinases (LIMK), myosin light chain (MLC), and MLOC phosphatase (MLCP). ROCK phosphorylates LIM kinases-1 and -2 (LIMK1 and LIMK2) at conserved Threonines in their activation loops, increasing LIMK activity and the subsequent phosphorylation of cofilin proteins, which blocks their F-actin-severing activity. ROCK also directly phosphorylates the myosin regulatory light chain, myosin light chain II (MLC), and the myosin binding subunit (MYPT1) of the MLC phosphatase to inhibit catalytic activity. Many of these effects are also amplified by ROCK-mediated phosphorylation and activation of the Zipper-interacting protein kinase (ZIPK), a serine/threonine kinase which is involved in the regulation of apoptosis, autophagy, transcription, translation, actin cytoskeleton reorganization, cell motility, smooth muscle contraction and mitosis, which phosphorylates many of the same substrates as ROCK.

The phosphorylation of MLC by ROCK provides the chemical energy for actin-myosin ratcheting, and also phosphorylates myosin light chain phosphatase (MLCP), thereby inactivating MLCP and preventing its dephosphorylation of MLC. Thus, ROCK promotes actin-myosin movement by activation and stabilization. Other known substrates of ROCK include the cytoskeleton related proteins such as the ERM proteins, and focal adhesion kinase (FAK). The ERM proteins function to connect transmembrane proteins to the cytoskeleton. (Street, C. A. and Bryan, B. A. “Rho Kinase Proteins-Pleiotropic Modulators of Cell Survival and Apoptosis” Anticancer Res. (2011) 31(11): 3645-3657, at 3650). ROCK has been linked to apoptosis, cell survival, and cell cycle progression.

Rho-ROCK signaling has been implicated in cell cycle regulation. Rho-ROCK signaling increases cyclin D1 and Cip1 protein levels, which stimulate G1/S cell cycle progression. (Morgan-Fisher, M. et al., 61:185-198, at 189). Polyploidization naturally occurs in megakaryocytes due to an incomplete mitosis, which is related to a partial defect in Rho-ROCK activation, and leads to an abnormal contractile ring lacking myosin IIA. Rho-ROCK signaling also has been linked to apoptosis and cell survival. During apoptosis, ROCK I and ROCK II are altered to become constitutively-active kinases. Through proteolytic cleavage by caspases (ROCK I) or granzyme B (ROCK II), a carboxyl-terminal portion is removed that normally represses activity. Interaction with phosphatidyl inositol (3,4,5)-triphosphate (PIP3) provides an additional regulatory mechanism by localizing ROCK II to the plasma membrane where it can undertake spatially restricted activities, i.e. the regulation by localization of enzymatic activity. Phosphorylation at multiple specific sites by polo-like kinase 1 was found to promote ROCK II activation by RhoA. (Olson, M. F., 20(2): 242-248, at 242). Additional Serine/Threonine and Tyrosine kinases may also regulate ROCK activity given that more phosphorylations have been identified. (Id.). Specifically, protein oligomerization induces N-terminal trans-phosphorylation. (Riento, K. and Ridley, A. J., “ROCKs: multifunction kinases in cell behavior.” Nat Rev Mol Cell Biol. (2003) 4:446-456). Other direct activators include intracellular second messengers such as arachodonic acid and sphingosylphosphorylcholine which can activate ROCK independently of Rho. Furthermore, ROCK1 activity can be induced during apoptosis. (Mueller, B. K. et al., “Rho Kinase, a promising drug target for neurological disorders.” (2005) Nat Rev Mol Cell Biol. 4(6): 387-398).

ROCK protein signaling reportedly acts in either a pro- or anti-apoptotic fashion depending on cell type, cell context and microenvironment. For instance, ROCK proteins are essential for multiple aspects of both the intrinsic and extrinsic apoptotic processes, including regulation of cytoskeletal-mediated cell contraction and membrane blebbing, nuclear membrane disintegration, modulation of Bcl2-family member and caspase expression/activation and phagocytosis of the fragmented apoptotic bodies (Mueller, B. K., et al., 4:387-398). ROCK signaling also exhibits pro-survival roles. Though a wealth of data exists to suggest both pro- and anti-survival roles for ROCK proteins, the molecular mechanisms that modulate these pleitropic roles are largely unknown. (Street, C. A. and Bryan, B. A., 31(11):3645-3657).

The term “sign” as used herein refers to something found during a physical exam or from a laboratory test that shows that a person may have a condition or disease. The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human. The term “subject in need of such treatment” as used herein refers to a patient who suffers from a disease, disorder, condition, or pathological process, e.g., a cancer. According to some embodiments, the term “subject in need of such treatment” also is used to refer to a patient whose cancer comprises a population of cancer cells sensitive to an EBP-modulating anti-cancer compound (i) who will be administered an EBP modulating anti-cancer compound; (ii) is receiving an EBP modulating anti-cancer compound; or (iii) has received an EBP-modulating anti-cancer compound, unless the context and usage of the phrase indicates otherwise.

The terms “substantial inhibition”, “substantially inhibited” and the like as used herein refer to inhibition of at least 50%, inhibition of at least 55%, inhibition of at least 60%, inhibition of at least 65%, inhibition of at least 70%, inhibition of at least 75%, inhibition of at least 80%, inhibition of at least 85%, inhibition of at least 90%, inhibition of at least 95%, or inhibition of at least 99%.

The term “survival rate” as used herein refers to the percent of individuals who survive a disease (e.g., cancer) for a specified amount of time. For example, if the 5-year survival rate for a particular cancer is 34%, this means that 34 out of 100 individuals initially diagnosed with that cancer would be alive after 5 years.

The term “sterol” as used herein refers to a steroid alcohol, which contains a common steroid nucleus (a fused, reduced 17-carbon-atom ring system, cyclopentanoperhydrophenantrene) and a hydroxyl group. The term “truncated” as used herein refers to shortened by cutting off residues; being cut short. The term “tumor” as used herein refers to a diseases involving abnormal cell growth in numbers (proliferation) or in size with the potential to invade or spread to other parts of the body (metastasis). The term “tumor burden” or “tumor load” are used interchangeably herein refers to the number of cancer cells, the size of a tumor, or the amount of cancer in the body.

The following example is provided to further illustrate the advantages and features of the present disclosure, but it is not intended to limit the scope of the disclosure. While this example is typical of those that might be used, other procedures, methodologies, or techniques known to those skilled in the art may alternatively be used.

### EXAMPLES

### Example 1

### Synthesis of the Tasin Derivatives

General Procedure. Unless otherwise specified, all commercially available reagents were used as received. All reactions using dried solvents were carried out under an atmosphere of argon in flame-dried glassware with magnetic stirring. Dry solvent was dispensed from a solvent purification system that passes solvent through two columns of dry neutral alumina. Silica gel chromatographic purifications were performed by flash chromatography with silica gel (Sigma, grade 60, 230-400 mesh) packed in glass columns (the eluting solvent was determined by thin layer chromatography, TLC), or with an Isco Combiflash system using Redisep®Rf Flash columns with size ranging from 4 to 80 grams. Analytical TLC was performed on glass plates coated with 0.25 mm silica gel using UV or by iodide or KMnO₄ staining for visualization. Melting points are uncorrected. Routine ¹H and proton-decoupled ¹³C NMR spectra were obtained on a Bruker 400 MHz NMR spectrometer. Chemical shifts (δ) are reported in parts per million (ppm) from low to high field relative to residual solvent. Multiplicities are given as: s (singlet), d (doublet), t (triplet), q (quartet), dd (doublet of doublets), m (multiplet). All synthetic compounds exhibited >95% purity as determined by LC-MS analysis performed on an Agilent 1100 HPLC system using an Eclipse XDB-C18 column (4.6×150 mm, 5 μm; Agilent) that was coupled to an Agilent G1956A (or 6120) ESI mass spectrometer run in the positive mode with a scan range of 100 to 1,100 m/z. Liquid chromatography was carried out at a flow rate of 0.5 mL/min at 20° C. with a 5 μL injection volume, using the gradient elution with aqueous acetonitrile containing 0.1% formic acid. The gradient was adjusted based on the different polarity of different compounds. HRMS data were obtained from the Shimadzu Center for Advanced Analytical Chemistry (SCAAC) at U.T. Arlington.

General procedure A for the preparation of sulfonamides from sulfonyl chlorides and amines (compounds 4-9, 11-24, 26-51, 57, 58, 71-91, 104, 115, 116, 124, and 126-128). A mixture of amine (1.0 eq.), sulfonyl chloride (1.1 eq.), and N,N-diisopropyl ethylamine (1.5 eq.) in CH₂Cl₂ (5 mL/mmol amine) was stirred at room temperature overnight. The reaction solution was then poured into a saturated aqueous NaHCO₃ solution (20 mL/mmol amine) and extracted with CH₂Cl₂ (3×20 mL/mmol amine). The combined organic layers were dried over Na₂SO₄, filtered and concentrated under reduced pressure. The residue was purified through flash chromatography or Isco Combiflash (MeOH/CH₂Cl₂, or MeOH/EtOAc eluent mixture; gradient adjusted based on the different polarity of different compounds), or by recrystallization to provide the corresponding sulfonamides with >95% purity.

General procedure B for the preparation of sulfonamides from sulfonyl chlorides and amine hydrochloride salts (3a-c, 103, 113). A biphasic mixture of sulfonyl chloride (1.2 eq.), amine hydrochloride salt (1.0 eq.) and K₂CO₃ (2.5 eq.) in CHCl₃ (2 mL/mmol amine hydrochloride salt) and water (2 mL/mmol amine hydrochloride salt) was stirred vigorously at room temperature for 20 h followed by the addition of saturated aqueous NaHCO₃(25 mL/mmol of amine hydrochloride salt). The resulting solution was extracted with CH₂Cl₂ (3×20 mL/mmol of amine hydrochloride salt) and the combined organic layers were dried over Na₂SO₄, filtered and concentrated under reduced pressure. The residue was purified through flash chromatography or Isco Combiflash (MeOH/CH₂Cl₂, or hexanes/EtOAc eluent mixture; gradient adjusted based on the different polarity of different compounds) to provide the corresponding sulfonamides with >95% purity.

General procedure C for the preparation of biarylsulfonamides via Suzuki cross-coupling (compounds 52-56 and 59-70). To a flame-dried flask equipped with a reflux condenser were added bromophenylsulfonyl)-4-methyl-1,4′-bipiperidine (1.0 eq.), phenylboronic acid (1.58 eq.), Pd(PPh₃)₄ (0.1 eq.), THF (14.5 mL/mmol sulfonamide) and aqueous Na₂CO₃ (2 M; 1.45 mL/mmol sulfonamide). The mixture was degassed through freeze-pump-thaw cycling and was refluxed for 3-12 h. After being cooled down to room temperature, the reaction suspension was diluted with water (45.5 mL/mmol sulfonamide), stirred for 10 min and was extracted with CH₂Cl₂ (3×54.5 mL/mmol sulfonamide). The combined organic layers were dried over Na₂SO₄, filtered and concentrated. The residue was purified through flash chromatography or Isco Combiflash (MeOH/CH₂Cl₂, or MeOH/EtOAc eluent mixture; gradient adjusted based on the different polarity of different compounds) to provide the corresponding biarylsulfonamides with >95% purity.

General procedure D for the reductive amination of N-arylsulfonyl-piperidin-4-ones 3a-c, or N-mesitylsulphonyl-4-aminocyclohexanone. A mixture of ketone (1.0 eq.), amine (1.0 e.q), AcOH (1.0 eq.), and CH₂Cl₂ (or DCE) (5 ml/mmol amine) was stirred at room temperature for 15 min before NaBH(OAc)₃ (1.5 eq.) was added. The resulting suspension was stirred at room temperature with a reaction time ranging from 20 h to 89 h. The reaction was then quenched by dropwise addition of saturated aqueous NaHCO₃(30 mL/mmol of amine) at 0° C. and the resulting biphasic solution was extracted with CH₂Cl₂ (3×30 ml/mmol amine). The combined organic layers were dried over Na₂SO₄, filtered and concentrated under reduced pressure. The residue was purified through flash chromatography or Isco Combiflash (MeOH/CH₂Cl₂, or hexanes/EtOAc eluent mixture; gradient adjusted based on the different polarity of different compounds) to provide the corresponding reductive amination product with >95% purity.

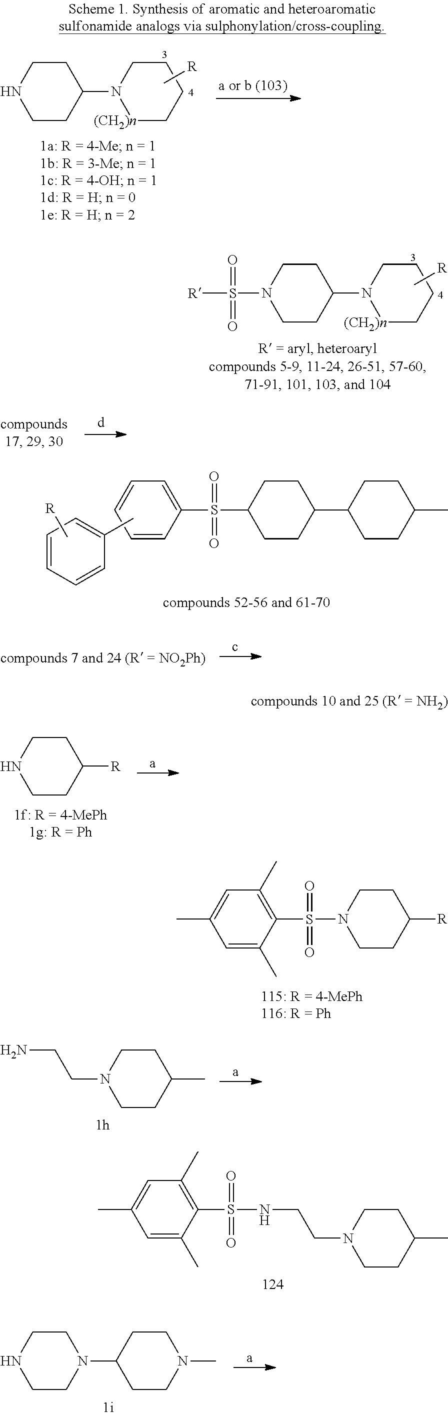

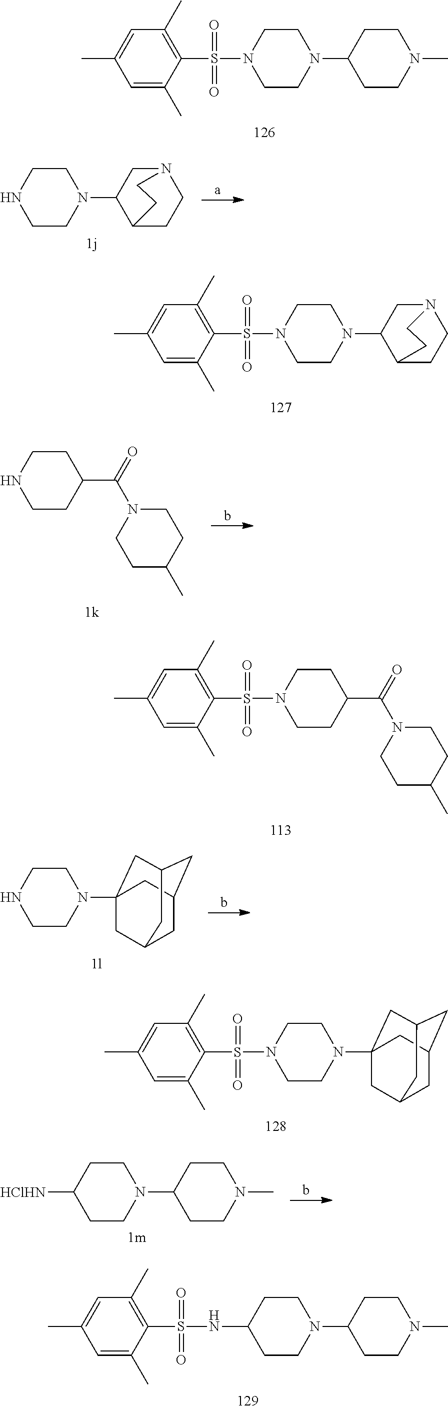

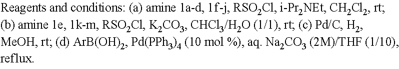

The synthesis of compounds 5-91, 101, 103, 104, 113, 115, 116, 124, 126-129 are shown in scheme 1. Standard sulfonylation of substituted 1,4′-bipiperidines 1a-c, 4-(pyrrolidin-1-yl)piperidine (1d), or 1-(piperidin-4-yl)azepane (1e) with a variety of commercially available aryl- or heteroarylsulfonyl chlorides at room temperature provided analogs 5-9, 11-24, 26-51, 57-60, 71-91, 101, 103, and 104. (Engler et al. (2000). J. Org. Chem. 65:2444-57). A subsequent hydrogenolysis of nitro-substituted analogs 7 and 24 over Pd/C at room temperature yielded aniline analogs 10 and 25. The 2-, 3-, or 4-bromophenylsulfonamide analogs 17, 29, and 30 offered viable starting materials for further diversification toward biaryl-substituted congeners 52-56 and 61-70 via Suzuki cross-coupling with commercially available arylboronic acids. (Han et al. (2000). J. Med. Chem. 23:4398-4415). Variants in the bipiperidinyl moiety were prepared via reaction of the corresponding heterocyclic amines 1f-l with 2,4,6-trimethylphenylsulfonyl chloride under the same standard sulfonylation conditions. Engler, Supra.

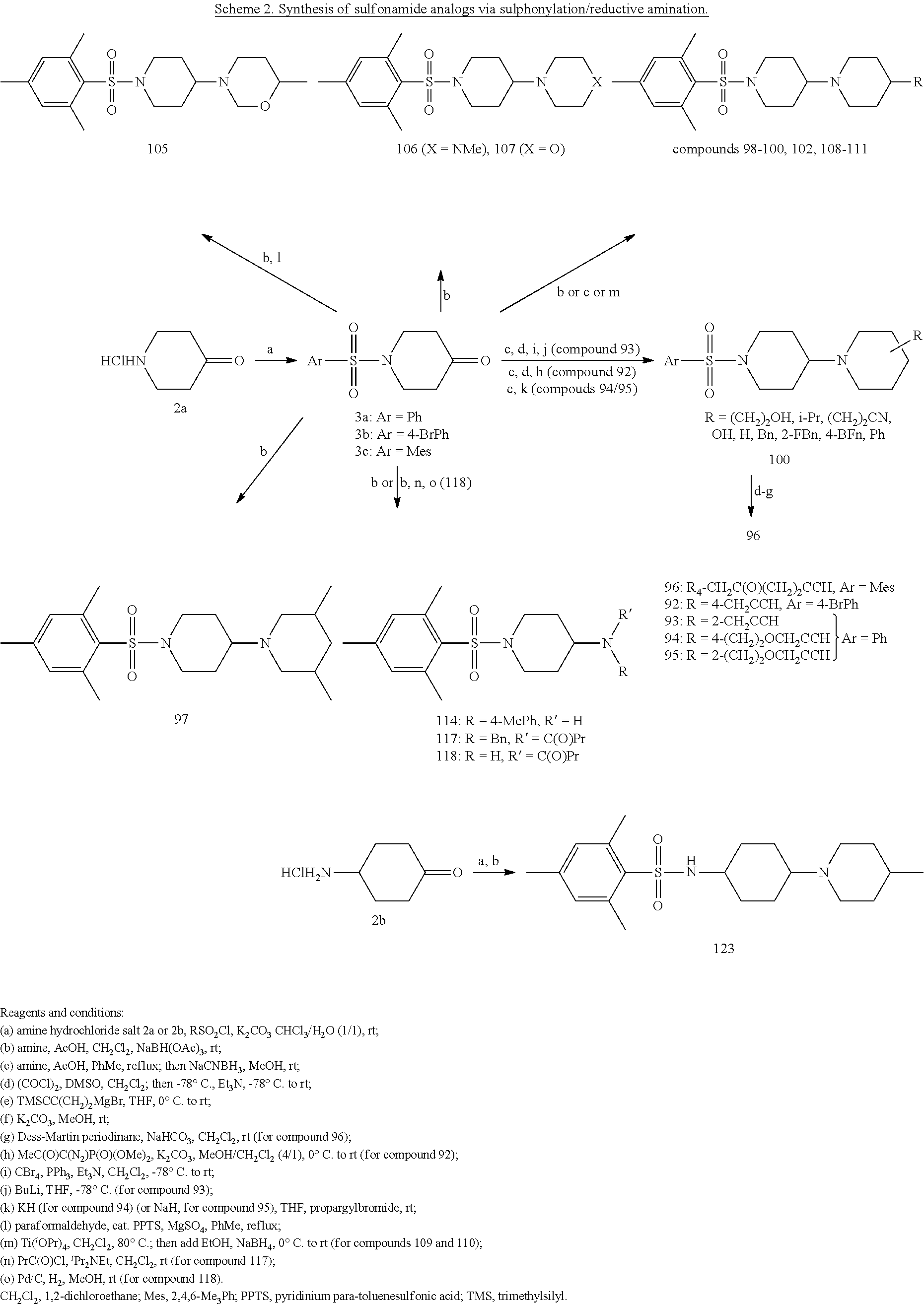

Additional sulfonamide analogs not directly available from direct sulfonylation of commercially available amines were synthesized via procedures outlined below in Scheme 2. First, reaction of piperidine-4-one (2a) or 4-aminocyclohexan-1-one (2b) with phenyl-, 4-bromophenyl-, or 2,4,6-trimethylphenylsulfonyl chloride provided intermediate sulfonamides 3a-c and 2,4,6-trimethyl-N-(4-oxocyclohexyl)benzenesulfonamide (not shown, derived from 2b). Id. Subsequent reductive amination (NaBH(OAc)₃ or NaCNBH₃) of these materials with a variety of amines yielded compounds 97-100, 102, 106-111, 114, and 123. (Abdel-Magid et al. (1996). J. Org. Chem. 61:3849-62). Compound 105 was obtained after an additional condensation of the intermediate 4-((1-(mesitylsulfonyl)piperidin-4-yl)amino)butan-2-ol with paraformaldehyde. Compound 117 derived from acylation of intermediate N-benzyl-1-(mesitylsulfonyl)piperidin-4-amine, which upon subsequent hydrogenolysis yielded analog 118. Compound 96 was made from hydroxyethyl-substituted compound 100 (R=4-(CH₂)₂OH) via a sequence of reactions including Swern oxidation, addition of (4-(trimethylsilyl)but-3-yn-1-yl)magnesium bromide to the thus obtained aldehyde, silyl-deprotection, and oxidation to the ketone with Dess-Martin periodinane. Compounds 92-94 are derived after reductive amination of arylsulfonylpiperidinone 3a or 3b with 2- or 4-(hydroxyethyl)piperidine, followed by alkylation with propargyl bromide (compounds 94, 95). Alternatively, the same reductive amination products were oxidized to the aldehyde, followed by Gilbert-Seyferth (compound 92) or Corey-Fuchs alkynylation (compound 93).

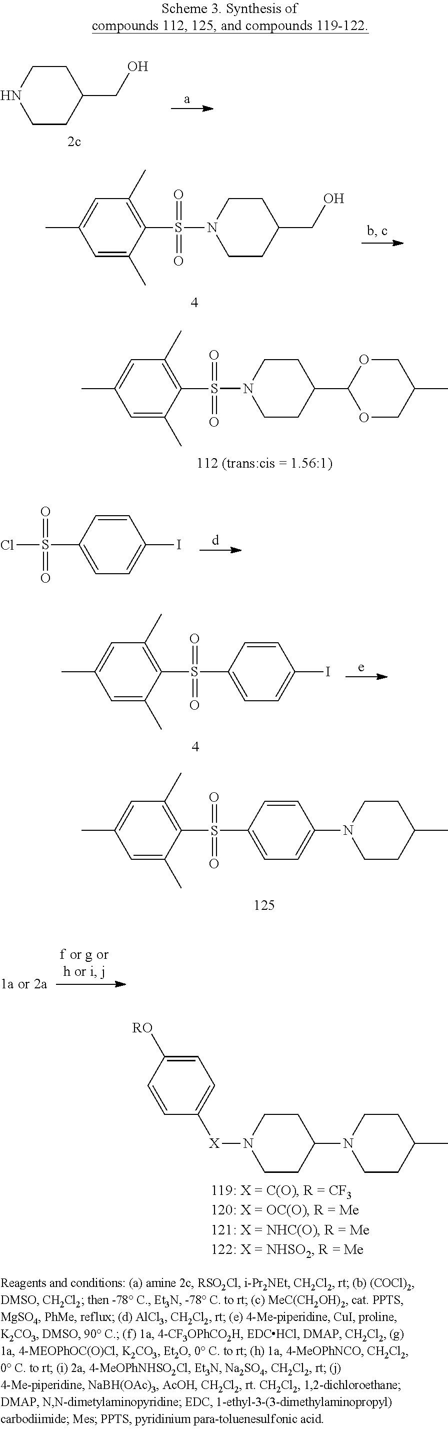

As shown in Scheme 3, the 1,3-dioxanyl-containing compound 112 (1.56:1 mixture of cis:trans isomers) was synthesized from 4-hydroxymethyl-piperidine 2c via sulfonylation (compound 4), followed by Swern oxidation and condensation with 2-methyl-1,3-propanediol (Scheme 3). The synthesis of compound 125 relied on a copper-catalyzed Buchwald amination of 1-iodo-4-(2,4,6-trimethylphenylsulfonyl)benzene with 4-methylpiperidine. (Ma et al. (2003) Org. Lett, 14:2453-55). We also explored analogs wherein the sulfonamide linker is replaced with various other functionality. As shown in Scheme 3, condensation of 4-methyl-1,4′-bipiperidine with 4-trifluoromethoxybenzoic acid provided amide compound 119, whereas reaction with 4-methoxyphenyl carbonochloridate or 4-methoxyphenylisocyanate yielded carbamate compound 120 and urea compound 121, respectively. Finally, reaction of piperidinone 2b with (4-methoxyphenyl)sulfamoyl chloride, followed by reductive amination with 4-methylpiperidine furnished compound 122.

Compound 3a: 1-(Phenylsulfonyl)piperidin-4-one. Reaction of amine hydrochloride salt 2a with benzenesulfonyl chloride (Procedure B) yielded 3a as a white solid (93%); mp 105-108° C. ¹H NMR (400 MHz, CDCl₃) δ 7.85-7.73 (m, 2H), 7.66-7.58 (m, 1H), 7.58-7.48 (m, 2H), 3.39 (t, J=6.2 Hz, 4H), 2.52 (t, J=6.2 Hz, 4H). ¹³C NMR (126 MHz, CDCl₃) δ 205.6, 136.3, 133.2, 129.3, 127.5, 45.9, 40.7. The analytical data were consistent with the literature report. (Ellis et al. (2008) J. Med. Chem. 51:2170-77).

Compound 3b: 1-(4-Bromophenylsulfonyl)piperidin-4-one. Reaction of amine hydrochloride salt 2a with 4-bromobenzene-1-sulfonyl chloride (Procedure B) yielded 3b as a white solid (82%); mp 155-158° C. ¹H NMR (400 MHz, CDCl₃) δ 7.79-7.57 (m, 4H), 3.39 (t, J=6.3 Hz, 4H), 2.54 (t, J=6.3 Hz, 4H). ¹³C NMR (100 MHz, CDCl₃) δ 205.1, 135.5, 132.6, 128.9, 128.4, 45.8, 40.6.

Compound 3c: 1-(Mesitylsulfonyl)piperidin-4-one. Reaction of amine hydrochloride salt 2a with 2,4,6-trimethylbenzene-1-sulfonyl chloride (Procedure B) yielded 3c as a white solid (95%); mp 102-105° C. ¹H NMR (400 MHz, CDCl₃) δ 6.95 (s, 2H), 3.50 (t, J=6.2 Hz, 4H), 2.61 (s, 6H), 2.52 (t, J=6.2 Hz, 4H), 2.29 (s, 3H). ¹³C NMR (100 MHz, CDCl₃) δ 201.6, 138.3, 135.6, 127.4, 126.8, 39.6, 36.3, 18.1, 16.2. LC-MS (ESI) calcd for C₁₄H₂₀NO₃S [M+H]⁺ 282.1, found 282.1.

Compound 4: (1-(Mesitylsulfonyl)piperidin-4-yl)methanol. Reaction of amine 2c with 2,4,6-trimethylbenzene-1-sulfonyl chloride (Procedure A) yielded 4 as a white solid (78%); mp 85-88° C. ¹H NMR (400 MHz, CDCl₃) δ 6.91 (s, 2H), 3.56 (d, J=12.3 Hz, 2H), 3.43 (d, J=6.3 Hz, 2H), 2.71 (td, J=12.3, 2.6 Hz, 2H), 2.57 (s, 6H), 2.26 (s, 3H), 2.00-1.80 (m, 1H), 1.73 (dd, J=13.6, 2.9 Hz, 2H), 1.63-1.48 (m, 1H), 1.18 (qd, J=11.9, 4.3 Hz, 2H). ¹³C NMR (100 MHz, CDCl₃) δ 142.5, 140.3, 131.9, 131.7, 67.0, 44.1, 38.3, 28.1, 22.8, 20.9. HRMS (ESI-TOF) calcd for C₁₅H₂₃NO₃SNa [M+Na]⁺ 320.1291, found 320.1280.

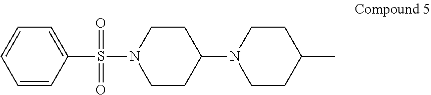 [SMILES: CC1CCN(C2CCN(S(=O)(=O)c3ccccc3)CC2)CC1]

Compound 5: 4-methyl-1′-(phenylsulfonyl)-1,4′-bipiperidine: Reaction of amine 1a with PhSO₂Cl (Procedure A) yielded 5 as a pale yellow solid (92%); mp 154-156° C. ¹H NMR (500 MHz, CDCl₃) δ 7.77 (d, J=7.3 Hz, 2H), 7.65-7.58 (m, 1H), 7.54 (t, J=7.7 Hz, 2H), 3.87 (d, J=11.9 Hz, 2H), 2.80 (d, J=11.8 Hz, 2H), 2.32-2.20 (m, 3H), 2.14 (t, J=11.4 Hz, 2H), 1.86 (d, J=11.7 Hz, 2H), 1.72-1.59 (m, 4H), 1.40-1.28 (m, 1H), 1.27-1.12 (m, 2H), 0.91 (d, J=6.4 Hz, 3H). ¹³C NMR (100 MHz, CDCl₃) δ 136.0, 132.7, 129.0, 127.6, 61.3, 49.1, 46.1, 33.9, 30.8, 26.9, 21.7. HRMS (ESI-TOF) calcd for C₁₇H₂₇N₂O₂S [M+H]⁺ 323.1788, found 323.1774.

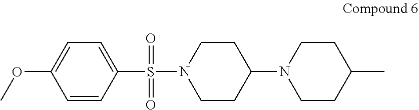

Compound 6: 1′-((4-methoxyphenyl)sulfonyl)-4-methyl-1,4′-bipiperidine: Reaction of amine 1a with 4-methoxybenzene-1-sulfonyl chloride (Procedure A) yielded 6 as a white solid (94%); mp 109-112° C. ¹H NMR (400 MHz, CDCl₃) δ 7.67 (d, J=8.8 Hz, 2H), 6.97 (d, J=8.8 Hz, 2H), 3.85 (s, 3H), 3.83-3.74 (d, J=12.0 Hz, 2H), 2.76 (d, J=11.7 Hz, 2H), 2.28-2.04 (m, 5H), 1.88-1.75 (d, J=12.9 Hz, 2H), 1.72-1.54 (m, 4H), 1.36-1.23 (m, 1H), 1.23-1.08 (m, 2H), 0.87 (d, J=6.3 Hz, 3H). ¹³C NMR (100 MHz, CDCl₃) δ 162.9, 129.8, 127.6, 114.1, 61.5, 55.6, 49.5, 46.2, 34.6, 31.0, 27.3, 21.9. HRMS (ESI-TOF) calcd for C₁₈H₂₉N₂O₃S [M+H]⁺ 353.1893, found 353.1883.

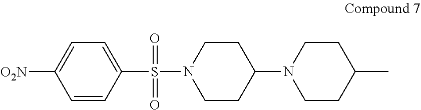 [SMILES: CC1CCN(C2CCN(S(=O)(=O)c3ccc([N+](=O)[O-])cc3)CC2)CC1]

Compound 7: 4-Methyl-1′-((4-nitrophenyl)sulfonyl)-1,4′-bipiperidine. Reaction of amine 1a with 4-nitrobenzene-1-sulfonyl chloride (Procedure A) yielded 7 as a yellow solid (76%); mp 165-168° C. ¹H NMR (400 MHz, CDCl₃) δ 8.35 (d, J=8.8 Hz, 2H), 7.91 (d, J=8.8 Hz, 2H), 3.85 (d, J=12.3 Hz, 2H), 2.76 (d, J=11.6 Hz, 2H), 2.31 (td, J=12.0, 2.5 Hz, 2H), 2.21 (tt, J=11.5, 3.7 Hz, 1H), 2.10 (td, J=11.5, 2.5 Hz, 2H), 1.84 (d, J=12.0 Hz, 2H), 1.62 (qd, J=12.0, 4.0 Hz, 4H), 1.38-1.21 (m, 1H), 1.14 (qd, J=12.0, 3.7 Hz, 2H), 0.86 (d, J=6.4 Hz, 3H). ¹³C NMR (100 MHz, CDCl₃) δ 150.1, 142.4, 128.7, 124.3, 61.2, 49.5, 46.1, 34.5, 30.9, 27.4, 21.8. HRMS (ESI-TOF) calcd for C₁₇H₂₆N₃O₄S [M+H]⁺ 368.1639, found 368.1633.

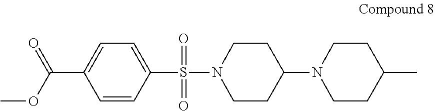

Compound 8: Methyl 4-((4-methyl-[1,4′-bipiperidin]-1′-yl)sulfonyl)benzoate. Reaction of amine 1a with methyl 4-(chlorosulfonyl)benzoate (Procedure A) yielded 8 as a white solid (54%). ¹H NMR (400 MHz, CDCl₃) δ 8.16 (d, J=8.1 Hz, 2H), 7.80 (d, J=8.7 Hz, 2H), 3.93 (s, 3H), 3.84 (d, J=12.1 Hz, 2H), 2.76 (d, J=11.0 Hz, 2H), 2.38-2.02 (m, 5H), 1.83 (d, J=11.9 Hz, 2H), 1.71-1.51 (m, 4H), 1.37-1.23 (m, 1H), 1.16 (qd, J=12.0, 3.8 Hz, 2H), 0.87 (d, J=6.3 Hz, 3H). ¹³C NMR (100 MHz, CDCl₃) δ 165.6, 140.2, 133.8, 130.2, 127.6, 61.4, 52.6, 49.4, 46.1, 34.4, 30.9, 27.3, 21.8. HRMS (ESI-TOF) calcd for C₁₉H₂₉N₂O₄S [M+H]⁺ 381.1843, found 381.1843.

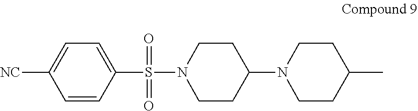 [SMILES: CC1CCN(C2CCN(S(=O)(=O)c3ccc(C#N)cc3)CC2)CC1]

Compound 9: 4-((4-Methyl-[1,4′-bipiperidin]-1′-yl)sulfonyl)benzonitrile. Reaction of amine 1a with 4-cyanobenzene-1-sulfonyl chloride (Procedure A) yielded 9 as a white solid (92%); mp 184-186° C. ¹H NMR (400 MHz, CDCl₃) δ 7.84 (q, J=8.5 Hz, 4H), 3.85 (d, J=12.1 Hz, 2H), 2.77 (d, J=11.6 Hz, 2H), 2.31 (t, J=12.0 Hz, 2H), 2.22 (tt, J=11.6, 3.6 Hz, 1H), 2.11 (t, J=12.2 Hz, 2H), 1.85 (d, J=11.6 Hz, 2H), 1.71-1.55 (m, 4H), 1.37-1.24 (m, 1H), 1.25-1.10 (m, 2H), 0.89 (d, J=6.4 Hz, 3H). ¹³C NMR (100 MHz, CDCl₃) δ 140.9, 132.8, 128.1, 117.3, 116.4, 61.2, 49.5, 46.1, 34.6, 31.0, 27.4, 21.8. HRMS (ESI-TOF) calcd for C₁₈H₂₆N₃O₂S [M+H]⁺ 348.1740, found 348.1737.

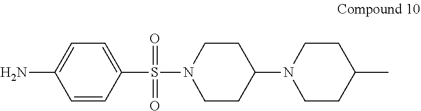 [SMILES: CC1CCN(C2CCN(S(=O)(=O)c3ccc(N)cc3)CC2)CC1]

Compound 10: 4-((4-Methyl-[1,4′-bipiperidin]-1′-yl)sulfonyl)aniline. To a 50 mL flask were added 4-methyl-1′-((4-nitrophenyl)sulfonyl)-1,4′-bipiperidine 7 (0.1004 g, 0.33 mmol), methanol (3 mL) and Pd/C (1 spatula, 10% on active carbon). The reaction flask was sealed by a septum and after the removal of air using vacuum, a hydrogen balloon was fitted on the top of the septum. The reaction suspension was then stirred at room temperature for 22 h and was filtered through a pad of celite. The filtrate was concentrated under reduced pressure to provide the desired product (0.09 g, >95%) as a colorless gel. ¹H NMR (400 MHz, CD₃OD) δ 7.41 (d, J=8.7 Hz, 2H), 6.69 (d, J=8.7 Hz, 2H), 3.84-3.63 (m, 2H), 2.83 (d, J=11.7 Hz, 2H), 2.31-2.06 (m, 5H), 1.87 (d, J=14.5 Hz, 2H), 1.62 (d, J=14.1 Hz, 2H), 1.51 (qd, J=12.2, 4.1 Hz, 2H), 1.41-1.23 (m, 1H), 1.16 (qd, J=12.4, 3.7 Hz, 2H), 0.89 (d, J=6.4 Hz, 3H). ¹³C NMR (100 MHz, CD₃OD) δ 153.1, 129.4, 121.2, 112.9, 61.3, 49.1, 45.9, 33.7, 30.7, 26.9, 20.8. HRMS (ESI-TOF) calcd for C₁₇H₂₇N₃O₂SNa [M+Na]⁺ 360.1716, found 360.1719.

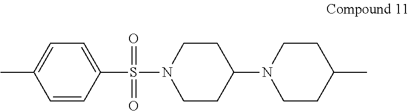

Compound 11: 4-Methyl-1′-tosyl-1,4′-bipiperidine. Reaction of amine 1a with 4-methylbenzene-1-sulfonyl chloride (Procedure A) yielded 11 as a white solid (67%); mp 139-142° C. ¹H NMR (400 MHz, CDCl₃) δ 7.59 (d, J=8.2 Hz, 2H), 7.28 (d, J=8.2 Hz, 2H), 3.80 (d, J=11.9 Hz, 2H), 2.79 (d, J=11.1 Hz, 2H), 2.39 (s, 3H), 2.30-2.07 (m, 5H), 1.84 (d, J=10.9 Hz, 2H), 1.69-1.53 (m, 4H), 1.40-1.09 (m, 3H), 0.86 (d, J=6.2 Hz, 3H). ¹³C NMR (100 MHz, CDCl₃) δ 143.5, 132.9, 129.6, 127.7, 61.6, 49.4, 46.0, 34.1, 30.8, 27.1, 21.7, 21.5. HRMS (ESI-TOF) calcd for C₁₈H₂₉N₂O₂S [M+H]⁺ 337.1944, found 337.1937.

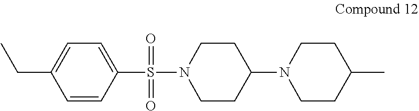 [SMILES: CCc1ccc(S(=O)(=O)N2CCC(N3CCC(C)CC3)CC2)cc1]

Compound 12: 1′-((4-Ethylphenyl)sulfonyl)-4-methyl-1,4′-bipiperidine. Reaction of amine 1a with 4-ethylbenzene-1-sulfonyl chloride (Procedure A) yielded 12 as a brown solid (>95%); mp 108-110° C. ¹H NMR (400 MHz, CDCl₃) δ 7.63 (d, J=7.9 Hz, 2H), 7.31 (d, J=7.9 Hz, 2H), 3.82 (d, J=11.6 Hz, 2H), 2.78 (d, J=11.2 Hz, 2H), 2.70 (q, J=7.6 Hz, 2H), 2.22 (t, J=12.1 Hz, 3H), 2.12 (t, J=10.8 Hz, 2H), 1.82 (d, J=12.2 Hz, 2H), 1.62 (td, J=12.3, 8.2 Hz, 4H), 1.39-1.08 (m, 6H), 0.87 (d, J=6.2 Hz, 3H). ¹³C NMR (100 MHz, CDCl₃) δ 149.5, 133.3, 128.4, 127.8, 61.5, 49.4, 46.1, 34.4, 30.9, 28.8, 27.2, 21.8, 15.1. HRMS (ESI-TOF) calcd for C₁₉H₃₁N₂O₂S [M+H]⁺ 351.2101, found 351.2093.

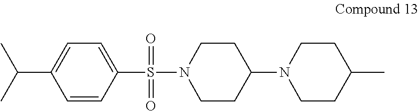 [SMILES: CC1CCN(C2CCN(S(=O)(=O)c3ccc(C(C)C)cc3)CC2)CC1]

Compound 13: 1′-((4-Isopropylphenyl)sulfonyl)-4-methyl-1,4′-bipiperidine. Reaction of amine 1a with 4-isopropylbenzene-1-sulfonyl chloride (Procedure A) yielded 13 as a yellow solid (79%); mp 144-147° C. ¹H NMR (400 MHz, CDCl₃) δ 7.63 (d, J=8.3 Hz, 2H), 7.33 (d, J=8.3 Hz, 2H), 3.81 (d, J=12.3 Hz, 2H), 2.94 (p, J=6.9 Hz, 1H), 2.75 (d, J=11.7 Hz, 2H), 2.31-2.15 (m, 3H), 2.09 (td, J=11.7, 2.7 Hz, 2H), 1.80 (d, J=11.7 Hz, 2H), 1.68-1.52 (m, 4H), 1.36-1.03 (m, 3H), 1.24 (d, J=6.9 Hz, 6H), 0.86 (d, J=6.3 Hz, 3H). ¹³C NMR (100 MHz, CDCl₃) δ 154.1, 133.4, 127.8, 127.0, 61.4, 49.4, 46.1, 34.5, 34.1, 30.9, 27.2, 23.6, 21.8. HRMS (ESI-TOF) calcd for C₂₀H₃₃N₂O₂S [M+H]⁺ 365.2257, found 365.2247.

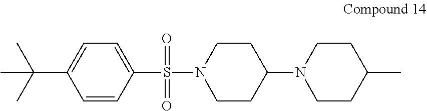 [SMILES: CC1CCN(C2CCN(S(=O)(=O)c3ccc(C(C)(C)C)cc3)CC2)CC1]

Compound 14: 1′-((4-(Tert-butyl)phenyl)sulfonyl)-4-methyl-1,4′-bipiperidine. Reaction of amine 1a with 4-(tert-butyl)benzene-1-sulfonyl chloride (Procedure A) yielded 14 as a yellow solid (85%); mp 173-176° C. ¹H NMR (400 MHz, CDCl₃) δ 7.64, 7.49 (ABq, J_{AB}=8.6 Hz, 4H), 3.83 (d, J=12.0 Hz, 2H), 2.77 (d, J=11.2 Hz, 2H), 2.31-2.18 (m, 3H), 2.13 (t, J=11.3 Hz, 2H), 1.82 (d, J=12.2 Hz, 2H), 1.70-1.51 (m, 4H), 1.31 (s, 9H), 1.30-1.25 (m, 1H), 1.24-1.11 (m, 2H), 0.86 (d, J=6.1 Hz, 3H). ¹³C NMR (100 MHz, CDCl₃) δ 156.4, 133.1, 127.5, 125.9, 61.5, 49.4, 46.1, 35.1, 34.3, 31.1, 30.9, 27.1, 21.8. HRMS (ESI-TOF) calcd for C₂₁H₃₅N₂O₂S [M+H]⁺ 379.2414, found 379.2418.

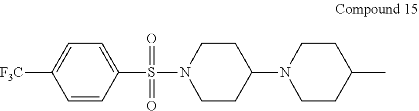 [SMILES: CC1CCN(C2CCN(S(=O)(=O)c3ccc(C(F)(F)F)cc3)CC2)CC1]

Compound 15: 4-Methyl-1′-((4-(trifluoromethyl)phenyl)sulfonyl)-1,4′-bipiperidine. Reaction of amine 1a with 4-(trifluoromethyl)benzene-1-sulfonyl chloride (Procedure A) yielded 15 as a yellow solid (87%); mp 175-178° C. ¹H NMR (400 MHz, CDCl₃) δ 7.86, 7.77 (ABq, J_{AB}=8.2 Hz, 4H), 3.85 (d, J=12.1 Hz, 2H), 2.75 (d, J=11.7 Hz, 2H), 2.28 (t, J=11.4 Hz, 3H), 2.19 (tt, J=11.5, 3.6 Hz, 1H), 2.09 (td, J=11.3, 2.4 Hz, 2H), 1.83 (d, J=11.5 Hz, 2H), 1.62 (qd, J=12.4, 4.0 Hz, 4H), 1.38-1.21 (m, 1H), 1.14 (qd, J=11.9, 3.8 Hz, 2H), 0.87 (d, J=6.4 Hz, 3H). ¹³C NMR (100 MHz, CDCl₃) δ 140.0, 134.4 (q, J=33.1 Hz), 128.1, 126.2 (q, J=3.7 Hz), 123.2 (q, J=272.9 Hz), 61.3, 49.4, 46.1, 34.4, 30.9, 27.3, 21.8. HRMS (ESI-TOF) calcd for C₁₈H₂₆F₃N₂O₂S [M+H]⁺ 391.1662, found 391.1654.

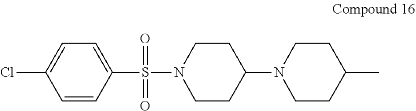 [SMILES: CC1CCN(C2CCN(S(=O)(=O)c3ccc(Cl)cc3)CC2)CC1]

Compound 16: 1′-((4-Chlorophenyl)sulfonyl)-4-methyl-1,4′-bipiperidine. Reaction of amine 1a with 4-chlorobenzene-1-sulfonyl chloride (Procedure A) yielded 16 as a white solid (67%); mp 152-155° C. ¹H NMR (400 MHz, CDCl₃) δ 7.66 (d, J=8.5 Hz, 2H), 7.47 (d, J=8.5 Hz, 2H), 3.93-3.69 (m, 2H), 2.80 (dt, J=11.6, 3.4 Hz, 2H), 2.23 (td, J=12.0, 2.5 Hz, 3H), 2.15 (t, J=11.3 Hz, 2H), 1.84 (d, J=12.6 Hz, 2H), 1.71-1.54 (m, 4H), 1.42-1.26 (m, 1H), 1.25-1.12 (m, 2H), 0.87 (d, J=6.2 Hz, 3H). ¹³C NMR (100 MHz, CDCl₃) δ 139.3, 134.6, 129.3, 129.0, 61.4, 49.4, 46.0, 34.2, 30.8, 27.2, 21.7. HRMS (ESI-TOF) calcd for C₁₇H₂₆ClN₂O₂S [M+H]⁺ 357.1398, found 357.1393.

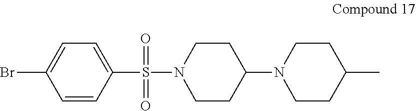 [SMILES: CC1CCN(C2CCN(S(=O)(=O)c3ccc(Br)cc3)CC2)CC1]

Compound 17: 1′-((4-Bromophenyl)sulfonyl)-4-methyl-1,4′-bipiperidine. Reaction of amine 1a with 4-bromobenzene-1-sulfonyl chloride (Procedure A) yielded 17 as a white solid (83%); mp 165-168° C. ¹H NMR (400 MHz, CDCl₃) δ 7.75-7.45 (m, 4H), 3.81 (d, J=11.9 Hz, 2H), 2.75 (d, J=11.5 Hz, 2H), 2.38-1.96 (m, 5H), 1.81 (d, J=11.8 Hz, 2H), 1.73-1.52 (m, 4H), 1.37-1.22 (m, 1H), 1.22-1.06 (m, 2H), 0.87 (d, J=6.4 Hz, 3H). ¹³C NMR (100 MHz, CDCl₃) δ 135.2, 132.3, 129.1, 127.7, 61.3, 49.5, 46.1, 34.6, 31.0, 27.3, 21.9. HRMS (ESI-TOF) calcd for C₁₇H₂₆BrN₂O₂S [M+H]⁺ 401.0893, found 401.0886.

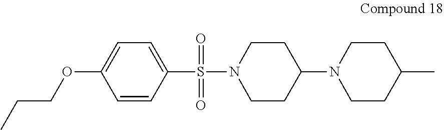 [SMILES: CCCOc1ccc(S(=O)(=O)N2CCC(N3CCC(C)CC3)CC2)cc1]

Compound 18: 4-Methyl-1′-((4-propoxyphenyl)sulfonyl)-1,4′-bipiperidine. Reaction of amine 1a with 4-propoxybenzene-1-sulfonyl chloride (Procedure A) yielded 18 as a white solid (>95%); mp 118-121° C. ¹H NMR (400 MHz, CDCl₃) δ 7.65 (d, J=8.9 Hz, 2H), 6.95 (d, J=8.9 Hz, 2H), 3.95 (t, J=6.6 Hz, 2H), 3.80 (d, J=11.4 Hz, 2H), 2.76 (d, J=10.9 Hz, 2H), 2.15 (dt, J=37.1, 11.5 Hz, 5H), 1.88-1.72 (m, 4H), 1.71-1.52 (m, 4H), 1.37-1.24 (m, 1H), 1.15 (q, J=11.8 Hz, 2H), 1.03 (t, J=7.4 Hz, 3H), 0.87 (d, J=6.4 Hz, 3H). ¹³C NMR (100 MHz, CDCl₃) δ 162.5, 129.7, 127.3, 114.5, 69.9, 61.5, 49.5, 46.2, 34.6, 31.0, 27.3, 22.4, 21.9, 10.5. HRMS (ESI-TOF) calcd for C₂₀H₃₃N₂O₃S [M+H]⁺ 381.2206, found 381.2210.

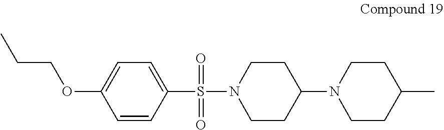 [SMILES: CCCOc1ccc(S(=O)(=O)N2CCC(N3CCC(C)CC3)CC2)cc1]

Compound 19: 1′-((4-Butoxyphenyl)sulfonyl)-4-methyl-1,4′-bipiperidine. Reaction of amine 1a with 4-butoxybenzene-1-sulfonyl chloride (Procedure A) yielded 19 as a pink solid (82%); mp 137-139° C. ¹H NMR (400 MHz, CDCl₃) δ 7.64 (d, J=8.7 Hz, 2H), 6.94 (d, J=8.7 Hz, 2H), 3.99 (t, J=6.5 Hz, 2H), 3.80 (d, J=12.3 Hz, 2H), 2.78 (d, J=11.3 Hz, 2H), 2.27-2.06 (m, 5H), 1.88-1.72 (m, 4H), 1.70-1.55 (m, 4H), 1.47 (h, J=7.4 Hz, 2H), 1.39-1.24 (m, 1H), 1.24-1.10 (m, 2H), 0.96 (t, J=7.4 Hz, 3H), 0.87 (d, J=6.3 Hz, 3H). ¹³C NMR (100 MHz, CDCl₃) δ 162.5, 129.7, 127.2, 114.5, 68.1, 61.6, 49.4, 46.1, 34.4, 31.0, 30.9, 27.2, 21.8, 19.1, 13.8. HRMS (ESI-TOF) calcd for C₂₁H₃₅N₂O₃S [M+H]⁺ 395.2363, found 395.2361.

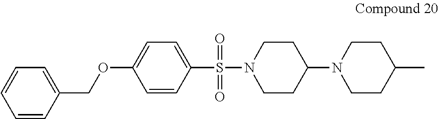 [SMILES: CC1CCN(C2CCN(S(=O)(=O)c3ccc(OCc4ccccc4)cc3)CC2)CC1]

Compound 20: 1′-((4-(Benzyloxy)phenyl)sulfonyl)-4-methyl-1,4′-bipiperidine. Reaction of amine 1a with 4-(benzyloxy)benzene-1-sulfonyl chloride (Procedure A) yielded 20 as a white solid (84%); mp 157-159° C. ¹H NMR (400 MHz, CDCl₃) δ 7.67 (d, J=8.8 Hz, 2H), 7.48-7.27 (m, 4H), 7.04 (d, J=8.8 Hz, 2H), 5.10 (s, 2H), 3.81 (d, J=11.5 Hz, 2H), 2.77 (d, J=10.9 Hz, 2H), 2.31-2.04 (m, 5H), 1.89-1.76 (m, 2H), 1.68-1.53 (m, 4H), 1.37-1.08 (m, 3H), 0.88 (d, J=6.4 Hz, 3H). ¹³C NMR (100 MHz, CDCl₃) δ 162.1, 135.8, 129.8, 128.7, 128.4, 127.9, 127.5, 115.0, 70.4, 61.5, 49.5, 46.2, 34.5, 31.0, 27.3, 21.9. HRMS (ESI-TOF) calcd for C₂₄H₃₃N₂O₃S [M+H]⁺ 429.2206, found 249.2195.

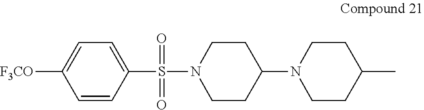 [SMILES: CC1CCN(C2CCN(S(=O)(=O)c3ccc(OC(F)(F)F)cc3)CC2)CC1]

Compound 21: 4-Methyl-1′-((4-(trifluoromethoxy)phenyl)sulfonyl)-1,4′-bipiperidine. Reaction of amine 1a with 4-(trifluoromethoxy)benzene-1-sulfonyl chloride (Procedure A) yielded 21 as a yellow solid (>95%); mp 168-170° C. ¹H NMR (400 MHz, CDCl₃) δ 7.79 (d, J=8.2 Hz, 2H), 7.34 (d, J=8.2 Hz, 2H), 3.84 (d, J=12.0 Hz, 2H), 2.79 (d, J=11.7 Hz, 2H), 2.35-2.19 (m, 3H), 2.13 (t, J=11.4 Hz, 2H), 1.86 (d, J=10.8 Hz, 2H), 1.71-1.55 (m, 4H), 1.39-1.11 (m, 3H), 0.88 (d, J=6.2 Hz, 3H). ¹³C NMR (100 MHz, CDCl₃) δ 152.1 (q, J=1.8 Hz), 134.7, 129.7, 120.8 (q, J=1.1 Hz), 120.2 (q, J=259.5 Hz), 61.3, 49.5, 46.1, 34.6, 31.0, 27.3, 21.8. HRMS (ESI-TOF) calcd for C₁₈H₂₆F₃N₂O₃S [M+H]⁺ 407.1611, found 407.115.

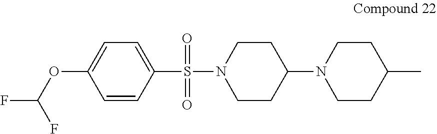 [SMILES: CC1CCN(C2CCN(S(=O)(=O)c3ccc(OC(F)F)cc3)CC2)CC1]

Compound 22: 1′-((4-(Difluoromethoxy)phenyl)sulfonyl)-4-methyl-1,4′-bipiperidine. Reaction of amine 1a with 4-(difluoromethoxy)benzene-1-sulfonyl chloride (Procedure A) yielded 22 as a light orange solid (69%); mp 132-135° C. ¹H NMR (500 MHz, CDCl₃) δ 7.76 (d, J=8.8 Hz, 2H), 7.24 (d, J=8.8 Hz, 2H), 6.61 (t, J=72.6 Hz, 1H), 3.83 (d, J=12.1 Hz, 2H), 2.77 (d, J=11.7 Hz, 2H), 2.33-2.16 (m, 3H), 2.11 (td, J=11.6, 2.5 Hz, 2H), 1.84 (d, J=13.2 Hz, 2H), 1.69-1.52 (m, 4H), 1.30 (dddt, J=13.3, 9.7, 6.5, 3.5 Hz, 1H), 1.16 (qd, J=12.0, 3.8 Hz, 2H), 0.89 (d, J=6.5 Hz, 3H). ¹³C NMR (100 MHz, CDCl₃) δ 154.1 (t, J=2.9 Hz), 132.9, 129.8, 119.3, 115.2 (t, J=262.5 Hz), 61.4, 49.5, 46.1, 34.6, 31.0, 27.3, 21.8. HRMS (ESI-TOF) calcd for C₁₈H₂₆F₂N₂O₃SNa [M+Na]⁺ 411.1524, found 411.1529.

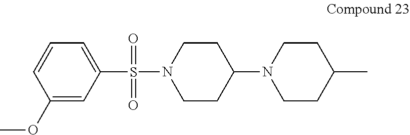

Compound 23: 1′-((3-Methoxyphenyl)sulfonyl)-4-methyl-1,4′-bipiperidine. Reaction of amine 1a with 3-methoxybenzene-1-sulfonyl chloride (Procedure A) yielded 23 as a yellow solid (85%); mp 81-83° C. ¹H NMR (400 MHz, CDCl₃) δ 7.40 (t, J=8.0 Hz, 1H), 7.29 (ddd, J=7.7, 1.6, 0.9 Hz, 1H), 7.22 (dd, J=2.6, 1.6 Hz, 1H), 7.08 (ddt, J=8.3, 2.6, 0.8 Hz, 1H), 3.83 (s, 3H), 3.82 (d, J=12.0 Hz, 2H), 2.79 (d, J=11.7 Hz, 2H), 2.25 (td, J=12.0, 2.6 Hz, 3H), 2.18-2.07 (m, 2H), 1.84 (d, J=12.3 Hz, 2H), 1.72-1.53 (m, 4H), 1.37-1.13 (m, 3H), 0.87 (d, J=6.2 Hz, 3H). ¹³C NMR (100 MHz, CDCl₃) δ 159.8, 137.2, 130.0, 119.7, 118.8, 112.5, 61.5, 55.6, 49.4, 46.1, 34.3, 30.9, 27.2, 21.8. HRMS (ESI-TOF) calcd for C₁₈H₂₉N₂O₃S [M+H]⁺ 353.1893, found 353.1898.

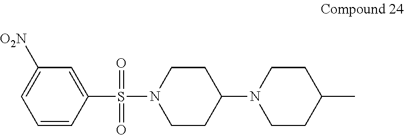 [SMILES: CC1CCN(C2CCN(S(=O)(=O)c3cccc([N+](=O)[O-])c3)CC2)CC1]

Compound 24: 4-Methyl-1′-((3-nitrophenyl)sulfonyl)-1,4′-bipiperidine. Reaction of amine 1a with 3-nitrobenzene-1-sulfonyl chloride (Procedure A) yielded 24 as a yellow solid (77%). ¹H NMR (400 MHz, CDCl₃) δ 8.55 (s, 1H), 8.42 (d, J=8.2 Hz, 1H), 8.06 (d, J=7.8 Hz, 1H), 7.74 (t, J=8.0 Hz, 1H), 3.86 (d, J=12.1 Hz, 2H), 2.75 (d, J=11.6 Hz, 2H), 2.32 (td, J=12.0, 2.5 Hz, 2H), 2.19 (tt, J=11.5, 3.6 Hz, 1H), 2.09 (td, J=11.6, 2.5 Hz, 2H), 1.84 (d, J=12.5 Hz, 2H), 1.71-1.52 (m, 4H), 1.37-1.20 (m, 1H), 1.13 (qd, J=12.1, 4.0 Hz, 2H), 0.86 (d, J=6.4 Hz, 3H). ¹³C NMR (100 MHz, CDCl₃) δ 148.3, 138.8, 133.0, 130.4, 127.1, 122.6, 61.2, 49.5, 46.1, 34.5, 31.0, 27.4, 21.8. HRMS (ESI-TOF) calcd for C₁₇H₂₆N₃O₄S [M+H]⁺ 368.1639, found 368.1630.

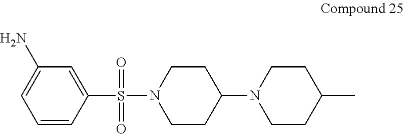 [SMILES: CC1CCN(C2CCN(S(=O)(=O)c3cccc(N)c3)CC2)CC1]

Compound 25: 3-((4-Methyl-[1,4′-bipiperidin]-1′-yl)sulfonyl)aniline. This compound was prepared as a yellow gel (55%) by hydrogenation of 24 in the same manner as for the preparation of 10. ¹H NMR (400 MHz, CD₃OD) δ 7.24 (t, J=7.9 Hz, 1H), 7.01 (t, J=2.0 Hz, 1H), 6.91 (dddd, J=23.6, 8.1, 2.0, 1.0 Hz, 2H), 3.76 (d, J=12.3 Hz, 2H), 3.29 (p, J=1.7 Hz, 1H), 2.83 (d, J=10.7 Hz, 2H), 2.28 (td, J=12.4, 2.4 Hz, 2H), 2.23-2.07 (m, 3H), 1.87 (d, J=12.9 Hz, 2H), 1.62 (dd, J=13.3, 3.5 Hz, 2H), 1.52 (qd, J=12.3, 4.1 Hz, 2H), 1.40-1.23 (m, 2H), 1.16 (qd, J=12.1, 3.8 Hz, 2H), 0.89 (d, J=6.4 Hz, 3H). ¹³C NMR (100 MHz, CD₃OD) δ 149.1, 136.3, 129.3, 118.5, 115.4, 112.5, 61.2, 49.1, 45.9, 33.7, 30.7, 26.9, 20.7. HRMS (ESI-TOF) calcd for C₁₇H₂₇N₃O₂SNa [M+Na]⁺ 360.1716, found 360.1703.

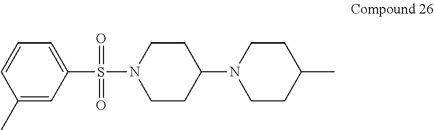 [SMILES: Cc1cccc(S(=O)(=O)N2CCC(N3CCC(C)CC3)CC2)c1]

Compound 26: 4-Methyl-1′-(m-tolylsulfonyl)-1,4′-bipiperidine. Reaction of amine 1a with 3-methylbenzene-1-sulfonyl chloride (Procedure A) yielded 26 as a yellow solid (86%); mp 90-93° C. ¹H NMR (400 MHz, CDCl₃) δ 7.62-7.43 (m, 2H), 7.41-7.31 (m, 2H), 3.81 (d, J=12.0 Hz, 2H), 2.80 (d, J=11.8 Hz, 2H), 2.39 (s, 3H), 2.34-2.06 (m, 5H), 1.85 (d, J=12.3 Hz, 2H), 1.68-1.55 (m, 4H), 1.37-1.13 (m, 3H), 0.86 (d, J=6.0 Hz, 3H). ¹³C NMR (100 MHz, CDCl₃) δ 139.2, 135.8, 133.5, 128.8, 127.9, 124.8, 61.6, 49.4, 46.0, 34.1, 30.8, 27.1, 21.7, 21.4. HRMS (ESI-TOF) calcd for C₁₈H₂₉N₂O₂S [M+H]⁺ 337.1944, found 337.1937.

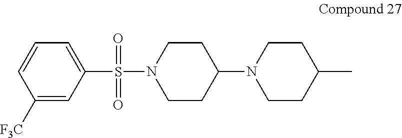 [SMILES: CC1CCN(C2CCN(S(=O)(=O)c3cccc(C(F)(F)F)c3)CC2)CC1]

Compound 27: 4-Methyl-1′-((3-(trifluoromethyl)phenyl)sulfonyl)-1,4′-bipiperidine. Reaction of amine 1a with 3-(trifluoromethyl)benzene-1-sulfonyl chloride (Procedure A) yielded 27 as a pink solid (90°/); mp 123-125° C. ¹H NMR (400 MHz, CDCl₃) δ 7.98 (s, 1H), 7.92 (d, J=7.9 Hz, 1H), 7.83 (d, J=7.8 Hz, 1H), 7.67 (t, J=7.9 Hz, 1H), 3.85 (d, J=12.0 Hz, 2H), 2.79 (d, J=11.6 Hz, 2H), 2.27 (tt, J=11.8, 3.7 Hz, 3H), 2.19-2.06 (m, 2H), 1.86 (d, J=11.5 Hz, 2H), 1.70-1.48 (m, 4H), 1.35-1.25 (m, 1H), 1.18 (qd, J=11.9, 3.7 Hz, 2H), 0.87 (d, J=6.3 Hz, 3H). ¹³C NMR (100 MHz, CDCl₃) δ 137.6, 131.8 (q, J=33.4 Hz), 130.8, 129.9, 129.4 (q, J=3.5 Hz), 124.5 (q, J=3.7 Hz), 121.8, 61.3, 49.4, 46.0, 34.2, 30.8, 27.2, 21.7. HRMS (ESI-TOF) calcd for C₁₈H₂₆F₃N₂O₂S [M+H]⁺ 391.1661, found 391.1666.

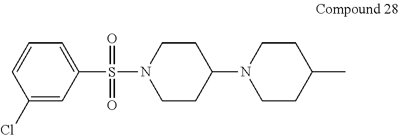 [SMILES: CC1CCN(C2CCN(S(=O)(=O)c3cccc(Cl)c3)CC2)CC1]

Compound 28. 1′-((3-Chlorophenyl)sulfonyl)-4-methyl-1,4′-bipiperidine. Reaction of amine 1a with 3-chlorobenzene-1-sulfonyl chloride (Procedure A) yielded 28 as a yellow solid (74%); mp 122-124° C. ¹H NMR (400 MHz, CDCl₃) δ 7.71 (s, 1H), 7.61 (d, J=7.7 Hz, 1H), 7.54 (d, J=8.1 Hz, 1H), 7.45 (t, J=7.9 Hz, 1H), 3.83 (d, J=11.7 Hz, 2H), 2.80 (d, J=10.9 Hz, 2H), 2.28 (td, J=11.8, 2.5 Hz, 3H), 2.15 (t, J=11.3 Hz, 2H), 1.87 (d, J=12.7 Hz, 2H), 1.72-1.56 (m, 4H), 1.40-1.14 (m, 3H), 0.88 (d, J=6.3 Hz, 3H). ¹³C NMR (100 MHz, CDCl₃) δ 138.0, 135.3, 132.8, 130.3, 127.5, 125.7, 61.4, 49.4, 46.1, 34.2, 30.8, 27.2, 21.7. HRMS (ESI-TOF) calcd for C₁₇H₂₆ClN₂O₂S [M+H]⁺ 357.1398 found 357.1386.

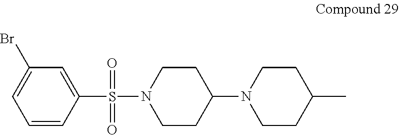 [SMILES: CC1CCN(C2CCN(S(=O)(=O)c3cccc(Br)c3)CC2)CC1]

Compound 29: 1′-((3-Bromophenyl)sulfonyl)-4-methyl-1,4′-bipiperidine. Reaction of amine 1a with 3-bromobenzene-1-sulfonyl chloride (Procedure A) yielded 29 as a white solid (65%); mp 125-126° C. ¹H NMR (400 MHz, CDCl₃) δ 7.85 (s, 1H), 7.66 (dd, J=13.3, 7.9 Hz, 2H), 7.37 (t, J=7.9 Hz, 1H), 3.80 (d, J=11.7 Hz, 2H), 2.74 (d, J=11.0 Hz, 2H), 2.16 (dt, J=73.4, 11.4 Hz, 5H), 1.81 (d, J=12.7 Hz, 2H), 1.60 (td, J=12.4, 8.1 Hz, 4H), 1.34-1.22 (m, 1H), 1.20-1.02 (m, 2H), 0.85 (d, J=6.2 Hz, 3H). ¹³C NMR (100 MHz, CDCl₃) δ 138.2, 135.7, 130.5, 130.3, 126.1, 123.1, 61.3, 49.5, 46.1, 34.6, 31.0, 27.4, 21.9. HRMS (ESI-TOF) calcd for C₁₇H₂₆BrN₂O₂S [M+H]⁺ 401.0893, found 401.0886.

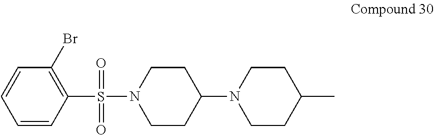 [SMILES: CC1CCN(C2CCN(S(=O)(=O)c3ccccc3Br)CC2)CC1]

Compound 30: 1′-((2-Bromophenyl)sulfonyl)-4-methyl-1,4′-bipiperidine. Reaction of amine 1a with 2-bromobenzene-1-sulfonyl chloride (Procedure A) yielded 30 as a white solid (>95%); mp 82-84° C. ¹H NMR (400 MHz, CDCl₃) δ 8.05 (dd, J=7.8, 1.8 Hz, 1H), 7.70 (dd, J=7.8, 1.4 Hz, 1H), 7.37 (dtd, J=24.2, 7.5, 1.6 Hz, 2H), 3.85 (d, J=12.8 Hz, 2H), 2.91-2.62 (m, 4H), 2.33 (tt, J=11.5, 3.5 Hz, 1H), 2.11 (td, J=11.5, 2.4 Hz, 2H), 1.80 (d, J=12.2 Hz, 2H), 1.65-1.50 (m, 4H), 1.35-1.22 (m, 1H), 1.15 (qd, J=12.2, 3.8 Hz, 2H), 0.86 (d, J=6.3 Hz, 3H). ¹³C NMR (100 MHz, CDCl₃) δ 138.0, 135.7, 133.5, 132.2, 127.4, 120.4, 61.7, 49.5, 45.6, 34.6, 31.0, 27.8, 21.9. HRMS (ESI-TOF) calcd for C₁₇H₂₆BrN₂O₂S [M+H]⁺ 401.0893, found 401.0891

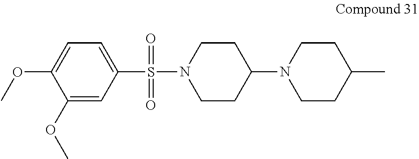

Compound 31: 1′-((3,4-Dimethoxyphenyl)sulfonyl)-4-methyl-1,4′-bipiperidine. Reaction of amine 1a with 3,4-dimethoxybenzene-1-sulfonyl chloride (Procedure A) yielded 31 as a white solid (78%); mp 132-134° C. ¹H NMR (400 MHz, CDCl₃) δ 7.34 (dd, J=8.4, 2.1 Hz, 1H), 7.18 (d, J=2.1 Hz, 1H), 6.92 (d, J=8.5 Hz, 1H), 3.91 (s, 3H), 3.90 (s, 3H), 3.80 (d, J=11.9 Hz, 2H), 2.77 (d, J=11.7 Hz, 2H), 2.30-2.04 (m, 5H), 1.82 (d, J=11.2 Hz, 2H), 1.69-1.54 (m, 4H), 1.37-1.25 (m, 1H), 1.16 (qd, J=12.1, 3.8 Hz, 2H), 0.87 (d, J=6.3 Hz, 3H), ¹³C NMR (100 MHz, CDCl₃) δ 152.5, 148.9, 127.8, 121.5, 110.5, 110.1, 61.5, 56.2, 56.1, 49.5, 46.2, 34.5, 31.0, 27.3, 21.8. HRMS (ESI-TOF) calcd for C₁₉H₃₁N₂O₄S [M+H]⁺ 383.1999, found 383.1991.

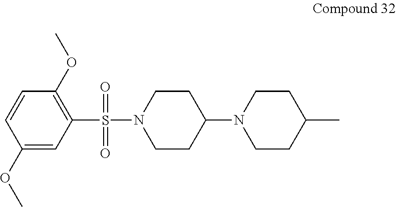 [SMILES: COc1ccc(OC)c(S(=O)(=O)N2CCC(N3CCC(C)CC3)CC2)c1]

Compound 32: 1′-((2,5-Dimethoxyphenyl)sulfonyl)-4-methyl-1,4′-bipiperidine. Reaction of amine 1a with 2,5-dimethoxybenzenesulfonyl chloride (Procedure A) yielded 32 as a white solid (95%); mp 75-78° C. ¹H NMR (400 MHz, CDCl₃) δ 7.39 (d, J=3.1 Hz, 1H), 7.00 (dd, J=9.0, 3.1 Hz, 1H), 6.90 (d, J=9.0 Hz, 1H), 3.87 (d, J=12.8 Hz, 2H), 3.83 (s, 3H), 3.75 (s, 3H), 2.77 (d, J=11.6 Hz, 2H), 2.57 (t, J=12.4 Hz, 2H), 2.27 (t, J=11.5 Hz, 1H), 2.10 (t, J=11.5 Hz, 2H), 1.77 (d, J=13.8 Hz, 2H), 1.64-1.47 (m, 4H), 1.36-1.22 (m, 1H), 1.21-1.08 (m, 2H), 0.86 (d, J=6.4 Hz, 3H). ¹³C NMR (100 MHz, CDCl₃) δ 152.9, 151.0, 127.3, 120.0, 115.9, 113.9, 61.9, 56.6, 56.0, 49.5, 45.9, 34.6, 31.0, 27.9, 21.9. HRMS (ESI-TOF) calcd for C₁₉H₃₁N₂O₄S [M+H]⁺ 383.1999, found 383.1991.

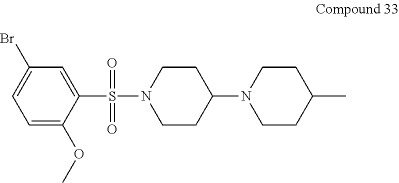 [SMILES: COc1ccc(Br)cc1S(=O)(=O)N1CCC(N2CCC(C)CC2)CC1]

Compound 33: 1′-((5-Bromo-2-methoxyphenyl)sulfonyl)-4-methyl-1,4′-bipiperidine. Reaction of amine 1a with 5-bromo-2-methoxybenzene-1-sulfonyl chloride (Procedure A) yielded 33 as a yellow solid (92%); mp 98-102° C. ¹H NMR (400 MHz, CDCl₃) δ 8.01 (d, J=2.6, Hz, 1H), 7.59 (dd, J=8.8, 2.6 Hz, 1H), 6.88 (d, J=8.8 Hz, 1H), 3.96-3.88 (d, J=13.6 Hz, 2H), 3.90 (s, 3H), 2.84 (d, J=10.9 Hz, 2H), 2.64 (td, J=12.5, 2.5 Hz, 2H), 2.38 (d, J=12.2 Hz, 1H), 2.17 (t, J=11.7 Hz, 2H), 1.85 (d, J=12.7 Hz, 2H), 1.68-1.52 (m, 4H), 1.40-1.14 (m, 3H), 0.91 (d, J=6.2 Hz, 3H). ¹³C NMR (100 MHz, CDCl₃) δ 155.9, 136.8, 133.8, 128.7, 114.1, 112.3, 61.8, 56.3, 49.5, 45.9, 34.6, 31.0, 28.0, 21.9. HRMS (ESI-TOF) calcd for C₁₈H₂₇BrN₂O₃SNa [M+Na]⁺ 453.0818, found 453.0805.

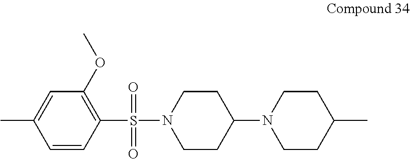

Compound 34: 1′-((2-Methoxy-4-methylphenyl)sulfonyl)-4-methyl-1,4′-bipiperidine. Reaction of amine 1a with 2-methoxy-4-methylphenylsulfonyl chloride (Procedure A) yielded 34 as a yellow solid (88%); mp 95-98° C. ¹H NMR (400 MHz, CDCl₃) δ 7.70 (d, J=7.9 Hz, 1H), 6.85-6.69 (m, 2H), 3.87 (d, J=12.4 Hz, 2H), 3.85 (s, 3H), 2.78 (d, J=11.1 Hz, 2H), 2.54 (d, J=12.4 Hz, 2H), 2.35 (s, 3H), 2.27 (tt, J=11.8, 3.6 Hz, 1H), 2.12 (td, J=11.5, 2.7 Hz, 2H), 1.78 (d, J=12.5 Hz, 2H), 1.64-1.47 (m, 4H), 1.37-1.23 (m, 1H), 1.22-1.08 (m, 2H), 0.86 (d, J=6.4 Hz, 3H). ¹³C NMR (100 MHz, CDCl₃) δ 156.8, 145.5, 131.6, 123.5, 121.0, 112.9, 61.9, 55.8, 49.5, 45.9, 34.5, 31.0, 27.9, 21.9, 21.8. HRMS (ESI-TOF) calcd for C₁₉H₃₁N₂O₃S [M+H]⁺ 367.2050, found 367.2061.

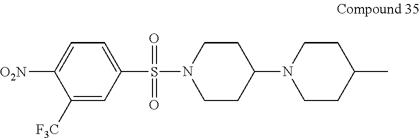 [SMILES: CC1CCN(C2CCN(S(=O)(=O)c3ccc([N+](=O)[O-])c(C(F)(F)F)c3)CC2)CC1]

Compound 35: 4-Methyl-1′-((4-nitro-3-(trifluoromethyl)phenyl)sulfonyl)-1,4′-bipiperidine. Reaction of amine 1a with 4-nitro-3-(trifluoromethyl)benzene-1-sulfonyl chloride (Procedure A) yielded 35 as a yellow solid (86%); mp 175-178° C. ¹H NMR (400 MHz, CDCl₃) δ 8.15 (s, 1H), 8.08 (dd, J=8.4, 1.8 Hz, 1H), 7.98 (d, J=8.3 Hz, 1H), 3.87 (d, J=11.7 Hz, 2H), 2.77 (d, J=11.7 Hz, 2H), 2.38 (td, J=12.0, 2.5 Hz, 2H), 2.25 (tt, J=11.4, 3.6 Hz, 1H), 2.11 (td, J=11.4, 2.6 Hz, 2H), 1.87 (d, J=12.1 Hz, 2H), 1.73-1.56 (m, 4H), 1.38-1.24 (m, 1H), 1.16 (qd, J=11.7, 3.5 Hz, 2H), 0.88 (d, J=6.4 Hz, 3H). ¹³C NMR (100 MHz, CDCl₃) δ 150.0, 141.4, 132.2, 127.1 (q, J=5.2 Hz), 125.9, 124.7 (d, J=35.3 Hz). 121.1 (d, J=274.2 Hz), 61.0, 49.5, 46.0, 34.5, 30.9, 27.4, 21.8. HRMS (ESI-TOF) calcd for C₁₈H₂₅F₃N₃O₄S [M+H]⁺ 436.1512, found 436.1515.

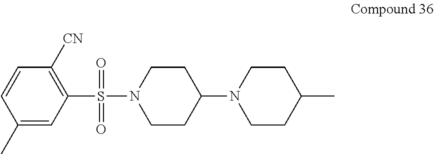 [SMILES: Cc1ccc(C#N)c(S(=O)(=O)N2CCC(N3CCC(C)CC3)CC2)c1]

Compound 36: 4-Methyl-2-((4-methyl-[1,4′-bipiperidin]-1′-yl)sulfonyl)benzonitrile. Reaction of amine 1a with 2-cyano-5-methylbenzene-1-sulfonyl chloride (Procedure A) yielded 36 as a light pink solid (94%); mp 118-121° C. ¹H NMR (400 MHz, CDCl₃) δ 7.80 (s, 1H), 7.71 (d, J=7.8 Hz, 1H), 7.44 (d, J=7.8 Hz, 1H), 3.92 (d, J=12.2 Hz, 2H), 2.78 (d, J=12.3 Hz, 2H), 2.59 (t, J=12.2 Hz, 2H), 2.47 (s, 3H), 2.31 (t, J=11.7 Hz, 1H), 2.12 (t, J=11.3 Hz, 2H), 1.84 (d, J=14.5 Hz, 2H), 1.71-1.51 (m, 4H), 1.37-1.24 (m, 1H), 1.17 (q, J=12.5 Hz, 2H), 0.87 (d, J=6.5 Hz, 3H). ¹³C NMR (100 MHz, CDCl₃) δ 144.4, 140.3, 135.4, 133.2, 130.8, 116.5, 107.8, 61.5, 49.5, 45.8, 34.5, 31.0, 27.5, 21.8. HRMS (ESI-TOF) calcd for C₁₉H₂₈N₃O₂S [M+H]⁺ 362.1897, found 362.1885.

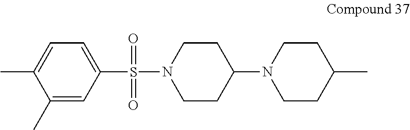

Compound 37: 1′-((3,4-Dimethylphenyl)sulfonyl)-4-methyl-1,4′-bipiperidine. Reaction of amine 1a with 3,4-dimethylbenzene-1-sulfonyl chloride (Procedure A) yielded 37 as a yellow solid (89%); mp 87-89° C. ¹H NMR (400 MHz, CDCl₃) δ 7.47 (s, 1H), 7.45 (d, J=7.9 Hz, 1H), 7.24 (d, J=7.8 Hz, 1H), 3.81 (d, J=12.0 Hz, 2H), 2.78 (d, J=11.5 Hz, 2H), 2.30 (s, 6H), 2.16 (m, 5H), 1.82 (d, J=11.5 Hz, 2H), 1.69-1.54 (m, 4H), 1.38-1.24 (m, 1H), 1.17 (qd, J=12.0, 3.6 Hz, 2H), 0.87 (d, J=6.2 Hz, 3H). ¹³C NMR (100 MHz, CDCl₃) δ 142.2, 137.7, 133.1, 130.1, 128.4, 125.3, 61.5, 49.4, 46.2, 34.4, 30.9, 27.2, 21.8, 19.9, 19.9. HRMS (ESI-TOF) calcd for C₁₉H₃₁N₂O₂S [M+H]⁺ 351.2101, found 351.2091.

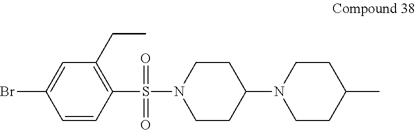 [SMILES: CCc1cc(Br)ccc1S(=O)(=O)N1CCC(N2CCC(C)CC2)CC1]

Compound 38: 1′-((4-Bromo-2-ethylphenyl)sulfonyl)-4-methyl-1,4′-bipiperidine. Reaction of amine 1a with 4-bromo-2-ethylbenzene-1-sulfonyl chloride (Procedure A) yielded 38 as a white solid (92%); mp 87-91° C. ¹H NMR (400 MHz, CDCl₃) δ 7.73 (d, J=8.4 Hz, 1H), 7.50 (d, J=2.0 Hz, 1H), 7.42 (dd, J=8.4, 2.0 Hz, 1H), 3.75 (d, J=12.4 Hz, 2H), 2.96 (q, J=7.6 Hz, 2H), 2.83 (d, J=7.6 Hz, 2H), 2.60 (td, J=12.4, 2.4 Hz, 2H), 2.40-2.27 (m, 1H), 2.20-2.05 (m, 2H), 1.84 (d, J=7.6 Hz, 2H), 1.68-1.48 (m, 4H), 1.38-1.13 (m, 6H), 0.89 (d, J=6.4 Hz, 3H). ¹³C NMR (100 MHz, CDCl₃) δ 145.2, 134.8, 133.9, 131.7, 129.0, 127.8, 61.6, 49.5, 45.1, 34.4, 30.9, 27.7, 25.9, 21.8, 15.4. HRMS (ESI-TOF) calcd for C₁₉H₃₀BrN₂O₂S [M+H]⁺ 429.1206, found 429.1202

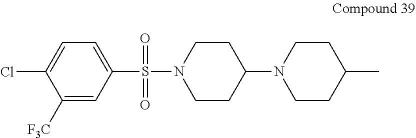 [SMILES: CC1CCN(C2CCN(S(=O)(=O)c3ccc(Cl)c(C(F)(F)F)c3)CC2)CC1]

Compound 39: 1′-((4-Chloro-3-(trifluoromethyl)phenyl)sulfonyl)-4-methyl-1,4′-bipiperidine. Reaction of amine 1a with 4-chloro-3-(trifluoromethyl)benzene-1-sulfonyl chloride (Procedure A) yielded 39 as a light brown solid (>95%); mp 118-120° C. ¹H NMR (400 MHz, CDCl₃) δ 8.03 (s, 1H), 7.84 (dd, J=8.5, 2.1 Hz, 1H), 7.66 (d, J=8.4 Hz, 1H), 3.84 (d, J=12.1 Hz, 2H), 2.78 (d, J=11.1 Hz, 2H), 2.37-2.18 (m, 3H), 2.12 (td, J=11.5, 2.7 Hz, 2H), 1.86 (d, J=11.8 Hz, 2H), 1.71-1.52 (m, 4H), 1.36-1.24 (m, 1H), 1.17 (qd, J=11.9, 3.8 Hz, 2H), 0.88 (d, J=6.3 Hz, 3H). ¹³C NMR (100 MHz, CDCl₃) δ 137.3, 135.7, 132.4, 131.7, 129.5 (q, J=32.6 Hz), 126.7 (q, J=5.3 Hz), 122.0 (q, J=274.1 Hz), 61.3, 49.4, 45.9, 34.1, 30.8, 27.1, 21.7. HRMS (ESI-TOF) calcd for C₁₈H₂₅ClF₃N₂O₂S [M+H]⁺ 425.1261, found 425.1272.

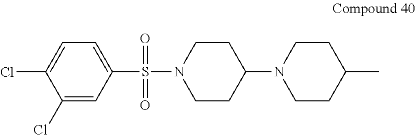 [SMILES: CC1CCN(C2CCN(S(=O)(=O)c3ccc(Cl)c(Cl)c3)CC2)CC1]

Compound 40: 1′-((3,4-Dichlorophenyl)sulfonyl)-4-methyl-1,4′-bipiperidine. Reaction of amine 1a with 3,4-dichlorobenzene-1-sulfonyl chloride (Procedure A) yielded 40 as a light brown solid (86%); mp 160-162° C. ¹H NMR (400 MHz, CDCl₃) δ 7.81 (d, J=1.9 Hz, 1H), 7.66-7.44 (m, 2H), 3.81 (d, J=12.1 Hz, 2H), 2.76 (d, J=11.2 Hz, 2H), 2.29 (td, J=11.9, 2.5 Hz, 2H), 2.20 (tt, J=11.5, 3.5 Hz, 1H), 2.10 (td, J=11.5, 2.4 Hz, 2H), 1.84 (d, J=11.5 Hz, 2H), 1.62 (ddt, J=16.3, 12.5, 5.6 Hz, 4H), 1.36-1.23 (m, 1H), 1.15 (qd, J=11.8, 11.2, 3.7 Hz, 2H), 0.87 (d, J=6.5 Hz, 3H). ¹³C NMR (100 MHz, CDCl₃) δ 137.5, 136.2, 133.8, 131.1, 129.4, 126.6, 61.3, 49.5, 46.1, 34.6, 31.0, 27.4, 21.8. HRMS (ESI-TOF) calcd for C₁₇H₂₅Cl₂N₂O₂S [M+H]⁺ 391.1008. found 391.1018.

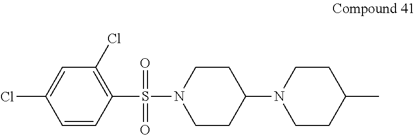 [SMILES: CC1CCN(C2CCN(S(=O)(=O)c3ccc(Cl)cc3Cl)CC2)CC1]

Compound 41: 1′-((2,4-Dichlorophenyl)sulfonyl)-4-methyl-1,4′-bipiperidine. Reaction of amine 1a with 2,4-dichlorobenzene-1-sulfonyl chloride (Procedure A) yielded 41 as a light orange solid (88%); mp 75-78° C. ¹H NMR (400 MHz, CDCl₃) δ 7.94 (d, J=8.6 Hz, 1H), 7.49 (d, J=2.0 Hz, 1H), 7.33 (dd, J=8.5, 2.0 Hz, 1H), 3.85 (d, J=12.9 Hz, 2H), 2.81 (d, J=11.7 Hz, 2H), 2.71 (td, J=12.5, 2.4 Hz, 2H), 2.37 (tt, J=11.7, 3.5 Hz, 1H), 2.14 (td, J=11.0, 2.2 Hz, 2H), 1.83 (d, J=12.1 Hz, 2H), 1.67-1.47 (m, 4H), 1.38-1.25 (m, 1H), 1.25-1.11 (m, 2H), 0.87 (d, J=6.3 Hz, 3H). ¹³C NMR (100 MHz, CDCl₃) δ 139.2, 135.2, 133.2, 132.9, 131.8, 127.2, 61.6, 49.5, 45.6, 34.4, 30.9, 27.8, 21.8. HRMS (ESI-TOF) calcd for C₁₇H₂₅Cl₂N₂O₂S [M+H]⁺ 391.1008 found 391.1013.

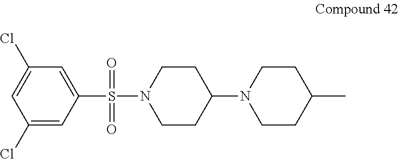 [SMILES: CC1CCN(C2CCN(S(=O)(=O)c3cc(Cl)cc(Cl)c3)CC2)CC1]

Compound 42: 1′-((3,5-Dichlorophenyl)sulfonyl)-4-methyl-1,4′-bipiperidine. Reaction of amine 1a with 3,5-dichlorobenzene-1-sulfonyl chloride (Procedure A) yielded 42 as a pink solid (92%); mp 128-131° C. ¹H NMR (500 MHz, CDCl₃) δ 7.61 (d, J=2.0 Hz, 2H), 7.57 (t, J=1.9 Hz, 1H), 3.92-3.76 (m, 2H), 2.83 (d, J=6.2 Hz, 2H), 2.47-2.25 (m, 3H), 2.17 (t, J=11.3 Hz, 2H), 1.90 (d, J=14.2 Hz, 2H), 1.65 (td, J=12.6, 4.1 Hz, 4H), 1.37-1.27 (m, 1H), 1.27-1.13 (m, 2H), 0.90 (d, J=6.0 Hz, 3H). ¹³C NMR (100 MHz, CDCl₃) δ 139.3, 136.1, 132.7, 125.8, 61.3, 49.5, 46.0, 34.2, 30.8, 27.2, 21.7. HRMS (ESI-TOF) calcd for C₁₇H₂₅Cl₂N₂O₂S [M+H]⁺ 391.1008, found 391.1001.

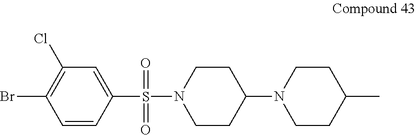 [SMILES: CC1CCN(C2CCN(S(=O)(=O)c3ccc(Br)c(Cl)c3)CC2)CC1]

Compound 43: 1′-((4-Bromo-3-chlorophenyl)sulfonyl)-4-methyl-1,4′-bipiperidine. Reaction of amine 1a with 4-bromo-3-chlorobenzene-1-sulfonyl chloride (Procedure A) yielded 43 as a white solid (95%); mp 169-171° C. ¹H NMR (400 MHz, CDCl₃) δ 7.87-7.70 (m, 2H), 7.46 (d, J=8.4 Hz, 1H), 3.81 (d, J=11.8 Hz, 2H), 2.76 (d, J=10.7 Hz, 2H), 2.29 (t, J=12.3 Hz, 2H), 2.20 (tt, J=11.5, 4.1 Hz, 1H), 2.15-2.05 (m, 2H), 1.84 (d, J=12.3 Hz, 2H), 1.74-1.52 (m, 4H), 1.38-1.23 (m, 1H), 1.22-1.07 (m, 2H), 0.87 (d, J=6.4 Hz, 3H). ¹³C NMR (100 MHz, CDCl₃) δ 136.9, 135.8, 134.4, 129.1, 127.9, 126.6, 61.3, 49.5, 46.1, 34.6, 31.0, 27.4, 21.8. HRMS (ESI-TOF) calcd for C₁₇H₂₅ClBrN₂O₂S [M+H]⁺ 435.0503, found 435.0515.

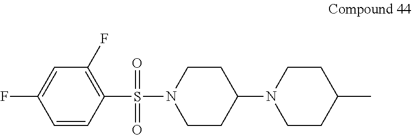 [SMILES: CC1CCN(C2CCN(S(=O)(=O)c3ccc(F)cc3F)CC2)CC1]

Compound 44: 1′-((2,4-Difluorophenyl)sulfonyl)-4-methyl-1,4′-bipiperidine. Reaction of amine 1a with 2,4-difluorobenzene-1-sulfonyl chloride (Procedure A) yielded 44 as a white solid (88%); mp 165-168° C. ¹H NMR (400 MHz, CDCl₃) δ 7.89-7.73 (m, 2H), 7.63 (dd, J=8.5, 1.6 Hz, 2H), 7.41 (td, J=8.7, 6.3 Hz, 1H), 7.05-6.83 (m, 2H), 3.87 (d, J=12.0 Hz, 2H), 2.77 (d, J=11.6 Hz, 2H), 2.31 (td, J=12.0, 2.5 Hz, 2H), 2.27-2.17 (m, 1H), 2.12 (td, J=11.6, 2.4 Hz, 2H), 1.84 (d, J=12.9 Hz, 2H), 1.71-1.53 (m, 4H), 1.37-1.24 (m, 1H), 1.24-1.09 (m, 2H), 0.88 (d, J=6.3 Hz, 3H). ¹³C NMR (100 MHz, CDCl₃) δ 162.9 (dd, J=251.0, 11.9 Hz), 159.8 (dd, J=251.8, 11.9 Hz), 139.4 (d, J=1.6 Hz), 135.4, 131.5 (dd, J=9.6, 4.6 Hz), 129.4 (d, J=3.1 Hz), 127.9, 123.5 (dd, J=13.3, 3.9 Hz), 112.0 (dd, J=21.3, 3.8 Hz), 104.7 (t, J=10.0 Hz), 61.4, 49.5, 46.2, 34.5, 31.0, 27.3, 21.8. MS (ESI) calcd for C₁₇H₂₅F₂N₂O₂S [M+H]⁺ 359.2 found 359.2.

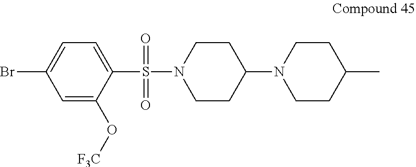 [SMILES: CC1CCN(C2CCN(S(=O)(=O)c3ccc(Br)cc3OC(F)(F)F)CC2)CC1]

Compound 45: 1′-((4-Bromo-2-(trifluoromethoxy)phenyl)sulfonyl)-4-methyl-1,4′-bipiperidine. Reaction of amine 1a with 4-bromo-2-(trifluoromethoxy)benzene-1-sulfonyl chloride (Procedure A) yielded 45 as a yellow solid (67%); mp 108-110° C. ¹H NMR (400 MHz, CDCl₃) δ 7.83 (d, J=8.9 Hz, 1H), 7.58-7.45 (m, 2H), 3.85 (d, J=12.7 Hz, 2H), 2.78 (dt, J=11.9, 3.3 Hz, 2H), 2.59 (td, J=12.5, 2.5 Hz, 2H), 2.29 (tt, J=11.5, 3.6 Hz, 1H), 2.11 (td, J=11.5, 2.4 Hz, 2H), 1.82 (dt, J=12.7, 2.7 Hz, 2H), 1.69-1.45 (m, 4H), 1.40-1.24 (m, 1H), 1.23-1.05 (m, 2H), 0.88 (d, J=6.3 Hz, 3H). ¹³C NMR (100 MHz, CDCl₃) δ 146.2, 132.7, 130.1, 129.8, 127.9, 123.90, 121.3, 61.5, 49.5, 45.7, 34.59, 31.0, 27.8, 21.8. HRMS (ESI-TOF) calcd for C₁₈H₂₅BrF₃N₂O₃S [M+H]⁺ 485.0716, found 485.0724.

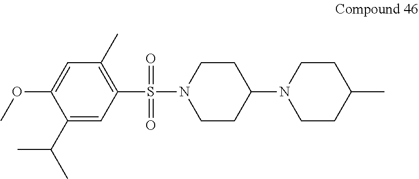

Compound 46: 1′-((5-Isopropyl-4-methoxy-2-methylphenyl)sulfonyl)-4-methyl-1,4′-bipiperidine. Reaction of amine 1a with 5-isopropyl-4-methoxy-2-methylbenzene-1-sulfonyl chloride (Procedure A) yielded 46 as a brown solid (94%). ¹H NMR (400 MHz, CDCl₃) δ 7.65 (s, 1H), 6.71 (s, 1H), 3.88-3.84 (d, J=12.8 Hz, 2H), 3.83 (s, 3H), 3.02 (p, J=6.9 Hz, 1H), 2.80 (d, J=11.0 Hz, 2H), 2.51 (td, J=12.5, 2.5 Hz, 2H), 2.34-2.26 (m, 1H), 2.32 (s, 3H), 2.15 (td, J=11.4, 2.5 Hz, 2H), 1.80 (d, J=11.8 Hz, 2H), 1.67-1.51 (m, 4H), 1.37-1.11 (m, 3H), 1.16 (d, J=6.8 Hz, 6H), 0.86 (d, J=6.2 Hz, 3H). ¹³C NMR (100 MHz, CDCl₃) δ 154.3, 142.3, 139.1, 128.0, 123.8, 114.1, 62.0, 56.0, 49.4, 45.8, 34.3, 30.9, 28.7, 27.7, 23.2, 21.8, 19.7. HRMS (ESI-TOF) calcd for C₂₂H₃₇N₂O₃S [M+H]⁺ 409.2519, found 409.2511.

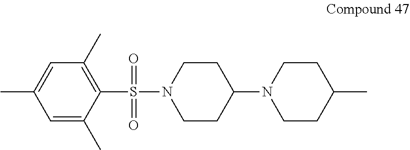

Compound 47: 1′-(Mesitylsulfonyl)-4-methyl-1,4′-bipiperidine. Reaction of amine 1a with 2,4,6-trimethylbenzene-1-sulfonyl chloride (Procedure A) yielded 47 as a yellow solid (>95%); mp 62-65° C. ¹H NMR (500 MHz, CDCl₃) δ 6.94 (s, 2H), 3.62 (d, J=12.5 Hz, 2H), 2.85 (d, J=12.4 Hz, 2H), 2.75 (t, J=12.4 Hz, 2H), 2.61 (s, 6H), 2.34 (t, J=11.5 Hz, 1H), 2.29 (s, 3H), 2.12 (t, J=11.4 Hz, 2H), 1.86 (d, J=11.1 Hz, 2H), 1.63 (d, J=13.4 Hz, 2H), 1.50 (qd, J=12.3, 4.0 Hz, 2H), 1.39-1.27 (br, 1H), 1.27-1.13 (m, 2H), 0.90 (d, J=6.6 Hz, 3H). ¹³C NMR (100 MHz, CDCl₃) δ 142.4, 140.4, 131.8, 131.7, 61.8, 49.6, 44.0, 34.6, 31.0, 27.7, 22.8, 21.9, 20.9. HRMS (ESI-TOF) calcd for C₂₀H₃₃N₂O₂S [M+H]⁺ 365.2257, found 365.2252.

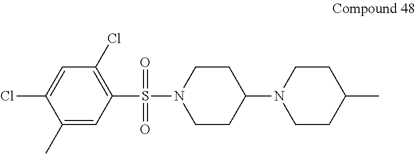 [SMILES: Cc1cc(S(=O)(=O)N2CCC(N3CCC(C)CC3)CC2)c(Cl)cc1Cl]

Compound 48: 1′-((2,4-Dichloro-5-methylphenyl)sulfonyl)-4-methyl-1,4′-bipiperidine. Reaction of amine 1a with 2,4-dichloro-5-methylbenzene-1-sulfonyl chloride (Procedure A) yielded 48 as a white solid (83%); ¹H NMR (400 MHz, CDCl₃) δ 7.89 (s, 1H), 7.49 (s, 1H), 3.87 (d, J=12.9 Hz, 2H), 2.82 (d, J=11.2 Hz, 2H), 2.71 (td, J=12.4, 2.4 Hz, 2H), 2.38 (s, 3H), 2.36-2.26 (m, 1H), 2.15 (t, J=11.2 Hz, 2H), 1.83 (d, J=11.6 Hz, 2H), 1.65-1.51 (m, 4H), 1.39-1.25 (m, 1H), 1.06 (t, J=12.0 Hz, 2H), 0.89 (d, J=6.0 Hz, 3H). ¹³C NMR (100 MHz, CDCl₃) δ 139.3, 135.5, 134.6, 133.6, 132.0, 129.9, 61.7, 49.5, 45.6, 34.5, 31.0, 27.7, 21.8, 19.6. HRMS (ESI-TOF) calcd for C₁₈H₂₇Cl₂N₂O₂S [M+H]⁺ 405.1165, found: 405.1165.

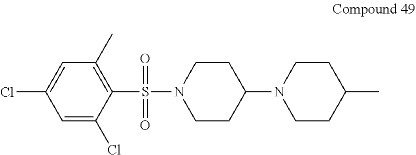 [SMILES: Cc1cc(Cl)cc(Cl)c1S(=O)(=O)N1CCC(N2CCC(C)CC2)CC1]

Compound 49: 1′-((2,4-Dichloro-6-methylphenyl)sulfonyl)-4-methyl-1,4′-bipiperidine. Reaction of amine 1a with 2,4-dichloro-6-methylbenzene-1-sulfonyl chloride (Procedure A) yielded 49 as a white solid (93%); mp 89-91° C. ¹H NMR (400 MHz, CDCl₃) δ 7.37 (d, J=2.3 Hz, 1H), 7.19 (dd, J=2.3, 0.9 Hz, 1H), 3.79 (d, J=12.7 Hz, 2H), 2.93-2.73 (m, 4H), 2.67 (s, 3H), 2.45-2.27 (m, 1H), 2.14 (td, J=11.3, 2.5 Hz, 2H), 1.83 (dd, J=12.3, 3.4 Hz, 2H), 1.57 (dtd, J=24.4, 11.6, 4.0 Hz, 4H), 1.30 (ddt, J=12.8, 6.4, 3.7 Hz, 1H), 1.18 (qd, J=12.0, 3.9 Hz, 2H), 0.89 (d, J=6.9 Hz, 3H). ¹³C NMR (100 MHz, CDCl₃) δ 143.7, 137.7, 135.3, 134.2, 131.6, 130.2, 61.7, 49.5, 45.0, 34.6, 31.0, 27.9, 24.0, 21.8. HRMS (ESI-TOF) calcd for C₁₈H₂₇Cl₂N₂O₂S [M+H]⁺ 405.1165, found 405.1161.

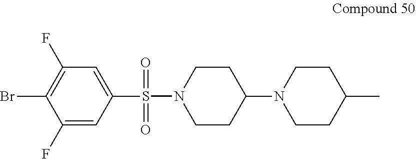 [SMILES: CC1CCN(C2CCN(S(=O)(=O)c3cc(F)c(Br)c(F)c3)CC2)CC1]

Compound 50: 1′-((4-Bromo-3,5-difluorophenyl)sulfonyl)-4-methyl-1,4′-bipiperidine. Reaction of amine 1a with 4-bromo-3,5-difluorobenzene-1-sulfonyl chloride (Procedure A) yielded 50 as a white solid (73%); mp 161-163° C. ¹H NMR (400 MHz, CDCl₃) δ 7.36-7.27 (m, 2H), 3.81 (d, J=12.0 Hz, 2H), 2.77 (d, J=11.7 Hz, 2H), 2.34 (td, J=12.0, 2.5 Hz, 2H), 2.22 (tt, J=11.4, 3.6 Hz, 1H), 2.10 (td, J=11.5, 2.5 Hz, 2H), 1.85 (d, J=11.4 Hz, 2H), 1.71-1.55 (m, 4H), 1.37-1.24 (m, 1H), 1.16 (qd, J=11.8, 3.8 Hz, 2H), 0.88 (d, J=6.3 Hz, 3H). ¹³C NMR (100 MHz, CDCl₃) δ 159.9 (dd, J=255.0, 3.8 Hz), 137.9 (t, J=7.6 Hz), 111.0 (m), 103.9, 103.7 (t, J=96.4 Hz), 61.2, 49.5, 46.1, 34.5, 31.0, 27.4, 21.8. HRMS (ESI-TOF) calcd for C₁₇H₂₄BrF₂N₂O₂S [M+H]⁺ 437.0704, found 437.0695.

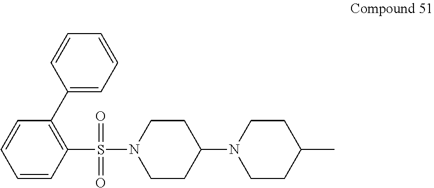 [SMILES: CC1CCN(C2CCN(S(=O)(=O)c3ccccc3-c3ccccc3)CC2)CC1]

Compound 51: 1′-([1,1′-Biphenyl]-2-ylsulfonyl)-4-methyl-1,4′-bipiperidine. Reaction of amine 1a with [1,1′-biphenyl]-2-sulfonyl chloride (Procedure A) yielded 51 as a yellow solid (>95%); mp 106-109° C. ¹H NMR (400 MHz, CDCl₃) δ 8.12 (dd, J=8.0, 1.6 Hz, 1H), 7.56 (td, J=7.6, 1.6 Hz, 1H), 7.46 (td, J=7.6, 1.6 Hz, 1H), 7.43-7.36 (m, 5H), 7.30 (dd, J=7.6, 1.6 Hz, 1H), 3.29 (d, J=12.8, 2H), 2.71 (d, J=10.8 Hz, 2H), 2.22 (td, J=12.6, 2.5 Hz, 3H), 2.09 (t, J=10.4 Hz, 2H), 1.90-1 49 (m, 4H), 1.36-1.13 (m, 5H), 0.88 (d, J=6.0 Hz, 3H). ¹³C NMR (100 MHz, CDCl₃) δ 141.6, 139.7, 137.1, 133.0, 132.2, 130.3, 129.6, 127.7, 127.5, 61.7, 49.3, 44.4, 34.4, 31.0, 27.2, 21.8. HRMS (ESI-TOF) calcd for C₂₃H₃₁N₂O₂S [M+H]⁺ 399.2101, found: 399.2101.

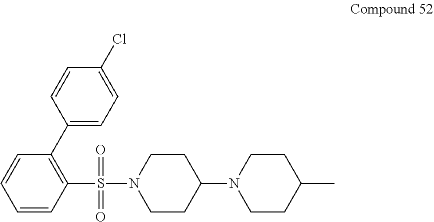 [SMILES: CC1CCN(C2CCN(S(=O)(=O)c3ccccc3-c3ccc(Cl)cc3)CC2)CC1]

Compound 52: 1′-((4′-Chloro-[1,1′-biphenyl]-2-yl)sulfonyl)-4-methyl-1,4′-bipiperidine. Reaction of compound 30 with 4-chlorophenyl)boronic acid (Procedure C) yielded compound 52 as a black gel (66%). ¹H NMR (400 MHz, CDCl₃) δ 8.08 (dd, J=8.0, 1.2 Hz, 1H), 7.55 (td, J=7.5, 1.4 Hz, 1H), 7.47 (td, J=7.8, 1.5 Hz, 1H), 7.34 (s, 4H), 7.26 (dd, J=7.6, 1.4 Hz, 1H), 3.32 (d, J=12.4 Hz, 2H), 2.70 (d, J=11.6 Hz, 2H), 2.36-2.15 (m, 3H), 2.08 (td, J=11.7, 2.5 Hz, 2H), 1.60 (s, 4H), 1.38-1.02 (m, 5H), 0.87 (d, J=6.3 Hz, 3H). ¹³C NMR (100 MHz, CDCl3) δ 140.3, 138.1, 137.0, 133.8, 132.8, 132.4, 131.0, 130.3, 127.8, 127.6, 61.5, 49.3, 44.6, 34.5, 31.0, 27.3, 21.8. LC-MS (ESI) calcd for C₂₃H₃₀ClN₂O₂S [M+H]⁺ 433.2, found 433.

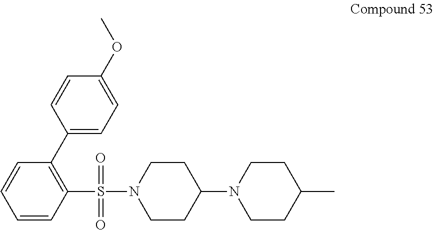 [SMILES: COc1ccc(-c2ccccc2S(=O)(=O)N2CCC(N3CCC(C)CC3)CC2)cc1]

Compound 53: 1′-((4′-Methoxy-[1,1′-biphenyl]-2-yl)sulfonyl)-4-methyl-1,4′-bipiperidine. Reaction of compound 30 with (4-methoxyphenyl)boronic acid (Procedure C) yielded compound 53 as a black gel (59%). ¹H NMR (400 MHz, CDCl₃) δ 8.08 (dd, J=7.9, 1.4 Hz, 1H), 7.52 (td, J=7.5, 1.4 Hz, 1H), 7.44-7.40 (m, 1H), 7.35-7.31 (m, 2H), 7.30-7.23 (m, 1H), 6.93-6.87 (m, 2H), 3.81 (s, 3H), 3.31 (d, J=12.9 Hz, 2H), 2.68 (d, J=11.6 Hz, 2H), 2.34-2.12 (m, 3H), 2.06 (td, J=11.6, 2.5 Hz, 2H), 1.57 (d, J=12.5 Hz, 4H), 1.39-1.01 (m, 5H), 0.86 (d, J=6.4 Hz, 3H). ¹³C NMR (100 MHz, CDCl₃) δ 159.2, 141.4, 137.1, 133.2, 132.2, 132.0, 130.9, 130.3, 128.5, 128.4, 127.2, 112.9, 61.6, 55.3, 49.3, 44.6, 34.6, 31.0, 27.3, 21.9. HRMS (ESI-TOF) calcd for C₂₄H₃₃N₂O₃S [M+H]⁺ 429.2206, found 429.2199.

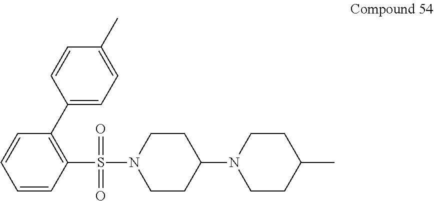 [SMILES: Cc1ccc(-c2ccccc2S(=O)(=O)N2CCC(N3CCC(C)CC3)CC2)cc1]

Compound 54: 4-Methyl-1′-((4′-methyl-[1,1′-biphenyl]-2-yl)sulfonyl)-1,4′-bipiperidine. Reaction of compound 30 with p-tolylboronic acid (Procedure C) yielded compound 54 as a brown gel (23%). ¹H NMR (400 MHz, CDCl₃) δ 8.08 (dd, J=8.0, 1.4 Hz, 1H), 7.54 (td, J=7.5, 1.4 Hz, 1H), 7.44 (td, J=7.7, 1.5 Hz, 1H), 7.28 (d, J=7.9 Hz, 3H), 7.18 (d, J=7.8 Hz, 2H), 3.33 (d, J=13.1 Hz, 2H), 2.84 (d, J=10.7 Hz, 2H), 2.43 (t, J=12.6 Hz, 1H), 2.37 (s, 3H), 2.22 (td, J=12.6, 2.4 Hz, 4H), 1.76-1.58 (m, 4H), 1.47-1.19 (m, 5H), 0.90 (d, J=4.8 Hz, 3H). ¹³C NMR (100 MHz, CDCl₃) δ 141.7, 137.5, 136.9, 136.7, 133.1, 132.3, 130.2, 129.4, 128.2, 127.3, 62.0, 49.1, 44.2, 33.4, 30.6, 26.7, 21.5, 21.2. HRMS (ESI-TOF) calcd for C₂₄H₃₃N₂O₂S [M+H]⁺ 413.2257, found 413.2264.

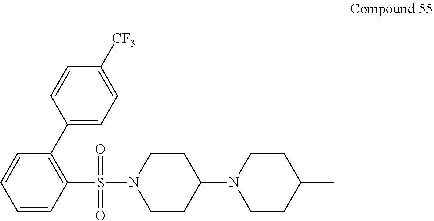 [SMILES: CC1CCN(C2CCN(S(=O)(=O)c3ccccc3-c3ccc(C(F)(F)F)cc3)CC2)CC1]

Compound 55: 4-Methyl-1′-((4′-(trifluoromethyl)-[1,1′-biphenyl]-2-yl)sulfonyl)-1,4′-bipiperidine. Reaction of compound 30 with (4-(trifluoromethyl)phenyl)boronic acid (Procedure C) yielded compound 55 as a black solid (12%). ¹H NMR (400 MHz, CDCl₃) δ 8.10 (dd, J=8.0, 1.4 Hz, 1H), 7.65 (d, J=8.1 Hz, 2H), 7.59 (tt, J=7.5, 1.1 Hz, 1H), 7.56-7.49 (m, 3H), 7.29 (dd, J=7.7, 1.4 Hz, 1H), 3.30 (d, J=12.8 Hz, 2H), 2.73 (d, J=11.0 Hz, 2H), 2.31 (td, J=12.6, 2.4 Hz, 3H), 2.10 (t, J=12.3 Hz, 2H), 1.64 (t, J=12.4 Hz, 4H), 1.35-1.12 (m, 5H), 0.88 (d, J=6.0 Hz, 3H). ¹³C NMR (100 MHz, CDCl₃) δ 143.4, 140.1, 136.9, 132.7, 132.5, 130.3, 130.1, 130.0, 129.8, 128.2, 124.4 (q, J=3.7 Hz), 61.6, 49.3, 44.5, 34.3, 30.9, 27.1, 21.7. HRMS (ESI-TOF) calcd for C₂₄H₃₀F₃N₂O₂S [M+H]⁺ 467.1975, found 467.1967.

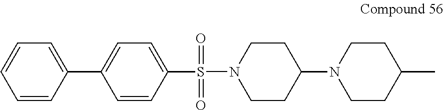 [SMILES: CC1CCN(C2CCN(S(=O)(=O)c3ccc(-c4ccccc4)cc3)CC2)CC1]

Compound 56: 1′-([1,1′-Biphenyl]-4-ylsulfonyl)-4-methyl-1,4′-bipiperidine. Reaction of compound 17 with phenylboronic acid (Procedure C) yielded compound 56 as a white solid (21%); mp 178-181° C. ¹H NMR (400 MHz, CDCl₃) δ 7.81 (d, J=8.4 Hz, 2H), 7.72 (d, J=8.4 Hz, 2H), 7.64-7.56 (m, 2H), 7.52-7.45 (m, 2H), 7.45-7.37 (m, 1H), 3.90 (d, J=12.1 Hz, 2H), 2.84 (d, J=10.8 Hz, 2H), 2.32 (td, J=12.1, 2.5 Hz, 3H), 2.20 (t, J=11.9 Hz, 2H), 1.91 (d, J=12.6 Hz, 2H), 1.77-1.57 (m, 4H), 1.42-1.12 (m, 3H), 0.90 (d, J=5.9 Hz, 3H). ¹³C NMR (100 MHz, CDCl₃) δ 145.6, 139.2, 134.6, 129.1, 129.1, 128.5, 128.2, 127.6, 127.3, 61.6, 49.4, 46.1, 34.1, 30.8, 27.1, 21.7. HRMS (ESI-TOF) calcd for C₂₃H₃₁N₂O₂S [M+H]⁺ 399.2101, found 399.2097.

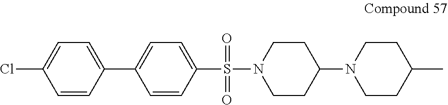 [SMILES: CC1CCN(C2CCN(S(=O)(=O)c3ccc(-c4ccc(Cl)cc4)cc3)CC2)CC1]

Compound 57: 1′-((4′-Cloro-[1,1′-biphenyl]-4-yl)sulfonyl)-4-methyl-1,4′-bipiperidine. Reaction of amine 1a with 4′-chloro-[1,1′-biphenyl]-4-sulfonyl chloride (Procedure A) yielded 57 as a white solid (87%); mp 205-207° C. ¹H NMR (400 MHz, CDCl₃) δ 7.88-7.75 (m, 2H), 7.67 (d, J=8.4 Hz, 2H), 7.51 (d, J=8.5 Hz, 2H), 7.43 (d, J=8.5 Hz, 2H), 3.87 (d, J=12.0 Hz, 2H), 2.76 (d, J=10.9 Hz, 2H), 2.41-2.02 (m, 5H), 1.83 (dd, J=12.3, 3.6 Hz, 2H), 1.70-1.46 (m, 4H), 1.37-1.05 (m, 3H), 0.87 (d, J=6.3 Hz, 3H). ¹³C NMR (100 MHz, CDCl₃) δ 144.2, 137.7, 135.1, 134.7, 129.2, 128.5, 128.3, 127.4, 61.4, 49.5, 46.2, 34.6, 31.0, 27.3, 21.8. HRMS (ESI-TOF) calcd for C₂₃H₃₀ClN₂O₂S [M+H]⁺ 433.1711, found 433.1706.

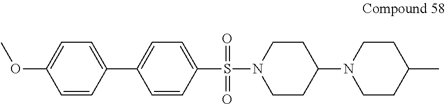

Compound 58: 1′-((4′-Methoxy-[1,1′-biphenyl]-4-yl)sulfonyl)-4-methyl-1,4′-bipiperidine. Reaction of amine 1a with 4′-methoxy-[1,1′-biphenyl]-4-sulfonyl chloride (Procedure A) yielded 58 as a white solid (92%); mp 195-198° C. ¹H NMR (400 MHz, CDCl₃) δ 7.85-7.72 (m, 2H), 7.66 (d, J=8.5 Hz, 2H), 7.53 (d, J=8.7 Hz, 2H), 7.04-6.88 (m, 2H), 3.88 (d, J=12.0 Hz, 2H), 3.84 (s, 3H), 2.76 (d, J=11.6 Hz, 2H), 2.40-1.95 (m, 5H), 1.83 (d, J=10.7 Hz, 2H), 1.71-1.48 (m, 4H), 1.36-1.23 (m, 1H), 1.23-1.04 (m, 2H), 0.87 (d, J=6.3 Hz, 3H). ¹³C NMR (100 MHz, CDCl₃) δ 160.0, 145.1, 133.9, 131.6, 128.4, 128.2, 126.9, 114.5, 61.5, 55.4, 49.5, 46.2, 34.6, 31.0, 27.4, 21.9. HRMS (ESI-TOF) calcd for C₂₄H₃₃N₂O₃S [M+H]⁺ 429.226 found 224.2203.

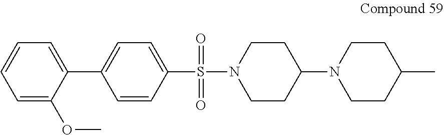 [SMILES: COc1ccccc1-c1ccc(S(=O)(=O)N2CCC(N3CCC(C)CC3)CC2)cc1]

Compound 59: 1′-((2′-Methoxy-[1,1′-biphenyl]-4-yl)sulfonyl)-4-methyl-1,4′-bipiperidine. Reaction of amine 1a with 2′-methoxy-[1,1′-biphenyl]-4-sulfonyl chloride (Procedure A) yielded 59 as a colorless gel (88%). ¹H NMR (400 MHz, CDCl₃) δ 7.74 (d, J=8.3 Hz, 2H), 7.66 (d, J=8.4 Hz, 2H), 7.35 (t, J=7.9 Hz, 1H), 7.29 (d, J=7.5 Hz, 1H), 7.07-6.91 (m, 2H), 3.86 (d, J=11.6 Hz, 2H), 3.80 (s, 3H), 2.77 (d, J=11.4 Hz, 2H), 2.32 (t, J=11.3 Hz, 2H), 2.22 (tt, J=11.6, 3.7 Hz, 1H), 2.11 (t, J=10.8 Hz, 2H), 1.83 (d, J=11.2 Hz, 2H), 1.73-1.52 (m, 4H), 1.37-1.23 (m, 1H), 1.16 (qd, J=11.9, 3.7 Hz, 2H), 0.87 (d, J=6.3 Hz, 3H). ¹³C NMR (100 MHz, CDCl₃) δ 156.3, 143.2, 134.2, 130.7, 130.0, 129.8, 128.6, 127.3, 121.0, 111.3, 61.4, 55.5, 49.4, 46.1, 34.5, 31.0, 27.3, 21.8. HRMS (ESI-TOF) calcd for C₂₄H₃₃N₂O₃S [M+H]⁺ 429.2206, found 429.2205.

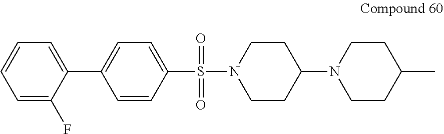 [SMILES: CC1CCN(C2CCN(S(=O)(=O)c3ccc(-c4ccccc4F)cc3)CC2)CC1]

Compound 60: 1′-((2′-Fluoro-[1,1′-biphenyl]-4-yl)sulfonyl)-4-methyl-1,4′-bipiperidine. Reaction of amine 1a with 2′-fluoro-[1,1′-biphenyl]-4-sulfonyl chloride (Procedure A) yielded 60 as a colorless gel (73%). ¹H NMR (400 MHz, CDCl₃) δ 7.85-7.76 (m, 2H), 7.72-7.64 (m, 2H), 7.42 (td, J=7.7, 1.8 Hz, 1H), 7.39-7.33 (m, 1H), 7.24 (dd, J=6.3, 1.3 Hz, 1H), 7.22-7.12 (m, 1H), 3.87 (d, J=12.0 Hz, 2H), 2.77 (d, J=11.5 Hz, 2H), 2.42-2.17 (m, 3H), 2.11 (td, J=11.7, 2.4 Hz, 2H), 1.84 (d, J=13.6 Hz, 2H), 1.72-1.54 (m, 4H), 1.36-1.23 (m, 1H), 1.24-1.09 (m, 2H), 0.87 (d, J=6.3 Hz, 3H). ¹³C NMR (100 MHz, CDCl₃) δ 159.6 (d, J=249.1 Hz), 140.3 (d, J=1.3 Hz), 135.2, 130.6 (d, J=3.1 Hz), 130.2 (d, J=8.3 Hz), 129.5 (d, J=3.2 Hz), 127.8, 127.2 (d, J=13.0 Hz), 124.7 (d, J=3.7 Hz), 116.3 (d, J=22.6 Hz), 61.4, 49.4, 46.2, 34.5, 31.0, 27.3, 21.8. HRMS (ESI-TOF) calcd for C₂₃H₃₀FN₂O₂S [M+H]⁺ 417.2007, found 417.1995.

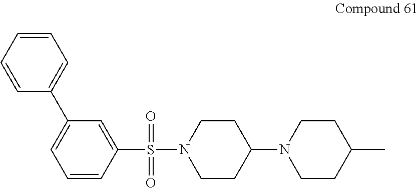 [SMILES: CC1CCN(C2CCN(S(=O)(=O)c3cccc(-c4ccccc4)c3)CC2)CC1]

Compound 61: 1′-([1,1′-Biphenyl]-3-ylsulfonyl)-4-methyl-1,4′-bipiperidine. Reaction of compound 29 with phenylboronic acid (Procedure C) yielded compound 61 as a black gel (58%). ¹H NMR (400 MHz, CDCl₃) δ 7.94 (t, J=1.8 Hz, 1H), 7.79 (ddd, J=7.8, 1.9, 1.1 Hz, 1H), 7.71 (ddd, J=7.8, 1.8, 1.1 Hz, 1H), 7.62-7.55 (m, 3H), 7.50-7.43 (m, 1H), 7.43-7.36 (m, 1H), 3.87 (d, J=12.0 Hz, 2H), 2.77 (d, J=11.7 Hz, 2H), 2.28 (td, J=12.0, 2.5 Hz, 2H), 2.20 (td, J=8.1, 4.1 Hz, 1H), 2.11 (td, J=11.5, 2.4 Hz, 2H), 1.83 (d, J=11.6 Hz, 2H), 1.72-1.54 (m, 4H), 1.37-1.23 (m, 1H), 1.17 (qd, J=12.1, 3.8 Hz, 2H), 0.87 (d, J=6.3 Hz, 3H). ¹³C NMR (100 MHz, CDCl₃) δ 142.3, 139.2, 136.7, 131.3, 129.4, 129.1, 128.3, 127.2, 126.2, 126.1, 61.4, 49.4, 46.2, 34.4, 31.0, 27.3, 21.8. HRMS (ESI-TOF) calcd for C₂₃H₃₁N₂O₂S [M+H]⁺ 399.2101, found 399.2096.

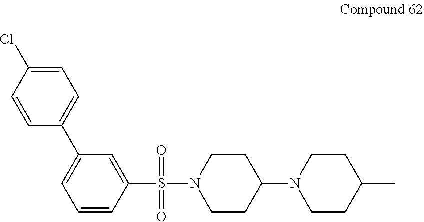 [SMILES: CC1CCN(C2CCN(S(=O)(=O)c3cccc(-c4ccc(Cl)cc4)c3)CC2)CC1]

Compound 62: 1′-((4′-Chloro-[1,1′-biphenyl]-3-yl)sulfonyl)-4-methyl-1,4′-bipiperidine. Reaction of compound 29 with 4-chlorophenyl)boronic acid (Procedure C) yielded compound 62 as a pink solid (58%); mp 124-126° C. ¹H NMR (400 MHz, CDCl₃) δ 7.89 (t, J=1.8 Hz, 1H), 7.72 (ddt, J=15.7, 7.9, 1.4 Hz, 2H), 7.58 (t, J=7.8 Hz, 1H), 7.54-7.48 (m, 2H), 7.45-7.39 (m, 2H), 3.86 (d, J=12.1 Hz, 2H), 2.80 (dt, J=11.5, 3.1 Hz, 2H), 2.27 (td, J=12.1, 2.4 Hz, 3H), 2.13 (t, J=10.6 Hz, 2H), 1.86 (d, J=13.3 Hz, 2H), 1.72-1.52 (m, 4H), 1.38-1.14 (m, 3H), 0.87 (d, J=6.1 Hz, 3H). ¹³C NMR (100 MHz, CDCl₃) δ 141.1, 137.6, 136.9, 134.5, 131.1, 129.6, 129.2, 128.5, 126.5, 125.8, 61.5, 49.4, 46.1, 34.2, 30.8, 27.1, 21.7. HRMS (ESI-TOF) calcd for C₂₃H₃₀ClN₂O₂S [M+H]⁺ 433.1711, found 433.1705.

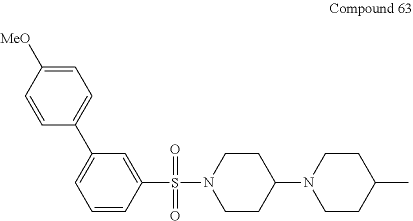 [SMILES: COc1ccc(-c2cccc(S(=O)(=O)N3CCC(N4CCC(C)CC4)CC3)c2)cc1]

Compound 63: 1′-((4′-Methoxy-[1,1′-biphenyl]-3-yl)sulfonyl)-4-methyl-1,4′-bipiperidine. Reaction of compound 29 with (4-methoxyphenyl)boronic acid (Procedure C) yielded compound 63 as a yellow solid (44%); mp 107-109° C. ¹H NMR (400 MHz, CDCl₃) δ 7.89 (s, 1H), 7.74 (d, J=7.8 Hz, 1H), 7.65 (d, J=8.0 Hz, 1H), 7.58-7.47 (m, 3H), 6.98 (d, J=8.5 Hz, 2H), 3.87 (d, J=12.2 Hz, 2H), 3.84 (s, 3H), 2.80 (d, J=10.0 Hz, 2H), 2.27 (td, J=12.0, 2.4 Hz, 3H), 2.15 (t, J=11.9 Hz, 2H), 1.87 (d, J=11.7 Hz, 2H), 1.75-1.53 (m, 4H), 1.38-1.19 (m, 3H), 0.88 (d, J=6.4 Hz, 3H). ¹³C NMR (100 MHz, CDCl₃) δ 159.9, 141.9, 136.6, 131.6, 130.8, 129.4, 128.3, 125.6, 125.5, 114.5, 61.5, 55.4, 49.4, 46.1, 34.2, 30.8, 27.1, 21.7. HRMS (ESI-TOF) calcd for C₂₄H₃₃N₂O₃S [M+H]⁺ 429.2206, found 429.2206.

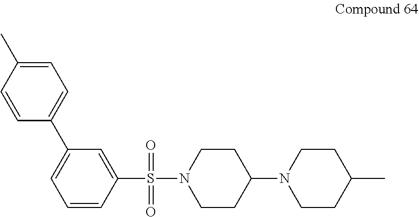 [SMILES: Cc1ccc(-c2cccc(S(=O)(=O)N3CCC(N4CCC(C)CC4)CC3)c2)cc1]

Compound 64: 4-Methyl-1′-((4′-methyl-[1,1′-biphenyl]-3-yl)sulfonyl)-1,4′-bipiperidine. Reaction of compound 29 with p-tolylboronic acid (Procedure C) yielded compound 64 as a yellow solid (95%); mp 103-105° C. ¹H NMR (400 MHz, CDCl₃) δ 7.92 (s, 1H), 7.78 (d, J=7.8 Hz, 1H), 7.68 (d, J=7.8 Hz, 1H), 7.57 (t, J=7.8 Hz, 1H), 7.49 (d, J=8.1 Hz, 2H), 7.27 (d, J=7.9 Hz, 2H), 3.87 (d, J=11.9 Hz, 2H), 2.81 (d, J=11.2 Hz, 2H), 2.40 (s, 3H), 2.31-2.20 (m, 3H), 2.17 (t, J=11.8 Hz, 2H), 1.86 (d, J=12.4 Hz, 2H), 1.74-1.55 (m, 4H), 1.40-1.12 (m, 3H), 0.88 (d, J=6.1 Hz, 3H). ¹³C NMR (100 MHz, CDCl₃) δ 142.2, 138.3, 136.6, 136.3, 131.1, 129.8, 129.4, 127.0, 125.9, 125.8, 61.5, 49.4, 46.1, 34.1, 30.8, 27.1, 21.7, 21.1. HRMS (ESI-TOF) calcd for C₂₄H₃₃N₂O₂S [M+H]⁺ 413.2257, found 413.2258.

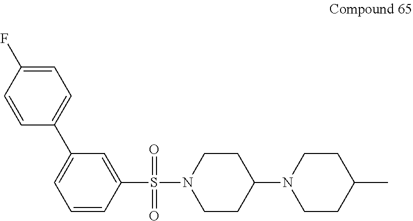 [SMILES: CC1CCN(C2CCN(S(=O)(=O)c3cccc(-c4ccc(F)cc4)c3)CC2)CC1]

Compound 65: 1′-((4′-Fluoro-[1,1′-biphenyl]-3-yl)sulfonyl)-4-methyl-1,4′-bipiperidine. Reaction of compound 29 with (4-fluorophenyl)boronic acid (Procedure C) yielded compound 65 as a white solid (63%); nip 131-134° C. ¹H NMR (400 MHz, CDCl₃) δ 7.88 (s, 1H), 7.76-7.66 (m, 2H), 7.60-7.50 (m, 3H), 7.14 (t, J=8.6 Hz, 2H), 3.86 (d, J=11.9 Hz, 2H), 2.75 (d, J=11.5 Hz, 2H), 2.28 (td, J=12.1, 2.5 Hz, 2H), 2.18 (tt, J=11.5, 3.6 Hz, 1H), 2.09 (td, J=11.5, 2.4 Hz, 2H), 1.82 (d, J=11.8 Hz, 2H), 1.70-1.51 (m, 4H), 1.38-1.20 (m, 1H), 1.21-1.06 (m, 2H), 0.86 (d, J=6.3 Hz, 3H). ¹³C NMR (100 MHz, CDCl₃) δ 162.94 (d, J=248.1 Hz), 141.27, 136.89, 135.39 (d, J=3.3 Hz), 131.11, 129.53, 128.89 (d, J=8.3 Hz), 126.26, 125.88, 116.02 (d, J=21.6 Hz), 61.40, 49.47, 46.20, 34.58, 31.01, 27.36, 21.86. HRMS (ESI-TOF) calcd for C₂₃H₃₀FN₂O₂S [M+H]⁺ 417.2007, found 417.2018.

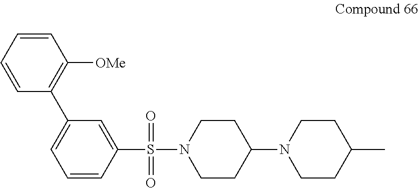 [SMILES: COc1ccccc1-c1cccc(S(=O)(=O)N2CCC(N3CCC(C)CC3)CC2)c1]

Compound 66: 1′-((2′-Methoxy-[1,1′-biphenyl]-3-yl)sulfonyl)-4-methyl-1,4′-bipiperidine. Reaction of compound 29 with (2-methoxyphenyl)boronic acid (Procedure C) yielded compound 66 as an orange gel (53%). ¹H NMR (400 MHz, CDCl₃) δ 7.93 (s, 1H), 7.73 (dt, J=7.8, 1.4 Hz, 1H), 7.67 (dt, J=8.0, 1.4 Hz, 1H), 7.52 (t, J=7.8 Hz, 1H), 7.39-7.27 (m, 2H), 7.08-6.94 (m, 2H), 3.86 (d, J=12.0 Hz, 2H), 3.78 (s, 3H), 2.77 (d, J=11.6 Hz, 2H), 2.43-2.17 (m, 3H), 2.13 (t, J=11.0 Hz, 2H), 1.82 (d, J=12.1 Hz, 2H), 1.71-1.52 (m, 4H), 1.38-1.24 (m, 1H), 1.24-1.10 (m, 2H), 0.87 (d, J=6.3 Hz, 3H). ¹³C NMR (100 MHz, CDCl₃) δ 156.3, 139.5, 135.5, 133.7, 130.7, 129.6, 128.7, 128.6, 128.5, 125.8, 121.0, 111.3, 61.5, 55.5, 49.4, 46.2, 34.5, 31.0, 27.3, 21.8. HRMS (ESI-TOF) calcd for C₂₄H₃₃N₂O₃S [M+H]⁺ 429.2206, found 429.2192.

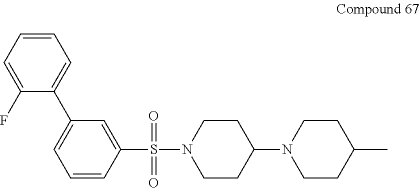 [SMILES: CC1CCN(C2CCN(S(=O)(=O)c3cccc(-c4ccccc4F)c3)CC2)CC1]

Compound 67: 1′-((2′-Fluoro-[1,1′-biphenyl]-3-yl)sulfonyl)-4-methyl-1,4′-bipiperidine. Reaction of compound 29 with (2-fluorophenyl)boronic acid (Procedure C) yielded compound 67 as a yellow gel (74%). ¹H NMR (400 MHz, CDCl₃) δ 7.89 (d, J=1.6 Hz, 1H), 7.79-7.68 (m, 2H), 7.57 (t, J=7.8 Hz, 1H), 7.41 (td, J=7.7, 1.9 Hz, 1H), 7.38-7.31 (m, 1H), 7.25-7.18 (m, 1H), 7.14 (ddd, J=10.8, 8.2, 1.2 Hz, 1H), 3.86 (d, J=12.1 Hz, 2H), 2.80 (d, J=11.8 Hz, 2H), 2.29 (td, J=12.1, 2.5 Hz, 3H), 2.21-2.08 (m, 2H), 1.87 (d, J=12.6 Hz, 2H), 1.70-1.46 (m, 4H), 1.39-1.05 (m, 3H), 0.86 (d, J=5.9 Hz, 3H). ¹³C NMR (100 MHz, CDCl₃) δ 159.58 (d, J=248.6 Hz), 136.83, 136.38, 133.19 (d, J=3.0 Hz), 132.03 (d, J=9.9 Hz), 130.55 (d, J=3.0 Hz), 130.06 (d, J=8.3 Hz), 129.15, 127.94 (d, J=3.1 Hz), 126.64, 124.69 (d, J=3.7 Hz), 116.27 (d, J=22.5 Hz), 61.50, 49.38, 46.06, 34.11, 30.78, 27.10, 21.71. HRMS (ESI-TOF) calcd for C₂₃H₃₀FN₂O₂S [M+H]⁺ 417.2007, found 417.1999.

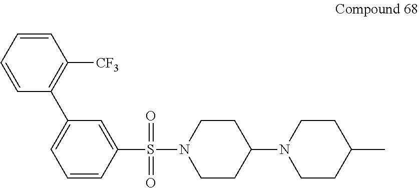 [SMILES: CC1CCN(C2CCN(S(=O)(=O)c3cccc(-c4ccccc4C(F)(F)F)c3)CC2)CC1]

Compound 68: 4-Methyl-1′-((2′-(trifluoromethyl)-[1,1′-biphenyl]-3-yl)sulfonyl)-1,4′-bipiperidine. Reaction of compound 29 with (2-(trifluoromethyl)phenyl)boronic acid (Procedure C) yielded compound 68 as a yellow solid (37%); mp 110-112° C. ¹H NMR (400 MHz, CDCl₃) δ 7.80-7.68 (m, 3H), 7.61-7.47 (m, 4H), 7.30 (d, J=8.0 Hz, 1H), 3.82 (d, J=12.1 Hz, 1H), 2.79 (d, J=11.7 Hz, 2H), 2.35-2.18 (m, 3H), 2.13 (td, J=11.5, 2.4 Hz, 2H), 1.84 (d, J=10.8 Hz, 2H), 1.70-1.53 (m, 4H), 1.38-1.25 (m, 1H), 1.25-1.12 (qd, J=12.0, 3.8 Hz, 2H), 0.87 (d, J=6.3 Hz, 3H). ¹³C NMR (100 MHz, CDCl₃) δ 140.7, 139.3 (q, J=2.1 Hz), 135.8, 133.2, 131.8, 131.7, 128.6, 128.2, 128.1 (q, J=1.6 Hz), 126.9, 126.2 (q, J=5.3 Hz), 124.0 (q, J=274.0 Hz), 61.5, 49.4, 46.1, 34.3, 30.9, 27.2, 21.8. HRMS (ESI-TOF) calcd for C₂₄H₃₀F₃N₂O₂S [M+H]⁺ 467.1975, found 467.1981.

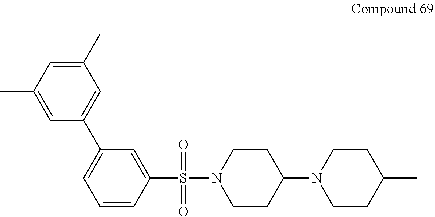

Compound 69: 1′-((3′,5′-Dimethyl-[1,1′-biphenyl]-3-yl)sulfonyl)-4-methyl-1,4′-bipiperidine. Reaction of compound 29 with (3,5-dimethylphenyl)boronic acid (Procedure C) yielded compound 69 as a yellow solid (45%); mp 120-123° C. ¹H NMR (400 MHz, CDCl₃) δ 7.92 (s, 1H), 7.77 (dt, J=7.7, 1.4 Hz, 1H), 7.68 (dd, J=7.8, 3.0 Hz, 1H), 7.55 (t, J=7.8 Hz, 1H), 7.19 (s, 2H), 7.03 (s, 1H), 3.86 (d, J=12.0 Hz, 2H), 2.77 (d, J=11.3 Hz, 2H), 2.37 (s, 6H), 2.33-2.16 (m, 3H), 2.11 (t, J=11.1 Hz, 2H), 1.83 (dt, J=12.2, 3.1 Hz, 2H), 1.72-1.54 (m, 4H), 1.39-1.08 (m, 3H), 0.87 (d, J=6.3 Hz, 3H). ¹³C NMR (100 MHz, CDCl₃) δ 142.5, 139.2, 138.7, 136.5, 131.3, 129.9, 129.3, 126.1, 125.1, 61.4, 49.4, 46.2, 34.4, 30.9, 27.2, 21.8, 21.4. HRMS (ESI-TOF) calcd for C₂₅H₃₅N₂O₂S [M+H]⁺ 427.2414, found 427.2407.

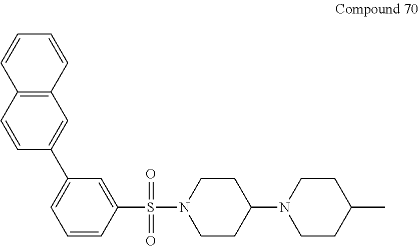 [SMILES: CC1CCN(C2CCN(S(=O)(=O)c3cccc(-c4ccc5ccccc5c4)c3)CC2)CC1]

Compound 70: 4-Methyl-1′-((3-(naphthalen-2-yl)phenyl)sulfonyl)-1,4′-bipiperidine. Reaction of compound 29 with naphthalen-2-ylboronic acid (Procedure C) yielded compound 70 as a yellow gel (29%). ¹H NMR (400 MHz, CDCl₃) δ 8.06 (d, J=9.7 Hz, 2H), 7.96-7.82 (m, 4H), 7.72 (ddd, J=10.5, 8.2, 1.6 Hz, 2H), 7.62 (t, J=7.8 Hz, 1H), 7.56-7.47 (m, 2H), 3.90 (d, J=11.9 Hz, 2H), 2.79 (d, J=11.2 Hz, 2H), 2.38-2.20 (m, 3H), 2.13 (t, J=10.8 Hz, 2H), 1.87 (d, J=12.5 Hz, 2H), 1.74-1.51 (m, 4H), 1.39-1.13 (m, 3H), 0.88 (d, J=6.0 Hz, 3H). ¹³C NMR (100 MHz, CDCl₃) δ 142.2, 136.8, 136.5, 133.5, 132.9, 131.5, 129.6, 128.9, 128.3, 127.7, 126.7, 126.6, 126.3, 126.3, 125.0, 61.5, 49.4, 46.2, 34.3, 30.9, 27.2, 21.8. HRMS (ESI-TOF) calcd for C₂₇H₃₃N₂O₂S [M+H]⁺ 449.2257, found 449.2254.

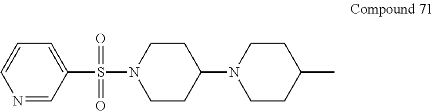 [SMILES: CC1CCN(C2CCN(S(=O)(=O)c3cccnc3)CC2)CC1]

Compound 71: 4-Methyl-1′-(pyridin-3-ylsulfonyl)-1,4′-bipiperidine. Reaction of amine 1a with pyridine-3-sulfonyl chloride (Procedure A) yielded 71 as a yellow solid (86%); mp 144-147° C. ¹H NMR (500 MHz, CDCl₃) δ 8.98 (s, 1H), 8.82 (d, J=4.9 Hz, 1H), 8.04 (d, J=8.1 Hz, 1H), 7.49 (dd, J=8.0, 4.9 Hz, 1H), 3.88 (d, J=12.3 Hz, 2H), 2.78 (d, J=11.9 Hz, 2H), 2.32 (td, J=12.0, 2.4 Hz, 2H), 2.22 (tt, J=11.6, 3.6 Hz, 1H), 2.12 (t, J=11.6 Hz, 2H), 1.86 (d, J=11.9 Hz, 2H), 1.75-1.55 (m, 4H), 1.38-1.23 (m, 1H), 1.17 (qd, J=12.0, 3.7 Hz, 2H), 0.89 (d, J=6.5 Hz, 3H). ¹³C NMR (100 MHz, CDCl₃) δ 153.3, 148.4, 135.2, 133.1, 123.7, 61.3, 49.5, 46.0, 34.5, 31.0, 27.3, 21.8. HRMS (ESI-TOF) calcd for C₁₆H₂₅ClN₃O₂SNa [M+Na]⁺346.1560, found 346.1566.

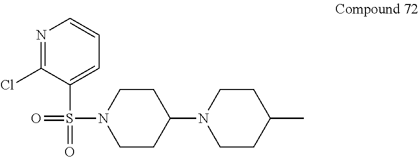 [SMILES: CC1CCN(C2CCN(S(=O)(=O)c3cccnc3Cl)CC2)CC1]

Compound 72: 1′-((2-Chloropyridin-3-yl)sulfonyl)-4-methyl-1,4′-bipiperidine. Reaction of amine 1a with 2-chloropyridine-3-sulfonyl chloride (Procedure A) yielded 72 as a white solid (71%); mp 122-124° C. ¹H NMR (500 MHz, CDCl₃) δ 8.55 (d, J=4.9 Hz, 1H), 8.38 (d, J=7.8 Hz, 1H), 7.40 (dd, J=7.8, 4.8 Hz, 1H), 3.92 (d, J=13.0 Hz, 2H), 2.83 (t, J=12.5 Hz, 4H), 2.38 (t, J=11.5 Hz, 1H), 2.15 (t, J=11.0 Hz, 2H), 1.86 (d, J=12.5 Hz, 2H), 1.72-1.53 (m, 4H), 1.38-1.26 (m, 1H), 1.25-1.13 (m, 2H), 0.90 (d, J=6.5 Hz, 3H). ¹³C NMR (100 MHz, CDCl₃) δ 148.4, 144.5, 136.9, 130.5, 118.5, 57.6, 45.6, 41.9, 30.7, 27.1, 24.0, 17.9. HRMS (ESI-TOF) calcd for C₁₆H₂₄ClN₃O₂SNa [M+Na]⁺380.1170, found 380.1160.

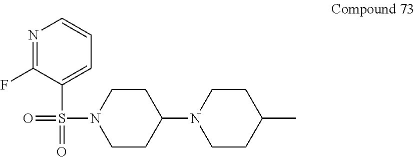 [SMILES: CC1CCN(C2CCN(S(=O)(=O)c3cccnc3F)CC2)CC1]

Compound 73: 1′-((2-Fluoropyridin-3-yl)sulfonyl)-4-methyl-1,4′-bipiperidine. Reaction of amine 1a with 2-fluoropyridine-3-sulfonyl chloride (Procedure A) yielded 73 as a white solid (>95%); mp 120-123° C. ¹H NMR (500 MHz, CDCl₃) δ 8.41 (d, J=4.9 Hz, 1H), 8.29 (dd, J=9.2, 7.6 Hz, 1H), 7.37 (dd, J=7.6, 4.9 Hz, 1H), 3.96 (d, J=12.6 Hz, 2H), 2.81 (d, J=11.5 Hz, 2H), 2.67 (t, J=12.4 Hz, 2H), 2.34 (ddd, J=11.5, 8.0, 3.5 Hz, 1H), 2.15 (t, J=10.4 Hz, 2H), 1.87 (d, J=12.1 Hz, 2H), 1.74-1.55 (m, 4H), 1.32 (ddt, J=10.3, 6.9, 4.1 Hz, 1H), 1.27-1.11 (m, 2H), 0.91 (d, J=6.4 Hz, 3H). ¹³C NMR (100 MHz, CDCl₃) δ 154.75 (d, J=244.0 Hz), 147.68 (d, J=14.3 Hz), 138.2, 118.2, 117.9, 57.5, 45.6, 41.9, 30.7, 27.1, 23.8, 17.9. HRMS (ESI-TOF) calcd for C₁₆H₂₄FN₃O₂SNa [M+Na]⁺364.1465, found 364.1462.

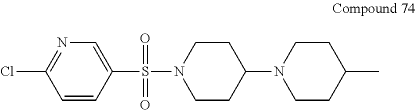 [SMILES: CC1CCN(C2CCN(S(=O)(=O)c3ccc(Cl)nc3)CC2)CC1]

Compound 74: 1′-((6-Chloropyridin-3-yl)sulfonyl)-4-methyl-1,4′-bipiperidine. Reaction of amine 1a with 6-chloropyridine-3-sulfonyl chloride (Procedure A) yielded 74 as a white solid (80%); mp 178-182° C. ¹H NMR (500 MHz, CDCl₃) δ 8.75 (s, 1H), 7.99 (dd, J=8.4, 2.3 Hz, 1H), 7.51 (d, J=8.3 Hz, 1H), 3.87 (d, J=12.0 Hz, 2H), 2.81 (d, J=8.5 Hz, 2H), 2.35 (t, J=12.0 Hz, 2H), 2.25 (t, J=11.4 Hz, 1H), 2.14 (t, J=11.3 Hz, 2H), 1.89 (d, J=11.3 Hz, 2H), 1.75-1.58 (m, 4H), 1.33 (br, 1H), 1.28-1.10 (m, 2H), 0.91 (d, J=6.6 Hz, 3H). ¹³C NMR (100 MHz, CDCl₃) δ 155.6, 148.6, 137.7, 132.1, 124.7, 61.3, 49.5, 46.0, 34.4, 30.9, 27.3, 21.8. HRMS (ESI-TOF) calcd for C₁₆H₂₄ClN₃O₂SNa [M+Na]⁺ 380.1170, found 380.1171.

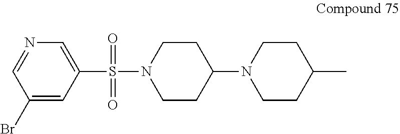 [SMILES: CC1CCN(C2CCN(S(=O)(=O)c3cncc(Br)c3)CC2)CC1]

Compound 75: 1′-((5-Bromopyridin-3-yl)sulfonyl)-4-methyl-1,4′-bipiperidine. Reaction of amine 1a with 5-bromopyridine-3-sulfonyl chloride (Procedure A) yielded 75 as a white solid (79%); mp 171-173° C. ¹H NMR (500 MHz, CDCl₃) δ 8.81 (s, 2H), 8.18 (t, J=2.0 Hz, 1H), 3.89 (d, J=11.6 Hz, 2H), 2.81 (d, J=11.0 Hz, 2H), 2.39 (td, J=12.0, 2.5 Hz, 2H), 2.25 (d, J=11.9 Hz, 1H), 2.14 (t, J=11.6 Hz, 2H), 1.90 (d, J=12.8 Hz, 2H), 1.78-1.57 (m, 4H), 1.43-1.29 (m, 1H), 1.28-1.11 (m, 2H), 0.91 (d, J=6.4 Hz, 3H). ¹³C NMR (100 MHz, CDCl₃) δ 154.4, 146.2, 137.4, 134.5, 121.0, 61.2, 49.5, 46.0, 34.5, 31.0, 27.3, 21.8. HRMS (ESI-TOF) calcd for C₁₆H₂₄BrN₃O₂SNa [M+Na]⁺424.0665, found 424.0668.

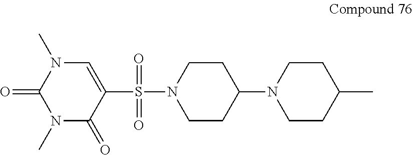 [SMILES: CC1CCN(C2CCN(S(=O)(=O)c3cn(C)c(=O)n(C)c3=O)CC2)CC1]

Compound 76: 1,3-Dimethyl-5-((4-methyl-[1,4′-bipiperidin]-1′-yl)sulfonyl)pyrimidine-2,4(1H,3H)-dione. Reaction of amine 1a with 1,3-dimethyl-2,4-dioxo-1,2,3,4-tetrahydropyrimidine-5-sulfonyl chloride (Procedure A) yielded 76 as a white solid (78%); mp 212-215° C. ¹H NMR (500 MHz, CDCl₃) δ 8.07 (s, 1H), 3.94 (d, J=12.8 Hz, 2H), 3.50 (s, 3H), 3.35 (s, 3H), 2.93 (d, J=11.6 Hz, 2H), 2.85 (t, J=12.7 Hz, 2H), 2.56 (t, J=11.7 Hz, 1H), 2.26 (t, J=11.9 Hz, 2H), 1.92 (d, J=11.9 Hz, 2H), 1.75-1.58 (m, 4H), 1.48-1.20 (m, 3H), 0.93 (d, J=6.0 Hz, 3H). ¹³C NMR (100 MHz, CDCl₃) δ 158.2, 150.8, 148.1, 112.6, 61.6, 49.2, 46.0, 37.9, 33.8, 30.7, 28.3, 27.5, 21.7. HRMS (ESI-TOF) calcd for C₁₇H₂₅N₄O₄SNa [M+Na]⁺ 407.1723, found 407.1725.

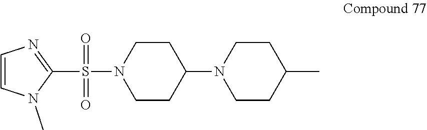 [SMILES: CC1CCN(C2CCN(S(=O)(=O)c3nccn3C)CC2)CC1]

Compound 77: 4-Methyl-1′-((1-methyl-1H-imidazol-2-yl)sulfonyl)-1,4′-bipiperidine. Reaction of amine 1a with 1-methyl-1H-imidazole-2-sulfonyl chloride (Procedure A) yielded 77 as a white solid (51%); mp 153-156° C. ¹H NMR (400 MHz, CDCl₃) δ 7.03 (d, J=1.1 Hz, 1H), 6.92 (d, J=1.1 Hz, 1H), 3.96 (d, J=12.2 Hz, 2H), 3.89 (s, 3H), 3.05 (t, J=12.5 Hz, 2H), 2.89 (d, J=10.8 Hz, 2H), 2.54 (s, 1H), 2.24 (s, 2H), 1.94 (d, J=12.5 Hz, 2H), 1.81-1.58 (m, 4H), 1.31 (s, 3H), 1.06-0.79 (d, J=8.0 Hz, 3H). ¹³C NMR (100 MHz, CDCl₃) δ 142.8, 128.2, 124.4, 61.8, 49.4, 46.6, 34.8, 34.1, 30.9, 27.3, 21.7. HRMS (ESI-TOF) calcd for C₁₅H₂₆N₄O₂SNa [M+Na]⁺ 349.1669, found 349.1673.

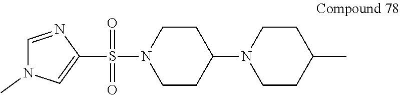

Compound 78: 4-Methyl-1′-((1-methyl-1H-imidazol-4-yl)sulfonyl)-1,4′-bipiperidine. Reaction of amine 1a with 1-methyl-1H-imidazole-4-sulfonyl chloride (Procedure A) yielded 78 as a yellow solid (61%); mp 139-142° C. ¹H NMR (500 MHz, CDCl₃) δ 7.48 (s, 1H), 7.42 (s, 1H), 3.90 (d, J=13.0 Hz, 2H), 3.75 (s, 3H), 2.80 (d, J=9.9 Hz, 2H), 2.57 (t, J=12.1 Hz, 2H), 2.26 (t, J=11.5 Hz, 1H), 2.13 (t, J=10.5 Hz, 2H), 1.83 (d, J=12.1 Hz, 2H), 1.69-1.57 (m, 4H), 1.31 (br, 1H), 1.26-1.12 (m, 2H), 0.90 (d, J=6.4 Hz, 3H). ¹³C NMR (100 MHz, CDCl₃) δ 139.0, 138.3, 124.4, 61.7, 49.5, 46.3, 34.6, 34.0, 31.1, 27.4, 21.9. HRMS (ESI-TOF) calcd for C₁₅H₂₆N₂₄O₂SNa [M+Na]⁺ 349.1669, found 349.1660.

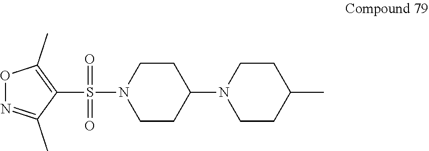 [SMILES: Cc1noc(C)c1S(=O)(=O)N1CCC(N2CCC(C)CC2)CC1]

Compound 79: 3,5-Dimethyl-4-((4-methyl-[1,4′-bipiperidin]-1′-yl)sulfonyl)isoxazole. Reaction of amine 1a with 3,5-dimethylisoxazole-4-sulfonyl chloride (Procedure A) yielded 79 as a white solid (28%); mp 158-161° C. ¹H NMR (400 MHz, CDCl₃) δ 3.78 (d, J=11.9 Hz, 2H), 2.80 (d, J=11.5 Hz, 2H), 2.61 (s, 3H), 2.50 (t, J=12.0 Hz, 2H), 2.38 (s, 3H), 2.27 (tt, J=11.4, 3.5 Hz, 1H), 2.14-2.06 (m, 2H), 1.87 (d, J=11.6 Hz, 2H), 1.72-1.50 (m, 4H), 1.38-1.24 (m, 1H), 1.25-1.09 (m, 2H), 0.89 (d, J=6.4 Hz, 3H). ¹³C NMR (100 MHz, CDCl₃) δ 173.5, 158.0, 113.7, 61.3, 49.6, 45.4, 34.6, 31.0, 27.5, 21.9, 13.0, 11.4. HRMS (ESI-TOF) calcd for C₁₆H₂₈N₃O₃S [M+H]⁺ 342.1846, found 342.1854.

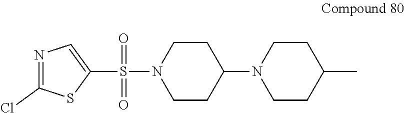 [SMILES: CC1CCN(C2CCN(S(=O)(=O)c3cnc(Cl)s3)CC2)CC1]

Compound 80: 2-Chloro-5-((4-methyl-[1,4′-bipiperidin]-1′-yl)sulfonyl)thiazole. Reaction of amine 1a with 2-chlorothiazole-5-sulfonyl chloride (Procedure A) yielded 80 as a pale yellow solid (51%); mp 141-143° C. ¹H NMR (500 MHz, CDCl₃) δ 7.87 (s, 1H), 3.82 (d, J=10.4 Hz, 2H), 2.83 (d, J=8.6 Hz, 2H), 2.45 (td, J=12.3, 2.7 Hz, 2H), 2.32 (t, J=11.4 Hz, 1H), 2.16 (t, J=11.5 Hz, 2H), 1.92 (d, J=12.3 Hz, 2H), 1.74-1.56 (m, 4H), 1.39-1.13 (m, 3H), 0.89 (d, J=6.3 Hz, 2H). ¹³C NMR (100 MHz, CDCl₃) δ 157.2, 144.9, 134.4, 61.2, 49.5, 46.0, 34.2, 30.8, 27.1, 21.7. HRMS (ESI-TOF) calcd for C₁₄H₂₂ClN₃O₂S₂Na [M+Na]⁺ 386.0734, found 386.0745.

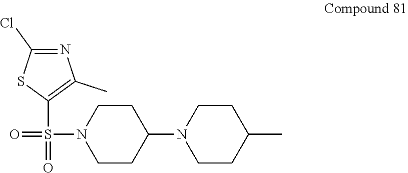 [SMILES: Cc1nc(Cl)sc1S(=O)(=O)N1CCC(N2CCC(C)CC2)CC1]

Compound 81: 2-Chloro-4-methyl-5-((4-methyl-[1,4′-bipiperidin]-1′-yl)sulfonyl)thiazole. Reaction of amine 1a with 2-chloro-4-methylthiazole-5-sulfonyl chloride (Procedure A) yielded 81 as a white solid (64%); mp 117-119° C. ¹H NMR (400 MHz, CDCl₃) δ 3.85 (d, J=13.5 Hz, 2H), 2.82 (d, J=11.0 Hz, 2H), 2.60 (s, 3H), 2.53 (t, J=12.0 Hz, 2H), 2.34 (t, J=11.9 Hz, 1H), 2.15 (t, J=11.4 Hz, 2H), 1.91 (d, J=12.7 Hz, 2H), 1.65 (m, 4H), 1.45-1.29 (m, 1H), 1.28-1.12 (m, 2H), 0.89 (d, J=6.2 Hz, 3H). ¹³C NMR (100 MHz, CDCl₃) δ 155.4, 154.4, 128.9, 61.3, 49.5, 46.0, 34.3, 30.9, 27.3, 21.8, 16.8. HRMS (ESI-TOF) calcd for C₁₅H₂₅ClN₃O₂S₂ [M+H]⁺ 378.1071, found 378.1060.

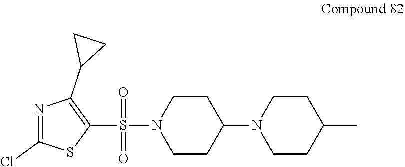 [SMILES: CC1CCN(C2CCN(S(=O)(=O)c3sc(Cl)nc3C3CC3)CC2)CC1]

Compound 82: 2-Chloro-4-cyclopropyl-5-((4-methyl-[1,4′-bipiperidin]-1′-yl)sulfonyl)thiazole. 2-Chloro-4-cyclopropylthiazole (0.3531 g, 2.21 mmol) was dissolved in anhydrous THF (7 mL) at −78° C., followed by dropwise addition of n-BuLi (2.5 M in hexane, 1.0 mL, 2.50 mmol) under argon in 10 min. The solution mixture was stirred for 20 min at −78° C. and anhydrous S02 gas (generated from dropwise addition of sodium sulfite to concentrated aq. HCl; the generated S02 gas was passed through concentrated H₂SO₄) was bubbled through the reaction solution at −78° C. for 10 min and then at room temperature for 1 h. Upon the evaporation of solvents, the residue was dissolved in anhydrous CH₂Cl₂ (6 mL) and NCS (0.4726 g, 3.54 mmol) was added. The resulting suspension was stirred at room temperature for 16 h. The suspension was then filtered and the filtrate was concentrated to about 10 mL, followed by addition of DIPEA (1.0 mL, 5.75 mmol) and 4-methyl-1,4′-bipiperidine 1a (0.4998 g, 2.75 mmol). The reaction mixture was stirred at room temperature for 5.5 h and then poured into saturated NaHCO₃ solution (60 mL). The biphasic solution was extracted with CH₂Cl₂ (3×60 mL). The combined organic layers were dried over Na₂SO₄, filtered and concentrated. The residue was purified through flash chromatography on silica gel (1:19 CH₃OH/CH₂Cl₂), followed by recrystallization from a mixture of CH₂Cl₂ and hexane to afford the desired product as a yellow solid (0.5398 g, 60% over two steps); mp 139-141° C. ¹H NMR (400 MHz, CDCl₃) δ 3.85 (d, J=12.2 Hz, 2H), 2.80 (d, J=11.6 Hz, 2H), 2.62-2.44 (m, 3H), 2.31 (tt, J=11.6, 3.5 Hz, 1H), 2.12 (td, J=11.6, 2.6 Hz, 2H), 1.88 (d, J=12.6 Hz, 2H), 1.70-1.53 (m, 4H), 1.38-1.24 (m, 1H), 1.18 (qd, J=11.8, 3.7 Hz, 2H), 1.11-0.99 (m, 4H), 0.87 (d, J=6.3 Hz, 3H). ¹³C NMR (100 MHz, CDCl₃) δ 161.2, 154.6, 126.3, 61.2, 49.4, 46.1, 34.4, 30.9, 27.3, 21.8, 11.4, 10.9. HRMS (ESI-TOF) calcd for C₁₇H₂₇ClN₃O₂S₂ [M+]⁺ 404.1228, found 404.1226.

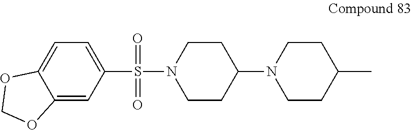 [SMILES: CC1CCN(C2CCN(S(=O)(=O)c3ccc4c(c3)OCO4)CC2)CC1]

Compound 83: 1′-(Benzo[d][1,3]dioxol-4-ylsulfonyl)-4-methyl-1,4′-bipiperidine. Reaction of amine 1a with benzo[d][1,3]dioxole-4-sulfonyl chloride (Procedure A) yielded 83 as a yellow solid (86%). ¹H NMR (400 MHz, CDCl₃) δ 7.12 (dd, J=8.1, 1.2 Hz, 1H), 6.96 (dd, J=7.8, 1.3 Hz, 1H), 6.89 (t, J=8.0 Hz, 1H), 6.06 (s, 2H), 3.88 (d, J=12.4 Hz, 2H), 2.79 (d, J=11.7 Hz, 2H), 2.43 (td, J=12.3, 2.4 Hz, 2H), 2.28 (tt, J=11.6, 3.6 Hz, 1H), 2.13 (td, J=11.6, 2.4 Hz, 2H), 1.84 (d, J=14.8 Hz, 2H), 1.71-1.50 (m, 4H), 1.36-1.25 (m, 1H), 1.19 (qd, J=11.9, 3.7 Hz, 2H), 0.87 (d, J=6.2 Hz, 3H). ¹³C NMR (100 MHz, CDCl₃) δ 148.9, 145.2, 121.7, 121.5, 118.8, 112.5, 102.2, 61.6, 49.4, 45.9, 34.3, 30.9, 27.3, 21.8. HRMS (ESI-TOF) calcd for C₁₈H₂₇N₂O₄S [M+H]⁺ 367.1686, found 367.1686.

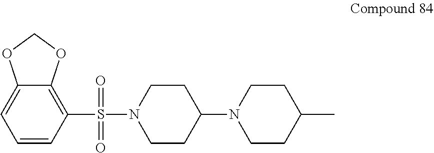 [SMILES: CC1CCN(C2CCN(S(=O)(=O)c3cccc4c3OCO4)CC2)CC1]

Compound 84: 1′-((3,4-Dimethoxyphenyl)sulfonyl)-4-methyl-1,4′-bipiperidine. Reaction of amine 1a with benzo[d][1,3]dioxole-5-sulfonyl chloride (Procedure A) yielded 84 as a light orange solid (82%). ¹H NMR (400 MHz, CDCl₃) δ 7.28 (dt, J=8.2, 1.6 Hz, 1H), 7.13 (t, J=1.6 Hz, 1H), 6.87 (dd, J=8.1, 1.2 Hz, 1H), 6.05 (s, 2H), 3.78 (d, J=10.9 Hz, 2H), 2.77 (d, J=11.0 Hz, 2H), 2.30-2.04 (m, 5H), 1.82 (d, J=10.7 Hz, 2H), 1.70-1.53 (m, 4H), 1.38-1.23 (m, 1H), 1.22-1.08 (m, 2H), 0.87 (d, J=6.4 Hz, 3H). ¹³C NMR (100 MHz, CDCl₃) δ 151.3, 148.1, 129.3, 123.2, 108.2, 107.9, 102.3, 61.5, 49.5, 46.2, 34.5, 31.0, 27.3, 21.8. HRMS (ESI-TOF) calcd for C₁₈H₂₇N₂O₄S [M+H]⁺ 367.1681, found 367.1686.

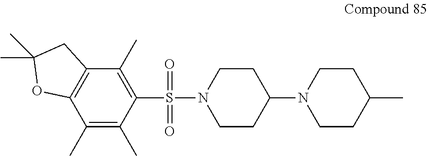

Compound 85: 4-Methyl-1′-((2,2,4,6,7-pentamethyl-2,3-dihydrobenzofuran-5-yl)sulfonyl)-1,4′-bipiperidine. Reaction of amine 1a with 2,2,4,6,7-pentamethyl-2,3-dihydrobenzofuran-5-sulfonyl chloride (Procedure A) yielded 85 as a pale yellow oil (95%). ¹H NMR (400 MHz, CDCl₃) δ 3.63 (d, J=12.0 Hz, 2H), 2.97 (s, 2H), 2.89 (br, 2H), 2.75 (dt, J=12.0, 4.0 Hz, 2H), 2.50 (s, 3H), 2.46 (s, 3H), 2.41 (br, 1H), 2.14 (br, 2H), 2.10 (s, 3H), 1.90 (d, J=12.0 Hz, 2H), 1.64 (d, J=12.0 Hz, 2H), 1.58-1.43 (m, 2H), 1.48 (s, 6H), 1.40-1.08 (m, 3H), 0.87 (d, J=4.0 Hz, 3H). ¹³C NMR (100 MHz, CDCl₃) δ 159.9, 140.8, 135.3, 125.8, 125.0, 117.9, 86.8, 61.9, 49.6, 43.9, 43.1, 34.5, 31.0, 28.6, 27.7, 21.8, 19.2, 17.6, 12.5. HRMS (ESI-TOF) calcd for C₂₄H₃₉N₂O₃S [M+H]⁺ 435.2676, found 435.2664.

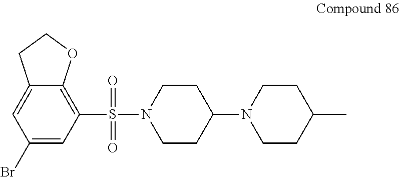 [SMILES: CC1CCN(C2CCN(S(=O)(=O)c3cc(Br)cc4c3OCC4)CC2)CC1]

Compound 86: 1′-((5-Bromo-2,3-dihydrobenzofuran-7-yl)sulfonyl)-4-methyl-1,4′-bipiperidine. Reaction of amine 1a with 5-bromo-2,3-dihydrobenzofuran-7-sulfonyl chloride (Procedure A) yielded 86 as a white solid (86%); mp 128-132° C. ¹H NMR (400 MHz, CDCl₃) δ 7.62 (s, 1H), 7.43 (s, 1H), 4.70 (t, J=8.9 Hz, 2H), 3.89 (d, J=12.3 Hz, 2H), 3.24 (t, J=9.0 Hz, 2H), 2.79 (d, J=11.9 Hz, 2H), 2.50 (t, J=12.4 Hz, 2H), 2.27 (tt, J=11.7, 3.7 Hz, 1H), 2.12 (t, J=11.5 Hz, 2H), 1.82 (d, J=11.2 Hz, 2H), 1.67-1.51 (m, 4H), 1.38-1.25 (m, 1H), 1.17 (qd, J=11.9, 3.7 Hz, 2H), 0.88 (d, J=6.3 Hz, 3H). ¹³C NMR (100 MHz, CDCl₃) δ 156.4, 132.3, 132.2, 130.7, 121.0, 111.8, 73.0, 61.7, 49.5, 46.0, 34.6, 31.0, 29.0, 27.6, 21.9. HRMS (ESI-TOF) calcd for C₁₉H₂₈BrN₂O₃S [M+H]⁺ 443.0999, found 443.0989.

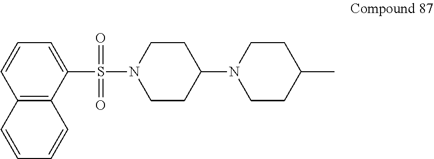 [SMILES: CC1CCN(C2CCN(S(=O)(=O)c3cccc4ccccc34)CC2)CC1]

Compound 87: 4-Methyl-1′-(naphthalen-1-ylsulfonyl)-1,4′-bipiperidine. Reaction of amine 1a with naphthalene-1-sulfonyl chloride (Procedure A) yielded 87 as a yellow gel (79%). ¹H NMR (400 MHz, CDCl₃) δ 8.74 (d, J=8.6 Hz, 1H), 8.20 (d, J=7.4 Hz, 1H), 8.05 (d, J=8.4 Hz, 1H), 7.91 (d, J=8.3 Hz, 1H), 7.69-7.44 (m, 3H), 3.91 (d, J=12.5 Hz, 2H), 2.74 (d, J=11.4 Hz, 2H), 2.53 (td, J=12.3, 2.1 Hz, 2H), 2.20 (tt, J=11.3, 3.5 Hz, 1H), 2.06 (t, J=11.5 Hz, 2H), 1.80 (d, J=12.3 Hz, 2H), 1.65-1.43 (m, 4H), 1.38-1.21 (m, 1H), 1.22-1.06 (m, 2H), 0.87 (d, J=6.4 Hz, 3H). ¹³C NMR (100 MHz, CDCl₃) δ 134.3, 133.0, 130.4, 128.9, 128.8, 128.0, 126.8, 125.2, 124.1, 61.5, 49.5, 45.5, 34.6, 31.0, 27.7, 21.9. HRMS (ESI-TOF) (ESI) calcd for C21H28N2O2S [M+H]⁺ 373.1950, found: 373.1944.

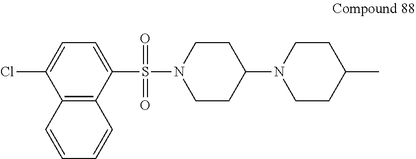 [SMILES: CC1CCN(C2CCN(S(=O)(=O)c3ccc(Cl)c4ccccc34)CC2)CC1]

Compound 88: 1′-((4-Chloronaphthalen-1-yl)sulfonyl)-4-methyl-1,4′-bipiperidine. Reaction of amine 1a with 4-chloronaphthalene-1-sulfonyl chloride (Procedure A) yielded 88 as a white solid (71%); mp 129-132° C. ¹H NMR (400 MHz, CDCl₃) δ 8.80-8.72 (m, 1H), 8.43-8.36 (m, 1H), 8.11 (d, J=8.0 Hz, 1H), 7.69 (dd, J=6.6, 3.3 Hz, 2H), 7.64 (d, J=8.0 Hz, 1H), 3.87 (d, J=12.4 Hz, 2H), 2.75 (d, J=11.0 Hz, 2H), 2.55 (td, J=12.2, 2.4 Hz, 2H), 2.23 (t, J=12.0 Hz, 1H), 2.06 (t, J=11.3 Hz, 2H), 1.81 (d, J=13.1 Hz, 2H), 1.66-1.45 (m, 4H), 1.35-1.21 (m, 1H), 1.15 (dd, J=13.4, 9.6 Hz, 2H), 0.87 (d, J=6.4 Hz, 3H). ¹³C NMR (100 MHz, CDCl₃) δ 138.4, 132.3, 131.5, 130.1, 130.0, 128.7, 127.9, 125.7, 125.4, 124.6, 61.5, 49.5, 45.5, 34.5, 31.0, 27.7, 21.8. HRMS (ESI-TOF) calcd for C₂₁H₂₈ClN₂O₂S [M+H]⁺ 407.1555, found: 407.1551.

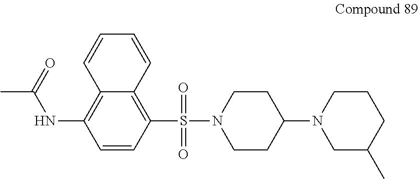 [SMILES: CC(=O)Nc1ccc(S(=O)(=O)N2CCC(N3CCCC(C)C3)CC2)c2ccccc12]

Compound 89: N-(4-((4-Methyl-[1,4′-bipiperidin]-1′-yl)sulfonyl)naphthalen-1-yl)acetamide. Reaction of amine 1a with 4-acetamidonaphthalene-1-sulfonyl chloride (Procedure A) yielded 89 as a yellow solid (64%); mp 113-117° C. ¹H NMR (400 MHz, CDCl₃) δ 8.51 (d, J=8.8 Hz, 1H), 8.17-8.05 (m, 2H), 7.87 (s, 1H), 7.66 (d, J=7.6 Hz, 1H), 7.57-7.37 (m, 2H), 3.86 (d, J=9.6 Hz, 2H), 2.80 (d, J=11.6 Hz, 2H), 2.52 (t, J=12.4 Hz, 2H), 2.36 (t, J=12.8 Hz, 1H), 2.31 (s, 3H), 2.15 (t, J=11.6 Hz, 3H), 1.83 (d, J=13.6 Hz, 2H), 1.66-1.45 (m, 4H), 1.35-1.20 (m, 3H), 0.88 (d, J=6.0 Hz, 3H). ¹³C NMR (100 MHz, CDCl₃) δ 169.5, 133.3, 133.2, 130.6, 129.5, 129.4, 128.1, 127.7, 124.3, 123.9, 123.4, 61.6, 49.3, 45.3, 33.7, 30.7, 27.2, 24.0, 21.6. HRMS (ESI-TOF) calcd for C₂₃H₃₂N₃O₃S [M+H]⁺ 430.2159, found: 430.2165

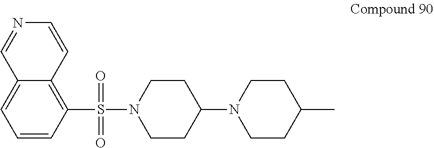 [SMILES: CC1CCN(C2CCN(S(=O)(=O)c3cccc4cnccc34)CC2)CC1]

Compound 90: 5-((4-Methyl-[1,4′-bipiperidin]-1′-yl)sulfonyl)isoquinoline. Reaction of amine 1a with isoquinoline-5-sulfonyl chloride (Procedure A) yielded 90 as a white solid (51%); mp 123-126° C. ¹H NMR (400 MHz, CDCl₃) δ 9.33 (s, 1H), 8.66 (d, J=6.0 Hz, 1H), 8.48 (d, J=6.0 Hz, 1H), 8.36 (dd, J=7.6, 1.6 Hz, 1H), 8.19 (d, J=6.0 Hz, 1H), 7.69 (dd, J=8.2, 7.4 Hz, 1H), 3.90 (d, J=12.4 Hz, 2H), 2.74 (d, J=10.8 Hz, 2H), 2.51 (td, J=12.0, 2.4 Hz, 2H), 2.26-2.15 (m, 1H), 2.12-2.03 (m, 2H), 1.81 (d, J=11.6 Hz, 2H), 1.64-1.45 (m, 4H), 1.36-1.20 (m, 1H), 1.19-1.06 (m, 2H), 0.86 (d, J=6.4 Hz, 3H). ¹³C NMR (100 MHz, CDCl₃) δ 153.2, 145.1, 134.0, 133.7, 132.6, 131.9, 129.1, 125.8, 117.7, 61.4, 49.9, 45.6, 34.5, 31.0, 27.7, 21.6. HRMS (ESI-TOF) calcd for C₂₀H₂₈N₃O₂S [M+H]⁺ 396.1716, found 396.1710.

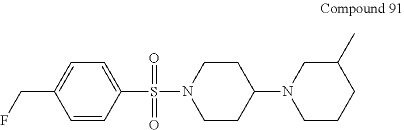 [SMILES: CC1CCCN(C2CCN(S(=O)(=O)c3ccc(CF)cc3)CC2)C1]

Compound 91: 1′-((4-(Difluoromethoxy)phenyl)sulfonyl)-3-methyl-1,4′-bipiperidine. Reaction of amine 1b with 4-(difluoromethoxy)benzenesulfonyl chloride (Procedure A) yielded 91 as a pale yellow solid (76%); mp 88-91° C. ¹H NMR (400 MHz, CDCl₃) δ 7.77 (d, J=8.8 Hz, 2H), 7.24 (d, J=8.8 Hz, 2H), 6.60 (t, J=72.6 Hz, 1H), 3.84 (d, J=11.9 Hz, 2H), 2.85-2.56 (m, 2H), 2.37-2.15 (m, 3H), 2.04 (t, J=11.4 Hz, 1H), 1.83 (d, J=11.3 Hz, 2H), 1.78-1.42 (m, 7H), 0.90-0.73 (m, 1H), 0.82 (d, J=6.4 Hz, 3H). ¹³C NMR (100 MHz, CDCl₃) δ 154.1, 133.0, 129.8, 119.3, 115.2 (t, J=262.7 Hz), 61.5, 57.6, 49.5, 46.2, 33.3, 31.5, 27.2, 27.1, 25.9, 19.8. HRMS (ESI-TOF) calcd for C₁₉H₂₆N₂O₃F₂S [M+H]⁺ 389.1699, found: 389.1705.

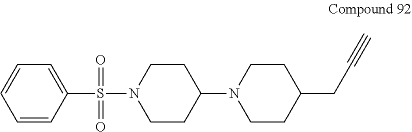 [SMILES: C#CCC1CCN(C2CCN(S(=O)(=O)c3ccccc3)CC2)CC1]

Compound 92: 1′-((4-Bromophenyl)sulfonyl)-4-(prop-2-yn-1-yl)-1,4′-bipiperidine. The synthesis of 92 is described in the Supporting Information. mp 161-164° C. ¹H NMR (400 MHz, CDCl₃) δ 7.78-7.54 (m, 4H), 3.83 (d, J=12.0, 2H), 2.83 (dt, J=11.8 Hz, 2H), 2.33-2.17 (m, 3H), 2.19-2.04 (m, 4H), 1.96 (t, J=2.7 Hz, 1H), 1.88-1.73 (m, 4H), 1.64 (qd, J=12.2, 4.1 Hz, 2H), 1.53-1.37 (m, 1H), 1.37-1.17 (m, 2H). ¹³C NMR (100 MHz, CDCl₃) δ 135.2, 132.3, 129.1, 127.8, 82.7, 69.4, 61.3, 49.2, 46.1, 35.5, 31.9, 27.3, 25.4. HRMS (ESI-TOF) calcd for C₁₉H₂₆BrN₂O₂S [M+H]⁺ 425.0893, found 425.0894.

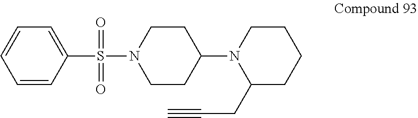 [SMILES: C#CCC1CCCCN1C1CCN(S(=O)(=O)c2ccccc2)CC1]

Compound 93: 1′-(Phenylsulfonyl)-2-(prop-2-yn-1-yl)-1,4′-bipiperidine. The synthesis of 93 is described in the Supporting Information. ¹H NMR (400 MHz, CDCl₃) δ 7.82-7.70 (m, 2H), 7.63-7.57 (m, 1H), 7.57-7.50 (m, 2H), 3.85 (d, J=12.1 Hz, 2H), 2.78-2.57 (m, 3H), 2.36-2.19 (m, 4H), 1.94 (t, J=2.7 Hz, 1H), 1.89-1.39 (m, 9H), 1.35-1.19 (m, 2H). ¹³C NMR (100 MHz, CDCl₃) δ 136.2, 232.7, 129.0, 127.6, 82.1, 70.0, 56.4, 55.5, 46.5, 46.0, 45.2, 31.8, 30.1, 26.0, 24.2, 23.2, 21.7. HRMS (ESI-TOF) calcd for C₁₉H₂₇N₂O₂S [M+H]⁺ 347.1788, found 347.1799.

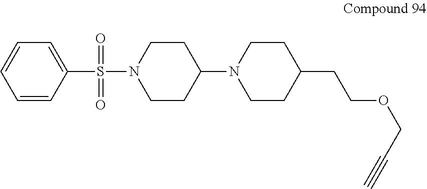 [SMILES: C#CCOCCC1CCN(C2CCN(S(=O)(=O)c3ccccc3)CC2)CC1]

Compound 94: 1′-(Phenylsulfonyl)-4-(2-(prop-2-yn-1-yloxy)ethyl)-1,4′-bipiperidine. To a solution of 2-(1′-(phenylsulfonyl)-[1,4′-bipiperidin]-4-yl)ethanol (0.0585 g, 0.17 mmol; prepared from 3a with 4-piperidineethanol, see SI) in anhydrous THF (1 mL) was added a suspension of KH (30% in mineral oil, 0.0275 g, 0.21 mmol) in anhydrous THF (0.5 mL) via syringe at room temperature. After stirring for 45 min, propargyl bromide (80% in toluene, 0.038 mL, 0.43 mmol) was added dropwise via syringe, and the solution was stirred at room temperature for 7.5 h. After the evaporation of solvents, the residue was purified by flash chromatography (1/19 CH₃OH/CH₂Cl₂) to afford the desired product 94 as a yellow gel (0.0072 g, 11%). ¹H NMR (400 MHz, CDCl₃) δ 7.83-7.69 (m, 2H), 7.63-7.57 (m, 1H), 7.57-7.49 (m, 2H), 4.11 (d, J=2.3 Hz, 2H), 3.95-3.80 (m, 2H), 3.53 (t, J=6.4 Hz, 2H), 2.85 (d, J=10.7 Hz, 2H), 2.41 (t, J=2.4 Hz, 1H), 2.30-2.13 (m, 5H), 1.87 (d, J=12.6 Hz, 2H), 1.79-1.59 (m, 4H), 1.52 (q, J=6.5 Hz, 2H), 1.45-1.36 (m, 1H), 1.32-1.22 (m, 2H). ¹³C NMR (100 MHz, CDCl₃) δ 136.1, 132.7, 129.0, 127.6, 79.9, 74.2, 67.6, 61.6, 58.1, 49.3, 46.1, 35.9, 32.6, 32.2, 27.1. HRMS (ESI-TOF) calcd for C₂₁H₃₀N₂O₃SNa [M+Na]⁺ 413.1869, found 413.1863.

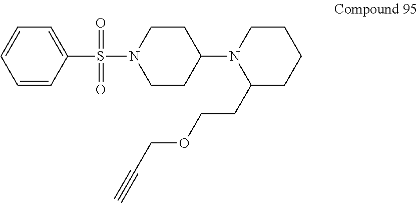 [SMILES: C#CCOCCC1CCCCN1C1CCN(S(=O)(=O)c2ccccc2)CC1]

Compound 95: 1′-(Phenylsulfonyl)-2-(2-(prop-2-yn-1-yloxy)ethyl)-1,4′-bipiperidine. To a solution of 2-(1′-(phenylsulfonyl)-[1,4′-bipiperidin]-2-yl)ethan-1-ol (0.0830 g, 0.24 mmol; prepared from 3a with 2-piperidineethanol, see SI) in anhydrous THF (2 mL) was added NaH (60%, 0.0192 g, 0.48 mmol) at 0° C. The reaction solution was stirred at 0° C. for 15 min and propargyl bromide (80% in toluene, 0.075 ml, 0.67 mmol) was added via syringe. The reaction solution was stirred at 0° C. for another 30 min and was allowed to warm up to room temperature and stirred for 3 h before a second portion of NaH (60%, 0.0175 g, 0.44 mmol) in anhydrous THF (1 mL) was introduced. The reaction solution was then stirred for 18 h at room temperature and water (10 mL) was added to quench the reaction, followed by the extraction with CH₂Cl₂ (3×10 mL). The combined organic layers were dried over Na₂SO₄, filtered and concentrated under reduced pressure. After the purification by flash chromatography (1/19 CH₃OH/CH₂Cl₂), the product 95 was obtained as a yellow gel (0.0132 g, 18%). ¹H NMR (400 MHz, CDCl₃) δ 7.82-7.69 (m, 2H), 7.64-7.56 (m, 1H), 7.56-7.48 (m, 2H), 4.06 (d, J=2.4 Hz, 2H), 3.83 (d, J=11.9 Hz, 2H), 3.58-3.37 (m, 2H), 2.73 (dd, J=39.3, 10.5 Hz, 3H), 2.35 (t, J=2.4 Hz, 1H), 2.33-2.16 (m, 3H), 1.88-1.70 (m, 4H), 1.69-1.19 (m, 8H). ¹³C NMR (100 MHz, CDCl₃) δ 136.0, 132.8, 129.1, 127.6, 79.6, 74.4, 67.1, 58.2, 55.5, 55.0, 46.4, 45.9, 45.4, 30.3, 29.8, 29.7, 25.6, 24.2, 23.0. HRMS (ESI-TOF) calcd for C₂₁H₃₁N₂O₃S [M+H]⁺ 391.2050, found 391.2048.

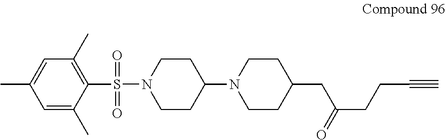 [SMILES: C#CCCC(=O)CC1CCN(C2CCN(S(=O)(=O)c3c(C)cc(C)cc3C)CC2)CC1]

Compound 96: 1-(1′-(Mesitylsulfonyl)-[1,4′-bipiperidin]-4-yl)hex-5-yn-2-one. The synthesis of 96 is described in the Supporting Information. ¹H NMR (400 MHz, CDCl₃) δ 6.91 (s, 2H), 3.61 (d, J=12.8 Hz, 2H), 2.88 (d, J=11.2 Hz, 2H), 2.71 (td, J=12.2, 2.4 Hz, 2H), 2.63-2.58 (m, 2H), 2.57 (s, 6H), 2.41 (qd, J=8.4, 7.0, 3.5 Hz, 3H), 2.32 (d, J=6.8 Hz, 2H), 2.26 (s, 3H), 2.19 (t, J=12.3 Hz, 2H), 1.90 (t, J=2.7 Hz, 1H), 1.84 (d, J=16.0 Hz, 3H), 1.67 (d, J=10.9 Hz, 2H), 1.49 (qd, J=12.2, 4.2 Hz, 2H), 1.34-1.22 (m, 2H). ¹³C NMR (100 MHz, CDCl₃) δ 207.7, 142.5, 140.4, 131.9, 131.6, 83.0, 68.7, 61.8, 49.2, 43.9, 41.9, 32.0, 31.7, 27.4, 22.8, 20.9, 12.9. HRMS (ESI-TOF) calcd for C₂₅H₃₇N₂O₃S [M+H]⁺ 445.2519, found 445.2524.

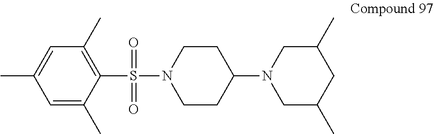

Compound 97: 1′-(Mesitylsulfonyl)-3,5-dimethyl-1,4′-bipiperidine. Reaction of 3c with 3,5-dimethylpiperidine (Procedure D) yielded 97 as a colorless oil (37%); ¹H NMR (400 MHz, CDCl₃) δ 6.92 (s, 2H), 3.61 (d, J=12.4 Hz, 2H), 2.87-2.66 (m, 4H), 2.59 (s, 6H), 2.47-2.29 (m, 1H), 2.27 (s, 3H), 1.82 (d, J=13.6 Hz, 2H), 1.74-1.39 (m, 7H), 0.81 (d, J=5.8 Hz, 6H), 0.47 (q, J=11.4 Hz, 1H). ¹³C NMR (100 MHz, CDCl₃) δ 142.4, 140.4, 131.8, 131.8, 61.8, 57.2, 44.1, 42.3, 31.4, 27.5, 22.8, 20.9, 19.7. HRMS (ESI-TOF) calcd for C₂₁H₃₅N₂O₂S [M+H]⁺ 379.2414, found 379.2406.

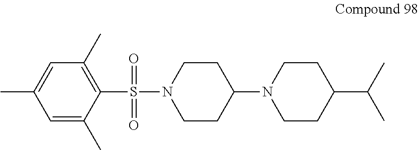 [SMILES: Cc1cc(C)c(S(=O)(=O)N2CCC(N3CCC(C(C)C)CC3)CC2)c(C)c1]

Compound 98: 4-Isopropyl-1′-(mesitylsulfonyl)-1,4′-bipiperidine. Reaction of 3c with 4-isopropylpiperidine (Procedure D) yielded 98 as a yellow oil (46%); ¹H NMR (400 MHz, CDCl₃) δ 6.91 (s, 2H), 3.60 (d, J=12.7 Hz, 2H), 2.90 (dd, J=11.5 Hz, 2H), 2.72 (t, J=12.4 Hz, 2H), 2.59 (d, J=2.3 Hz, 6H), 2.38-2.29 (m, 1H), 2.28 (s, 3H), 2.06 (t, J=10.6 Hz, 2H), 1.85 (d, J=11.2 Hz, 2H), 1.63 (d, J=11.3 Hz, 2H), 1.49 (td, J=12.1, 4.1 Hz, 2H), 1.44-1.32 (m, 1H), 1.30-1.14 (m, 2H), 1.01-0.87 (m, 1H), 0.83 (d, J=6.7 Hz, 6H). ¹³C NMR (100 MHz, CDCl₃) δ 142.4, 140.4, 131.8, 131.7, 61.8, 50.0, 44.0, 42.6, 32.4, 29.6, 27.7, 22.8, 20.9, 19.8. HRMS (ESI-TOF) calcd for C₂₂H₃₇N₂O₂S [M+H]⁺ 393.2570, found 393.2567.

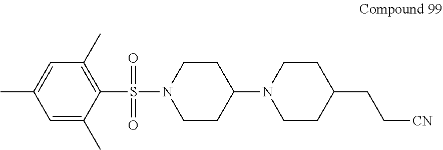 [SMILES: Cc1cc(C)c(S(=O)(=O)N2CCC(N3CCC(CCC#N)CC3)CC2)c(C)c1]

Compound 99: 3-(1′-(Mesitylsulfonyl)-[1,4′-bipiperidin]-4-yl)propanenitrile. Tert-butyl 4-(2-cyanoethyl)piperidine-1-carboxylate (0.328 g, 1.38 mmol) was stirred with HCl (1 mL) in 1, 4-dioxane (4 mL) at room temperature for 2 hours. After the evaporation of the solvents, the 4-(2-cyanoethyl)piperidine hydrochloride acid salt was neutralized by shaking with saturated NaHCO₃ solution at 0° C., followed by extraction with CH₂Cl₂ (3×25 mL). The combined organic layers were dried over Na₂SO₄, filtered and concentrated under reduced pressure to provide 3-(piperidin-4-yl)propanenitrile which was used for the next step without further purification. Reaction of the above prepared 3-(piperidin-4-yl)propanenitrile and 1-(mesitylsulfonyl)piperidin-4-one (3c) (Procedure D) yielded 99 as a white gel. ¹H NMR (400 MHz, CDCl₃) δ 6.94 (s, 2H), 3.63 (d, J=12.3 Hz, 2H), 2.90 (d, J=10.7 Hz, 2H), 2.74 (td, J=12.4, 2.4 Hz, 2H), 2.60 (s, 6H), 2.35 (t, J=7.2 Hz, 3H), 2.29 (s, 3H), 2.14 (t, J=9.6 Hz, 2H), 1.84 (d, J=12.7 Hz, 2H), 1.71 (d, J=12.3 Hz, 2H), 1.63-1.34 (m, 5H), 1.27-1.16 (m, 2H). ¹³C NMR (100 MHz, CDCl₃) δ 142.5, 140.4, 131.9, 131.7, 119.7, 61.8, 49.2, 44.0, 34.8, 31.8, 31.7, 27.6, 22.8, 21.0, 14.6. HRMS (ESI-TOF) calcd for C₂₂H₃₄N₃O₂S [M+H]⁺ 404.2366, found 404.2361.

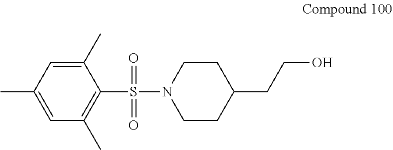 [SMILES: Cc1cc(C)c(S(=O)(=O)N2CCC(CCO)CC2)c(C)c1]

Compound 100: 2-(1′-(Mesitylsulfonyl)-[1,4′-bipiperidin]-4-yl)ethanol. Reaction of 3c with 4-piperidineethanol (Procedure D) yielded 100 as a white solid (66%); mp 82-85° C. ¹H NMR (400 MHz, CDCl₃) δ 6.93 (s, 2H), 3.66 (t, J=6.6 Hz, 2H), 3.62 (d, J=12.6 Hz, 2H), 2.86 (d, J=11.6 Hz, 2H), 2.73 (td, J=12.5, 2.4 Hz, 2H), 2.60 (s, 6H), 2.40-2.28 (m, 1H), 2.28 (s, 3H), 2.11 (td, J=11.6, 2.4 Hz, 2H), 1.84 (d, J=12.7 Hz, 2H), 1.69 (d, J=13.2 Hz, 2H), 1.63 (s, 1H), 1.55-1.33 (m, 5H), 1.21 (qd, J=11.7, 11.0, 3.8 Hz, 2H). ¹³C NMR (100 MHz, CDCl₃) δ 142.4, 140.4, 131.9, 131.7, 61.8, 60.4, 49.5, 44.0, 39.4, 32.6, 32.5, 27.7, 22.8, 21.0. HRMS (ESI-TOF) calcd for C₂₁H₃₅N₂O₃S [M+H]⁺ 395.2363, found 395.2365.

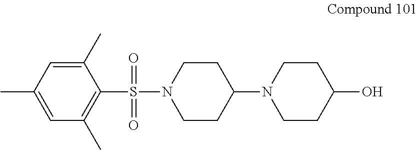 [SMILES: Cc1cc(C)c(S(=O)(=O)N2CCC(N3CCC(O)CC3)CC2)c(C)c1]

Compound 101: 1′-(Mesitylsulfonyl)-[1,4′-bipiperidin]-4-ol. To a mixture of [1,4′-bipiperidin]-4-ol (1c, 0.2075 g, 1.13 mmol), and ⁱPr₂NEt (0.27 ml, 1.55 mmol) in CH₂Cl₂ (3 mL) was dropwise added 2,4,6-trimethylbenzene-1-sulfonyl chloride (0.2154 g, 0.98 mmol, in 2 mL CH₂Cl₂) over 10 min. The reaction mixture was stirred at room temperature overnight and then poured into saturated aqueous NaHCO₃(20 ml). The bi-phasic solution was extracted with CH₂Cl₂ (3×20 ml). The combined organic layers were dried by Na₂SO₄, filtered and concentrated under reduced pressure. The residue was purified via flash chromatography (1/19 MeOH/CH₂Cl₂) to afford the desired product (0.214 g, 65%) as a white solid; mp 120-123° C. ¹H NMR (400 MHz, CDCl₃) δ 6.92 (s, 2H), 3.74-3.59 (br, 1H), 3.60 (d, J=12.9 Hz, 2H), 2.85-2.65 (m, 4H), 2.59 (s, 6H), 2.46-2.22 (m, 3H), 2.28 (s, 3H), 1.96-1.78 (m, 4H), 1.64-1.30 (m, 5H). ¹³C NMR (100 MHz, CDCl₃) δ 142.5, 140.3, 131.9, 131.6, 67.6, 61.4, 46.7, 43.9, 34.5, 27.6, 22.8, 20.9. HRMS (ESI-TOF) calcd for C₁₉H₃₁N₂O₃S [M+H]⁺ 367.2050, found 367.2058.

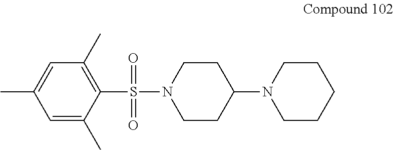 [SMILES: Cc1cc(C)c(S(=O)(=O)N2CCC(N3CCCCC3)CC2)c(C)c1]

Compound 102: 1′-(Mesitylsulfonyl)-1,4′-bipiperidine. Reaction of 3c with piperidine (Procedure D) yielded 102 as a colorless gel (67%). ¹H NMR (500 MHz, CDCl₃) δ 6.93 (s, 2H), 3.62 (d, J=11.7 Hz, 2H), 2.74 (t, J=12.1 Hz, 2H), 2.60 (s, 6H), 2.47 (s, 4H), 2.38-2.30 (m, 1H), 2.28 (s, 3H), 1.85 (d, J=11.5 Hz, 2H), 1.52-1.60 (m, 4H), 1.52-1.44 (m, 2H), 1.44-1.37 (m, 2H). ¹³C NMR (100 MHz, CDCl₃) δ 142.4, 140.4, 131.8, 131.8, 62.2, 50.2, 44.1, 27.5, 26.3, 24.6, 22.8, 20.9. HRMS (ESI-TOF) calcd for C₁₉H₃₀N₂O₂SNa [M+Na]⁺373.1920, found 373.1910.

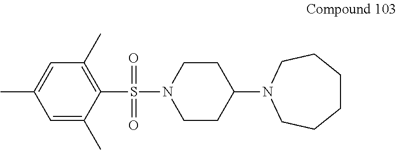 [SMILES: Cc1cc(C)c(S(=O)(=O)N2CCC(N3CCCCCC3)CC2)c(C)c1]

Compound 103: 1-(1-(Mesitylsulfonyl)piperidin-4-yl)azepane. Reaction of amine hydrochloride salt 1e with 2,4,6-trimethylbenzene-1-sulfonyl chloride (Procedure B) yielded 103 as an orange gel (90%). ¹H NMR (400 MHz, CDCl₃) δ 6.94 (s, 2H), 3.71 (d, J=11.8 Hz, 2H), 3.62 (br, 1H), 3.18-2.89 (br, 4H), 2.77 (t, J=12.7 Hz, 2H), 2.58 (s, 6H), 2.29 (s, 3H), 2.10 (d, J=12.5 Hz, 2H), 1.82 (s, 4H), 1.76-1.54 (m, 6H). ¹³C NMR (100 MHz, CDCl₃) δ 142.8, 140.3, 132.0, 132.0, 132.0, 131.5, 63.3, 51.2, 43.8, 26.9, 26.6, 26.4, 22.8, 21.0. HRMS (ESI-TOF) calcd for C₂₀H₃₃N₂O₂S [M+H]⁺ 365.2257, found 365.2253.

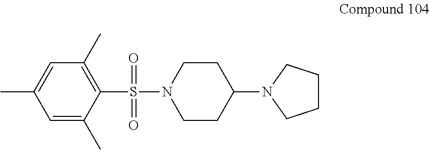 [SMILES: Cc1cc(C)c(S(=O)(=O)N2CCC(N3CCCC3)CC2)c(C)c1]

Compound 104: 1-((4-Methoxyphenyl)sulfonyl)-4-(pyrrolidin-1-yl)piperidine. Reaction of amine Id with 4-methoxybenzene-1-sulfonyl chloride (Procedure A) yielded 104 as a pale yellow oil (70%). ¹H NMR (400 MHz, CDCl₃) δ 7.68 (d, J=8.0 Hz, 2H), 6.97 (d, J=8.0 Hz, 2H), 3.86 (s, 3H), 3.68 (d, J=12.0 Hz, 2H), 2.51 (s, 4H), 2.36 (dt, J=12.0, 4.0 Hz, 2H), 2.04-1.86 (m, 3H), 1.75 (s, 4H), 1.61 (q, J=12.0 Hz, 1H). ¹³C NMR (100 MHz, CDCl₃) δ 162.9, 129.8, 127.8, 114.1, 60.8, 55.6, 51.3, 45.0, 30.4, 23.2. HRMS (ESI-TOF) calcd for C₁₆H₂₅N₂O₃S [M+H]⁺ 325.1580, found 325.1571.

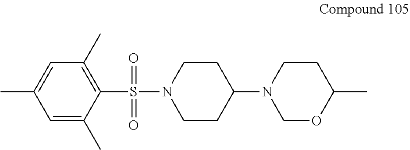

Compound 105: 3-(1-(Mesitylsulfonyl)piperidin-4-yl)-6-methyl-1,3-oxazinane. Reaction of 3c with 4-aminobutan-2-ol (Procedure D) yielded 4-((1-(mesitylsulfonyl)piperidin-4-yl)amino)butan-2-ol as a white solid (82%); mp 105-108° C. ¹H NMR (500 MHz, CDCl₃) δ 6.95 (s, 2H), 3.96 (ddd, J=8.9, 5.8, 2.4 Hz, 1H), 3.55 (d, J=12.5 Hz, 2H), 3.05 (dt, J=11.9, 4.2 Hz, 1H), 2.83 (tt, J=12.5, 3.3 Hz, 2H), 2.76 (td, J=11.1, 2.9 Hz, 1H), 2.61 (s, 7H), 2.30 (s, 3H), 1.96 (t, J=12.8 Hz, 2H), 1.63 (d, J=14.9 Hz, 1H), 1.53-1.42 (m, 1H), 1.39-1.27 (m, 2H), 1.16 (d, J=6.2 Hz, 3H). ¹³C NMR (100 MHz, CDCl₃) δ 142.6, 140.4, 131.9, 131.6, 69.5, 54.4, 45.5, 43.1, 43.0, 37.0, 31.8, 31.5, 23.6, 22.8, 21.0. HRMS (ESI-TOF) calcd for C₁₈H₃₀N₂O₃SNa [M+Na]⁺377.1869, found 377.1856.

A solution of the above prepared 4-((1-(mesitylsulfonyl)piperidin-4-yl)amino)butan-2-ol (0.119 g, 0.34 mmol), paraformaldehyde (0.0143 g, 0.48 mmol), Mg₂SO₄ (0.2091 g, 1.74 mmol), and pyridinium p-toluenesulfonate (PPTS) (0.0025 g, 0.01 mmol) in anhydrous toluene (4 mL) was refluxed for 3 h and then cooled to room temperature. The suspension was poured into saturated aqueous NaHCO₃(30 mL) and extracted with CH₂Cl₂ (3×30 mL). The combined organic layers were dried over Na₂SO₄, filtered and concentrated under reduced pressure. The residue was purified via flash chromatography (1/19 MeOH/CH₂Cl₂) to provide the desired product 105 as a colorless gel (0.0555 g, 45%). ¹H NMR (400 MHz, CDCl₃) δ 6.91 (s, 2H), 4.61 (dd, J=10.0, 2.3 Hz, 1H), 4.16 (d, J=10.1 Hz, 1H), 3.63-3.50 (m, 3H), 3.11 (ddt, J=13.4, 4.4, 2.2 Hz, 1H), 2.89-2.68 (m, 4H), 2.58 (s, 6H), 2.27 (s, 3H), 1.92 (dp, J=12.2, 2.8 Hz, 2H), 1.65-1.29 (m, 4H), 1.15 (d, J=6.1 Hz, 3H). ¹³C NMR (100 MHz, CDCl₃) δ 142.5, 140.4, 131.9, 131.6, 81.6, 73.6, 55.0, 46.5, 43.4, 30.3, 29.8, 29.2, 22.8, 21.8, 20.9. HRMS (ESI-TOF) calcd for C₁₉H₃₀N₂O₃SNa [M+Na]⁺ 389.1869, found 389.1874.

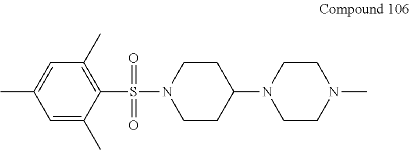

Compound 106: 1-(1-(Mesitylsulfonyl)piperidin-4-yl)-4-methylpiperazine. Reaction of 3c with 1-methylpiperazine (Procedure D) yielded 106 as a colorless gel (74%). ¹H NMR (400 MHz, CDCl₃) δ 6.91 (s, 2H), 3.59 (d, J=12.6 Hz, 2H), 2.73 (t, J=12.3 Hz, 2H), 2.58 (s, 6H), 2.55 (s, 4H), 2.49-2.35 (br, 4H), 2.36-2.29 (s, 1H), 2.26 (s, 3H), 2.25 (s, 3H), 1.84 (d, J=11.6 Hz, 2H), 1.59-1.34 (m, 2H). ¹³C NMR (100 MHz, CDCl₃) δ 142.5, 140.4, 131.8, 131.7, 61.2, 55.3, 48.9, 45.9, 43.7, 27.8, 22.8, 20.9. HRMS (ESI-TOF) calcd for C₁₉H₃₂N₃O₂S [M+H]⁺ 366.2210, found 366.2199.

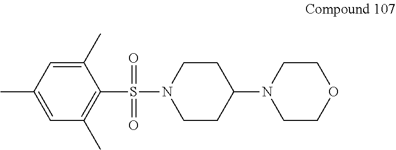 [SMILES: Cc1cc(C)c(S(=O)(=O)N2CCC(N3CCOCC3)CC2)c(C)c1]

Compound 107: 4-(1-(Mesitylsulfonyl)piperidin-4-yl)morpholine. Reaction of 3c with morpholine (Procedure D) yielded 107 as a colorless gel (74%). ¹H NMR (500 MHz, CDCl₃) δ 6.92 (s, 2H), 3.68 (s, 4H), 3.60 (d, J=12.9 Hz, 2H), 2.75 (t, J=12.3 Hz, 2H), 2.59 (s, 6H), 2.50 (s, 4H), 2.27 (s, 4H), 1.87 (d, J=14.5 Hz, 2H), 1.46 (qd, J=11.6, 3.4 Hz, 2H). ¹³C NMR (100 MHz, CDCl₃) δ 142.5, 140.4, 140.4, 131.9, 131.6, 67.1, 61.5, 49.7, 43.6, 27.7, 22.8, 20.9. HRMS (ESI-TOF) calcd for C₁₈H₂₉N₂O₃S [M+H]⁺ 353.1893, found 353.1889.

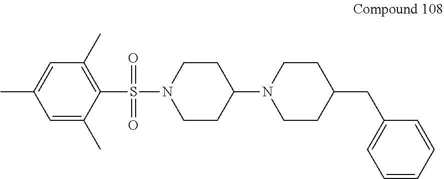 [SMILES: Cc1cc(C)c(S(=O)(=O)N2CCC(N3CCC(Cc4ccccc4)CC3)CC2)c(C)c1]

Compound 108: 4-Benzyl-1′-(mesitylsulfonyl)-1,4′-bipiperidine. Reaction of 3c with 4-benzylpiperidine (Procedure D) yielded 108 as a light brown oil (68%). ¹H NMR (400 MHz, CDCl₃) δ 7.36-7.22 (m, 2H), 7.21-7.06 (m, 3H), 6.92 (s, 2H), 3.61 (d, J=12.4 Hz, 2H), 2.92 (s, 2H), 2.72 (td, J=12.5, 2.4 Hz, 2H), 2.58 (s, 6H), 2.50 (d, J=7.0 Hz, 2H), 2.40-2.26 (br, 1H), 2.28 (s, 3H), 2.08 (t, J=17.9 Hz, 1H), 1.85 (d, J=12.8 Hz, 2H), 1.64 (d, J=12.8 Hz, 2H), 1.49 (d, J=11.5 Hz, 3H), 1.41-1.12 (br, 2H). ¹³C NMR (100 MHz, CDCl₃) δ 142.5, 140.5, 140.4, 131.9, 131.7, 129.1, 128.2, 125.8, 61.9, 49.6, 44.0, 43.1, 38.1, 32.3, 27.6, 22.8, 21.0. HRMS (ESI-TOF) calcd for C₂₆H₃₇N₂O₂S [M+H]⁺ 441.2570, found 441.2567.

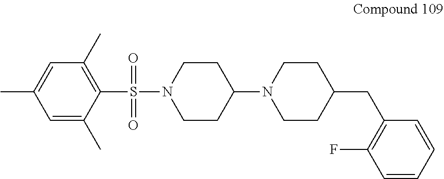 [SMILES: Cc1cc(C)c(S(=O)(=O)N2CCC(N3CCC(Cc4ccccc4F)CC3)CC2)c(C)c1]

Compound 109: 4-(2-Fluorobenzyl)-1′-(mesitylsulfonyl)-1,4′-bipiperidine. To a solution of 1-(mesitylsulfonyl)piperidin-4-one (3c, 0.2855 g, 1.02 mmol) and 4-(2-fluorobenzyl)piperidine (0.1936 g, 1.00 mmol) in anhydrous DCE (6 mL) was added Ti(Oi-Pr)₄ (0.60 mL, 2.05 mmol) and the solution was stirred at 80° C. for 6.5 h. After cooling 0° C., NaBH₄ (0.1271 g, 3.34 mmol) in EtOH (6 mL) was added dropwise at the solution was stirred at room temperature overnight. The reaction was quenched with saturated aqueous NaHCO₃(30 mL), extracted with CH₂Cl₂ (3×30 mL), dried over Na₂SO₄, filtered and concentrated. The residue was purified via flash chromatography (1/19 MeOH/CH₂Cl₂ then 100% EtOAc) to afford the product 109 as a light brown gel (0.1444 g, 35%). ¹H NMR (400 MHz, CDCl₃) δ 7.17-7.05 (m, 2H), 7.03-6.92 (m, 2H), 6.90 (s, 2H), 3.59 (d, J=12.5 Hz, 2H), 2.86 (d, J=11.7 Hz, 2H), 2.70 (td, J=12.4, 2.4 Hz, 2H), 2.57 (s, 6H), 2.55-2.48 (m, 2H), 2.44-2.30 (m, 1H), 2.25 (s, 3H), 2.09 (td, J=11.7, 2.4 Hz, 2H), 1.83 (d, J=14.5 Hz, 2H), 1.61 (d, J=13.1 Hz, 2H), 1.58-1.41 (m, 3H), 1.39-1.22 (m, 2H). ¹³C NMR (100 MHz, CDCl₃) δ 161.2 (d, J=244.6 Hz), 142.5, 140.4, 131.9, 131.7, 131.5 (d, J=5.1 Hz), 127.6 (d, J=8.1 Hz), 127.2 (d, J=16.2 Hz), 123.7 (d, J=3.5 Hz), 115.1 (d, J=22.5 Hz), 61.8, 49.4, 43.9, 36.8, 35.9, 32.0, 27.5, 22.8, 20.9. HRMS (ESI-TOF) calcd for C₂₆H₃₆FN₂O₂S [M+H]⁺ 459.2476, found 459.2470.

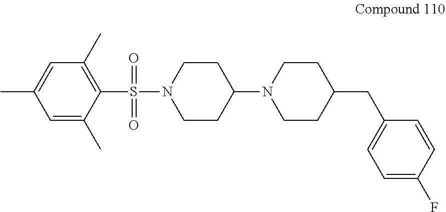 [SMILES: Cc1cc(C)c(S(=O)(=O)N2CCC(N3CCC(Cc4ccc(F)cc4)CC3)CC2)c(C)c1]

Compound 110: 4-(4-Fluorobenzyl)-1′-(mesitylsulfonyl)-1,4′-bipiperidine. Reaction of 3c and 4-(4-fluorobenzyl)piperidine in the same manner as for the preparation of compound 109 yielded compound 110 as a colorless gel (31%). ¹H NMR (400 MHz, CDCl₃) δ 7.09-7.00 (m, 2H), 6.97-6.87 (m, 4H), 3.60 (d, J=12.6 Hz, 2H), 2.86 (d, J=11.5 Hz, 2H), 2.72 (td, J=12.5, 2.4 Hz, 2H), 2.58 (s, 6H), 2.46 (d, J=7.0 Hz, 2H), 2.35 (t, J=11.8 Hz, 1H), 2.27 (s, 3H), 2.08 (dd, J=12.8, 10.2 Hz, 2H), 1.83 (d, J=12.1 Hz, 2H), 1.61 (d, J=12.8 Hz, 2H), 1.55-1.36 (m, 3H), 1.34-1.15 (m, 2H). ¹³C NMR (100 MHz, CDCl₃) δ 161.2 (d, J=243.4 Hz), 142.5, 140.4, 136.1 (d, J=3.3 Hz), 131.9, 131.7, 130.3 (d, J=7.7 Hz), 114.9 (d, J=21.0 Hz), 61.8, 49.5, 44.0, 42.2, 38.1, 32.2, 27.6, 22.8, 20.9. HRMS (ESI-TOF) calcd for C₂₆H₃₆FN₂O₂S [M+H]⁺ 459.2476, found 459.2477.

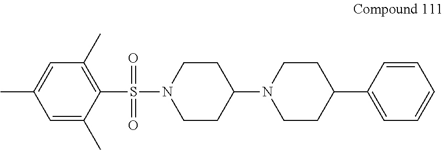 [SMILES: Cc1cc(C)c(S(=O)(=O)N2CCC(N3CCC(c4ccccc4)CC3)CC2)c(C)c1]

Compound 111: 1′-(Mesitylsulfonyl)-4-phenyl-1,4′-bipiperidine. Reaction of 3c with 4-phenylpiperidine (Procedure D) yielded 111 as a white solid (90%); mp 141-144° C. ¹H NMR (400 MHz, CDCl₃) δ 7.34-7.26 (m, 2H), 7.25-7.16 (m, 3H), 6.95 (s, 2H), 3.66 (d, J=12.5 Hz, 2H), 3.03 (d, J=11.0 Hz, 2H), 2.78 (t, J=12.5 Hz, 2H), 2.62 (s, 6H), 2.54-2.37 (m, 2H), 2.30 (s, 3H), 2.30-2.20 (m, 2H), 1.99-1.66 (m, 6H), 1.56 (qd, J=12.2, 4.3 Hz, 2H). ¹³C NMR (100 MHz, CDCl₃) δ 142.5, 140.5, 131.9, 131.7, 128.4, 126.8, 126.2, 61.9, 50.0, 44.0, 42.9, 33.7, 27.7, 22.8, 21.0. HRMS (ESI-TOF) calcd for C₂₅H₃₅N₂O₂S [M+H]⁺ 427.2414, found 427.2405

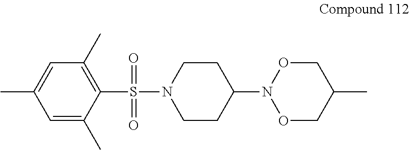

Compound 112: 1-(Mesitylsulfonyl)-4-(5-methyl-1,3-dioxan-2-yl)piperidine. The synthesis of compound 112, obtained as a mixture of cis:trans isomers (1:1.56), is described in the Supporting Information. Trans: ¹H NMR (400 MHz, CDCl₃) δ 6.91 (s, 2H), 4.16 (d, J=5.4 Hz, 1H), 3.97 (dd, J=11.8, 4.7 Hz, 2H), 3.55 (d, J=10.2 Hz, 2H), 3.20 (t, J=11.5 Hz, 2H), 2.70 (t, J=12.4 Hz, 2H), 2.58 (s, 6H), 2.26 (s, 3H), 2.07-1.90 (m, 1H), 1.77 (d, J=14.4 Hz, 2H), 1.64-1.54 (m, 1H), 1.42-1.24 (m, 2H), 0.66 (d, J=6.7 Hz, 3H). ¹³C NMR (100 MHz, CDCl₃) δ 142.3, 140.5, 140.4, 131.8, 103.4, 71.8, 44.0, 40.3, 29.5, 26.1, 22.8, 20.9, 12.3. Cis: ¹H NMR (400 MHz, CDCl₃) δ 6.91 (s, 2H), 4.26 (d, J=5.1 Hz, 1H), 3.84 (d, J=11.5 Hz, 2H), 3.74 (d, J=10.1 Hz, 2H), 3.55 (d, J=10.2 Hz, 2H), 2.70 (t, J=12.4 Hz, 2H), 2.58 (s, 6H), 2.26 (s, 3H), 1.77 (d, J=14.4 Hz, 2H), 1.64-1.54 (m, 1H), 1.54-1.47 (m, 1H), 1.42-1.24 (m, 2H), 1.20 (d, J=7.0 Hz, 3H). ¹³C NMR (100 MHz, CDCl₃) δ 142.3, 140.5, 140.4, 103.9, 73.2, 44.0, 40.3, 29.1, 25.9, 22.8, 20.9, 15.8. HRMS (ESI-TOF) (ESI) calcd for C₁₉H₂₉NO₄SNa [M+Na]⁺390.1710, found 390.1706.

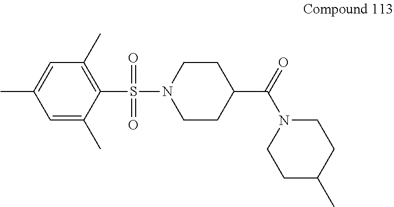

Compound 113: (1-(Mesitylsulfonyl)piperidin-4-yl 4-methylpiperidin-1-yl)methanone. Reaction of amine 1k with 2,4,6-trimethylbenzene-1-sulfonyl chloride (Procedure B) yielded 113 as a light brown solid (86%); mp 122-124° C. ¹H NMR (400 MHz, CDCl₃) δ 6.89 (s, 2H), 4.49 (d, J=13.2 Hz, 1H), 3.77 (d, J=15.5 Hz, 1H), 3.53 (dt, J=12.1, 3.6 Hz, 2H), 2.94 (td, J=12.5, 11.6, 2.3 Hz, 1H), 2.85-2.70 (m, 2H), 2.56 (s, 6H), 2.54-2.38 (m, 2H), 2.24 (s, 3H), 1.82-1.45 (m, 7H), 1.08-0.92 (m, 2H), 0.88 (d, J=6.7 Hz, 3H). ¹³C NMR (100 MHz, CDCl₃) δ 172.1, 142.6, 140.5, 131.8, 131.3, 45.7, 43.6, 42.2, 38.0, 34.9, 33.8, 31.1, 28.1, 27.8, 22.7, 21.6, 20.9; HRMS (ESI-TOF) calcd for C₂₁H₃₂N₂O₃Na [M+Na]⁺ 415.2026, found 4

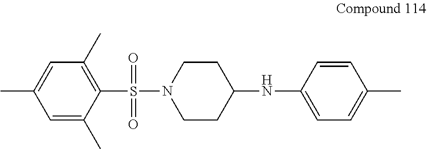

Compound 114: 1-(Mesitylsulfonyl)-N-(p-tolyl)piperidin-4-amine. Reaction of 3c with p-toluidine (Procedure D) yielded 114 as a white solid (61%); mp 148-151° C. ¹H NMR (400 MHz, CDCl₃) δ 7.03-6.89 (m, 4H), 6.51 (d, J=8.3 Hz, 2H), 3.56 (dt, J=13.2, 3.5 Hz, 2H), 3.36 (tt, J=10.1, 3.9 Hz, 1H), 2.91 (ddd, J=12.8, 11.2, 2.7 Hz, 2H), 2.61 (s, 6H), 2.29 (s, 3H), 2.21 (s, 3H), 2.06 (dd, J=13.2, 3.8 Hz, 2H), 1.54-1.32 (m, 2H). ¹³C NMR (100 MHz, CDCl₃) δ 144.0, 142.6, 140.5, 131.9, 131.6, 129.9, 127.2, 113.8, 50.0, 43.2, 31.8, 22.8, 21.0, 20.4. HRMS (ESI-TOF) calcd for C₂₁H₂₈N₂O₂SNa [M+Na]⁺395.1764, found 395.1771.

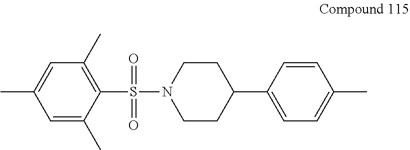

Compound 115: 1-(Mesitylsulfonyl)-4-(p-tolyl)piperidine. Reaction of amine if with 2,4,6-trimethylbenzene-1-sulfonyl chloride (Procedure A) yielded 115 as a pale yellow solid (73%); mp 87-90° C. ¹H NMR (400 MHz, CDCl₃) δ 7.08 (q, J=8.1 Hz, 4H), 6.95 (s, 2H), 3.69 (d, J=12.1 Hz, 2H), 2.86 (t, J=12.5 Hz, 2H), 2.64 (s, 6H), 2.62-2.51 (m, 1H), 2.30 (d, J=1.9 Hz, 6H), 1.86 (d, J=13.0 Hz, 2H), 1.67 (dtd, J=13.4, 12.2, 4.1 Hz, 2H). ¹³C NMR (100 MHz, CDCl₃) δ 142.4, 142.1, 140.5, 136.0, 131.9, 131.8, 129.2, 126.6, 44.9, 41.8, 32.7, 22.9, 21.0. HRMS (ESI-TOF) calcd for C₂₁H₂₇NO₂SNa [M+Na]⁺380.1655, found 380.1652.

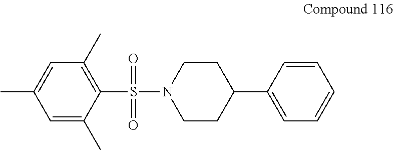 [SMILES: Cc1cc(C)c(S(=O)(=O)N2CCC(c3ccccc3)CC2)c(C)c1]

Compound 116: 1-(Mesitylsulfonyl)-4-phenylpiperidine. Reaction of amine 1g with 2,4,6-trimethylbenzene-1-sulfonyl chloride (Procedure A) yielded 116 as a yellow solid (93%); mp 88-90° C. ¹H NMR (400 MHz, CDCl₃) δ 7.36-7.26 (m, 2H), 7.23-7.14 (m, 3H), 6.96 (s, 2H), 3.71 (d, J=12.3 Hz, 2H), 2.87 (t, J=12.4 Hz, 2H), 2.65 (s, 6H), 2.64-2.54 (m, 1H), 2.30 (s, 3H), 1.88 (d, J=13.2 Hz, 2H), 1.70 (dtd, J=13.7, 12.1, 4.1 Hz, 2H). ¹³C NMR (100 MHz, CDCl₃) δ 145.1, 142.5, 140.5, 131.9, 131.8, 128.5, 126.7, 126.5, 44.9, 42.2, 32.6, 22.9, 21.0. HRMS (ESI-TOF) calcd for C₂₀H₂₅NO₂SNa [M+Na]⁺366.1498, found 366.1489.

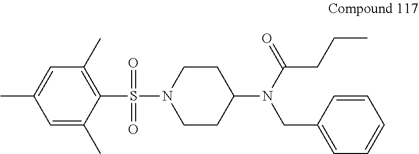 [SMILES: CCCC(=O)N(Cc1ccccc1)C1CCN(S(=O)(=O)c2c(C)cc(C)cc2C)CC1]

Compound 117: N-Benzyl-N-(1-(mesitylsulfonyl)piperidin-4-yl)butyramide. Reaction of 3c with benzylamine (Procedure D) yielded N-benzyl-1-(mesitylsulfonyl)piperidin-4-amine as a yellow oil (>95%). ¹H NMR (400 MHz, CDCl₃) δ 7.45-7.19 (m, 5H), 6.92 (s, 2H), 3.77 (s, 2H), 3.52 (d, J=13.3 Hz, 2H), 3.52 (d, J=13.3 Hz, 2H), 2.68-2.58 (m, 1H), 2.59 (s, 6H), 2.27 (s, 3H), 1.90 (d, J=12.1 Hz, 2H), 1.45-1.22 (m, 2H). ¹³C NMR (100 MHz, CDCl₃) δ 142.5, 140.4, 131.9, 131.7, 128.6, 128.5, 128.0, 127.8, 127.1, 53.7, 50.8, 42.9, 31.7, 22.8, 21.0. HRMS (ESI-TOF) calcd for C₂₁H₂₉N₂O₂S [M+H]⁺ 373.1944, found 373.1942.

A mixture of the above prepared N-benzyl-1-(mesitylsulfonyl)piperidin-4-amine (0.4135 g, 1.11 mmol), butyryl chloride (0.13 mL, 1.24 mmol), and ⁱPr₂NEt (0.28 mL, 1.70 mmol) in CH₂Cl₂ (4 mL) was stirred at room temperature for 23 h. CH₂Cl₂ (25 mL) was added and the solution was washed with saturated aqueous NaHCO₃(25 mL). The organic layer was dried over Na₂SO₄, filtered and concentrated. The residue was purified via flash chromatography (1/9 MeOH/CH₂Cl₂) to afford the desired product 117 as a yellow gel (4:1 ratio of two rotamers; 0.2351 g, 48%). ¹H NMR (400 MHz, CDCl₃ at 50° C.) δ 7.62-7.18 (m, 3H), 7.15 (d, J=7.4 Hz, 2H), 6.90 (s, 2H), 4.70-4.35 (m, 1H), 4.48 (s, 2H), 3.63 (d, J=11.6 Hz, 2H), 2.96-2.68 (m, 2H), 2.56 (s, 6H), 2.35-2.10 (br, 2H), 2.26 (s, 3H), 1.65 (s, 6H), 0.89 (s, 3H). ¹³C NMR (100 MHz, CDCl₃) Major Rotamer δ 174.0, 142.5, 140.2, 138.1, 131.9, 131.8, 128.8, 127.3, 125.7, 51.7, 46.9, 44.2, 35.7, 29.1, 22.8, 20.9, 18.7, 13.8. HRMS (ESI-TOF) calcd for C₂₅H₃₄N₂O₃SNa [M+Na]⁺ 465.2182, found 465.2190.

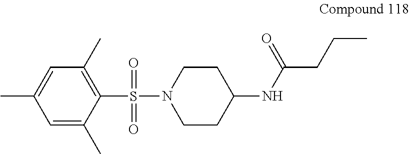 [SMILES: CCCC(=O)NC1CCN(S(=O)(=O)c2c(C)cc(C)cc2C)CC1]

Compound 118: N-(1-(Mesitylsulfonyl)piperidin-4-yl)butyramide. To a solution of N-benzyl-N-(1-(mesitylsulfonyl)piperidin-4-yl)butyramide compound 117 (0.1338 g, 0.30 mmol) in methanol (4 mL) was added palladium on carbon (10 wt %). The flask was evacuated by vacuum and refilled by a hydrogen balloon. This evacuation/refill was repeated three times and the reaction suspension was stirred under hydrogen at room temperature for 18 h. The reaction suspension was then filtered through a pad of celite and washed with methanol. Upon evaporation of the solvent compound 118 was obtained as a yellow solid (0.0662 g, 62%); mp 144-147° C. ¹H NMR (400 MHz, CDCl₃) δ 6.94 (s, 2H), 5.82-5.25 (br, 1H), 3.95 (s, 1H), 3.58 (d, J=11.4 Hz, 2H), 2.90 (s, 2H), 2.60 (s, 6H), 2.29 (s, 3H), 2.25-2.06 (br, 2H), 1.95 (s, 2H), 1.73-1.59 (br, 2H), 1.58-1.40 (br, 2H), 1.04-0.83 (br, 3H). ¹³C NMR (100 MHz, CDCl₃) δ 172.5, 142.6, 140.3, 132.0, 131.7, 46.1, 43.4, 39.0, 31.7, 22.9, 21.0, 19.3, 13.8. HRMS (ESI-TOF) calcd for C₁₈H₂₈N₂O₃SNa [M+Na]⁺375.1713, found 375.1715.

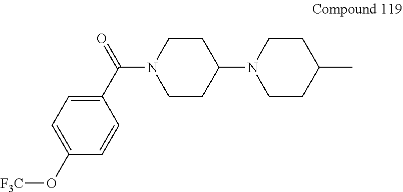 [SMILES: CC1CCN(C2CCN(C(=O)c3ccc(OC(F)(F)F)cc3)CC2)CC1]

Compound 119: (4-Methyl-[1,4′-bipiperidin]-1′-yl)(4-(trifluoromethoxy)phenyl)methanone. A mixture of 4-(trifluoromethoxy)benzoic acid (0.2024 g, 0.98 mmol), DMAP (0.0200 g, 0.16 mmol), and EDC hydrochloride (0.2166 g, 1.13 mmol) in CH₂Cl₂ (6 mL) was stirred at room temperature for 0.5 h. 4-Methyl-1,4′-bipiperidine (0.1847 g, 1.01 mmol) was added and the resulting solution was stirred at room temperature for 26.5 h. The reaction solution was diluted with CH₂Cl₂ (60 mL), washed with brine (60 mL) and saturated aqueous NaHCO₃(60 mL). The organic layer was dried over Na₂SO₄, filtered and concentrated. The residue was purified by flash chromatography (1:19 MeOH/CH₂Cl₂) to afford compound 119 (0.2434 g, 67%) as a white solid; mp 72-75° C. ¹H NMR (400 MHz, CDCl₃) δ 7.42 (d, J=8.6 Hz, 2H), 7.23 (d, J=8.3 Hz, 2H), 4.73 (d, J=12.8 Hz, 1H), 3.75 (d, J=13.6 Hz, 1H), 3.00 (s, 1H), 2.87 (d, J=11.4 Hz, 2H), 2.75 (s, 1H), 2.50 (t, J=11.4 Hz, 1H), 2.14 (t, J=11.5 Hz, 2H), 1.87 (d, J=49.0 Hz, 2H), 1.70-1.28 (m, 5H), 1.20 (qd, J=11.8, 3.8 Hz, 2H), 0.90 (d, J=6.3 Hz, 3H). ¹³C NMR (100 MHz, CDCl₃) δ 168.8, 149.9 (q, J=1.8 Hz), 134.7, 128.7, 120.9, 120.3 (q, J=258.0 Hz), 62.1, 49.8 and 49.5 (rotamers), 47.4 and 42.0 (rotamers), 34.6, 31.0, 29.2 and 27.8 (rotamers), 21.9. HRMS (ESI-TOF) (ESI) calcd for C₁₉H₂₅N₂O₂F₃ [M+Na]⁺ 393.1754, found: 393.1760.

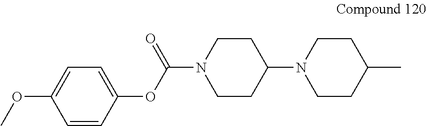

Compound 120: 4-Methoxyphenyl 4-methyl-[1,4′-bipiperidine]-1′-carboxylate. To a solution of 4-methyl-1,4′-bipiperidine (0.1831 g, 1.01 mmol), and K₂CO₃ (0.1522 g, 1.10 mmol) in Et₂O (15 mL) was added at 0° C. 4-methoxyphenyl carbonochloridate (0.15 mL, 1.00 mmol). The resulting suspension was allowed to warm up to room temperature gradually and stirred for 5 h. The suspension was then poured into H₂O (30 mL), extracted with CH₂Cl₂ (3×30 mL). The combined organic layers were dried over Na₂SO₄, filtered and concentrated. The residue was purified by flash chromatography (9/1 CH₂Cl₂/CH₃OH) to afford compound 120 as a white solid (0.2647 g, 79%). ¹H NMR (400 MHz, CDCl₃) δ 6.97 (d, J=9.1 Hz, 2H), 6.83 (d, J=9.1 Hz, 2H), 4.33 (s, 2H), 3.75 (s, 3H), 3.09 (d, J=11.0 Hz, 2H), 2.93 (s, 1H), 2.79 (t, J=11.8 Hz, 2H), 2.41 (s, 2H), 2.05 (s, 2H), 1.84-1.37 (m, 7H), 0.94 (d, J=6.1 Hz, 3H). ¹³C NMR (100 MHz, CDCl₃) δ 156.9, 153.8, 144.8, 122.5, 114.3, 62.7, 55.6, 49.6, 49.2, 43.7, 32.8, 30.3, 27.5, 26.8, 21.4. HRMS (ESI-TOF) (ESI) calcd for C₁₉H₂₉N₂O₃ [M+H]⁺ 333.2173, found 333.2189.

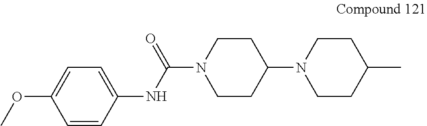

Compound 121: N-(4-Methoxyphenyl)-4-methyl-[1,4′-bipiperidine]-1′-carboxamide. To a 15 mL flame-dried flask were added 4-methyl-1,4′-bipiperidine (0.1889 g, 1.04 mmol), DCM (6 mL) and 1-isocyanato-4-methoxybenzene (0.1674 g, 1.12 mmol) at 0° C. sequentially. The resulting solution was then allowed to warm to room temperature and stirred for 22 hours. After the evaporation of solvents, the residue was purified through flash chromatography on silica gel (1:19 MeOH/CH₂Cl₂) to afford the entitled product as a white solid (0.2032 g, 59%). ¹H NMR (400 MHz, CDCl₃) δ 7.21 (d, J=8.9 Hz, 2H), 6.80 (d, J=8.9 Hz, 2H), 6.38 (s, 1H), 4.08 (d, J=13.2 Hz, 2H), 3.75 (s, 3H), 2.93-2.72 (m, 4H), 2.53-2.33 (m, 1H), 2.14 (t, J=11.6 Hz, 2H), 1.83 (d, J=11.9 Hz, 2H), 1.64 (d, J=13.8 Hz, 1H), 1.49 (qd, J=12.4, 4.2 Hz, 2H), 1.42-1.26 (m, 1H), 1.20 (qd, J=11.9, 3.5 Hz, 2H), 0.90 (d, J=6.3 Hz, 3H). ¹³C NMR (100 MHz, CDCl₃) δ 155.7, 155.4, 132.2, 122.3, 114.0, 62.2, 55.5, 49.6, 44.0, 34.7, 31.1, 28.0, 21.9. HRMS (ESI-TOF) (ESI) calcd for C₁₉H₃₀N₃O₂ [M+H]⁺ 332.2333, found 332.236

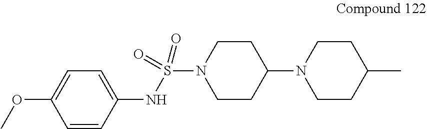

Compound 122: N-(4-Methoxyphenyl)-4-oxopiperidine-1-sulfonamide. A solution of 4-methoxyaniline (1.8932 g, 15.39 mmol) in CH₂Cl₂ (18 mL) was cooled to −10° C. and chlorosulfonic acid (0.34 mL, 5.13 mmol) was added dropwise in 10 min. The resulting suspension was allowed to warm to room temperature and stirred for 3 h. After filtration, the solid was dried under reduced pressure and suspended in toluene (15 mL), followed by addition of PCl₅ (1.0239 g, 4.92 mmol). The reaction mixture was stirred at 75° C. for 3.5 h and filtered. The filtrate was concentrated under reduced pressure to afford 4-MeOPhNHSO₂Cl which was used without further purification.

A suspension of piperidin-4-one hydrochloride hydrate (2a, 0.7689 g, 5.03 mmol), Na₂SO₄ (1.2132 g, 7.19 mmol), and Et₃N (2.9 mL, 2.1054 g, 20.85 mmol) in CH₂Cl₂ (15 mL) was stirred vigorously, followed by addition of 4-MeOPhNHSO₂Cl as prepared above. The reaction mixture was stirred at room temperature for 20 h and poured into 0.5 N aqueous HCl (50 mL) and extracted with CH₂Cl₂ (3×50 mL). The combined organic layers were dried over Na₂SO₄, filtered and concentrated. The residue was purified by flash chromatography (1/1 EtOAc/hexane) to afford N-(4-methoxyphenyl)-4-oxopiperidine-1-sulfonamide as a brown gel (0.1552 g, 11% over two steps). ¹H NMR (400 MHz, CDCl₃) δ 7.17 (d, J=8.9 Hz, 2H), 7.02 (s, 1H), 6.84 (d, J=8.9 Hz, 2H), 3.77 (s, 3H), 3.53 (t, J=6.2 Hz, 4H), 2.42 (t, J=6.2 Hz, 4H). ¹³C NMR (100 MHz, CDCl₃) δ 206.4, 157.7, 129.2, 124.2, 114.6, 55.5, 46.0, 40.8. MS (ESI) calcd for Cl₂H₁₇N₂O₄S [M+H]⁺ 285.1, found 285.1.

Reaction of the above prepared N-(4-methoxyphenyl)-4-oxopiperidine-1-sulfonamide (0.1102 g, 0.39 mmol) with 4-methylpiperidine (Procedure D) yielded 122 as a pale yellow gel (0.0217 g, 15%). ¹H NMR (400 MHz, CDCl₃) δ 7.21 (d, J=8.8 Hz, 2H), 6.84 (dd, J=8.9, 2.3 Hz, 2H), 3.88 (d, J=12.7 Hz, 2H), 3.79 (d, J=1.8 Hz, 3H), 3.16 (d, J=11.7 Hz, 2H), 2.92 (s, 1H), 2.74 (t, J=12.2 Hz, 2H), 2.50 (t, J=11.5 Hz, 2H), 2.13-1.93 (m, 3H), 1.88-1.56 (m, 6H), 1.55-1.39 (m, 1H), 0.97 (d, J=6.5 Hz, 3H). ¹³C NMR (100 MHz, CDCl₃) δ 157.2, 130.1, 123.9, 114.4, 62.3, 55.5, 49.0, 45.5, 31.4, 29.6, 25.9, 21.0. HRMS (ESI-TOF) calcd for C₁₈H₃₀N₃O3S [M+H]⁺ 368.2002, found 368.1991.

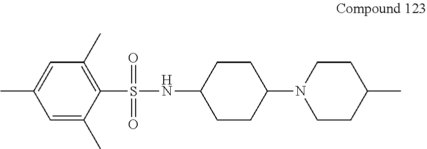

Compound 123: 2,4,6-Trimethyl-N-(4-(4-methylpiperidin-1-yl)cyclohexyl)benzenesulfonamide. A mixture of tert-butyl (4-oxocyclohexyl)carbamate (0.4296 g, 2.02 mmol), HCl (37%, 2 mL) and 1,4-dioxane (6 mL) was stirred at room temperature for 3.5 hours. Upon evaporation of solvents, the residue (2b) was reacted with 2-mesitylenesulfonyl chloride according to Procedure B to yield 2,4,6-trimethyl-N-(4-oxocyclohexyl)benzenesulfonamide as a light yellow gel (83%). ¹H NMR (400 MHz, CDCl₃) δ 6.94 (s, 2H), 5.36-5.26 (br, 1H), 3.64-3.40 (m, 1H), 2.63 (s, 6H), 2.40-2.31 (m, 2H), 2.30-2.20 (m, 5H), 2.04-1.93 (m, 2H), 1.79-1.67 (m, 2H). ¹³C NMR (100 MHz, CDCl₃) δ 142.4, 138.9, 134.3, 132.1, 49.6, 38.4, 32.2, 22.9, 20.9.

Reaction of the above prepared 2,4,6-trimethyl-N-(4-oxocyclohexyl)benzenesulfonamide with 4-methylpiperidine (Procedure D) yielded 123 as a light yellow gel (13%). ¹H NMR (400 MHz, CDCl₃) δ 6.91 (s, 2H), 6.36 (s, 1H), 3.45-3.20 (m, 3H), 3.02 (t, J=11.7, 1H), 2.74 (dt, J=13.9, 7.9 Hz, 2H), 2.59 (s, 6H), 2.26 (s, 3H), 2.10-1.65 (m, 10H), 1.62-1.35 (m, 3H), 0.94 (d, J=6.5 Hz, 3H). ¹³C NMR (100 MHz, CDCl₃) δ 142.4, 139.0, 133.7, 132.1, 64.0, 49.1, 47.1, 30.9, 29.9, 23.0, 20.9, 20.8. HRMS (ESI-TOF) calcd for C₂₁H₃₅N₂O₂S [M+H]⁺379.2414, found 379.2427.

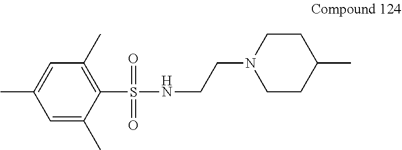

Compound 124: 2,4,6-Trimethyl-N-(2-(4-methylpiperidin-1-yl)ethyl)benzenesulfonamide. Reaction of amine 1h with 2,4,6-trimethylbenzene-1-sulfonyl chloride (Procedure A) yielded 124 as a yellow gel (>95%). ¹H NMR (400 MHz, CDCl₃) δ 6.91 (s, 2H), 2.86 (d, J=5.2 Hz, 2H), 2.61 (s, 6H), 2.52 (d, J=11.6 Hz, 2H), 2.28 (d, J=5.9 Hz, 2H), 2.26 (s, 3H), 1.82 (td, J=11.9, 2.6 Hz, 2H), 1.52 (d, J=13.1 Hz, 2H), 1.36-1.20 (m, 1H), 1.07 (qd, J=12.5, 3.7 Hz, 2H), 0.86 (d, J=6.5 Hz, 3H). ¹³C NMR (100 MHz, CDCl₃) δ 142.0, 139.0, 133.3, 131.8, 55.8, 53.3, 39.0, 34.2, 30.6, 22.9, 21.8, 20.9. HRMS (ESI-TOF) calcd for C₁₇H N₂₉N₂O₂S [M+H]⁺ 325.1944 found 325.1947.

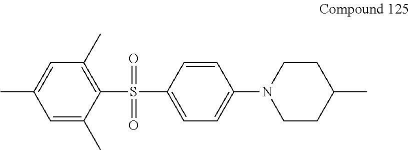

Compound 125: 1-(4-(Mesitylsulfonyl)phenyl)-4-methylpiperidine. On the basis of a literature report, a mixture of 4-iodobenzenesulfonyl chloride (0.9232 g, 3.02 mmol), mesitylene (120 mg, 3.09 mmol), and AlCl₃ (0.4956 g, 3.73 mmol) in CH₂Cl₂ (15 mL) was stirred for 3 h at room temperature. (Chen et al. (2013) J. Med. Chem. 56:952-62). The mixture was then poured into 45 mL of 5% aqueous HCl and extracted with CH₂Cl₂ (3×30 mL). The combined organic layers were concentrated under reduced pressure to about 50 mL and were then washed with saturated aqueous NaHCO₃(45 mL) and brine (45 mL). The organic layer was dried over anhydrous Na₂SO₄, filtered and concentrated. The residue was purified by flash chromatography (10/1 hexane/EtOAc) to give the desired product 2-((4-iodophenyl)sulfonyl)-1,3,5-trimethylbenzene (0.5707 mg, 48%) as pale yellow solid. mp 118-121° C. ¹H NMR (400 MHz, CDCl₃) δ 7.91-7.72 (d, J=8.0 Hz, 2H), 7.57-7.35 (d, J=7.8 Hz, 2H), 6.93 (s, 2H), 2.56 (s, 6H), 2.27 (s, 3H). ¹³C NMR (100 MHz, CDCl₃) δ 143.7, 143.2, 140.0, 138.1, 133.3, 132.3, 127.6, 99.9, 22.8, 21.0.

To a solution of the above prepared 2-((4-iodophenyl)sulfonyl)-1,3,5-trimethylbenzene (0.2879 g, 0.75 mmol), CuI (0.0145 g, 0.076 mmol), L-proline (0.0176 g, 0.15 mmol), and K₂CO₃ (0.2160 g, 1.57 mmol) in DMSO (6 mL) was added 4-methylpiperidine (0.18 mL, 1.52 mmol) and the reaction suspension was stirred vigorously at 90° C. for 67 h. After cooling to room temperature, water (30 mL) was added followed by extraction with CH₂Cl₂ (3×30 mL). The combined organic layers were dried over Na₂SO₄, filtered and concentrated, and the residue was purified by flash chromatography (10/1 hexane/EtOAc) to afford the title compound 125 as a white solid (0.0636 g, 24%); mp 103-105° C. ¹H NMR (400 MHz, CDCl₃) δ 7.62 (d, J=8.9 Hz, 2H), 6.90 (s, 2H), 6.84 (d, J=8.6 Hz, 2H), 3.79 (d, J=12.9 Hz, 2H), 2.84 (t, J=13.4 Hz, 2H), 2.61 (s, 6H), 2.27 (s, 3H), 1.71 (d, J=13.0 Hz, 2H), 1.64-1.47 (m, 1H), 1.25 (q, J=12.3 Hz, 2H), 0.96 (d, J=6.5 Hz, 3H). ¹³C NMR (100 MHz, CDCl₃) δ 142.5, 139.6, 135.3, 132.0, 128.2, 113.7, 48.1, 33.5, 30.7, 22.9, 21.8, 21.0. HRMS (ESI-TOF) calcd for C₂₁H₂₇NO₂SNa [M+Na]⁺380.1655, found 380.1660.

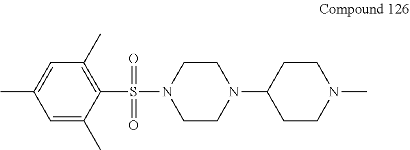

Compound 126: 1-(Mesitylsulfonyl)-4-(1-methylpiperidin-4-yl)piperazine. Reaction of amine 1i with 2,4,6-trimethylbenzenesulfonyl chloride (Procedure A) yielded 126 as a colorless gel (>95%). ¹H NMR (400 MHz, CDCl₃) δ 6.91 (s, 2H), 3.23-3.04 (m, 4H), 2.85 (d, J=12.2 Hz, 2H), 2.59 (s, 6H), 2.57-2.48 (m, 4H), 2.26 (s, 3H), 2.22 (s, 3H), 2.22-2.15 (m, 1H), 1.89 (t, J=12.0 Hz, 2H), 1.69 (d, J=11.0 Hz, 2H), 1.51 (qd, J=12.3, 3.9 Hz, 2H). ¹³C NMR (100 MHz, CDCl₃) δ 142.5, 140.4, 131.9, 131.3, 61.4, 55.3, 48.4, 46.1, 44.7, 28.0, 23.0, 21.0. LC-MS (ESI) calcd for C₂₁H₃₂N₃O₂S [M+H]⁺ 366.2, found 366.2.

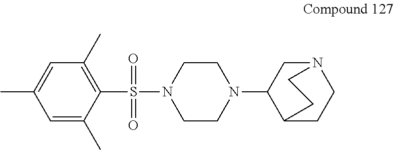 [SMILES: Cc1cc(C)c(S(=O)(=O)N2CCN(C3CN4CCC3CC4)CC2)c(C)c1]

Compound 127: 3-(4-(Mesitylsulfonyl)piperazin-1-yl)quinuclidine. Reaction of amine 1j with 2,4,6-trimethylbenzenesulfonyl chloride (Procedure A) yielded 127 as a white solid (42%); mp >300° C. ¹H NMR (400 MHz, CDCl₃) δ 6.96 (s, 2H), 3.34-3.12 (m, 9H), 3.04 (dd, J=12.0, 4.0 Hz, 1H), 2.60 (s, 6H), 2.45 (s, 5H), 2.30 (s, 4H), 2.16-1.95 (m, 2H), 1.85-1.75 (m, 1H), 1.74-1.62 (m, 1H). ¹³C NMR (100 MHz, CDCl₃) δ 142.8, 140.5, 132.0, 131.0, 59.1, 52.8, 50.3, 46.6, 45.6, 44.1, 23.0, 22.9, 22.1, 20.9, 17.7. HRMS (ESI-TOF) calcd for C₂₀H₃₂N₃O₂S [M+H]⁺ 378.2210, found: 378.2200.

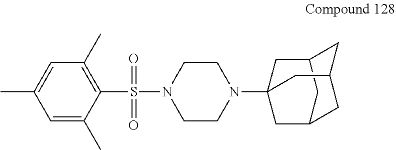 [SMILES: Cc1cc(C)c(S(=O)(=O)N2CCN(C34CC5CC(CC(C5)C3)C4)CC2)c(C)c1]

Compound 128: 1-(Adamantan-1-yl)-4-(mesitylsulfonyl)piperazine. Reaction of amine 11 with 2,4,6-trimethylbenzene-1-sulfonyl chloride (Procedure B) yielded 128 as a yellow solid (70%); mp 182-184° C. ¹H NMR (400 MHz, CDCl₃) δ 6.92 (s, 2H), 3.20-3.02 (m, 4H), 2.68-2.61 (m, 4H), 2.60 (s, 6H), 2.27 (s, 3H), 2.05 (s, 3H), 1.70-1.49 (m, 14H). ¹³C NMR (100 MHz, CDCl₃) δ 142.4, 140.4, 131.9, 131.4, 54.0, 45.2, 43.7, 38.5, 36.8, 29.5, 23.1, 20.9. HRMS calcd for C₂₃H₃₅N₂O₂S [M+H]⁺ 403.2414, found 403.2407.

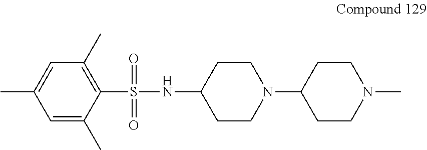

Compound 129: 2,4,6-Trimethyl-N-(1′-methyl-[1,4′-bipiperidin]-4-yl)benzenesulfonamide. Reaction of amine hydrochloride salt 1m with 2,4,6-trimethylbenzene-1-sulfonyl chloride (Procedure B) yielded 129 as a yellow gel (81%). ¹H NMR (400 MHz, CDCl₃) δ 6.89 (s, 2H), 4.73 (s, 1H), 3.04 (s, 1H), 2.84 (d, J=12.1 Hz, 2H), 2.69 (d, J=12.0 Hz, 2H), 2.59 (s, 6H), 2.25 (s, 3H), 2.20 (s, 3H), 2.19-2.05 (m, 3H), 1.87 (t, J=10.6 Hz, 2H), 1.76-1.59 (m, 4H), 1.50 (qd, J=12.2, 3.8 Hz, 2H), 1.44-1.31 (m, 2H). ¹³C NMR (100 MHz, CDCl₃) δ 142.0, 138.7, 135.0, 131.9, 61.4, 55.4, 50.7, 47.6, 46.0, 33.2, 27.7, 22.9, 20.9. HRMS (ESI-TOF) calcd for C₂₀H₃₄N₃O₂S [M+H]⁺ 380.2366, found 380.2359.

Compounds 130-170 can be obtained following synthetic procedures E, F or G as presented below.

General procedure for the preparation of sulfonamides from sulfonyl chlorides and amine hydrochloride salts (Procedure E). A biphasic mixture of sulfonyl chloride (1.2 eq.), amine hydrochloride salt (1.0 eq.) and K₂CO₃ (4 eq.) in CHCl₃ (2 mL/mmol amine hydrochloride salt) and water (2 mL/mmol amine hydrochloride salt) was stirred vigorously at room temperature for 20 h followed by the addition of saturated aqueous NaHCO₃(25 mL/mmol of amine hydrochloride salt). The resulting solution was extracted with CH₂Cl₂ (3×20 mL/mmol of amine hydrochloride salt) and the combined organic layers were dried over Na₂SO₄, filtered and concentrated under reduced pressure. The residue was purified through flash chromatography or Isco Combiflash (1:19 MCOH/CH₂Cl₂, or 1:1 hexanes/EtOAc eluent mixture) to provide the corresponding sulfonamides with >95% purity.

General procedure for the reductive amination of N-arylsulfonyl-piperidin-4ones, or 1-(mesitylsulfonyl)piperidine-4-carbaldehyde (Procedure F). A mixture of ketone (1.0 eq.), amine (1.0 e.q) (if it was an amine salt, it was neutralized by 1 N NaOH solution, followed by DCM extraction to afford the corresponding free amine for the reaction), AcOH (1.0 eq.), and CH₂Cl₂ (or DCE) (5 ml/mmol amine) was stirred at room temperature or 50° C. for 15 min before NaBH(OAc)₃ (1.5 eq.) was added. The resulting suspension was stirred at room temperature or 50° C. with a reaction time ranging from 20 h to 89 h. The reaction was then quenched by dropwise addition of saturated aqueous NaHCO₃ (30 ×mL/mmol of amine) at 0° C. and the resulting biphasic solution was extracted with CH₂Cl₂ (3×30 ml/mmol amine). The combined organic layers were dried over Na₂SO₄, filtered and concentrated under reduced pressure. The residue was purified through flash chromatography or Isco Combiflash (1:19 MeOH/CH₂Cl₂) to provide the corresponding reductive amination product with >95% purity.

General procedure for the preparation of biarylsulfonamides via Suzuki cross-coupling (Procedure G). To a flame-dried flask equipped with a reflux condenser were added bromophenyl)sulfonyl-4-((4-methylpiperidin-1-yl)methyl)piperidine (1.0 eq.), phenylboronic acid (1.58 eq.), Pd(PPh₃)₄ (0.1 eq.), THF (14.5 mL/mmol sulfonamide) and aqueous Na₂CO₃ (2 M; 1.45 mL/mmol sulfonamide). The mixture was degassed through freeze-pump-thaw cycling and was refluxed for 3 h-12 h. After being cooled down to room temperature, the reaction suspension was diluted with water (45.5 mL/mmol sulfonamide), stirred for 10 min and was extracted with CH₂Cl₂ (3×54.5 mL/mmol sulfonamide). The combined organic layers were dried over Na₂SO₄, filtered and concentrated. The residue was purified through flash chromatography or Isco Combiflash (1:19 MeOH/CH₂Cl₂ or 1:19 MeOH/EtOAc) to provide the corresponding biarylsulfonamides with >95% purity.

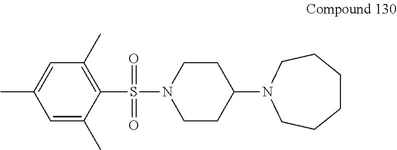 [SMILES: Cc1cc(C)c(S(=O)(=O)N2CCC(N3CCCCCC3)CC2)c(C)c1]

Compound 130: 1-(1-(mesitylsulfonyl)piperidin-4-yl)azepane. Reaction of 2,4,6-trimethylbenzene-1-sulfonyl chloride with 1-piperidin-4-yl-azepane dihydrochloride (Procedure E) yielded the entitled compound as an orange gel (90%); ¹H NMR (400 MHz, CDCl₃) δ 6.94 (s, 2H), 3.71 (d, J=11.8 Hz, 2H), 3.62 (br, 1H), 3.18-2.89 (br, 4H), 2.77 (t, J=12.7 Hz, 2H), 2.58 (s, 6H), 2.29 (s, 3H), 2.10 (d, J=12.5 Hz, 2H), 1.82 (s, 4H), 1.76-1.54 (m, 6H). ¹³C NMR (100 MHz, CDCl₃) δ 142.8, 140.3, 132.0, 132.0, 132.0, 131.5, 63.3, 51.2, 43.8, 26.9, 26.6, 26.4, 22.8, 21.0. HRMS calcd for: C₂₀H₃₃N₂O₂S (MH⁺): 365.2257, found 365.2253.

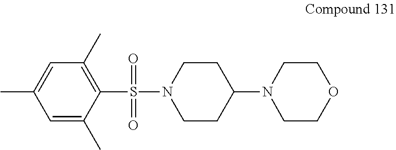 [SMILES: Cc1cc(C)c(S(=O)(=O)N2CCC(N3CCOCC3)CC2)c(C)c1]

Compound 131: 4-(1-(mesitylsulfonyl)piperidin-4-yl)morpholine. Reaction of 1-(mesitylsulfonyl)piperidin-4-one with morpholine (Procedure F) yielded the entitled compound as a colorless gel (74%); ¹H NMR (500 MHz, CDCl₃) δ 6.92 (s, 2H), 3.68 (s, 4H), 3.60 (d, J=12.9 Hz, 2H), 2.75 (t, J=12.3 Hz, 2H), 2.59 (s, 6H), 2.50 (s, 4H), 2.27 (s, 4H), 1.87 (d, J=14.5 Hz, 2H), 1.46 (qd, J=11.6, 3.4 Hz, 2H). ¹³C NMR (100 MHz, CDCl₃) δ 142.5, 140.4, 140.4, 131.9, 131.6, 67.1, 61.5, 49.7, 43.6, 27.7, 22.8, 20.9. HRMS calcd for: C₁₈H₂₉N₂O₃S (MH⁺): 353.1893, found 353.1889.

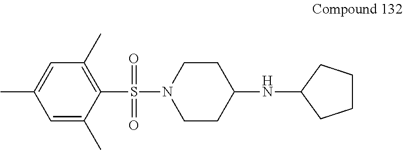 [SMILES: Cc1cc(C)c(S(=O)(=O)N2CCC(NC3CCCC3)CC2)c(C)c1]

Compound 132: N-cyclopentyl-1-(mesitylsulfonyl)piperidin-4-amine. Reaction of 1-(mesitylsulfonyl)piperidin-4-one with cyclopentanamine (Procedure F) yielded the entitled compound as a yellow solid (80%); mp 88-92° C. ¹H NMR (400 MHz, CDCl₃) δ 6.91 (s, 2H), 3.51 (d, J=12.4 Hz, 2H), 3.13 (p, J=7.1 Hz, 1H), 2.78 (td, J=12.2, 2.6 Hz, 2H), 2.63-2.55 (m, 1H), 2.58 (s, 6H), 2.26 (s, 3H), 2.00-1.72 (m, 4H), 1.68-1.56 (m, 2H), 1.54-1.43 (m, 2H), 1.41-1.11 (m, 5H). ¹³C NMR (100 MHz, CDCl₃) δ 142.4, 140.5, 131.8, 131.7, 56.4, 53.0, 43.1, 33.6, 32.3, 23.9, 22.7, 20.9. HRMS calcd for: C₁₉H₃₁N₂O₂S (MH⁺): 351.2101, found 351.2096.

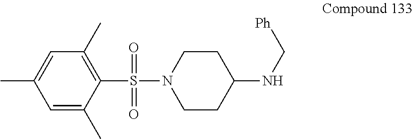 [SMILES: Cc1cc(C)c(S(=O)(=O)N2CCC(NCc3ccccc3)CC2)c(C)c1]

Compound 133: N-benzyl-1-(mesitylsulfonyl)piperidin-4-amine. Reaction of 1-(mesitylsulfonyl)piperidin-4-one with benzylamine (Procedure F) yielded the entitled compound as a yellow oil (>95%); ¹H NMR (400 MHz, CDCl₃) δ 7.45-7.19 (m, 5H), 6.92 (s, 2H), 3.77 (s, 2H), 3.52 (d, J=13.3 Hz, 2H), 3.52 (d, J=13.3 Hz, 2H), 2.68-2.58 (m, 1H), 2.59 (s, 6H), 2.27 (s, 3H), 1.90 (d, J=12.1 Hz, 2H), 1.45-1.22 (m, 2H). ¹³C NMR (100 MHz, CDCl₃) δ 142.5, 140.4, 131.9, 131.7, 128.6, 128.5, 128.0, 127.8, 127.1, 53.7, 50.8, 42.9, 31.7, 22.8, 21.0. HRMS calcd for: C₂₁H₂₉N₂O₂S (MH⁺): 373.1944, found 373.1942.

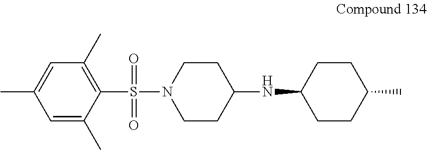

Compound 134: 1-(mesitylsulfonyl)-N-(trans-4-methylcyclohexyl)piperidin-4-amine. Reaction of 1-(mesitylsulfonyl)piperidin-4-one with trans-4-methylcyclohexanamine hydrochloride (Procedure F) yielded the entitled compound as a yellow oil (59%); ¹H NMR (400 MHz, CDCl₃) δ 6.91 (s, 2H), 3.52 (d, J=12.4 Hz, 2H), 2.78 (t, J=12.1 Hz, 2H), 2.74-2.65 (m, 1H), 2.58 (s, 6H), 2.54-2.40 (m, 1H), 2.27 (s, 3H), 1.84 (t, J=14.2 Hz, 4H), 1.66 (d, J=11.7 Hz, 2H), 1.36-1.18 (m, 3H), 1.21-0.87 (m, 5H), 0.84 (d, J=6.5 Hz, 3H). ¹³C NMR (100 MHz, CDCl₃) δ 142.4, 140.4, 131.8, 131.7, 53.0, 50.9, 43.2, 34.0, 32.5, 32.4, 22.8, 22.3, 20.9. HRMS calcd for: C₂₁H₃₅N₂O₂S (MH⁺): 379.2414, found 379.2413.

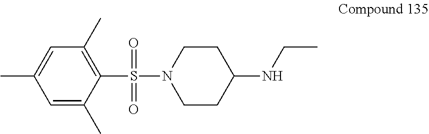 [SMILES: CCNC1CCN(S(=O)(=O)c2c(C)cc(C)cc2C)CC1]

Compound 135: N-ethyl-1-(mesitylsulfonyl)piperidin-4-amine. Reaction of 1-(mesitylsulfonyl)piperidin-4-one with ethanamine (Procedure F) yielded the entitled compound as a yellow oil (65%); ¹H NMR (500 MHz, CDCl₃) δ 6.93 (s, 2H), 3.55 (d, J=12.7 Hz, 2H), 2.82 (t, J=12.2 Hz, 2H), 2.66 (q, J=7.1 Hz, 2H), 2.63-2.55 (m, 1H), 2.61 (s, 6H), 2.29 (s, 3H), 1.91 (d, J=13.4 Hz, 2H), 1.50 (s, 1H), 1.42-1.18 (m, 2H), 1.10 (t, J=7.1 Hz, 3H). ¹³C NMR (100 MHz, CDCl₃) δ 142.5, 140.5, 131.8, 131.7, 54.4, 43.0, 40.9, 31.8, 22.8, 20.9, 15.4. HRMS calcd for: C₁₆H₂₇N₂O₂S (MH⁺): 311.1788, found 311.1796.

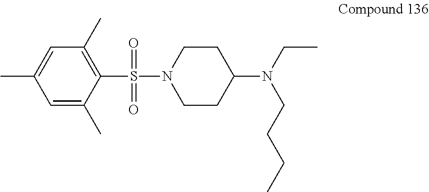 [SMILES: CCCCN(CC)C1CCN(S(=O)(=O)c2c(C)cc(C)cc2C)CC1]

Compound 136: N-butyl-N-ethyl-1-(mesitylsulfonyl)piperidin-4-amine. N-ethyl-1-(mesitylsulfonyl)piperidin-4-amine (0.0656 g, 0.21 mmol), CH₃CN (2 mL), K₂CO₃ (0.0478 g, 0.35 mmol) and TBAI (0.0081 g, 0.02 mmol) were mixed and stirred at room temperature, followed by dropwise addition of 1-bromobutane (0.026 mL, 0.24 mmol). The reaction mixture was stirred at room temperature for 24 hours and was filtered and concentrated under reduced pressure. The residue was purified through flash chromatography (1:9 MeOH/CH₂Cl₂) to afford the title product (0.0053 g, 7% yield). ¹H NMR (400 MHz, CDCl₃) δ 6.94 (s, 2H), 3.64 (d, J=12.2 Hz, 2H), 2.74 (td, J=12.4, 2.4 Hz, 1H), 2.61 (s, 6H), 2.54 (s, 2H), 2.43 (s, 2H), 2.30 (s, 3H), 1.79 (d, J=11.3 Hz, 2H), 1.62-1.34 (m, 4H), 1.27 (dq, J=14.6, 7.5 Hz, 3H), 1.02 (s, 3H), 0.90 (t, J=7.3 Hz, 3H). ¹³C NMR (100 MHz, CDCl₃) δ 142.4, 140.4, 131.9, 131.8, 57.9, 49.7, 44.2, 44.2, 27.9, 22.8, 21.0, 20.6, 14.0. HRMS calcd for: C₂₀H₃₅N₂O₂S (MH⁺): 367.2414, found 367.2412.

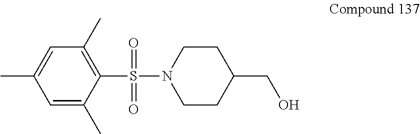 [SMILES: Cc1cc(C)c(S(=O)(=O)N2CCC(CO)CC2)c(C)c1]

Compound 137: (1-(mesitylsulfonyl)piperidin-4-yl)methanol. To a mixture of piperidin-4-ylmethanol (1.0820 g, 9.39 mmol), N, N-diisopropyl ethylamine (2.40 mL, 14.55 mmol), and CH₂Cl₂ (8 mL) was dropwise added 2,4,6-trimethylbenzene-1-sulfonyl chloride (1.8200 g, 8.32 mmol) in 30 min. The reaction solution was stirred at room temperature overnight and was diluted with CH₂Cl₂ (40 mL). The resulting solution was washed by saturated NaHCO₃ solution (40 mL). The organic layer was dried over Na₂SO₄, filtered and concentrated under reduced pressure. The residue was purified through flash chromatography on silica gel (1:19 CH₃OH/CH₂Cl₂) to afford the desired product (1.9342 g, 78%) as a white solid. mp 85-88° C. ¹H NMR (400 MHz, CDCl₃) δ 6.91 (s, 2H), 3.56 (d, J=12.3 Hz, 2H), 3.43 (d, J=6.3 Hz, 2H), 2.71 (td, J=12.3, 2.6 Hz, 2H), 2.57 (s, 6H), 2.26 (s, 3H), 2.00-1.80 (m, 1H), 1.73 (dd, J=13.6, 2.9 Hz, 2H), 1.63-1.48 (m, 1H), 1.18 (qd, J=11.9, 4.3 Hz, 2H). ¹³C NMR (100 MHz, CDCl₃) δ 142.5, 140.3, 131.9, 131.7, 67.0, 44.1, 38.3, 28.1, 22.8, 20.9. HRMS calcd for: C₁₅H₂₃NO₃SNa (MNa⁺): 320.1291. found 320.1280.

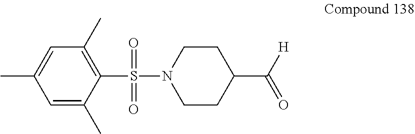 [SMILES: Cc1cc(C)c(S(=O)(=O)N2CCC(C=O)CC2)c(C)c1]

Compound 138: 1-(mesitylsulfonyl)piperidine-4-carbaldehyde. A 25 mL flame-dried flask equipped with a magnetic stirring bar was purged by argon and was then sealed with a rubber septum fitted with an argon balloon. Anhydrous CH₂Cl₂ (2 mL) and oxalyl chloride (0.40 mL, 2 M in CH₂Cl₂, 0.8 mmol) were added via syringe sequentially. The resulting solution was cooled to −78° C. in a dry ice-acetone bath. Anhydrous DMSO (0.12 mL, 1.69 mmol) was introduced and the solution was stirred for 30 min at −78° C. (1-(mesitylsulfonyl)piperidin-4-yl)methanol (0.1975 g, 0.66 mmol) in 2 mL CH₂Cl₂ was added dropwise. After the reaction mixture was stirred at −78° C. for 2 h, Et₃N (0.35 mL, 2.52 mmol) was added and after 10 min, the reaction solution was allowed to warm up to room temperature and stirred for 2 h before being quenched by 25 mL saturated NaHCO₃ solution and the resulting solution was exacted by CH₂Cl₂ (3×25 mL). The combined organic layers were dried over Na₂SO₄, filtered and concentrated under reduced pressure. The residue was purified through flash chromatography on silica gel (1:19 CH₃OH/CH₂Cl₂) to afford the desired product (0.1742 g, 89% yield) as a white solid; mp 71-74° C. ¹H NMR (400 MHz, CDCl₃) δ 9.64 (s, 1H), 6.94 (s, 2H), 3.52 (dt, J=12.6, 4.1 Hz, 2H), 2.90 (ddd, J=12.4, 10.8, 2.9 Hz, 2H), 2.60 (s, 6H), 2.45-2.32 (m, 1H), 2.29 (s, 3H), 2.07-1.90 (m, 2H), 1.62 (dtd, J=14.5, 10.7, 4.0 Hz, 2H). ¹³C NMR (100 MHz, CDCl₃) δ 202.4, 142.7, 140.4, 131.9, 131.5, 47.4, 43.3, 24.8, 22.8, 21.0. LC-MS (ESI) calcd for: C₁₅H₂₂NO₃S (MH⁺): 296.1, found 296.1.

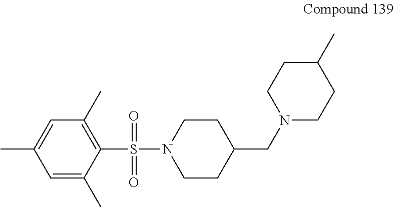

Compound 139: 1-(mesitylsulfonyl)-4-((4-methylpiperidin-1-yl)methyl)piperidine. Reaction of 1-(mesitylsulfonyl)piperidine-4-carbaldehyde with 4-methylpiperidine (Procedure F) yielded the entitled compound as a pale yellow solid (59%); ¹H NMR (500 MHz, CDCl₃) δ 6.94 (s, 2H), 3.57 (d, J=12.7 Hz, 2H), 2.99-2.68 (m, 4H), 2.62 (s, 6H), 2.30 (s, 3H), 2.17 (s, 2H), 1.91 (s, 2H), 1.81 (d, J=12.0 Hz, 2H), 1.74-1.53 (m, 3H), 1.42-1.08 (m, 5H), 0.91 (d, J=6.1 Hz, 3H). ¹³C NMR (100 MHz, CDCl₃) δ 142.3, 140.4, 131.9, 131.8, 64.5, 54.5, 44.3, 34.1, 33.3, 30.7, 30.3, 22.8, 21.8, 20.9. HRMS calcd for: C₂₁H₃₅N₂O₂S (MH⁺): 379.2414, found 379.2411.

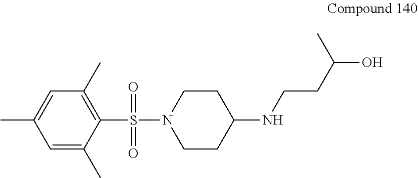 [SMILES: Cc1cc(C)c(S(=O)(=O)N2CCC(NCCC(C)O)CC2)c(C)c1]

Compound 140: 4-((1-(mesitylsulfonyl)piperidin-4-yl)amino)butan-2-ol. Reaction of 1-(mesitylsulfonyl)piperidin-4-one with 4-aminobutan-2-ol (Procedure F) yielded the entitled compound as a white solid (82%); mp 105-108° C. ¹H NMR (500 MHz, CDCl₃) δ 6.95 (s, 2H), 3.96 (ddd, J=8.9, 5.8, 2.4 Hz, 1H), 3.55 (d, J=12.5 Hz, 2H), 3.05 (dt, J=11.9, 4.2 Hz, 1H), 2.83 (tt, J=12.5, 3.3 Hz, 2H), 2.76 (td, J=11.1, 2.9 Hz, 1H), 2.61 (s, 7H), 2.30 (s, 3H), 1.96 (t, J=12.8 Hz, 2H), 1.63 (d, J=14.9 Hz, 1H), 1.53-1.42 (m, 1H), 1.39-1.27 (m, 2H), 1.16 (d, J=6.2 Hz, 3H). ¹³C NMR (100 MHz, CDCl₃) δ 142.6, 140.4, 131.9, 131.6, 69.5, 54.4, 45.5, 43.1, 43.0, 37.0, 31.8, 31.5, 23.6, 22.8, 21.0. HRMS calcd for: C₁₈H₃₀N₂O₃SNa (MNa⁺): 377.1869, found 377.1856.

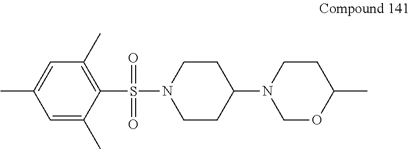

Compound 141: 3-(1-(mesitylsulfonyl)piperidin-4-yl)-6-methyl-1,3-oxazinane. To a 15 mL flask equipped with a reflux condenser were added 4-((1-(mesitylsulfonyl)piperidin-4-yl)amino)butan-2-ol (0.1192 g, 0.34 mmol), paraformaldehyde (0.0143 g, 0.48 mmol), Mg₂SO₄ (0.2091 g, 1.74 mmol), pyridinium p-toluenesulfonate (PPTS) (0.0025 g, 0.01 mmol), and anhydrous toluene (4 mL). The suspension was stirred under reflux for 3 h and was cooled down to room temperature. The suspension was then poured into a separatory funnel containing 30 mL saturated NaHCO₃ solution, followed by extraction with CH₂Cl₂ (3×30 mL). The combined organic layers were dried over Na₂SO₄, filtered and concentrated under reduced pressure. The residue was purified through flash chromatography on silica gel (1:19 MeOH/CH₂Cl₂) to provide the desired product as colorless gel (0.0555 g, 45% yield); ¹H NMR (400 MHz, CDCl₃) δ 6.91 (s, 2H), 4.61 (dd, J=10.0, 2.3 Hz, 1H), 4.16 (d, J=10.1 Hz, 1H), 3.63-3.50 (m, 3H), 3.11 (ddt, J=13.4, 4.4, 2.2 Hz, 1H), 2.89-2.68 (m, 4H), 2.58 (s, 6H), 2.27 (s, 3H), 1.92 (dp, J=12.2, 2.8 Hz, 2H), 1.65-1.29 (m, 4H), 1.15 (d, J=6.1 Hz, 3H). ¹³C NMR (100 MHz, CDCl₃) δ 142.5, 140.4, 131.9, 131.6, 81.6, 73.6, 55.0, 46.5, 43.4, 30.3, 29.8, 29.2, 22.8, 21.8, 20.9. HRMS calcd for: C₁₉H₃₀N₂O₃SNa (MNa⁺): 389.1869, found 389.1874.

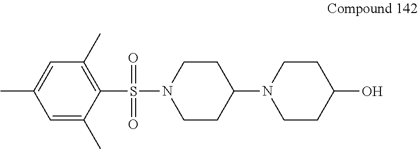 [SMILES: Cc1cc(C)c(S(=O)(=O)N2CCC(N3CCC(O)CC3)CC2)c(C)c1]

Compound 142: 1′-(mesitylsulfonyl)-[1,4′-bipiperidin]-4-ol. To a mixture of [1,4′-bipiperidin]-4-ol (0.2075 g, 1.13 mmol), N, N-diisopropyl ethylamine (0.27 ml, 1.55 mmol), and CH₂Cl₂ (3 mL) was dropwise added 2,4,6-trimethylbenzene-1-sulfonyl chloride (0.2154 g, 0.98 mmol, in 2 mL CH₂Cl₂) in 10 min. The reaction mixture was stirred at room temperature overnight and then poured into saturated NaHCO₃ solution (20 ml). The bi-phase solution was extracted by CH₂Cl₂ (3×20 ml). The combined organic layers were dried by Na₂SO₄, filtered and concentrated under reduced pressure. The residue was purified through flash chromatography on silica gel (1:19 MeOH/CH₂Cl₂) to afford the desired product (0.2138 g, 65% yield) as a white solid; mp 120-123° C. ¹H NMR (400 MHz, CDCl₃) δ 6.92 (s, 2H), 3.74-3.59 (br, 1H), 3.60 (d, J=12.9 Hz, 2H), 2.85-2.65 (m, 4H), 2.59 (s, 6H), 2.46-2.22 (m, 3H), 2.28 (s, 3H), 1.96-1.78 (m, 4H), 1.64-1.30 (m, 5H). ¹³C NMR (100 MHz, CDCl₃) δ 142.5, 140.3, 131.9, 131.6, 67.6, 61.4, 46.7, 43.9, 34.5, 27.6, 22.8, 20.9. HRMS calcd for: C₁₉H₃₁N₂O₃S (MH⁺): 367.2050, found 367.2058.

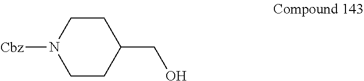 [SMILES: O=C(OCc1ccccc1)N1CCC(CO)CC1]

Compound 143: Benzyl 4-(hydroxymethyl)piperidine-1-carboxylate was prepared according to the literature report. (Boyer, N. et al. (2008) Eur. J. Org. Chem. 25:4277-95). To a 500 mL flask were added piperidin-4-ylmethanol (4.8561 g, 42.15 mmol), CH₂Cl₂ (70 mL), water (70 mL), and Na₂CO₃ (22.7943 g, 215.04 mmol). The bi-phase solution was stirred vigorously at 0° C., followed by dropwise addition of CbzCl (7.0 mL, 49.03 mmol). The reaction solution was then allowed to warm up to room temperature and stirred for 19 h. The reaction solution was diluted with water (70 mL) and extracted with CH₂Cl₂ (2×150 mL). The combined organic layers were dried over Na₂SO₄, filtered and concentrated under reduced pressure. The residue was purified through flash chromatography on silica gel (1:19 CH₃OH/CH₂Cl₂) to afford the desired product (8.3160 g, 79% yield) as a light yellow gel. ¹H NMR (400 MHz, CDCl₃) δ 7.41-7.15 (m, 5H), 5.10 (s, 2H), 4.19 (s, 2H), 3.47 (d, J=6.1 Hz, 2H), 2.77 (d, J=13.3 Hz, 2H), 1.91-1.53 (m, 4H), 1.26-1.00 (m, 2H). ¹³C NMR (100 MHz, CDCl₃) δ 155.3, 136.8, 128.5, 127.9, 127.8, 67.2, 67.0, 43.9, 38.6, 28.6.

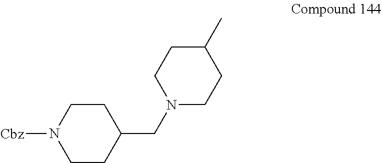 [SMILES: CC1CCN(CC2CCN(C(=O)OCc3ccccc3)CC2)CC1]

Compound 144: Benzyl 4-((4-methylpiperidin-1-yl)methyl)piperidine-1-carboxylate. A 500 mL flame-dried flask equipped with a magnetic stirring bar was purged by argon and was then sealed with a rubber septum fitted with an argon balloon. Anhydrous CH₂Cl₂ (100 mL) and oxalyl chloride (20 mL, 2 M in CH₂Cl₂, 40.00 mmol) were added via syringe sequentially. The resulting solution was cooled to −78° C. in a dry ice-acetone bath. Anhydrous DMSO (5.80 mL, 81.79 mmol) was introduced and the solution was stirred for 30 min at −78° C. Benzyl 4-(hydroxymethyl)piperidine-1-carboxylate (8.3160 g, 33.40 mmol) in anhydrous CH₂Cl₂ (16 mL) was added dropwise. After the reaction mixture was stirred at −78° C. for 1.5 h, Et₃N (17 mL, 122.20 mmol) was added and after 5 min, the reaction solution was allowed to warm up to room temperature and stirred for 1 h before being quenched by 200 mL saturated NaHCO₃ and the resulting solution was exacted by CH₂Cl₂ (3×200 mL). The combined organic layers were dried by Na₂SO₄, filtered and concentrated under reduced pressure to afford the benzyl 4-formylpiperidine-1-carboxylate intermediate which was used in next step without further purification.

A mixture of 4-methylpiperidine (3.3795 g, 34.14 mmol), benzyl 4-formylpiperidine-1-carboxylate intermediate above, DCE (70 mL), AcOH (1.9 mL, 33.22 mmol) and NaBH(OAc)₃ (11.0123 g, 51.94 mmol) was stirred at room temperature for 63 h. The reaction was then quenched by saturated NaHCO₃ solution at 0° C. and extracted with CH₂Cl₂ (3×200 mL). The combined organic layers were dried by Na₂SO₄, filtered and concentrated under reduced pressure. The residue was purified through flash chromatography on silica gel (1:19 CH₃OH/CH₂Cl₂) to provide the desired product as a brown gel (7.3174 g, 66% yield over two steps); ¹H NMR (400 MHz, CDCl₃) δ 7.38-7.25 (m, 5H), 5.08 (s, 2H), 4.14 (s, 2H), 2.85 (d, J=11.0 Hz, 2H), 2.75 (d, J=13.3 Hz, 2H), 2.18 (d, J=6.6 Hz, 2H), 1.94 (t, J=10.9 Hz, 2H), 1.82-1.63 (m, 3H), 1.61-1.52 (m, 2H), 1.28 (qd, J=14.2, 11.5, 8.0 Hz, 3H), 1.07 (qd, J=14.6, 13.9, 4.7 Hz, 2H), 0.89 (d, J=5.6 Hz, 3H). ¹³C NMR (100 MHz, CDCl₃) δ 155.2, 136.9, 128.4, 127.9, 127.7, 66.9, 64.6, 54.4, 44.0, 33.8, 33.4, 30.8, 30.6, 21.8. HRMS calcd for: C₂₀H₃₁N₂O₂S (MH⁺): 331.2380. found 331.2371.

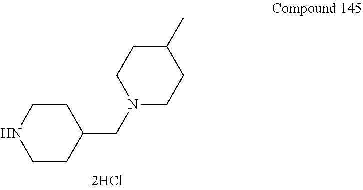

Compound 145: 4-methyl-1-(piperidin-4-ylmethyl)piperidine dihydrochloride. To a 50 mL flask were added benzyl 4-((4-methylpiperidin-1-yl)methyl)piperidine-1-carboxylate (0.4904 g, 1.48 mmol), methanol (8 mL) and Pd/C (3 spatula, 20% on active carbon). The reaction flask was sealed by a septum and after the removal of air using vacuum, a hydrogen balloon was fitted on the top of the septum. The reaction suspension was then stirred at room temperature for 22 h and was filtered through a pad of celite. The filtrate was concentrated under reduced pressure and the generated residue was dissolved in CH₂Cl₂ (6 mL), followed by addition of HCl in 1,4-dioxane (8 mL, 4 M, 32.00 mmol). After being stirred for 10 min, the reaction solution was concentrated under reduced pressure to provide the desired product (0.3829 g, >95% yield) as a yellow gel; ¹H NMR (400 MHz, D20) δ 3.48-3.24 (m, 4H), 3.03-2.70 (m, 6H), 2.18-1.96 (m, 1H), 1.91-1.69 (m, 4H), 1.60-1.18 (m, 5H), 0.78 (d, J=6.6 Hz, 3H). ¹³C NMR (100 MHz, D20) δ 61.1, 53.6, 43.0, 30.7, 28.5, 27.9, 26.0, 20.2. LC-MS (ESI) calcd for: C₁₂H₂₅N₂O₂ (M+H−2Cl)⁺: 197.2, found 197.2.

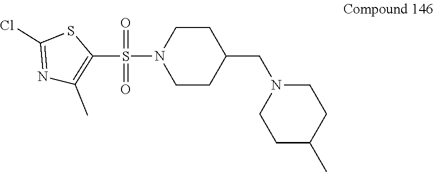 [SMILES: Cc1nc(Cl)sc1S(=O)(=O)N1CCC(CN2CCC(C)CC2)CC1]

Compound 146: 2-chloro-4-methyl-5-((4-((4-methylpiperidin-1-yl)methyl)piperidin-1-yl)sulfonyl)thiazole. Reaction of 2-chloro-4-methylthiazole-5-sulfonyl chloride with 4-methyl-1-(piperidin-4-ylmethyl)piperidine dihydrochloride (Procedure E) yielded the entitled compound as a brown gel (37% yield); ¹H NMR (400 MHz, CDCl₃) δ 3.78 (d, J=11.6 Hz, 2H), 2.88 (d, J=10.9 Hz, 2H), 2.58 (s, 3H), 2.51 (t, J=11.5 Hz, 2H), 2.25 (d, J=6.8 Hz, 2H), 2.03 (s, 2H), 1.93-1.84 (m, 2H), 1.61 (t, J=12.7 Hz, 3H), 1.42-1.19 (m, 5H), 0.89 (d, J=5.1 Hz, 3H). ¹³C NMR (100 MHz, CDCl₃) δ 155.3, 154.3, 128.9, 63.9, 54.4, 46.2, 33.4, 32.5, 30.4, 30.2, 21.6, 16.8. HRMS calcd for: C₁₆H₂₇ClN₃O₂S (MH⁺): 392.1228, found 392.1237.

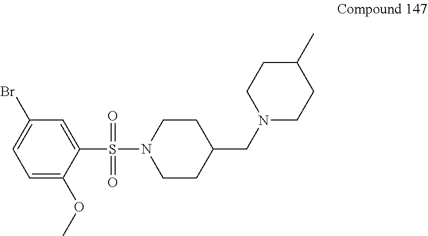 [SMILES: COc1ccc(Br)cc1S(=O)(=O)N1CCC(CN2CCC(C)CC2)CC1]

Compound 147: 1-((5-bromo-2-methoxyphenyl)sulfonyl)-4-((4-methylpiperidin-1-yl)methyl)piperidine. Reaction of 5-bromo-2-methoxybenzene-1-sulfonyl chloride with 4-methyl-1-(piperidin-4-ylmethyl)piperidine dihydrochloride (Procedure E) yielded the entitled compound as a brown gel (63% yield); ¹H NMR (400 MHz, CDCl₃) δ 7.97 (s, 1H), 7.56 (d, J=8.8 Hz, 1H), 6.86 (d, J=8.8 Hz, 1H), 3.87 (s, 3H), 3.81 (d, J=12.5 Hz, 2H), 2.79 (d, J=9.2 Hz, 2H), 2.58 (t, J=12.3 Hz, 2H), 2.14 (d, J=6.9 Hz, 2H), 1.89 (t, J=11.3 Hz, 2H), 1.77 (d, J=11.8 Hz, 2H), 1.61-1.48 (m, 3H), 1.39-1.10 (m, 5H), 0.87 (d, J=6.1 Hz, 3H). ¹³C NMR (100 MHz, CDCl₃) δ 155.9, 136.8, 133.9, 128.7, 114.0, 112.3, 64.5, 56.3, 54.5, 46.1, 33.9, 33.2, 30.8, 30.6, 21.8. HRMS calcd for: C₁₉H₃₀BrN₂O₃S (MH⁺): 445.1155, found 445.1165.

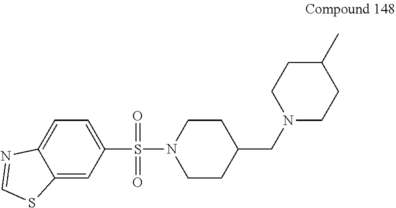 [SMILES: CC1CCN(CC2CCN(S(=O)(=O)c3ccc4ncsc4c3)CC2)CC1]

Compound 148: 6-((4-((4-methylpiperidin-1-yl)methyl)piperidin-1-yl)sulfonyl)benzo[d]thiazole. Reaction of benzo[d]thiazole-6-sulfonyl chloride with 4-methyl-1-(piperidin-4-ylmethyl)piperidine dihydrochloride (Procedure E) yielded the entitled compound as a yellow solid (39%); mp 137-139° C. ¹H NMR (400 MHz, CDCl₃) δ 9.18 (s, 1H), 8.41 (s, 1H), 8.23 (d, J=8.6 Hz, 1H), 7.86 (d, J=8.7 Hz, 1H), 3.82 (d, J=10.0 Hz, 2H), 2.72 (d, J=10.5 Hz, 2H), 2.27 (td, J=11.7, 2.5 Hz, 2H), 2.10 (d, J=6.8 Hz, 2H), 1.93-1.69 (m, 4H), 1.52 (d, J=10.2 Hz, 2H), 1.40 (s, 1H), 1.33-1.10 (m, 5H), 0.85 (d, J=6.4 Hz, 3H). ¹³C NMR (100 MHz, CDCl₃) δ 157.7, 155.5, 134.2, 133.6, 125.2, 124.1, 122.4, 64.3, 54.4, 46.4, 34.0, 32.8, 30.7, 30.2, 21.8. HRMS calcd for: C₁₉H₂₈N₃O₂S₂ (MH⁺): 394.1617, found 394.1617.

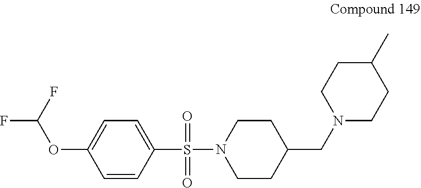 [SMILES: CC1CCN(CC2CCN(S(=O)(=O)c3ccc(OC(F)F)cc3)CC2)CC1]

Compound 149: 1-((4-(difluoromethoxy)phenyl)sulfonyl)-4-((4-methylpiperidin-1-yl)methyl)piperidine. Reaction of 4-(difluoromethoxy)benzene-1-sulfonyl chloride with 4-methyl-1-(piperidin-4-ylmethyl)piperidine dihydrochloride (Procedure E) yielded the entitled compound as a brown gel (50%); ¹H NMR (400 MHz, CDCl₃) δ 7.74 (d, J=6.9 Hz, 2H), 7.22 (d, J=7.1 Hz, 2H), 6.68 (t, J=72.3 Hz, 1H), 3.75 (d, J=3.6 Hz, 2H), 2.73 (d, J=11.5 Hz, 2H), 2.22 (td, J=12.0, 2.5 Hz, 2H), 2.11 (d, J=6.9 Hz, 2H), 1.89-1.73 (m, 4H), 1.53 (d, J=13.1 Hz, 2H), 1.43 (tt, J=8.0, 3.9 Hz, 1H), 1.33-1.11 (m, 5H), 0.86 (d, J=6.3 Hz, 3H). ¹³C NMR (100 MHz, CDCl₃) δ 154.1, 132.9, 129.8, 119.3, 115.2 (t, J=262.5 Hz), 64.2, 54.4, 46.3, 33.8, 32.8, 30.6, 30.2, 21.7. HRMS calcd for: C₁₉H₂₉F₂N₂O₃S (MH⁺): 403.1861, found 403.1858.

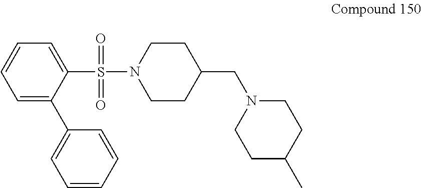 [SMILES: CC1CCN(CC2CCN(S(=O)(=O)c3ccccc3-c3ccccc3)CC2)CC1]

Compound 150: 1-([1,1′-biphenyl]-2-ylsulfonyl)-4-((4-methylpiperidin-1-yl)methyl)piperidine. Reaction of [1,1′-biphenyl]-2-sulfonyl chloride with 4-methyl-1-(piperidin-4-ylmethyl)piperidine dihydrochloride (Procedure E) yielded the entitled compound as a brown gel (51%); ¹H NMR (400 MHz, CDCl₃) δ 8.09 (d, J=8.0 Hz, 1H), 7.54 (t, J=7.5 Hz, 1H), 7.45 (t, J=7.7 Hz, 1H), 7.42-7.32 (m, 5H), 7.31-7.23 (m, 1H), 3.22 (d, J=12.9 Hz, 2H), 2.84 (s, 2H), 2.30-1.71 (m, 6H), 1.65-1.42 (m, 5H), 1.34 (s, 3H), 0.99-0.77 (m, 5H). ¹³C NMR (100 MHz, CDCl₃) δ 141.6, 139.7, 137.0, 133.0, 132.2, 130.3, 129.6, 127.7, 127.5, 127.4, 64.2, 54.4, 44.5, 33.3, 32.6, 30.4, 30.3, 21.6. HRMS calcd for: C₂₄H₃₃N₂O₂S (MH⁺): 413.2257, found 413.2266.

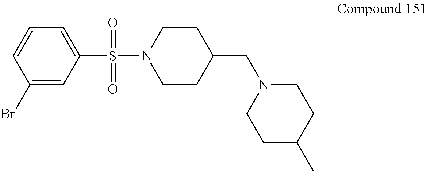 [SMILES: CC1CCN(CC2CCN(S(=O)(=O)c3cccc(Br)c3)CC2)CC1]

Compound 151: 1-((3-bromophenyl)sulfonyl)-4-((4-methylpiperidin-1-yl)methyl)piperidine. Reaction of 3-bromobenzene-1-sulfonyl chloride with 4-methyl-1-(piperidin-4-ylmethyl)piperidine dihydrochloride (Procedure E) yielded the entitled compound as a brown gel (87%); ¹H NMR (400 MHz, CDCl₃) δ 7.86 (s, 1H), 7.66 (dd, J=14.5, 7.8 Hz, 2H), 7.38 (td, J=7.9, 1.3 Hz, 1H), 3.74 (d, J=12.1 Hz, 2H), 2.76 (d, J=7.3 Hz, 2H), 2.32-2.18 (m, 2H), 2.13 (d, J=6.8 Hz, 2H), 1.96-1.71 (m, 4H), 1.58-1.38 (m, 3H), 1.38-1.12 (m, 5H), 0.86 (d, J=5.1 Hz, 3H). ¹³C NMR (100 MHz, CDCl₃) δ 138.1, 135.6, 130.5, 130.4, 126.1, 123.0, 64.2, 54.4, 46.3, 33.8, 32.7, 30.6, 30.2, 21.7. HRMS calcd for: C₁₈H₂₈BrN₂O₂S (MH⁺) 415.1049, found 415.1044.

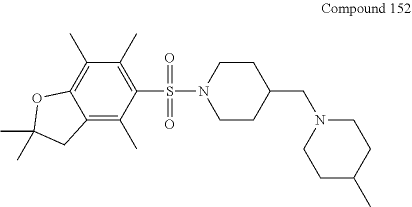

Compound 152: 4-methyl-1-((1-((2,2,4,6,7-pentamethyl-2,3-dihydrobenzofran-5-yl)sulfonyl)piperidin-4-yl)methyl)piperidine. Reaction of 2,2,4,6,7-pentamethyl-2,3-dihydrobenzofuran-5-sulfonyl chloride with 4-methyl-1-(piperidin-4-ylmethyl)piperidine dihydrochloride (Procedure E) yielded the entitled compound as a yellow gel (44%); ¹H NMR (400 MHz, CDCl₃) δ 3.53 (d, J=12.5 Hz, 2H), 2.94 (s, 2H), 2.84 (s, 2H), 2.71 (td, J=12.3, 2.5 Hz, 2H), 2.47 (s, 3H), 2.43 (s, 3H), 2.20 (s, 2H), 2.07 (s, 3H), 1.96 (s, 2H), 1.79 (d, J=15.1 Hz, 2H), 1.70-1.51 (m, 3H), 1.45 (s, 6H), 1.30 (s, 3H), 1.20-1.05 (m, 2H), 0.88 (d, J=5.4 Hz, 3H). ¹³C NMR (100 MHz, CDCl₃) δ 159.9, 140.8, 135.3, 125.7, 125.0, 117.9, 86.8, 64.1, 54.3, 46.7, 44.0, 43.1, 33.4, 33.0, 30.4, 30.3, 28.6, 21.6, 19.2, 17.6, 12.5. HRMS calcd for: C₂₅H₄₁N₂O₃S (MH⁺): 449.2832, found 449.2830.

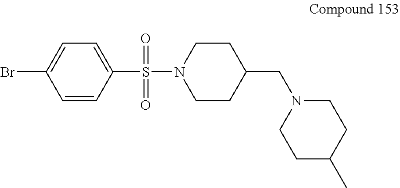 [SMILES: CC1CCN(CC2CCN(S(=O)(=O)c3ccc(Br)cc3)CC2)CC1]

Compound 153: 1-((4-bromophenyl)sulfonyl)-4-((4-methylpiperidin-1-yl)methyl)piperidine. Reaction of 4-bromobenzene-1-sulfonyl chloride with 4-methyl-1-(piperidin-4-ylmethyl)piperidine dihydrochloride (Procedure E) yielded the entitled compound as a yellow gel (63%); ¹H NMR (400 MHz, CDCl₃) δ 7.67 (d, J=8.6 Hz, 2H), 7.61 (d, J=8.6 Hz, 2H), 3.76 (d, J=11.8 Hz, 2H), 2.75 (s, 2H), 2.24 (td, J=12.0, 2.6 Hz, 2H), 2.13 (s, 2H), 1.82 (d, J=13.8 Hz, 4H), 1.56 (d, J=11.9 Hz, 2H), 1.45 (s, 1H), 1.36-1.10 (m, 5H), 0.89 (d, J=6.2 Hz, 3H). ¹³C NMR (100 MHz, CDCl₃) δ 135.2, 132.2, 129.1, 127.6, 64.3, 54.5, 46.3, 34.0, 32.8, 30.7, 30.2, 21.8. HRMS calcd for: C₁₈H₂₈BrN₂O₂S (MH⁺): 415.1049, found 415.1044.

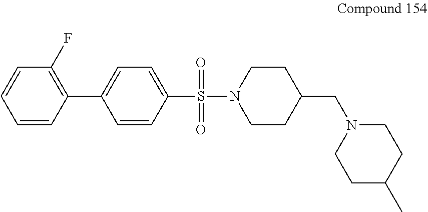 [SMILES: CC1CCN(CC2CCN(S(=O)(=O)c3ccc(-c4ccccc4F)cc3)CC2)CC1]

Compound 154: 1-((2′-fluoro-[1,1′-biphenyl]-4-yl)sulfonyl)-4-((4-methylpiperidin-1-yl)methyl)piperidine. Reaction of 1-((4-bromophenyl)sulfonyl)-4-((4-methylpiperidin-1-yl)methyl)piperidine with (2-fluorophenyl)boronic acid (Procedure G) yielded the entitled compound as a yellow solid (16%); mp 136-139° C. ¹H NMR (400 MHz, CDCl₃) δ 7.79 (d, J=8.4 Hz, 2H), 7.68 (dd, J=8.5, 1.6 Hz, 2H), 7.43 (td, J=7.7, 1.9 Hz, 1H), 7.40-7.32 (m, 1H), 7.27-7.22 (m, 1H), 7.22-7.13 (m, 1H), 3.80 (d, J=11.8 Hz, 2H), 2.80 (s, 2H), 2.30 (td, J=12.0, 2.5 Hz, 2H), 2.18 (s, 1H), 1.84 (d, J=13.1 Hz, 3H), 1.56 (d, J=10.3 Hz, 3H), 1.40-1.23 (m, 4H), 0.88 (d, J=5.4 Hz, 3H). ¹³C NMR (100 MHz, CDCl₃) δ 159.8 (d, J=249.1 Hz), 137.8 (d, J=519.5 Hz), 132.2 (d, J=9.8 Hz), 130.8 (d, J=3.0 Hz), 130.3 (d, J=8.3 Hz), 129.6 (d, J=3.2 Hz), 128.6 (d, J=12.1 Hz), 127.9, 127.4 (d, J=13.0 Hz), 124.8 (d, J=3.8 Hz), 116.5 (d, J=22.5 Hz), 64.4, 54.6, 46.5, 33.9, 32.9, 30.7, 30.5, 21.8. HRMS calcd for: C₂₄H₃₂FN₂O₂S (MH⁺) 431.2163, found 431.2160.

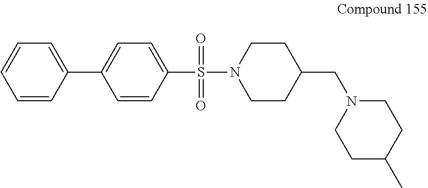 [SMILES: CC1CCN(CC2CCN(S(=O)(=O)c3ccc(-c4ccccc4)cc3)CC2)CC1]

Compound 155: 1-([1,1′-biphenyl]-4-ylsulfonyl)-4-((4-methylpiperidin-1-yl)methyl)piperidine. Reaction of [1,1′-biphenyl]-4-sulfonyl chloride with 4-methyl-1-(piperidin-4-ylmethyl)piperidine dihydrochloride (Procedure E) yielded the entitled compound as a yellow solid (58%); mp 148-151° C. ¹H NMR (400 MHz, CDCl₃) δ 7.80 (d, J=8.4 Hz, 2H), 7.70 (d, J=8.4 Hz, 2H), 7.59 (d, J=7.0 Hz, 2H), 7.46 (t, J=7.4 Hz, 2H), 7.43-7.37 (m, 1H), 3.80 (d, J=12.0 Hz, 2H), 2.72 (d, J=10.9 Hz, 2H), 2.28 (td, J=11.9, 2.6 Hz, 2H), 2.09 (d, J=6.9 Hz, 2H), 1.80 (d, J=10.2 Hz, 4H), 1.52 (d, J=14.9 Hz, 2H), 1.47-1.34 (m, 1H), 1.34-1.24 (m, 3H), 1.24-1.06 (m, 2H), 0.86 (d, J=6.2 Hz, 3H). ¹³C NMR (100 MHz, CDCl₃) δ 145.4, 139.3, 134.7, 129.0, 128.4, 128.2, 127.5, 127.3, 64.5, 54.5, 46.4, 34.2, 32.9, 30.8, 30.2, 21.9. HRMS calcd for: C₂₄H₃₃N₂O₂S (MH⁺): 413.2257, found 413.2252.

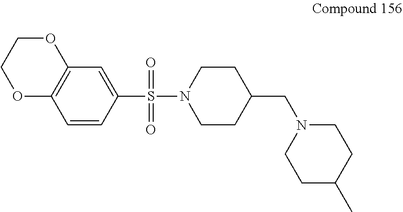 [SMILES: CC1CCN(CC2CCN(S(=O)(=O)c3ccc4c(c3)OCCO4)CC2)CC1]

Compound 156: 1-((2,3-dihydrobenzo[b][1,4]dioxin-6-yl)sulfonyl)-4-((4-methylpiperidin-1-yl)methyl)piperidine. Reaction of 2,3-dihydrobenzo[b][1,4]dioxine-6-sulfonyl chloride with 4-methyl-1-(piperidin-4-ylmethyl)piperidine dihydrochloride (Procedure E) yielded the entitled compound as a yellow gel (41%); was obtained as a yellow gel (41% yield); ¹H NMR (400 MHz, CDCl₃) δ 7.24-7.15 (m, 2H), 6.92 (d, J=8.4 Hz, 1H), 4.27 (q, J=5.2 Hz, 4H), 3.69 (d, J=12.1 Hz, 2H), 2.74 (d, J=11.6 Hz, 2H), 2.20 (td, J=12.0, 2.5 Hz, 2H), 2.11 (d, J=6.9 Hz, 2H), 1.87 (t, J=11.7 Hz, 2H), 1.77 (dd, J=13.6, 3.7 Hz, 2H), 1.53 (d, J=11.7 Hz, 2H), 1.49-1.35 (m, 1H), 1.34-1.11 (m, 5H), 0.85 (d, J=6.1 Hz, 3H). ¹³C NMR (100 MHz, CDCl₃) δ 147.3, 143.4, 128.4, 121.3, 117.5, 117.2, 64.5, 64.3, 64.1, 54.4, 46.4, 33.9, 32.8, 30.6, 30.2, 21.8. HRMS calcd for: C₂₀H₃₁N₂O₄S (MH⁺): 395.1999, found 395.1990.

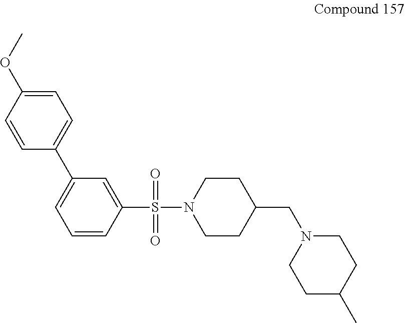 [SMILES: COc1ccc(-c2cccc(S(=O)(=O)N3CCC(CN4CCC(C)CC4)CC3)c2)cc1]

Compound 157: 1-((4′-methoxy-[1,1′-biphenyl]-3-yl)sulfonyl)-4-((4-methylpiperidin-1-yl)methyl)piperidine. Reaction of 1-((3-bromophenyl)sulfonyl)-4-((4-methylpiperidin-1-yl)methyl)piperidine with (4-methoxyphenyl)boronic acid (Procedure G) yielded the entitled compound as a light green gel (27%); ¹H NMR (400 MHz, CDCl₃) δ 7.88 (t, J=1.8 Hz, 1H), 7.74 (dt, J=7.7, 1.4 Hz, 1H), 7.64 (dt, J=7.9, 1.4 Hz, 1H), 7.58-7.48 (m, 3H), 6.98 (d, J=8.7 Hz, 2H), 3.84 (s, 3H), 3.78 (d, J=11.7 Hz, 2H), 2.75 (d, J=11.6 Hz, 2H), 2.25 (td, J=11.9, 2.5 Hz, 2H), 2.13 (d, J=6.8 Hz, 2H), 1.92-1.73 (m, 4H), 1.53 (d, J=11.9 Hz, 2H), 1.48-1.37 (m, 1H), 1.37-1.13 (m, 5H), 0.86 (d, J=6.2 Hz, 3H). ¹³C NMR (100 MHz, CDCl₃) δ 159.8, 141.8, 136.5, 131.7, 130.7, 129.3, 128.3, 125.6, 125.5, 114.5, 64.3, 55.4, 54.4, 46.4, 33.9, 32.8, 30.6, 30.2, 21.8. HRMS calcd for: C₂₅H₃₅N₂O₃S (MH⁺): 443.2363, found 443.2355.

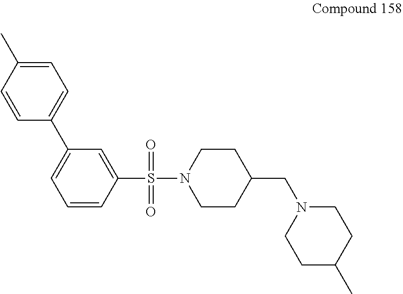 [SMILES: Cc1ccc(-c2cccc(S(=O)(=O)N3CCC(CN4CCC(C)CC4)CC3)c2)cc1]

Compound 158: 4-methyl-1-((1-((4′-methyl-[1,1′-biphenyl]-3-yl)sulfonyl)piperidin-4-yl)methyl)piperidine. Reaction of 1-((3-bromophenyl)sulfonyl)-4-((4-methylpiperidin-1-yl)methyl)piperidine with p-tolylboronic acid (Procedure G) yielded the entitled compound as a light yellow gel (41%); ¹H NMR (400 MHz, CDCl₃) δ 7.92 (s, 1H), 7.77 (d, J=7.9 Hz, 1H), 7.67 (d, J=8.1 Hz, 1H), 7.56 (t, J=7.8 Hz, 1H), 7.49 (d, J=7.9 Hz, 2H), 7.30-7.23 (m, 2H), 3.78 (d, J=11.8 Hz, 2H), 2.75 (d, J=7.7 Hz, 2H), 2.39 (s, 3H), 2.24 (td, J=11.9, 2.5 Hz, 2H), 2.13 (d, J=6.8 Hz, 2H), 1.95-1.75 (m, 4H), 1.58-1.37 (m, 4H), 1.37-1.12 (m, 5H), 0.85 (d, J=6.0 Hz, 3H). ¹³C NMR (100 MHz, CDCl₃) δ 142.1, 138.2, 136.5, 136.4, 131.0, 129.8, 129.4, 127.0, 126.0, 125.8, 64.2, 54.4, 46.4, 33.8, 32.6, 30.5, 30.2, 21.7, 21.2. HRMS calcd for: C₂₅H₃₅N₂O₂S (MH⁺): 427.2414, found 427.2410.

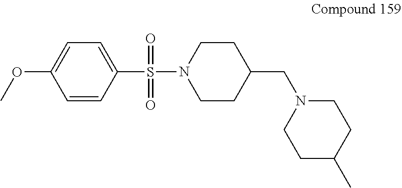 [SMILES: COc1ccc(S(=O)(=O)N2CCC(CN3CCC(C)CC3)CC2)cc1]

Compound 159: 1-((4-methoxyphenyl)sulfonyl)-4-((4-methylpiperidin-1-yl)methyl)piperidine. Reaction of 4-methoxybenzene-1-sulfonyl chloride with 4-methyl-1-(piperidin-4-ylmethyl)piperidine dihydrochloride (Procedure E) yielded the entitled compound as a brown gel (58%); ¹H NMR (400 MHz, CDCl₃) δ 7.66 (d, J=9.0 Hz, 2H), 6.96 (d, J=8.8 Hz, 2H), 3.84 (s, 3H), 3.72 (d, J=11.7 Hz, 2H), 2.73 (d, J=11.0 Hz, 2H), 2.19 (td, J=11.9, 2.6 Hz, 2H), 2.10 (d, J=7.4 Hz, 2H), 1.92-1.60 (m, 4H), 1.53 (d, J=13.5 Hz, 2H), 1.40 (s, 1H), 1.33-1.05 (m, 5H), 0.86 (d, J=6.3 Hz, 3H). ¹³C NMR (100 MHz, CDCl₃) δ 162.8, 129.7, 127.6, 114.1, 64.3, 55.6, 54.4, 46.3, 34.0, 32.8, 30.6, 30.2, 21.8. HRMS calcd for: C₁₉H₃₁N₂O₃S (MH⁺): 367.2050. found 367.2058.

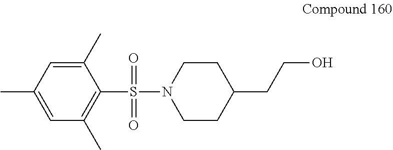 [SMILES: Cc1cc(C)c(S(=O)(=O)N2CCC(CCO)CC2)c(C)c1]

Compound 160: 2-(1-(mesitylsulfonyl)piperidin-4-yl)ethanol. To a mixture of piperidin-4-ylethanol (0.5409 g, 4.19 mmol), N, N-diisopropyl ethylamine (1.0 mL, 6.06 mmol), and CH₂Cl₂ (6 mL) was dropwise added 2,4,6-trimethylbenzene-1-sulfonyl chloride (0.7880 g, 3.60 mmol). The reaction solution was stirred at room temperature for 23 h. The reaction was quenched by saturated NaHCO₃ solution (30 mL). The resulting solution was extracted with CH₂Cl₂ (3×30 mL) and the combined organic layers were dried over Na₂SO₄, filtered and concentrated under reduced pressure. The residue was purified through flash chromatography on silica gel (1:19 MeOH/CH₂Cl₂) to afford the title product as a yellow gel (0.8803 g, 78% yield); ¹H NMR (400 MHz, CDCl₃) δ 6.89 (s, 2H), 3.60 (t, J=6.4 Hz, 2H), 3.50 (d, J=12.1 Hz, 2H), 2.68 (td, J=12.3, 2.6 Hz, 2H), 2.56 (s, 6H), 2.25 (s, 3H), 1.68 (d, J=12.6 Hz, 2H), 1.59-1.36 (m, 3H), 1.25-1.04 (m, 2H). ¹³C NMR (100 MHz, CDCl₃) δ 142.4, 140.3, 131.8, 131.7, 59.9, 44.3, 38.8, 32.1, 31.5, 22.8, 20.9. HRMS calcd for: C₁₆H₂₅NO₃SNa (MNa⁺): 334.1447 found 334.1445.

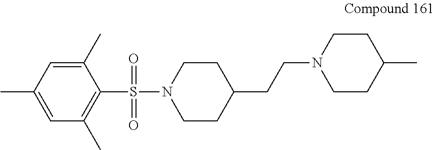

Compound 161: 1-(mesitylsulfonyl)-4-(2-(4-methylpiperidin-1-yl)ethyl)piperidine. A 25 mL flame-dried flask equipped with a magnetic stirring bar was purged by argon and was then sealed with a rubber septum fitted with an argon balloon. Anhydrous CH₂Cl₂ (4 mL) and oxalyl chloride (0.76 ML, 2 M in CH₂Cl₂, 1.52 mmol) were added via syringe sequentially. The resulting solution was cooled to −78° C. in a dry ice-acetone bath. Anhydrous DMSO (0.22 mL, 3.10 mmol) was introduced and the solution was stirred for 30 min at −78° C. (1-(mesitylsulfonyl)piperidin-4-yl)ethanol (0.3844 g, 1.24 mmol) in 2 mL anhydrous CH₂Cl₂ was added dropwise. After the reaction mixture was stirred at −78° C. for 2 h, Et₃N (0.65 mL, 4.67 mmol) was added. The reaction solution was then allowed to warm up to room temperature and stirred for 2 h before being quenched by 25 mL saturated NaHCO₃ solution and the resulting solution was exacted with CH₂Cl₂ (3×25 mL). The combined organic layers were dried by Na₂SO₄, filtered and concentrated under reduced pressure to afford the corresponding aldehyde intermediate.

A mixture of 4-methylpiperidine (0.15 mL, 1.27 mmol), the aldehyde intermediate above, DCE (7 mL), AcOH (0.40 mL, 6.99 mmol) and MgSO₄ (0.4258 g, 3.55 mmol) was stirred at room temperature for 20 min, followed by addition of NaBH(OAc)₃ (0.4254 g, 2.01 mmol). The resulting suspension was stirred at room temperature for 24 h. The reaction was then quenched by saturated NaHCO₃ solution (30 mL) at 0° C. and extracted with CH₂Cl₂ (3×30 mL). The combined organic layers were dried by Na₂SO₄, filtered and concentrated under reduced pressure. The residue was purified through flash chromatography on silica gel (1:19 MeOH/CH₂Cl₂) to afford the title product as a yellow solid (0.1405 g, 29% yield over two steps); ¹H NMR (400 MHz, CDCl₃) δ 6.88 (s, 2H), 3.48 (d, J=12.6 Hz, 2H), 3.01 (d, J=11.0 Hz, 2H), 2.66 (td, J=12.3, 2.5 Hz, 2H), 2.54 (s, 6H), 2.48 (t, J=8.2 Hz, 2H), 2.23 (s, 3H), 2.11 (t, J=11.0 Hz, 2H), 1.72-1.58 (m, 4H), 1.54 (q, J=7.0 Hz, 2H), 1.49-1.29 (m, 4H), 1.25-1.07 (m, 2H), 0.88 (d, J=4.9 Hz, 3H). ¹³C NMR (100 MHz, CDCl₃) δ 142.4, 140.3, 131.8, 131.7, 55.8, 53.5, 44.2, 34.0, 32.8, 32.0, 31.4, 30.1, 22.8, 21.4, 20.9. HRMS calcd for: C₂₂H₃₇N₂O₂S (MH): 393.2570. found 393.2570.

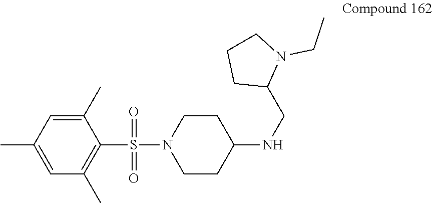 [SMILES: CCN1CCCC1CNC1CCN(S(=O)(=O)c2c(C)cc(C)cc2C)CC1]

Compound 162: N-((1-ethylpyrrolidin-2-yl)methyl)-1-(mesitylsulfonyl)piperidin-4-amine. Reaction of 1-(mesitylsulfonyl)piperidin-4-one with (1-ethylpyrrolidin-2-yl)methanamine (Procedure F) yielded the entitled compound as a colorless gel (74%); ¹H NMR (400 MHz, CDCl₃) δ 6.88 (s, 2H), 3.47 (d, J=11.9 Hz, 2H), 3.08 (t, J=6.1 Hz, 1H), 2.87-2.68 (m, 3H), 2.63 (dd, J=11.1, 3.6 Hz, 1H), 2.56 (s, 6H), 2.53-2.45 (m, 2H), 2.39 (s, 1H), 2.23 (d, J=2.6 Hz, 3H), 2.20-2.01 (m, 2H), 1.82 (s, 3H), 1.72-1.61 (m, 2H), 1.60-1.52 (m, 1H), 1.30 (s, 3H), 1.02 (t, J=7.2 Hz, 3H). ¹³C NMR (100 MHz, CDCl₃) δ 142.3, 140.4, 131.8, 131.7, 64.1, 54.8, 53.7, 50.3, 48.9, 42.9, 31.9, 31.9, 29.1, 22.7, 20.9, 13.9. HRMS calcd for: C₂₁H₃₆N₃O₂S (MH⁺): 394.2523, found 394.2513.

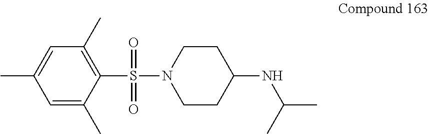 [SMILES: Cc1cc(C)c(S(=O)(=O)N2CCC(NC(C)C)CC2)c(C)c1]

Compound 163: N-isopropyl-1-(mesitylsulfonyl)piperidin-4-amine. Reaction of 1-(mesitylsulfonyl)piperidin-4-one with isopropylamine (Procedure F) yielded the entitled compound as a white foam (74%); ¹H NMR (400 MHz, acetone-d₆) δ 7.04 (s, 2H), 3.49 (dt, J=12.4, 3.4 Hz, 2H), 2.92 (hept, J=6.2 Hz, 1H), 2.82 (dt, J=12.4, 2.8 Hz, 2H), 2.72 (ddd, J=13.6, 9.8, 4.0 Hz, 1H), 2.59 (s, 6H), 2.44 (br s, 1H), 2.29 (s, 3H), 1.89-1.84 (m, 2H), 1.25 (dddd, J=13.4, 10.9, 9.8, 4.0 Hz, 2H), 0.98 (d, J=6.2 Hz, 6H). ¹³C NMR (101 MHz, acetone-d₆) δ 143.3, 141.0, 133.4, 132.6, 51.5, 45.2, 43.8, 33.1, 23.7, 22.9, 20.9. LC-MS (ESI) calcd for: C₁₇H₂₉N₂O₂S (MH⁺): 325.2; found 325.2.

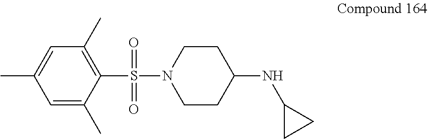 [SMILES: Cc1cc(C)c(S(=O)(=O)N2CCC(NC3CC3)CC2)c(C)c1]

Compound 164: N-cyclopropyl-1-(mesitylsulfonyl)piperidin-4-amine. Reaction of 1-(mesitylsulfonyl)piperidin-4-one with cyclopropylamine (Procedure F) yielded the entitled compound as a white foam (96%); ¹H NMR (400 MHz, acetone-d₆) δ 7.04 (s, 2H), 3.54-3.39 (m, 2H), 2.84 (ddd, J=12.2, 10.8, 2.9 Hz, 2H), 2.78 (br s, 1H), 2.73 (tt, J=9.7, 3.9 Hz, 2H), 2.59 (s, 6H), 2.29 (s, 3H), 2.09 (tt, J=6.7, 3.6 Hz, 1H), 1.97-1.88 (m, 2H), 1.33 (dddd, J=13.3, 10.6, 9.6, 3.9 Hz, 2H), 0.37 (dd, J=6.6, 4.4 Hz, 1H), 0.35 (dd, J=6.2, 3.8 Hz, 1H), 0.21 (dd, J=6.1, 3.8 Hz, 1H), 0.20 (dd, J=6.6, 4.2 Hz, 1H). ¹³C NMR (101 MHz, acetone-d) δ 143.3, 141.0, 133.4, 132.6, 55.2, 43.6, 32.7, 28.6, 22.9, 20.9, 6.8. HRMS (ESI) calcd for: C₁₇H₂₆N₂O₂S (MH⁺): 323.1780, found: 323.1788.

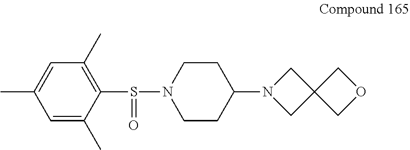 [SMILES: Cc1cc(C)c(S(=O)N2CCC(N3CC4(COC4)C3)CC2)c(C)c1]

Compound 165: 6-(1-(mesitylsulfonyl)piperidin-4-yl)-2-oxa-6-azaspiro[3.3]heptane. Reaction of 1-(mesitylsulfonyl)piperidin-4-one with 2-oxa-6-azaspiro[3.3]-heptane oxalate (Procedure F) yielded the entitled compound as a white foam (35%); ¹H NMR (400 MHz, acetone-d₆) δ 7.04 (s, 2H), 4.59 (s, 4H), 3.36 (ddd, J=11.1, 6.4, 4.2 Hz, 2H), 3.26 (s, 4H), 2.87 (ddd, J=12.4, 9.1, 3.4 Hz, 2H), 2.58 (s, 6H), 2.29 (s, 3H), 2.12 (tt, J=8.1, 3.8 Hz, 1H), 1.65 (dddd, J=13.0, 6.3, 3.3, 3.3 Hz, 2H), 1.28 (m, 2H). ¹³C NMR (101 MHz, acetone-d6) δ 143.3, 141.0, 133.3, 132.7, 81.2, 63.1, 62.1, 42.5, 39.1, 28.6, 22.9, 20.9. HRMS (ESI) calcd for: C₁₉H₂₈N₂O₃S (MH⁺): 365.1894, found: 365.1893.

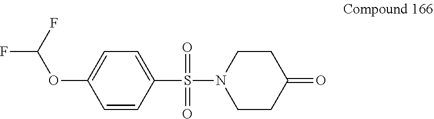 [SMILES: O=C1CCN(S(=O)(=O)c2ccc(OC(F)F)cc2)CC1]

Compound 166: 1-((4-(difluoromethoxy)phenyl)sulfonyl)piperidin-4-one. Reaction of 4-(difluoromethoxy)benzene-1-sulfonyl chloride with 4-piperidone monohydrate hydrochloride (Procedure E) yielded the entitled compound as a white solid (86%); ¹H NMR (400 MHz, CDCl₃) δ 7.78 (d, J=8.8 Hz, 2H), 7.25 (d, J=8.8 Hz, 2H), 6.61 (t, J=72.5 Hz, 1H), 3.37 (t, J=6.2 Hz, 4H), 2.52 (t, J=6.3 Hz, 4H). ¹³C NMR (100 MHz, CDCl₃) δ 205.2, 154.5 (t, J=2.9 Hz), 133.0, 129.6, 119.6, 115.1 (t, J=262.9 Hz), 45.8, 40.6. LC-MS (ESI) calcd for: C₁₂H₁₄F₂NO₄S (MH⁺): 306.1, found: 306.1.

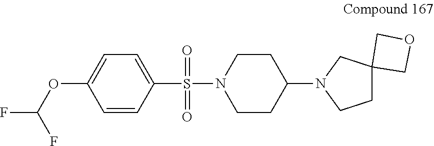 [SMILES: O=S(=O)(c1ccc(OC(F)F)cc1)N1CCC(N2CCC3(COC3)C2)CC1]

Compound 167: 6-(1-((4-(difluoromethoxy)phenyl)sulfonyl)piperidin-4-yl)-2-oxa-6-azaspiro[3.4]octane. Reaction of 1-((4-(difluoromethoxy)phenyl)sulfonyl)piperidin-4-one with 2-oxa-6-azaspiro[3.4]octane oxalic acid (Procedure F) yielded the entitled compound as a white solid (65%); ¹H NMR (400 MHz, CDCl₃) δ 7.75 (d, J=8.7 Hz, 2H), 7.23 (d, J=8.7 Hz, 2H), 6.60 (t, J=72.6 Hz, 1H), 4.54 (dd, J=15.3, 6.0 Hz, 4H), 3.61 (d, J=12.0 Hz, 2H), 2.79 (s, 2H), 2.54-2.36 (m, 4H), 2.05 (t, J=7.0 Hz, 2H), 2.03-1.91 (m, 1H), 1.86 (dd, J=13.3, 3.6 Hz, 2H), 1.66-1.51 (m, 2H). ¹³C NMR (100 MHz, CDCl₃) δ 154.1, 132.9, 129.8, 119.3, 115.1 (t, J=262.7 Hz), 83.7, 62.4, 59.9, 50.8, 44.6, 44.6, 35.8, 30.1. HRMS (ESI) calcd for: C₁₉H₂₄N₂O₄F₂S (MH⁺): 403.1498, found 403.1498.

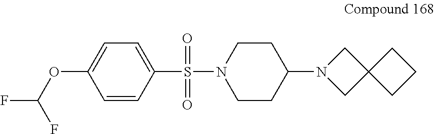 [SMILES: O=S(=O)(c1ccc(OC(F)F)cc1)N1CCC(N2CC3(CCC3)C2)CC1]

Compound 168: 2-(1-((4-(difluoromethoxy)phenyl)sulfonyl)piperidin-4-yl)-2-azaspiro[3.3]heptane. Reaction of 1-((4-(difluoromethoxy)phenyl)sulfonyl)piperidin-4-one with 2-azaspiro[3.3]heptane hemioxalate (Procedure F) yielded the entitled compound as a white solid (69%); ¹H NMR (400 MHz, CDCl₃) δ 7.75 (d, J=8.9 Hz, 2H), 7.22 (d, J=8.9 Hz, 2H), 6.60 (t, J=72.7 Hz, 1H), 3.55 (d, J=12.1 Hz, 2H), 3.05 (s, 4H), 2.46 (t, J=11.2 Hz, 2H), 2.03 (t, J=7.7 Hz, 4H), 1.93-1.82 (m, 1H), 1.81-1.71 (m, 2H), 1.64 (s, 2H), 1.42-1.25 (m, 2H). ¹³C NMR (100 MHz, CDCl₃) δ 154.06, 133.27, 129.69, 119.27, 115.17 (t, J=262.4 Hz), 64.82, 63.39, 44.28, 38.19, 32.98, 28.26, 16.63. HRMS (ESI) calcd for: C₁₈H₂₄N₂O₃F₂S (MH⁺): 387.1548, found: 387.1547.

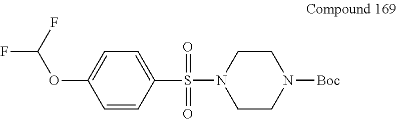 [SMILES: CC(C)(C)OC(=O)N1CCN(S(=O)(=O)c2ccc(OC(F)F)cc2)CC1]

Compound 169: tert-butyl 4-((4-(difluoromethoxy)phenyl)sulfonyl)piperazine-1-carboxylate. A mixture of tert-butyl piperazine-1-carboxylate (0.7555 g, 4.06 mmol), 4-(difluoromethoxy)benzene-1-sulfonyl chloride (1.1034 g, 4.55 mmol), N,N-diisopropyl ethylamine (1.1 mL, 6.33 mmol), and CH₂Cl₂ (12 mL) was stirred at room temperature overnight. The reaction solution was then poured into saturated NaHCO₃ solution (100 mL) and extracted with CH₂Cl₂ (3×100 mL). The combined organic layers were dried over Na₂SO₄, filtered and concentrated under reduced pressure. The residue was purified either through flash chromatography on silica gel (4:1 hexane/EtOAc) to provide the desired sulfonamide as a colorless gel (1.4070 g, 88%); ¹H NMR (400 MHz, CDCl₃) δ 7.76 (d, J=8.8 Hz, 2H), 7.26 (d, J=9.1 Hz, 2H), 6.61 (t, J=72.5 Hz, 1H), 3.50 (t, J=5.2 Hz, 4H), 2.97 (t, J=5.1 Hz, 4H), 1.40 (s, 9H). ¹³C NMR (100 MHz, CDCl₃) δ 154.4, 154.1, 132.2, 129.9, 119.5, 115.1 (t, J=263.3 Hz), 80.5, 45.8, 28.3. LC-MS (ESI) calcd for: C₁₁H₁₄F₂N₂O₃S (M−Boc+H⁺): 293.1, found: 293.1.

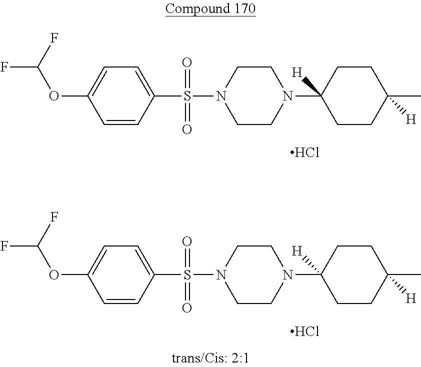 [SMILES: C[C@H]1CC[C@H](N2CCN(S(=O)(=O)c3ccc(OC(F)F)cc3)CC2)CC1 ; Cl ; C[C@H]1CC[C@@H](N2CCN(S(=O)(=O)c3ccc(OC(F)F)cc3)CC2)CC1 ; Cl]

Compound 170: 1-((4-(difluoromethoxy)phenyl)sulfonyl)-4-(4-methylcyclohexyl)piperazine hydrochloride. tert-butyl 4-((4-(difluoromethoxy)phenyl)sulfonyl)piperazine-1-carboxylate (0.2735 g, 0.70 mmol) was dissolved in anhydrous CH₂Cl₂ (6 mL), followed by addition of trifluoroacetic acid (1.5 mL, 19.59 mmol). The resulting solution was stirred at room temperature for 1.5 h and then was slowly poured into 30 mL saturated K₂CO₃ solution at 0° C. The biphasic solution was extracted with CH₂Cl₂ (3×30 mL). The combined organic layers were dried over Na₂SO₄, filtered and concentrated under reduced pressure to afford the corresponding deprotected amine which was used directly for next step without further purification.

A mixture of the deprotected amine above, 4-methylcyclohexan-1-one (0.0944 g, 0.84 mmol), DCE (6 ml), AcOH (50 μl, 0.87 mmol) was stirred at room temperature for 50 min, followed by addition of NaBH(OAc)₃ (0.2431 g, 1.15 mmol). The resulting suspension was stirred at room temperature for 24 h and was then slowly pouring into saturated NaHCO₃ solution (30 mL) and extracted with DCM (3×30 mL). The combined organic layers were dried over Na₂SO₄, filtered and concentrated under reduced pressure. The residue was purified through flash chromatography on silica gel (1:1 EtOAc/Hexane) to afford a mixture of the crude product (Rf: 0.85) mixed with some impurities. The mixture of the crude product and impurities was dissolved in Et₂O (6 mL) and conc. HCl was dropwise added. The precipitated 1-((4-(difluoromethoxy)phenyl)sulfonyl)-4-(4-methylcyclohexyl)piperazine hydrochloride salt (0.0621 g, 21%) as a mixture of trans and cis (ratio 2:1) was obtained after filtration; ¹H NMR (400 MHz, CD₃OD) δ 7.89 (d, J=8.9 Hz, 2H), 7.41 (d, J=8.9 Hz, 1H), 7.05 (t, J=72.9 Hz, 1H), 3.95 (d, J=13.1 Hz, 2H), 3.62 (t, J=16.9 Hz, 2H), 3.29-3.12 (m, 3H), 2.76 (t, J=13.4 Hz, 2H), 2.14-1.59 (m, 8H), 1.59-1.31 (m, 1H), 1.07 (dq, J=13.1, 3.0 Hz, 1H), 0.99 (d, J=7.2 Hz, 2H), 0.91 (d, J=6.5 Hz, 1H). ¹³C NMR (100 MHz, CD₃OD) δ 155.4, 131.1, 130.1, 119.0, 118.4, 115.8, 113.2, 66.0, 65.7, 43.4, 43.3, 32.8, 31.3, 29.6, 26.3, 26.3, 21.3, 20.6, 16.3. LC-MS (ESI) calcd for: C₁₈H₂₇F₂N₂O₃S (M−Cl⁺): 389.2, found: 389.2.

### Example 2

### Identification of Small Molecules that Target Truncated Apc Proteins

Mutations in the human APC tumor suppressor gene are linked to Familial Adenomatous Polyposis (FAP), an inherited cancer-prone condition in which numerous polyps are formed in the epithelium of the large intestine. The development of colorectal cancer (CRC) is initiated by the aberrant outgrowth of adenomatous polyps from the colonic epithelium that ultimately evolve into aggressive carcinomas. About 85% of sporadic colorectal cancers have been reported to harbor APC truncating mutations. The growth of the polyps is associated in most cases with alterations of both alleles of the Adenomatous Polyposis Coli (APC) gene. A first mutational hit occurs roughly in the middle of the open reading frame, generating a truncated APC molecule lacking the C-terminal half. Such truncation mutations are located in the so-called mutation cluster region (MCR). The second mutational hit involves either deletion of the second allele or a mutation that leads to the synthesis of a truncated product, almost never occurring after the MCR. Thus, colon cancer cells express at least a truncated APC molecule whose length is defined by the position of the MCR and, occasionally, an additional but shorter fragment. Adenomatous Polyposis Coli (APC) Gene. APC, which does not act as a classical tumor suppressor, influences Wnt signaling thereby regulating gene transcription. Wnts are a family of secreted cysteine-rich glycoproteins that have been implicated in the regulation of stem cell maintenance, proliferation, and differentiation during embryonic development. Canonical Wnt signaling increases the stability of cytoplasmic β-catenin by receptor-mediated inactivation of GSK-3 kinase activity and promotes β-catenin translocation into the nucleus. The canonical Wnt signaling pathway also functions as a stem cell mitogen via the stabilization of intracellular β-catenin and activation of the β-catenin/TCF/LEF transcription complex, resulting in activated expression of cell cycle regulatory genes, such as Myc, cyclin D1, EPhrinB (EPhB) and Msx1, which promote cell proliferation.

APC is the negative regulator of Wnt signaling. Without this negative regulation, the Wnt pathway is more active and is important in cancer. Studies comparing tumor cells with mutations in both APC alleles to correlate levels of Wnt signaling and severity of disease in both humans and mice have aided in establishing a model in which gene dosage effects generate a defined window of enhanced Wnt signaling, leading to polyp formation in the intestine. Combinations of ‘milder’ APC mutations, associated with weaker enhancement of Wnt signaling, give rise to tumors in extra-intestinal tissues. According to this model, the nature of the germline mutation in APC determines the type of somatic mutation that occurs in the second allele.

APC Protein. The APC gene product is a 312 kDa protein consisting of multiple domains, which bind to various proteins, including beta-catenin, axin, C-terminal binding protein (CtBP), APC-stimulated guanine nucleotide exchange factors (Asefs), Ras GTPase-activating-like protein (IQGAP1), end binding-1 (EB1) and microtubules. Studies using mutant mice and cultured cells demonstrated that APC suppresses canonical Wnt signaling, which is essential for tumorigenesis, development and homeostasis of a variety of cell types, including epithelial and lymphoid cells. Further studies have suggested that the APC protein functions in several other fundamental cellular processes. These cellular processes include cell adhesion and migration, organization of actin and microtubule networks, spindle formation and chromosome segregation. Deregulation of these processes caused by mutations in APC is implicated in the initiation and expansion of colon cancer.

The APC protein functions as a signaling hub or scaffold, in that it physically interacts with a number of proteins relevant to carcinogenesis. Loss of APC influences cell adhesion, cell migration, the cytoskeleton, and chromosome segregation.

Most investigators believe that APC mutations cause a loss of function change in colon cancer. Missense mutations yield point mutations in APC, while truncation mutations cause the loss of large portions of the APC protein, including defined regulatory domains. A significant number of APC missense mutations have been reported in tumors originating from various tissues, and have been linked to worse disease outcome in invasive urothelial carcinomas, suggesting the functional relevance of point mutated APC protein in the development of extra-intestinal tumors. The molecular basis by which these mutations interfere with the function of APC remains unresolved.

APC mutation resulting in a change of function can influence chromosome instability in at least three manners: by diminishing kinetochore-microtubule interaction, by the loss of mitotic checkpoint function and by generating polyploid cells. For example, studies have shown that APC bound to microtubules increased microtubule stability in vivo and in vitro, suggesting a role of APC in microtubule stability. Truncated APC led to chromosomal instability in mouse embryonic stem cells, interfered with microtubule plus-end attachments, and caused a dramatic increase in mitotic abnormalities. Studies have shown that cancer cells with APC mutations have a diminished capacity to correct erroneous kinetochore-microtubule attachments, which account for the wide-spread occurrence of chromosome instability in tumors. In addition, abrogation of the spindle checkpoint function was reported with APC loss of function. Knockdown of APC with siRNA indicated that loss of APC causes loss of mitotic spindle checkpoint function by reducing the association between the kinetochore and checkpoint proteins Bub1 and BubR1. Thus, loss of APC reduces apoptosis and induces polyploidy. Polyploidy is a major source for aneuploidy since it can lead to multipolar mitosis.

While loss of function due to APC may be partially correct, there are reports showing that a large fraction of colon cancer patients have at least one APC gene product that is truncated, and that this has a gain of function. Thus truncated APC proteins may play an active role in colon cancer initiation and progression as opposed to being recessive; for example, truncated APC, but not full-length APC may activate Asef and promote cell migration.

HCECs isolated from normal colonic biopsies were immortalized by successive infections of CDK4 and hTERT followed by selection with respective antibiotics-G418 (250 μg/mL) and blastocidin (2.5 μg/mL). shRNAs against p53 were introduced with retroviruses and p53 knockdown efficiency was verified by Western analysis. Human colon cancer cell lines (HCT116, DLD-1, RKO) and virus-producing cell lines (293FT, Phoenix A) were cultured in basal medium supplemented with 10% serum. The identity of all cell lines was verified by DNA fingerprinting. 1 μg of shRNA together with 1 μg of helper plasmids (0.4 μg pMD2G and 0.6 μg psPAX2) were transfected into 293FT cells with Polyjet reagent (SignaGen). Viral supernatants were collected 48 hours after transfection and cleared through a 0.45-μm filter. Cells were infected with viral supernatants containing 4 μg/mL polybrene (Sigma) at a multiplicity of infection (MOI) of approximately 1. Successfully infected cells were selected with 1 ug/mL puromycin for 3 days, as described in: Eskiocak U, Kim S B, Ly P, Roig A I, Biglione S, Komurov K, Cornelius C, Wright W E, White M A, Shay J W. Functional parsing of driver mutations in the colorectal cancer genome reveals numerous suppressors of anchorage-independent growth. Cancer Res 2011; 71:4359-65, which is incorporated herein by reference.

Mitotic Index. For determination of the mitotic index, DLD1 cells were methanol fixed 24 h after treatment with TASIN-1 at a concentration of 2.5 L or 10 L or Pitstop2 (abcam, ab120687) at a concentration of 10 μL, DNA was visualized by Hoechst 33342 staining, and cells were imaged on a microscope (Axiovert 200M; Carl Zeiss) using a LD 40×/NA 0.75/Ph2 Plan-Neofluor objective. Mitotic cells were identified in the UV channel by their condensed DNA content.

HCECs with TP53, APC knockdown, KRASV12 mutation (1CTRPA) together with ectopic expression of APC truncation 1309 (hereinafter “1CTRPA A1309”) (Table 2) have been developed. This APC mutation is strongly selected for in colon cancers and has been shown to be more resistant to caspase cleavage than other truncated forms of APC. APC-truncated HCEC cell line 1CTRPA A1309 exhibits an increase in growth rate, enhancement of soft agar growth and invasion through Matrigel® compared to matched parental HCECs (1CTRPA). However, knockdown of wt APC alone (1CTRPA) did not cause HCECs to gain oncogenic properties.

These isogenic cell lines with defined genetic alterations have been used as a cellular model for identification of small molecules that target truncated APC proteins.

TABLE 2
Summary of the isogenic human colonic epithelial
cells (HCECs) used in this screen.

| Cell Lines | Genetic Alterations |
|---|---|
| 1CT | HCECs immortalized with CDK4 and hTERT |
| 1CTRPA | Kras^{v12}, shTP53, shAPC |
| 1CTRPA A1309 | Kras^{v12}, shTP53, shAPC, APC mutation (aa 1-1309) |

C: CDK4;
T: hTERT;
R: Kras^{v12};
P: shTP53:
A: shAPC

Isogenic cell lines were used to carry out a cell-based high-throughput screen designed to identify small molecules and/or natural product fractions from within the University of Texas Southwestern (UTSW) compound file that can selectively inhibit cell growth of APC-truncated HCECs. This compound library encompasses ˜200,000 synthetic compounds that represent a large chemical space from several commercial vendors, including 1200 marketed drugs from the Prestwick Chemical Library®, and 600 compounds that went to pre-clinical tests from the NIH library. The isogenic cell lines used in the screen are listed in Table 2.

A primary screen was performed in 1CTRPA A1309. For the screen, cells were seeded as a monolayer at a density of 400 cells/well in 384 well plates [in Colonic Epithelial Cell Medium (CoEpiCM (ScienCell Research Laboratories; Innoprot, etc.)], which are commercially available (Invitrogen; BioRad; Corning etc.). Twenty four hours later candidate compounds were added at a concentration of 2.5 μM per well and cells were incubated for 4 days at physiologic oxygen conditions (˜3-5% 02). A luminescence-based Celltiter-Glo® assay was performed to measure cell viability, using ATP levels as the readout. In brief, opaque-walled multiwell plates with mammalian cells in culture medium (25 μl per well, 384-well plates) were prepared. Control wells containing medium without cells were prepared to obtain a value for background luminescence. Test compounds were added to experimental wells, and incubated according to culture protocol. The plate and its contents were incubated at room temperature for approximately 30 minutes. An ATP standard curve was generated immediately prior to adding the CellTiter-Glo® Reagent. A volume of CellTiter-Glo® Reagent equal to the volume of cell culture medium present in each well (25 μl of reagent to 25 μl of medium containing cells for a 384-well plate) was added. The contents were mixed for 2 minutes on an orbital shaker to induce cell lysis. The plate was allowed to incubate at room temperature for 10 minutes to stabilize the luminescent signal and luminescence recorded. (e.g. GloMax®, Lumistar, SPECTROstar, PHERAstar FS). The primary screen yielded 6704 positive hits (based on a z-score of <−3, which means that the z-score of −3 was 3 standard deviations below the mean).

Compounds that inhibited >40% of the proliferation of normal human epithelial cells were excluded based on the screening facility database and previous experience. The remaining 5381 compounds were re-screened against 1CTRPA A1309 (to validate the primary screen results) and 1CTRPA (to exclude those compounds that are not specific to APC truncations). To eliminate the possible general toxicity properties of these compounds, the compounds were also counter screened against normal diploid HCECs (1CT). This counter screen identified 126 compounds that inhibit cell growth of CTRPA A1309 >50% more than that of 1CTRPA and 1CT. An additional screen of these selectively toxic compounds was carried out against the same panel of HCECs at a 1:3 fold dilution series of concentrations, ranging from 2.5 um to 30 nm. This secondary counter screen yielded 14 candidate compounds that showed selective inhibition of 1CTRPA A1309 cells at concentrations of 30 nm or 90 nm but without noticeable impact on 1CTRPA or 1CT cells.

The overall screening strategy is shown in the flow chart below:

These 14 compounds then were obtained commercially and their IC₅₀ determined by performing dose response studies with half log dilution series at 12 concentration points in two authentic CRC lines: HCT116 (wt APC) and DLD1 (truncated APC). Anti-cancer compounds A and B showed selective toxicity towards DLD1 with IC₅₀ 63 nm and 131 nm, respectively. These two compounds served as initial lead compounds for analog development and for additional studies.

In Vitro APC Inhibition Assay. The small molecule anti-cancer compounds were evaluated for the ability to inhibit the activity of APC in an in vitro assay. Reagents required for the assay are: (3-[4,5-Dimethylthiazol-2-yl]-2,5-diphenyltetrazolium bromide; Thiazolyl blue; RPI corp., cat #M92050); RPMI-1640 or Medium of Choice (i.e. DMEM) without phenol red; 1M Hepes; 100 mM NaPyruvate; 1000× Gentamicin (50 mg/ml); 100× Penicillin/Streptomycin/Fungizone; 1×PBS; Triton X-100; 1N HCl and Isopropanol. To make the MTT solution (10×), MTT powder was dissolved into complete RPMI (or DMEM solution) to a final concentration of 5 mg/mL and was sterilized by filtration with a 0.2 μm filter. The MTT solubilization solution contained 10% Triton X-100, 0.1N and 80% isopropanol. MTT solution was diluted to 1× with complete medium at 12 mL per plate. The culture dishes (96 wells) were removed from the incubator and the media was discarded. The plates were washed three times with 1×PBS. 100 μl of 1×MTT solution was added to each well and the plates were incubated in a tissue culture incubator for 2-4 hours, depending on the cell line. The cells were removed from the incubator and the MTT solution was discarded. 200 μl of 1×MTT solubilization solution was added to each well using a multi-channel pipetor and the cells were placed on an orbital shaker for 10 minutes. The results were read on a microtiter plate reader (absorbance=570 nm, reference=700 nm) and data was exported.

Percent inhibition was determined relative to control reactions without inhibitor, and half maximal inhibitory concentration (IC₅₀) values were determined using a standard four parameter fit to the inhibition data. The term “IC₅₀” as used herein refers to a quantitative measure of the effectiveness of a compound in inhibiting biological or biochemical function that indicates the amount that is needed to inhibit a given biological process (or component of a process) by 50%.

Truncated APC Selective Inhibitor-1 (TASIN-1/compound 6) kills CRC lines with APC truncations while sparing normal human colonic epithelial cells (HCECs) and other cancer cells with wild type (WT) APC, interferes with proper mitotic spindle formation, and induces JNK-dependent apoptotic cell death in APC truncated cells.

Dose response analysis in two authentic CRC cell lines: HCT116 (WT APC) and DLD1 (truncated APC), led to identification of the lead compound TASIN-1 (compound 6, truncated APC selective inhibitor):

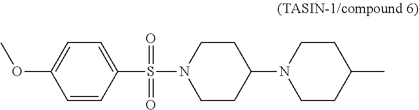

As previously reported, this compound exhibited potent and selective toxicity towards DLD1 cells (IC50=63 nM) but not towards HCT116 cells (IC50>10 μM). Sustained treatment of TASIN-1 (compound 6) inhibited soft agar growth in DLD1 but not in HCT116 cells.

In vivo antitumor activity of TASIN-1 (compound 6) in a xenograft mouse model. The in vivo antitumor activity of TASIN-1 (compound 6) was examined in a xenograft mouse model. Nude mice with established DLD1 tumors were injected intraperitoneally with either solvent (control) or 40 mg/kg of TASIN-1 twice daily for 18 days. TASIN-1 (compound 6) treatment reduced the size of tumor xenografts and reduced tumor growth rates compared with control mice. As previously reported, no overt toxicity and no statistically significant differences were observed in the body weights of mice between control group and TASIN-1 treated group. Similar antitumor activity was observed in HT29 xenografts, which also harbors truncated APC and demonstrated a similar sensitivity as DLD1 in vitro. However, TASIN-1 (compound 6) did not inhibit tumor growth in HCT116 (WT APC) xenografts, demonstrating that TASIN-1 (compound 6) maintains selectivity in vivo. Immunohistochemistry analysis of excised tumors showed that TASIN-1 (compound 6) induced a significant increase in the apoptotic marker, cleaved caspase 3 in TASIN-1 treated DLD1 tumors compared with control tumors. Induction of apoptosis was confirmed by detection of cleaved PARP1 in tumor lysates from control and TASIN-1 (compound 6) treated DLD1 xenografts (data not shown).

Anti-tumor effects of TASIN-1 in CPC;Apc mice. The antitumor effects of TASIN-1 in CPC;Apc mice, a genetically engineered mouse model that mainly develops colorectal tumors, were further tested. These mice carry a CDX2P-NLS Cre recombinase transgene and a loxP-targeted Apc allele that deletes exon 14, leading to a frame shift at codon 580 and a truncated APC protein. Mice ˜110 days old were injected intraperitoneally with either solvent or 20 mg/kg/injection of TASIN-1 twice a week for 90 days. Weights were measured every 15 days over the treatment period. These studies were performed according to the guidelines of the UT Southwestern Institutional Animal Care and Use Committee.

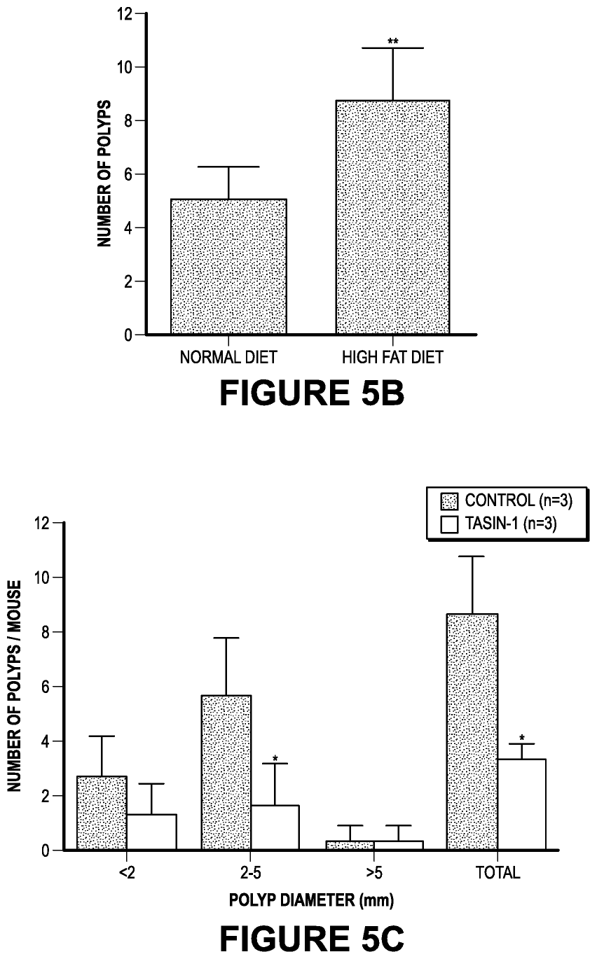

As previously reported, TASIN-1 treatment resulted in significant reduction in tumor formation in the colon of CPC;Apc mice. Benign tumors (polyps) that developed in TASIN-1 treated CPC;Apc mice were much smaller compared to the control group (FIG. 5). Additionally, TASIN-1 treated mice with less tumor burden gained weight to a level similar to WT mice over the 90 days' treatment Finally, TASIN-1 treated mice showed suppressed expression of a panel of inflammatory response genes and reduced staining for Ki67 and cyclin D1, accompanied by increased staining for cleaved caspase 3 in colon tumor sections (data not shown). Taken together, these in vivo experiments show that TASIN-1 efficiently attenuates tumorigenesis in both human xenografts and genetically engineered CRC mouse models without noticeable toxicity.

### Example 3

### TASIN-1 is Selectively Toxic to HCECS and CRC Cell Lines with APC Truncation when Tested in Physiological Levels of Serum

DLD-1 cells normally are cultured in 10% serum medium, and they are rapidly shifted to 0.2% serum medium for dose response studies. All experiments were performed in normoxic condition (21% 02).

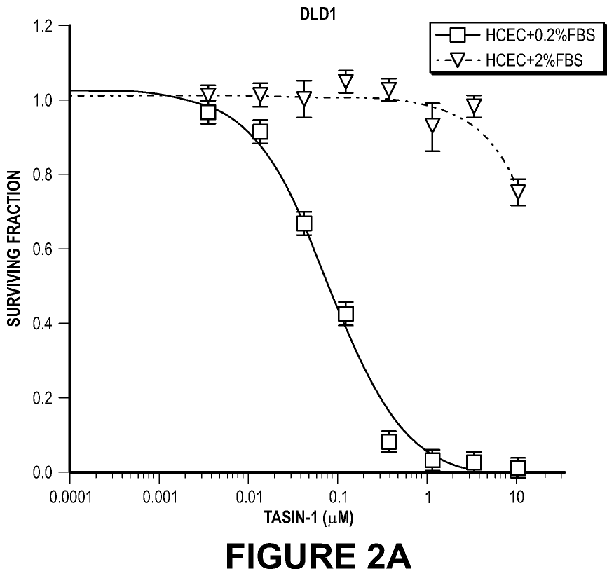

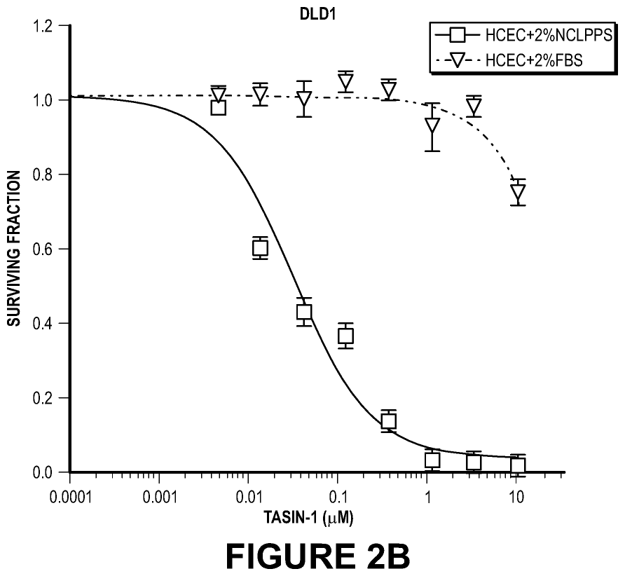

FIGS. 2A-B show that TASIN-1 is active only in 0.2% serum or 2% lipoprotein deficient serum (LPPS) media conditions. DLD-1 cells have a truncated APC and are sensitive to TASIN-1 only in low (0.2%) serum (FIG. 2A). However, if one uses 2% lipoprotein deficient newborn calf lipoprotein-poor serum (NCLPPS) (FIG. 2B), the cells remain sensitive to TASIN-1.

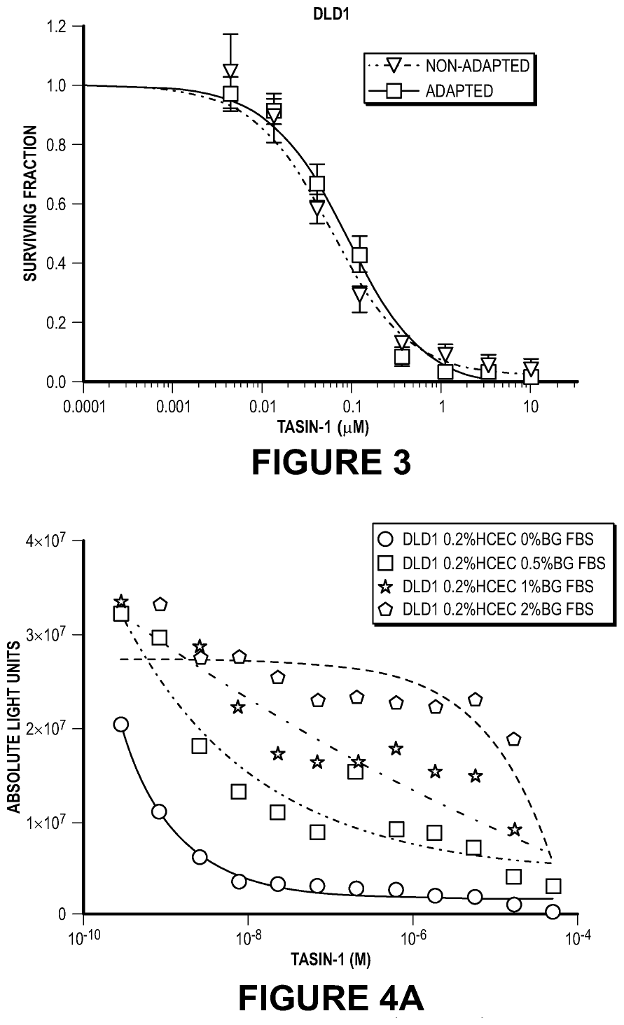

As shown in FIG. 3, the sensitivity of DLD1 cells TASIN-1 was the same if the cells were not gradually adapted to low serum but instead the serum content was rapidly changed from 10% to low serum during drug testing. Adapted DLD1 cells were adapted to medium containing 0.2% serum by gradually decreasing serum from 10%, 5%, 2% to 0.2%. Non-adapted DLD1 cells were rapidly changed from 10% serum to low serum. It was concluded that the effects of TASIN-1 are not due to rapid shifting of cells from high to low serum. It is believed that the tumor microenvironment is only exposed to very low cerum amounts.

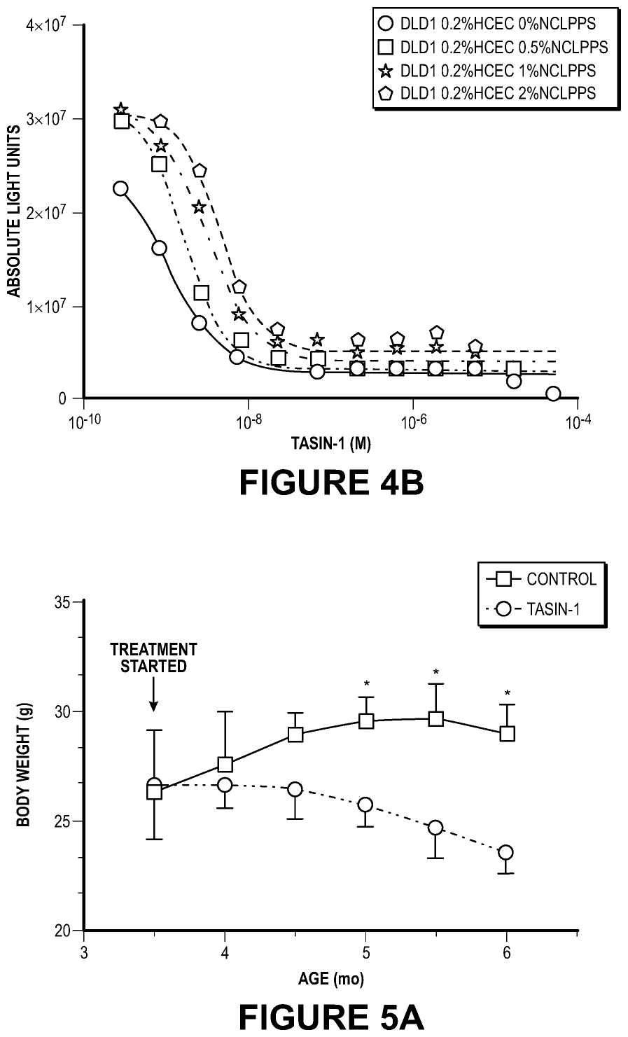

As shown in FIG. 4, TASIN-1 treated cells are rescued with FBS but not with LPPS. FIG. 4A shows that the sensitivity of DLD1 cells to TASIN-1 is gradually lost by increasing serum level using fetal bovine serum (FBS). FIG. 4B shows that increasing the amount of lipoprotein poor serum does not change the sensitivity of DLD1 cells to TASIN-1 concentration.

### Example 4

### TASIN-1 Prevents Colon Cancer Progression Under High Fat Diet Conditions

Normal diet for CPC;Apc mice is 5.7% fat, while a high fat atherogenic diet is 12.8% fat CPC;Apc mice were fed a high fat diet for about 10 weeks; half the mice received TASIN-1 (dose), the other half did not (control). As shown in FIG. 5A, the body weight for the control group decreased over time, compared to the TASIN-1 group. As shown in FIG. 5B, colon cancer progression was accelerated by the high fat diet. As shown in FIG. 5C, TASIN-1 reduced polyp formation and size, even in mice fed a high fat diet.

### Example 5

### EBP is the Direct Target of TASIN-1

Emodampil binding protein (EBP) was identified as the target of TASIN-1 using photoaffinity probes. Photoaffinity probes containing an alkyne group (to be used for click chemistry) and either benzophenone moieties or aryl azides, which in response to UV light have the potential to form a covalent bond with the protein target.

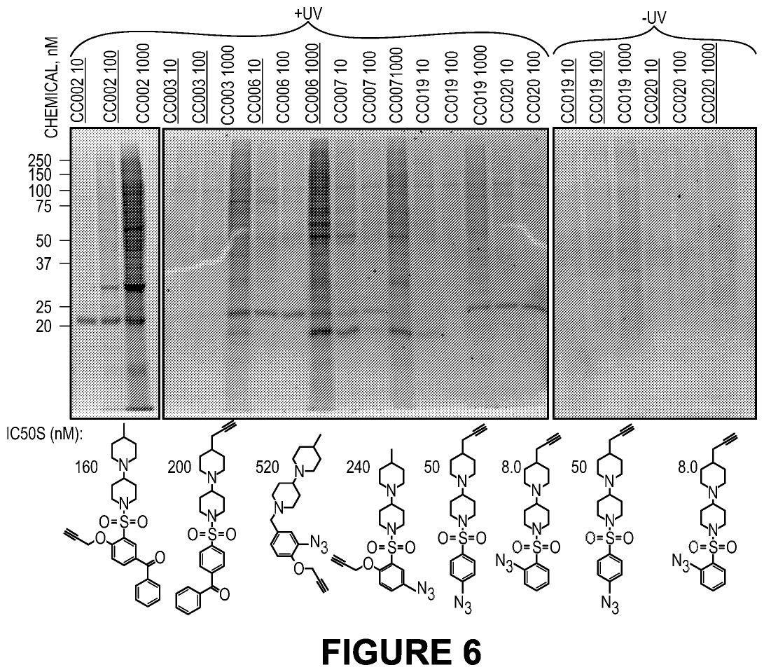

DLD-1 cells were incubated with a photoaffinity probe at varying concentrations and then exposed to UV light Lysate was collected from the cells, conjugated to a alexa fluor 532 dye azide using standard protocols for copper dependent click chemistry, and then analyzed by SDS-PAGE. FIG. 6 shows the SDS PAGE profile of DLD-1 cells treated with a TASIN probe as shown in the presence and absence of UV light. A 22 kD band representing a protein bound to the compound in multiple photocrosslinkers was detected. This band was dependent on the UV treatment, thus reflecting a non-covalent bond.

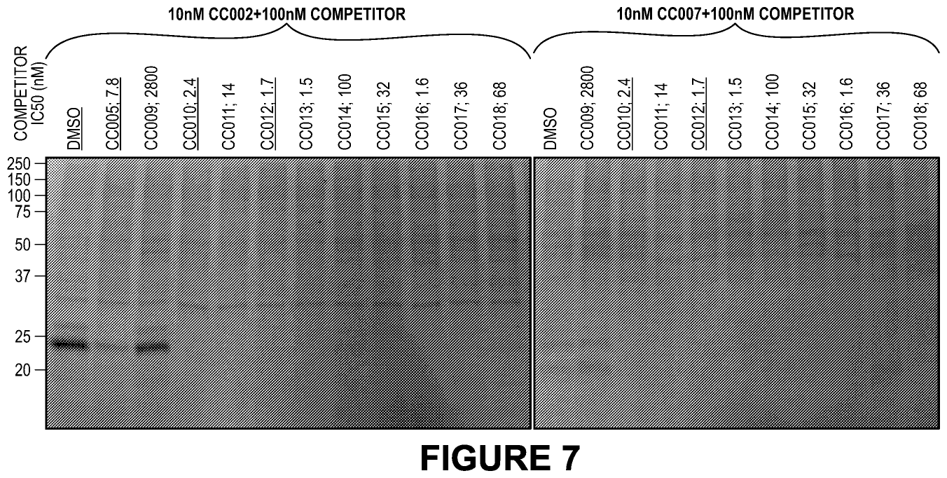

To determine whether this 22 kD band reflected the functional target of the TASIN probes, the TASIN probe (10 nM) was co-incubated with 100 nM of various competitor probes as shown. Since the amount of each competitor probe is in excess, binding of the competitor probe is detected as loss of signal or reduced intensity of the TASIN probe. FIG. 7 shows that co-incubation with active analogues of TASIN blocked probe binding whereas less active analogues did not. These results suggested that the functional activity of the compounds correlated with binding to the 22 kD protein band.

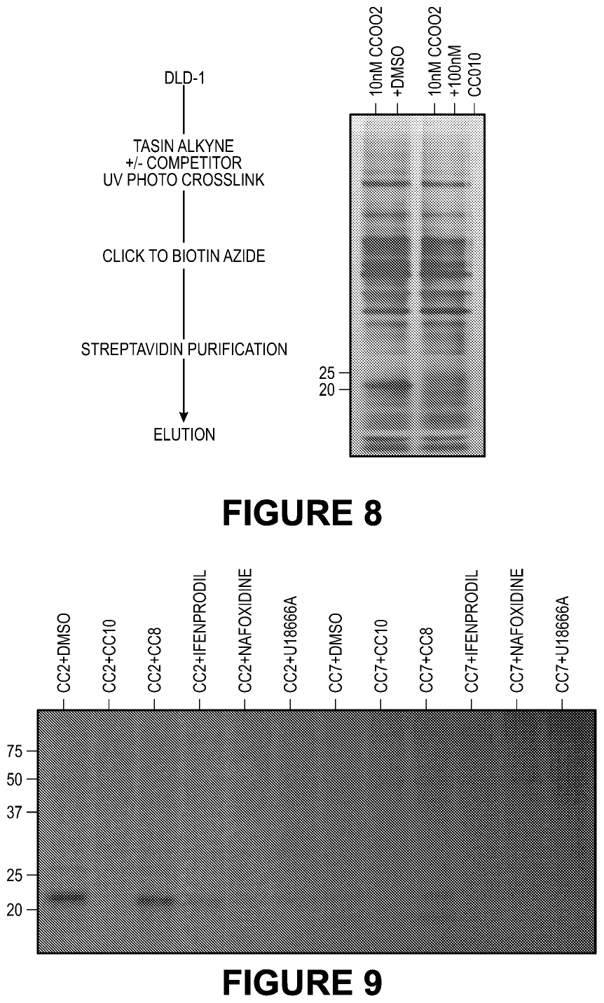

As shown in FIG. 8, lysate from DLD-1 cells treated with the TASIN probe +/− an excess amount of competitor was collected and conjugated to a biotin azide using copper dependent click chemistry. Streptavidin beads were used to purify biotin bound proteins and the resulting precipitate was analyzed by SDS-PAGE stained with silver. The results shown in the right panel revealed a 22 kD protein whose signal intensity was reduced when a competitor was added, consistent with the predicted functional target. The 22 kD band, along with the analogous region in the competitor lane was excised from the SDS-PAGE gel and submitted to trypsin digestion and peptide analysis by tandem mass spectrometry. Proteins were identified based on mapping peptides and ranked by the ratio of spectral index between the sample with competitor and the sample without competitor. Spectral index is a semi-quantitative analysis of protein abundance. Table 3 shows that the highest ranking protein (i.e., most abundant) with a ratio of 0.01 was EBP).

TABLE 3
3-beta-hydroxysteroid-delta(8), delta(7)-isomerase (EBP) is a top
hit for competition and one of the top hits for spectral intensity.

| | | Spectral Index | | |
|---|---|---|---|---|
| | | (MIC Sin) | | Ratio |
| | | CC002 + | CC002 + | +comp/ |
| Description | Mw (Da) | CC010 | DMSO | −comp |
| EBP_HUMAN 2-beta- | 26389.80 | 8.37E−06 | 5.95E−04 | 0.01 |
| hydroxysteroid-Delta(8), | | | | |
| Delta(7)-isomerase | | | | |
| OS = Homo sapiens | | | | |
| GN = EBP PE = 1 | | | | |
| SV = 3 | | | | |
| TPIS_HUMAN | 26724.80 | 1.58E−07 | 1.17E−06 | 0.14 |
| Triosephosphate isomerase | | | | |
| OS = Homo sapiens | | | | |
| GN = TPI1 | | | | |
| PE = 1, SV = 3 | | | | |
| LGUL_HUMAN | 19066.60 | 7.93E−07 | 2.99E−06 | 0.27 |
| Lactoylglutathione lyuase | | | | |
| OS = Homo sapiens | | | | |
| GN = GLO1 PE = 1 | | | | |
| SV = 4 | | | | |
| RAB8A_HUMAN Ras- | 23707.20 | 4.68E−07 | 1.7E−06 | 0.27 |
| related protein Rab-8A | | | | |
| OS = Homo sapiens | | | | |
| GN = RAB8A PE = 1 | | | | |
| SV = 1 | | | | |
| B3KSH1_HUMAN | 37630.20 | 1.36E−07 | 5.03E−07 | 0.27 |
| Eukaryotic translation | | | | |
| initiation factor 3 | | | | |
| subunit F | | | | |
| OS = Homo sapiens | | | | |
| PGN = EIF3F PE = 2 | | | | |
| SV = 1 | | | | |

To confirm that EBP was the target of TASIN, chemically distinct scaffolds that are known to bind EBP (e.g., Ifenprodil, Nafoxidine, and U18666A) were used as competitors for TASIN probes CC002 and CC007. In all three cases, these known EBP binders competed for CC002 or CC007 binding (FIG. 9). These results confirmed that the 22 kD protein band that bound to the TASIN probes is EBP.

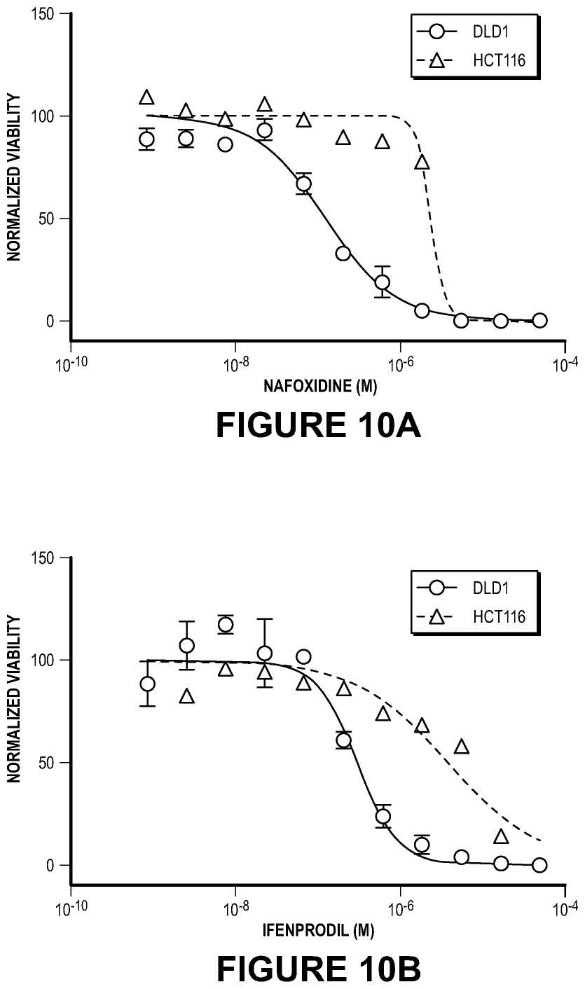

To test whether inhibitors of EBP generally confer the same selectivity to cancer cell lines that harbor APC truncation mutations, HCT-116 and DLD-1 cells were incubated with Nafoxidine and Ifenprodil at varying doses. As shown in FIGS. 10A-B, DLD-1 cells were selectively sensitive to these EBP inhibitors, just as they were to TASIN.

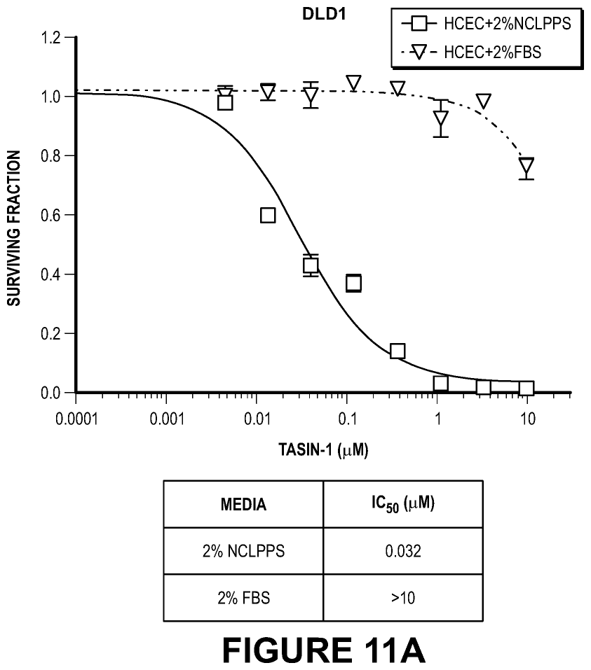

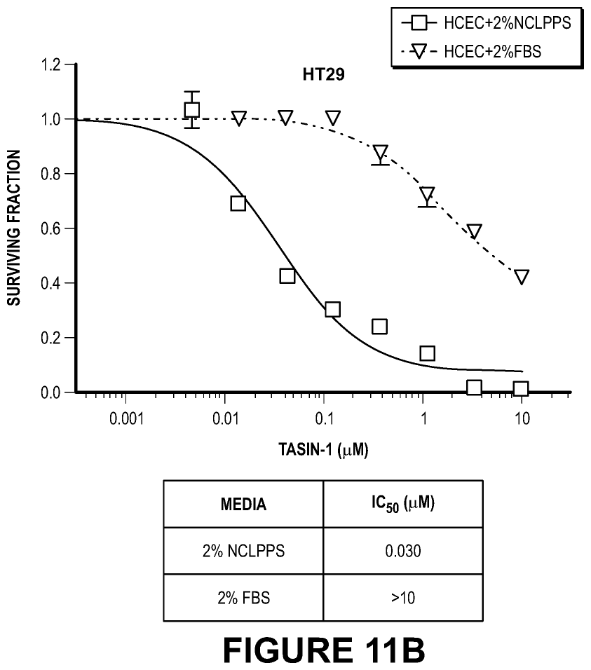

Without being bound by theory, these results suggest that DLD-1 cells may die as a result of cholesterol deficiency. To determine whether DLD-1 cells die as a result of cholesterol deficiency, DLD-1 cells were incubated with standard media containing increasing concentrations of Fetal Bovine Serum (FBS) or lipoprotein-depleted FBS. FBS is a rich source of low-density lipoprotein (LDL) receptor and cholesterol. The results of this experiment showed that FBS rescued DLD-1 cells from the toxicity of TASIN whereas lipoprotein-depleted FBS did not (FIG. 4). These results indicate that DLD-1 cells die as a result of a block in cholesterol synthesis. As shown in FIGS. 11A-B, HT29 cells, another cancer cell line with an APC truncation, are also rescued from TASIN by FBS but not lipoprotein-deficient serum.

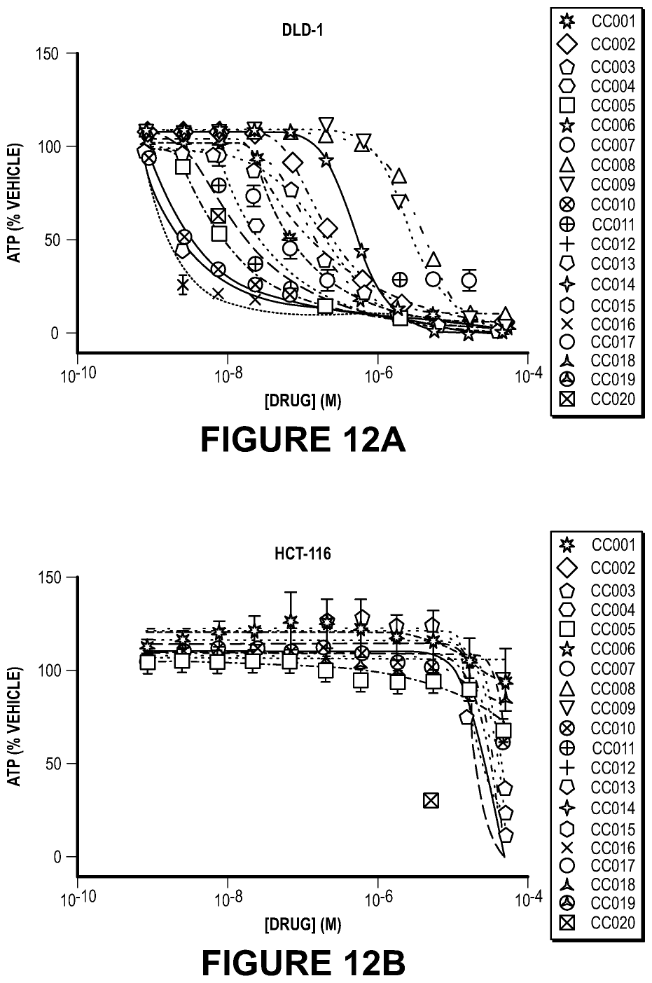

FIGS. 12A-B show that exemplary TASIN analogues are toxic and selective for DLD-1 in 0.2% HCEC medium.

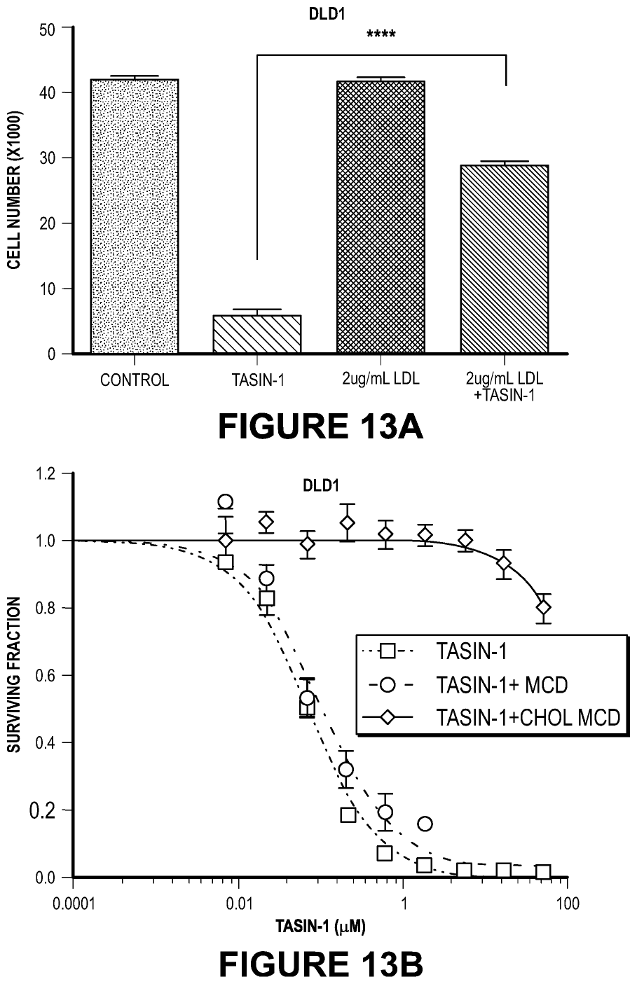

FIGS. 13A-B show that exogenous addition of LDL or cholesterol rescues TASIN-1 induced cell death. Adding purified lipoproteins to the medium (FIG. 13A) or cholesterol (FIG. 13B) decreases the sensitivity of DLD-1 cells to TASIN-1. These results implicate cholesterol biosynthesis or metabolism as being important, and help explain that TASIN-1 targeting EBP upstream of cholesterol is effective, but not if cholesterol downstream of EBP is provided.

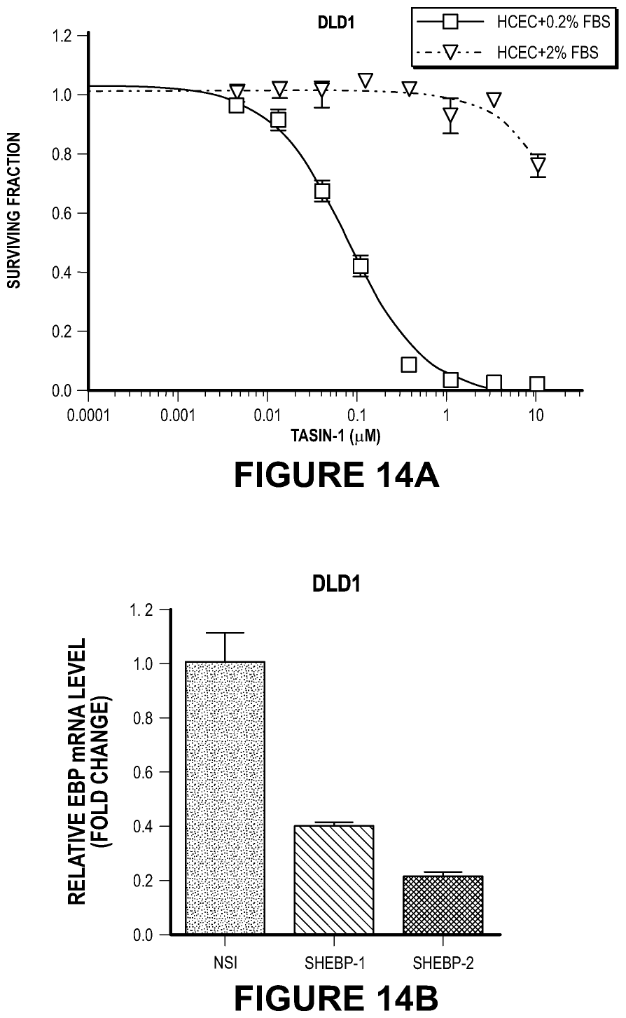

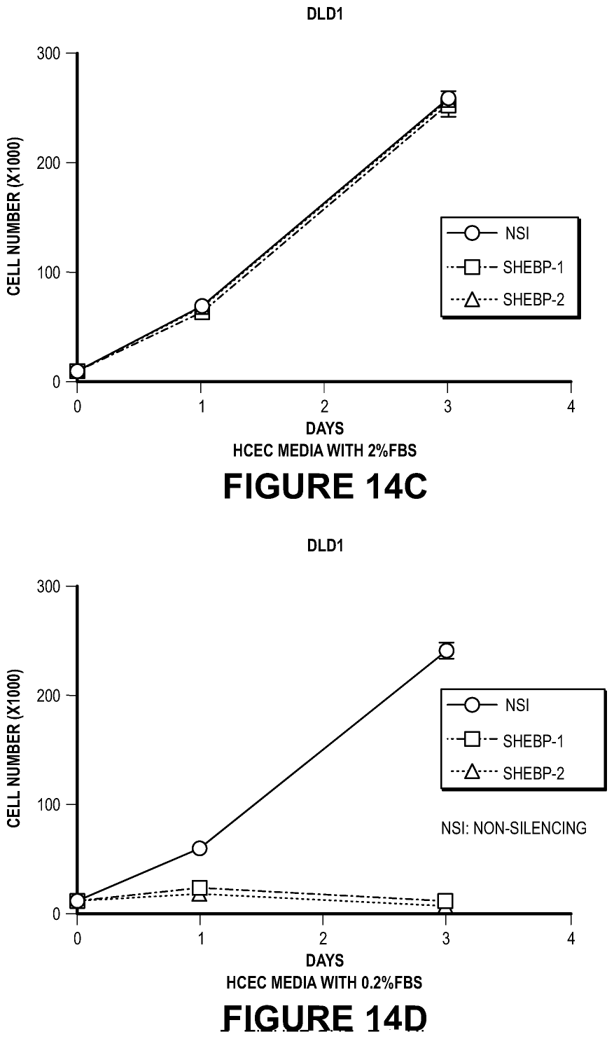

Stable knockdown of EBP recapitulates the effects of TASIN-1 in DLD-1 cells. FIGS. 14A-D show that shRNA knockdown of EBP has no effect on cells grown in 2% FBS, but does affect cell growth in 0.2% FBS. FIG. 14A is a plot of surviving fraction (y-axis) versus μM TASIN-1 (x axis) for HCEC cells in 0.2% (triangles) and 2% (squares) FBS. FIG. 14B is a bar graph showing fold change of relative EBP mRNA level for shEBP-1, shEBP-2, and a nonsilencing control. FIG. 14C is a plot of cell number (y axis) versus days in HCEC media with 2% FBS) for shEBP-1, shEBP-2, and a nonsilencing control. FIG. 14D is a plot of cell number (y axis) versus days in HCEC media with 0.2% FBS for shEBP-1, shEBP-2, and a nonsilencing control.

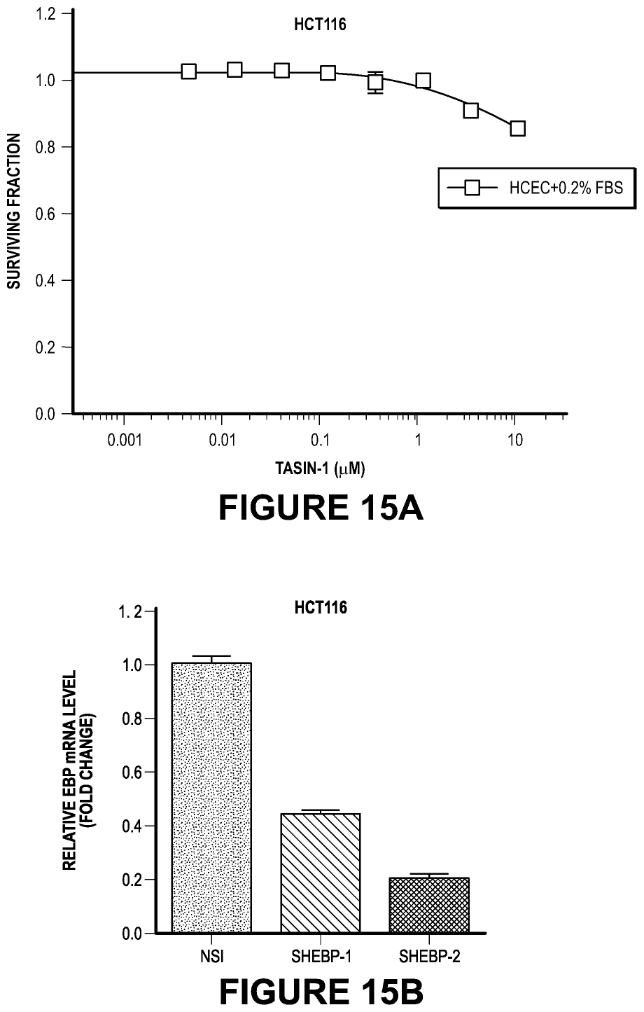

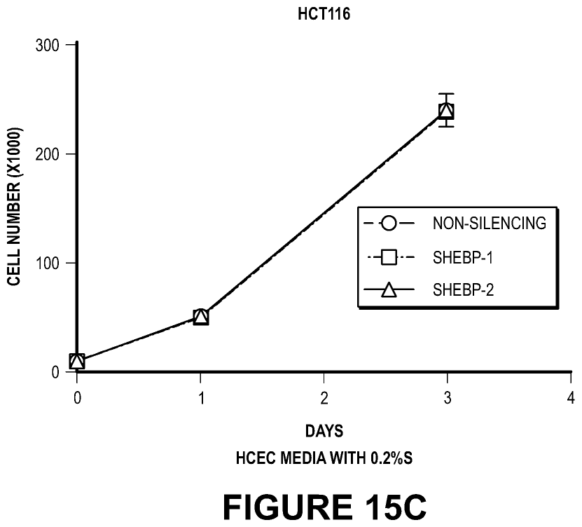

FIGS. 15A-C show that in the absence of truncated APC, stable knockdown of EBP has no effect on HCT116 cell growth rates. FIG. 15A is a plot of surviving fraction (y-axis) vs. μM TASIN-1 (x axis) for HCT116 HCEC cells in 0.2% FBS. FIG. 15B is a bar graph showing fold change of relative EBP mRNA level for shEBP-1, shEBP-2, and a nonsilencing control. FIG. 15C is a plot of cell number (y axis) versus days in HCEC media with 0.2% FBS) for shEBP-1, shEBP-2, and a nonsilencing control.

BP also was confirmed as the functional target of TASIN by demonstrating that ectropic over-expression of EBP leads to TASIN resistance.

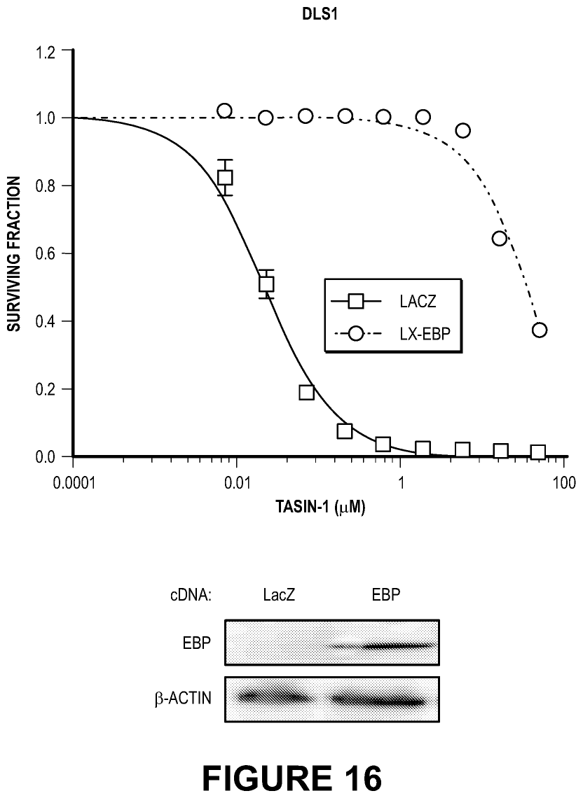

FIG. 16 shows that overexpression of EBP confers resistance to TASIN-1 in DLD-1 cells.

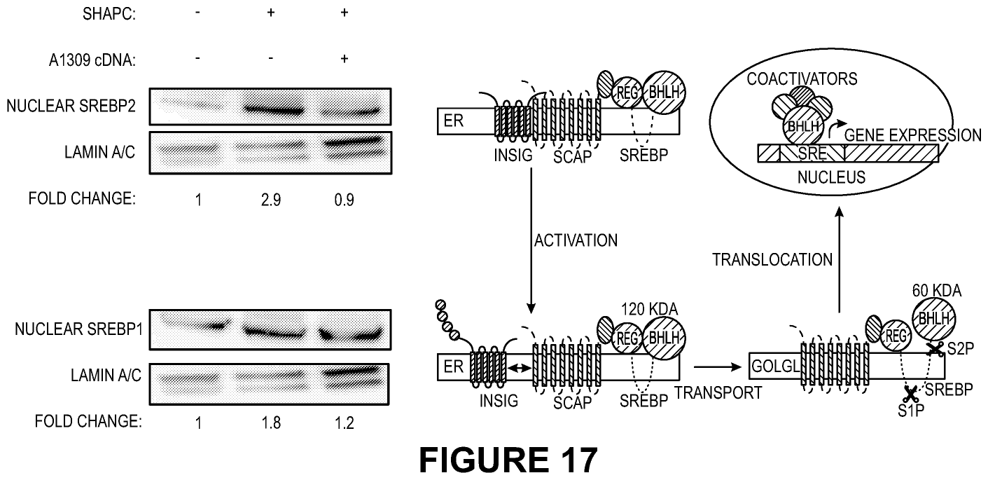

FIG. 17 shows that APC truncation expression reduces SREBP1 & 2 cleavage in DLD-1 cells.

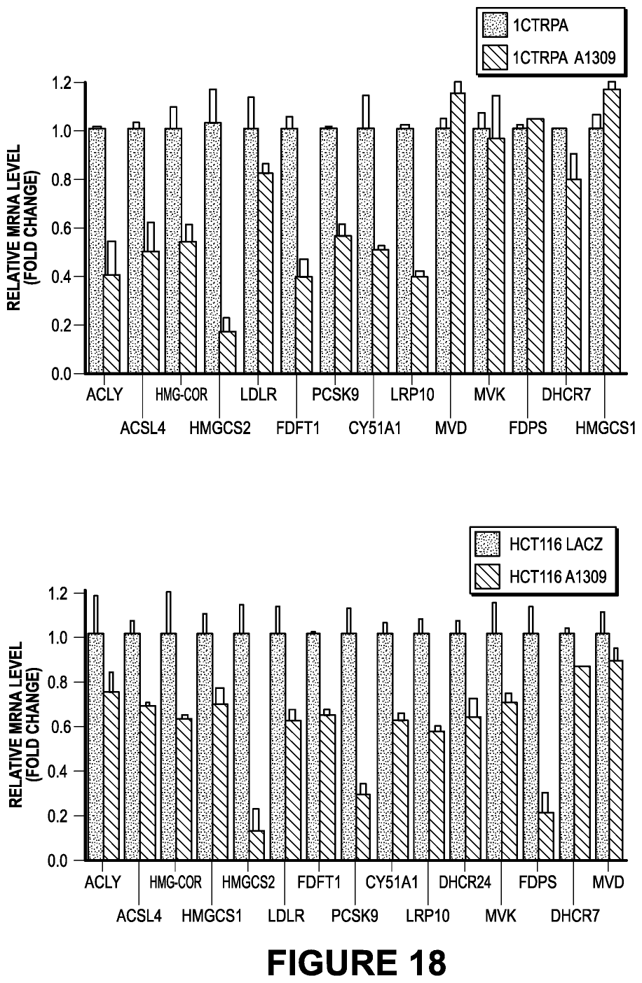

FIG. 18 shows that APC truncation expression down-regulates a panel of genes involved in cholesterol homeostasis.

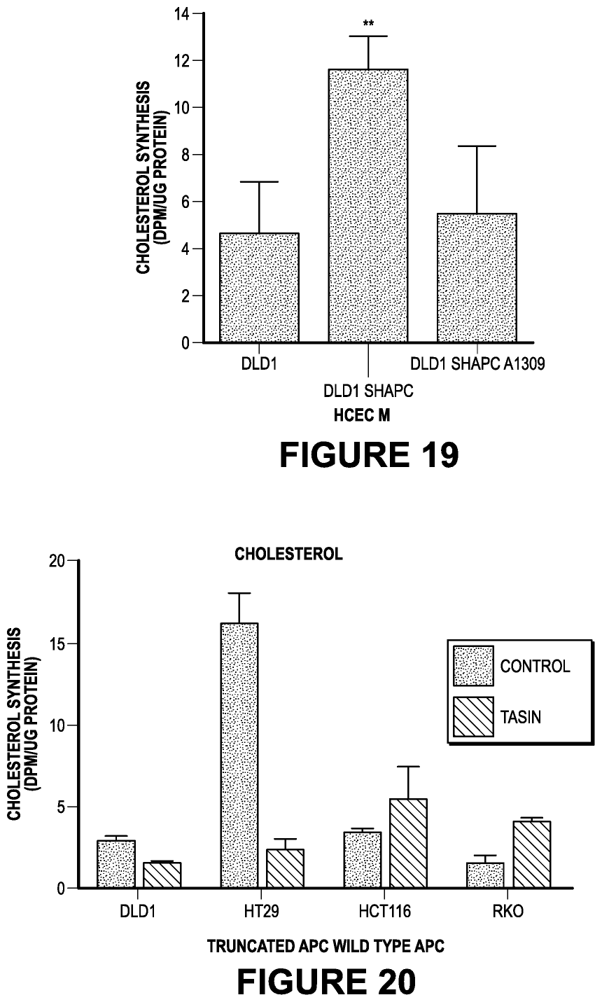

FIG. 19, a bar graph showing the effects of knockdown of truncated APC on endogenous cholesterol biosynthesis, is a plot of cholesterol synthesis (dpm/μg protein) versus untreated DLD1 cells, DLD1 cells treated with shAPC, and DLD1 cells treated with shAPC A1309 in HCEC media with 0.2% FBS. APC truncation reduced the endogenous cholesterol biosynthesis rate in DLD cells. Knockdown of truncated APC significantly increased endogenous cholesterol biosynthesis, but reintroduction of truncated APC returned the cholesterol synthesis rate back to DLD levels.

FIG. 20 shows that TASIN-1 further reduces endogenous cholesterol biosynthesis in cells containing truncated APC. Two cell lines expressing a truncated APC (DLD1 and HT29), and two cell lines expressing wild type APC (HCT116 and RKO colon cancer cells) were incubated with TASIN-1 for 40h, and then labeled for 8 hours with 14C acetate. Cholesterol was extracted from the cells and cholesterol synthesis quantitated (need more details). The result show that TASIN-1 reduces cholesterol biosynthesis in cells expressing truncated APC, but not in cells expressing wild type APC.

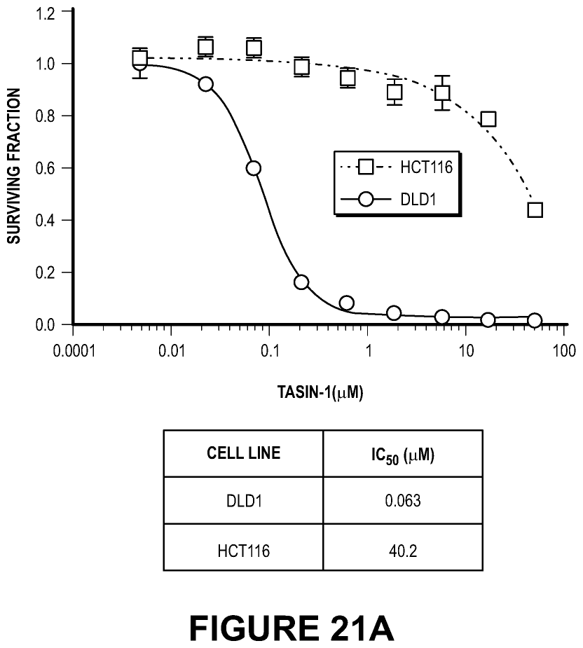

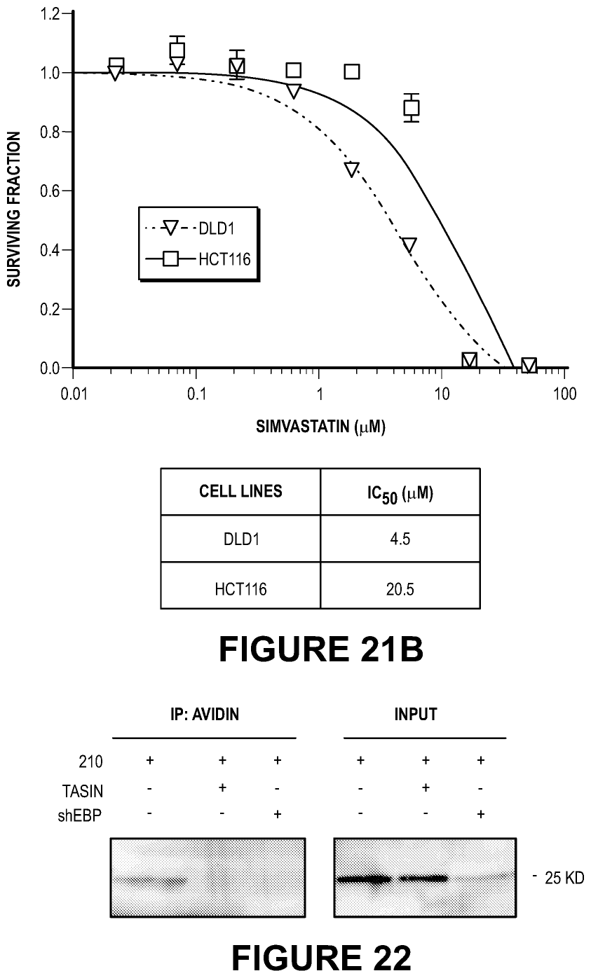

As shown in FIGS. 21A-B, a known cholesterol lowering drug (simvastatin, IC50 4.5 μM) has a slight effect on DLD1 cells (truncated APC) as shown FIG. 21A, and is significantly less potent than TASIN-1 (IC50 0.063 μM) as shown FIG. 21B. HCT116 cells (wild type APC) served as a control.

FIG. 22 shows that 210, a biotin-labeled potent TASIN analog, interacts with EBP in DLD1 cells. DLD1 cells were incubated with 210 in the presence or absence of TASIN-1 and pulled down by streptavidin beads. Bound EBP was detected by Western Blot. EBP is not pulled down in DLD1shEBP cells. These results confirm the interaction between TASIN-1 and EBP in DLD-1 cells.

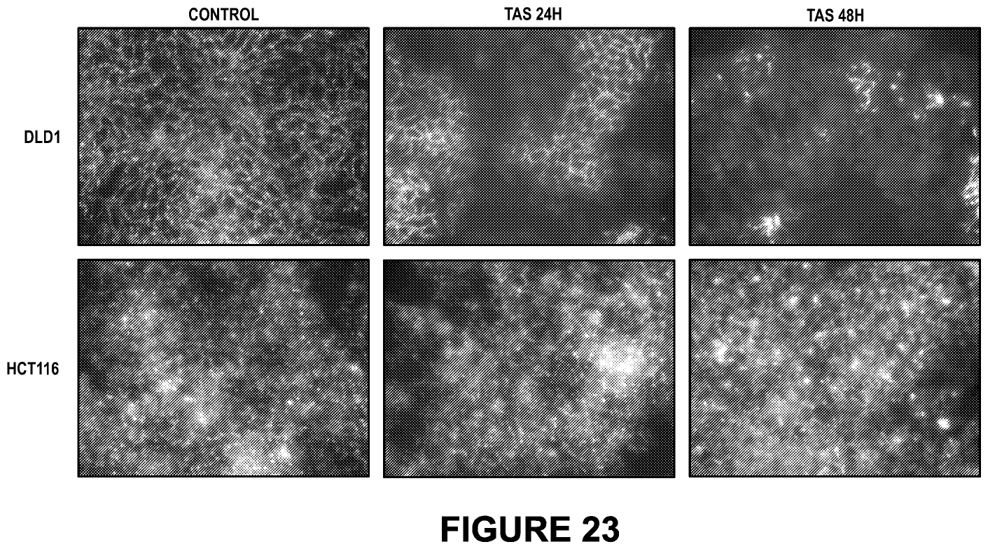

FIG. 23 shows that TASIN-1 decreases intracellular cholesterol level in DLD1 but not in HCT116 cells. Cells were treated with DMSO or 2.5 μM of TASIN-1 for 24 or 48 hours. Cholesterol levels were determined by Filipin III staining. Fipipin is a fluorescent chemical that specifically binds to cholesterol.

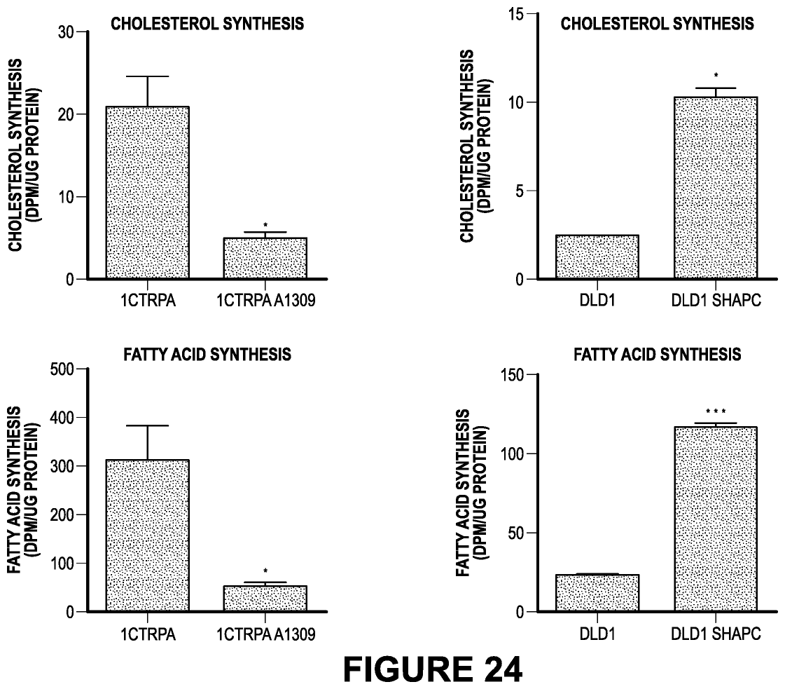

FIG. 24 shows that APC truncated protein is involved in cholesterol homeostasis. Cholesterol and fatty acid synthesis rates were measured in isogenic HCEC (1CTRPA, 1CTRPA A1309) and DLD1 cell lines (DLD1, DLD1 APC knockdown). Data represent mean±s.d., n=2. Student's t-test, *P<0.05, **P<0.01. APC truncation expression affects cholesterol and fatty acid biosynthesis rate.

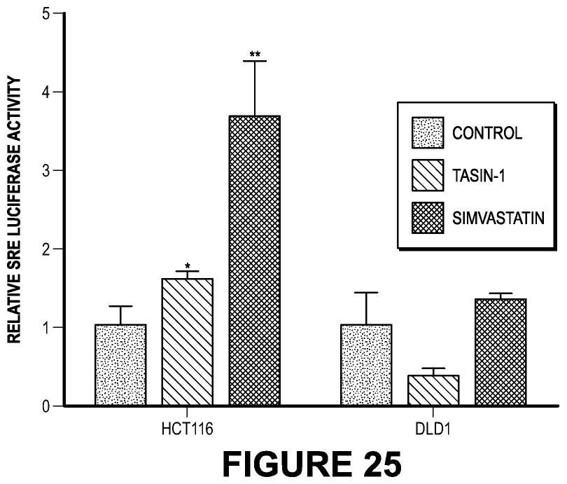

FIG. 25 shows the relative SRE luciferase activity in HCT116 and DLD1 cells treated with 2.5 μM of TASIN-1 or 10 μM of Simvastatin for 24 hours. Data represent mean±s.d., n=2. Student's t-test, **P<0.01. TASIN-1 treatment increased sterol response element (SRE) luciferase activity only in HCT116 cells.

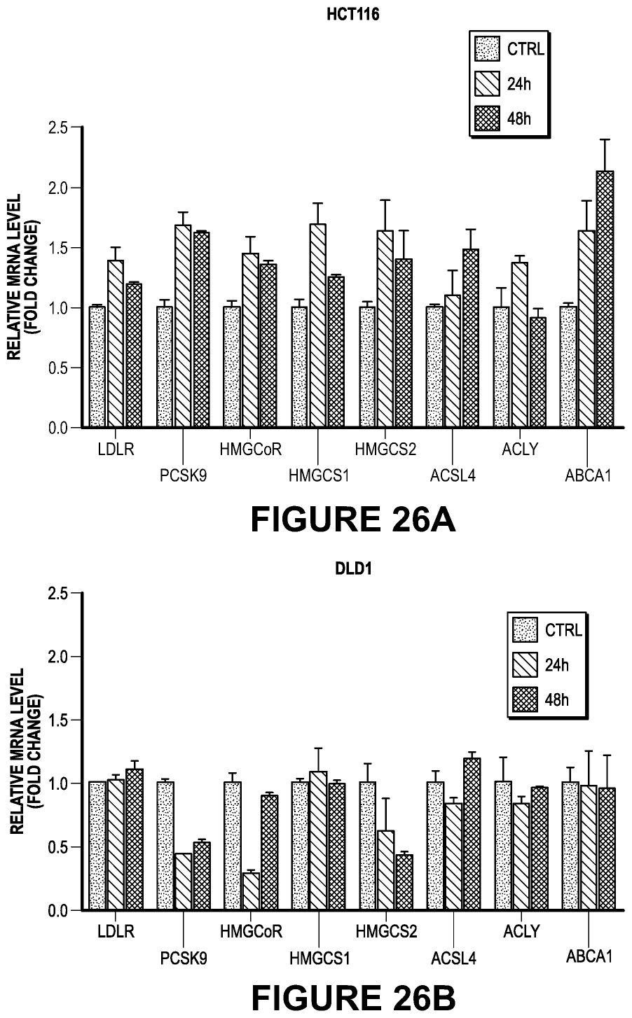

FIGS. 26A-B show the results of Quantitative PCR analysis of the major target genes regulated by SREBP2 in HCT116 cells (FIG. 26A) or DLD1 cells (FIG. 26B) treated with 2.5 μM of TASIN-1 for 24 and 48 hours. Expression level was normalized to the control cells. Data represent mean±s.d., n=2. TASIN-1 treatment leads to up-regulation of SREBP2 target genes only in HCT116 cells.

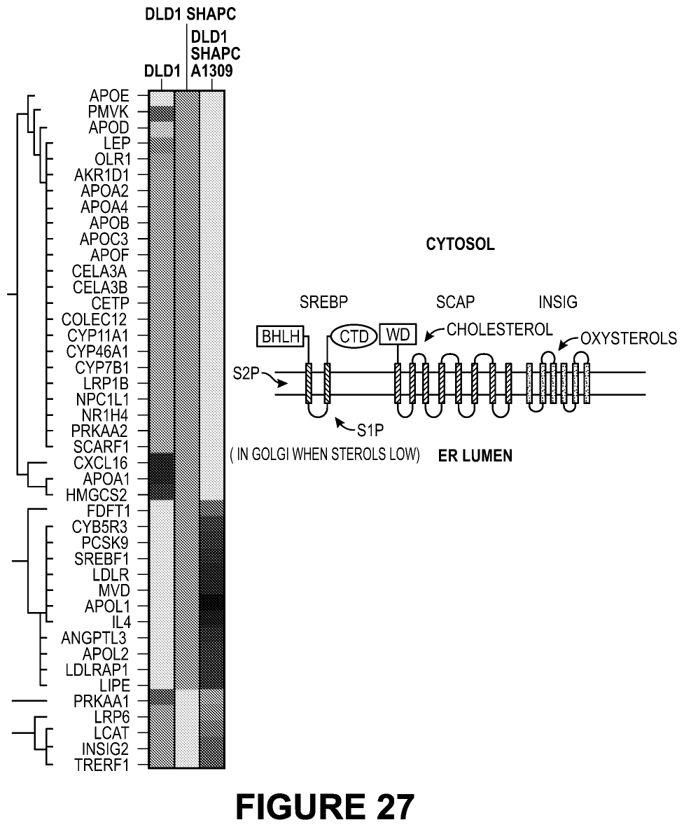

FIG. 27 is a lipoprotein signaling PCR array (Qiagen, 90 genes) showing upregulation and downregulation of a panel of cholesterol signaling related genes in APC knockdown DLD1 cells, which are reversed by ectopic expression of APC1309. The results demonstrate gain-of-Function of APC truncation in cholesterol signaling and metabolism.

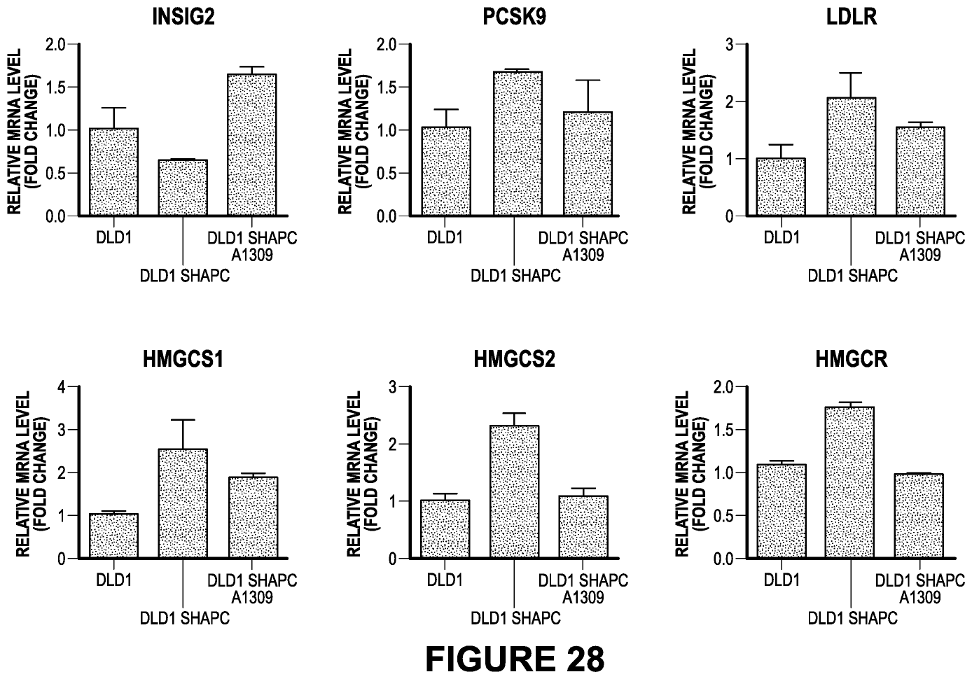

FIG. 28 shows that APC truncation affects expression of SREBP2 target genes. Quantitative PCR was performed on the isogenic DLD1 cell lines with primers against the major target genes regulated by SREBP2. Expression level was normalized to that in DLD1 cells. Data represent mean±s.d., n=2.

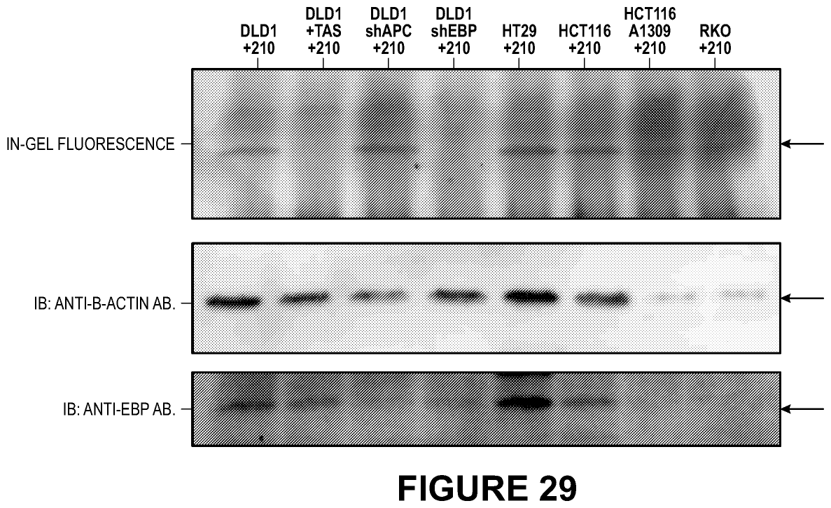

FIG. 29 confirms the interaction between TASIN-1 and EBP in colorectal cancer (CRC) cells. CRC cells were incubated with TASIN-1 analog #210 and labeled with Alexa532 after UV crosslinking via click reaction. Proteins were precipitated using cold acetone and resuspended in Laemmli buffer, followed by in-gel fluorescence and Western blot analysis.

### Example 6

### Pharmacological Data of the Tasin Analogs

The starting point for the described Structure Activity Relationship (SAR) studies is the HTS hit-compound TASIN-1. TASIN-1 (compound 6) is typified by an arylsulfonamide attached to a 1,4′-bipiperidine. The objectives for the enclosed SAR studies encompassed exploration of the following structural characteristics: (i) functionalization and substitution patterns in the sulfonylated aromatic ring, or replacement with biaryl or heteroaryl groups; (ii) substitution patterns in the terminal piperidine ring; (iii) replacement of the terminal piperidine ring with other heterocycles, aromatic rings, or acyclic substituents; (iv) replacement of the central sulfonylated piperidine ring with other ring systems or an acyclic tether; (v) replacement of the sulfonamide with an amide, carbamate, urea, sulfone, or a sulfamamide. All new compounds were initially evaluated in a cell proliferation assay (CellTiter-Glo®; Promega) using two human colorectal cancer cell lines, one with a truncating mutation in the APC gene (DLD-1) and one with wildtype APC status (HCT116). The assay was performed under low serum conditions (HCEC medium supplemented with 0.2% Fetal Bovine Serum). A select set of compounds was additionally evaluated against another human CRC cell line with truncating mutations in the APC gene (HT29), and a pair of diploid isogenic HCEC-derived cell lines (CTRPA, APC^{WT}; CTRPA A1309, APC^{TR}). Finally, select compounds were evaluated for in vitro metabolic stability using mouse liver S9 fractions and in vivo PK properties.

Cytotoxicity assay. DLD-1, HT29, HCT116, CTRPA, or CTRPA A1309 cells were seeded in 96-well plate in triplicate at a density of 3,000 cells/well in HCEC medium supplemented with 0.2% fetal bovine serum and treated with compound at 9-point 3-fold dilution series for 72 hours. Cell viability was determined using the CellTiter-Glo® (Promega) assay per manufacturer's instruction. Each value was normalized to cells treated with DMSO and the IC₅₀ values were calculated using Graphpad Prism software.

In vitro liver S9 stability. Female ICR/CD-1 mouse S9 fractions were purchased from Bioreclamation/IVT (Chestertown, Md.). 0.025 mL (0.5 mg) of S9 protein was added on ice to a 15 mL glass screw cap tube followed by 0.350 mL of a 50 mM Tris, pH 7.5 solution, containing the compound of interest. The tube was then placed in a 37° C. shaking water bath for 5 min and 0.125 mL of an NADPH-regenerating system (1.7 mg/mL NADP, 7.8 mg/mL glucose 6-phosphate, 6 U/mL glucose 6-phosphate dehydrogenase in 2% w/v NaHCO₃/10 mm MgCl₂) was added for analysis of phase I metabolism. After addition of all reagents, the final concentration of compound was 2 μM and S9 protein 1 mg/mL. At varying time points after addition of phase I cofactors, the reaction was stopped by the addition of 0.5 mL of methanol containing 0.2% formic acid and either tolbutamide or n-benzylbenzamide as internal standard. Time 0 samples were stopped prior to placing the samples at 37° C. and the NADPH-regenerating system was added immediately thereafter. The samples were incubated for 10 min at rt and then spun at 16,100 g for 5 min in a microcentrifuge. The supernatant was analyzed by LC-MS/MS using a Sciex 3200 or 4000 Qtrap mass spectrometer coupled to a Shimadzu Prominence LC with the mass spectrometer in MRM (multiple reaction monitoring) mode. The method described by McNaney (McNaney, et al. (2008) Assay Drug Dev. Technol. 6:121-129) was used with modification for determination of metabolic stability half-life by substrate depletion. A “% remaining” value was used to assess metabolic stability of a compound over time. The LC-MS/MS peak area of the incubated sample at each time point was divided by the LC-MS/MS peak area of the time 0 (T0) sample and multiplied by 100. The natural logarithm (ln) of the % remaining of compound was then plotted versus time (in min) and a linear regression curve plotted going through y-intercept at ln(100). The metabolism of some compounds failed to show linear kinetics at a later time point, so those time points were excluded. The half-life (T_{1/2}) was calculated as T_{1/2}=0.693/slope. Compound stability was also evaluated at 0 and 240 min in reaction buffer only minus S9 protein to determine chemical stability.

Protein Binding. Protein binding was determined using either ultrafiltration (compound 22) or rapid equilibrium dialysis (RED; compound 92) as described in Wang (Wang, et al. (2013) J. Pharm. Biomed. Anal. 75:112-117). Binding in plasma was evaluated using undiluted plasma and plasma diluted with 1 part plasma and 3 parts PBS, while binding in large intestine was evaluated using large intestinal tissue homogenates prepared using a 3-fold volume of PBS. Values were corrected for dilution as described (Kalvass, et al. (2002) Biopharm. Drug Dispos. 23:327-338) using the following equation:

$$Undiluted fu = \frac{1 \text{/} D}{( ( 1 \text{/} {fu}_{2} ) - 1 ) + 1 \text{/} D}$$

$$Where D = dilution factor of 4 and$$

$${fu}_{2} = free fraction using diluted matrix$$

Mouse PK analysis. Female CD-1 mice (5-6 weeks of age) were obtained from Charles River. The animals were housed in standard microisolator cages and were administered inhibitor compounds in 0.2 mL by IP (10 mg/kg) or IV (5 or 10 mg/kg) injection or oral gavage (20 mg/kg) formulated as follows: compounds 16 and 92: 10% DMSO/10% PEG-400/80% 50 mM citrate buffer, pH 5.4; compound 22: 10% ethanol/10% PEG-400/80% 50 mM citrate buffer, pH 4.4. Animals were euthanized by inhalation overdose of CO₂ in groups of 3 at 10, 30, 90, 180, 360, 960, and 1440 min post dose and blood collected by cardiac puncture, using acidified citrate dextrose (ACD) as the anticoagulant. In some cases, large intestine was also isolated, intestinal contents flushed, and the tissue snap frozen. Plasma was isolated from blood by centrifugation at 9600 g for 10 min and stored at −80° C. until analysis. Tissues were homogenized in a 3-fold volume of PBS (final homogenate volume in ml=weight of tissue in g×4). 0.1 mL of plasma or tissue homogenate was precipitated with 0.2 mL of an organic crash solution containing either methanol or acetonitrile, 0.15% formic acid, and an internal standard (n-benzylbenzamide). Extraction conditions were optimized prior to PK analysis for efficient and reproducible recovery over a 3 log range of concentrations. The solution was centrifuged twice at 16,100 g twice for 5 min. The final supernatant was analyzed by LC-MS/MS as described above, and compound concentrations were determined in reference to a standard curve prepared by addition of the appropriate compound to blank plasma or tissue homogenate. A value of 3× above the signal obtained in the blank plasma was designated the limit of detection (LOD). The limit of quantitation (LOQ) was defined as the lowest concentration at which back calculation yielded a concentration within 20% of the theoretical value and above the LOD signal. The LOQ values were as follows: 0.5 ng/ml for compounds 16, and 92; and 5 ng/ml for compound 22. Compound concentrations in large intestine were calculated by subtracting the amount of compound in the residual blood in that tissue based on reference values for large intestinal vasculature (Kwon, Y. (2001). The Handbook of Essential Pharmacokinetics. Pharmacodynamics, and Drug Metabolism for Industrial Scientists. Kluwer Academic/Plenum Publishers, New York. pp 231-232). Pharmacokinetic parameters were determined using the noncompartmental analysis tool in Phoenix WinNonlin (Certara, Corp., Princeton, N.J.).

TABLE 4
The pharmacological data of the chemical
compounds disclosed herein.

| Compound No. | DLD-1 IC₅₀ (nM)^{a} | S9 T_{1/2} (min)^{b} | ClogD_{7.4}^{c} |
|---|---|---|---|
| 5 | 294 ± 7.6 | — | 0.69 |
| 6 | 63 ± 5.6 | 182 | 0.52 |
| 7 | 981 ± 22.6 | — | 0.66 |
| 8 | >5,000 | — | 0.72 |
| 9 | 2,800 | — | 2.28 |
| 10 | >5,000 | — | −0.17 |
| 11 | 9.1 ± 0.6 | 19 | 1.2 |
| 12 | >5,000 | — | 1.64 |
| 13 | 685 ± 24 | — | 1.93 |
| 14 | >5,000 | — | 2.23 |
| 15 | 185 ± 8.7 | — | 1.57 |
| 16 | 2.2 ± 0.04 | 41 | 1.3 |
| 17 | 3.1 ± 0.08 | 32 | 1.46 |
| 18 | 452 ± 2.6 | — | 1.4 |
| 19 | 2,300 | — | 1.85 |
| 20 | 385 ± 9.4 | — | 2.25 |
| 21 | 16 ± 0.7 | — | 2.12 |
| 22 | 4.8 ± 0.5 | >240 | 1.46 |
| 23 | >5,000 | — | 0.54 |
| 24 | >5,000 | — | 0.67 |
| 25 | 3,400 | — | −0.14 |
| 26 | 25 ± 2.1 | — | 1.2 |
| 27 | >5,000 | — | 1.58 |
| 28 | >5,000 | — | 1.31 |
| 29 | 3.2 ± 0.06 | 13 | 1.47 |
| 30 | 3 ± 0.25 | 6 | 1.48 |
| 31 | 138 ± 12 | — | 0.37 |
| 32 | 4.5 ± 0.2 | 9.4 | 0.41 |
| 33 | 2 ± 0.006 | 9 | 1.33 |
| 34 | >5,000 | — | 1.06 |
| 35 | 56 ± 1.6 | — | 1.55 |
| 36 | 285 ± 3.9 | — | 1.09 |
| 37 | 24 ± 1.2 | — | 1.71 |
| 38 | 0.6 ± 0.02 | 9.1 | 2.47 |
| 39 | 17 ± 1.2 | 32 | 2.18 |
| 40 | 2.9 ± 0.34 | 33 | 1.91 |
| 41 | 1 ± 0.002 | 15 | 1.93 |
| 42 | 3,400 | — | 1.92 |
| 43 | 12 ± 0.5 | 43 | 2.08 |
| 44 | 102 ± 3.5 | — | 1.02 |
| 45 | 5 ± 0.07 | 5 | 2.93 |
| 46 | 3 ± 0.23 | 8 | 2.27 |
| 47 | 0.03 ± 0.0001 | 5 | 2.2 |
| 48 | 2 ± 0.05 | 6.5 | 2.44 |
| 49 | 0.6 ± 0.01 | 7.2 | 2.43 |
| 50 | 107 ± 5.3 | — | 1.78 |
| 51 | 0.96 ± 0.002 | <6 | 2.35 |
| 52 | 202 ± 7.6 | — | 2.95 |
| 53 | 1,000 | — | 2.19 |
| 54 | 366 ± 9.6 | — | 2.86 |
| 55 | 122 ± 8.6 | — | 3.22 |
| 56 | 258 ± 14 | — | 2.34 |
| 57 | 105 ± 7.6 | — | 2.94 |
| 58 | 263 ± 25 | — | 2.18 |
| 59 | 26 ± 0.5 | 289 | 2.19 |
| 60 | 5 ± 0.06 | 210 | 2.49 |
| 61 | 2 ± 0.3 | 31 | 2.34 |
| 62 | 15 ± 2.1 | 147 | 2.95 |
| 63 | 23 ± 0.9 | 144 | 2.19 |
| 64 | 11 ± 1.0 | 115 | 2.86 |
| 65 | 12 ± 0.6 | 77 | 2.49 |
| 66 | 21 ± 0.2 | 15 | 2.19 |
| 67 | 10 ± 0.9 | 17 | 2.49 |
| 68 | 10 ± 0.03 | 37 | 3.22 |
| 69 | 41 ± 1.1 | >240 | 3.37 |
| 70 | 2 ± 0.0001 | 204 | 3.33 |
| 71 | 2,400 | — | −0.49 |
| 72 | >5,000 | — | 0.35 |
| 73 | >5,000 | — | 0.08 |
| 74 | 3,700 | — | 0.33 |
| 75 | >5,000 | — | 0.28 |
| 76 | >5,000 | — | −1.71 |
| 77 | >5,000 | — | −0.5 |
| 78 | >5,000 | — | −0.55 |
| 79 | >5,000 | — | −0.53 |
| 80 | 1,800 | — | 0.53 |
| 81 | 1.6 ± 0.03 | 10 | 0.63 |
| 82 | >5,000 | — | 1.42 |
| 83 | 853 ± 43 | — | 0.35 |
| 84 | 62 ± 4.3 | — | 0.32 |
| 85 | 1.2 ± 0.03 | 67 | 2.78 |
| 86 | 96 ± 7.3 | 5 | 1.37 |
| 87 | 0.65 ± 0.002 | <5 | 1.69 |
| 88 | 0.1 ± 0.02 | 14 | 2.3 |
| 89 | 1,100 | — | 0.93 |
| 90 | 225 ± 13 | — | 0.47 |
| 91 | 105 ± 21 | — | 1.41 |
| 92 | 0.2 ± 0.003 | — | 1.55 |
| 93 | >5,000 | — | 1.05 |
| 94 | 85 ± 6.8 | — | 0.67 |
| 95 | >5,000 | — | 0.44 |
| 96 | 3 ± 0.04 | — | 3.21 |
| 97 | 17 ± 0.9 | — | 2.22 |
| 98 | 0.3 ± 0.002 | — | 2.83 |
| 99 | >5,000 | — | 1.97 |
| 100 | 19 ± 0.8 | — | 1.31 |
| 101 | 106 ± 7.9 | — | 1.01 |
| 102 | 2,200 | — | 2.1 |
| 103 | 5.3 ± 2.0^{d} | — | 2.09 |
| 104 | 3,203 | — | −0.15 |
| 105 | 92 ± 1.23 | — | 2.94 |
| 106 | 1,100 | — | 1.57 |
| 107 | 3,100 | — | 2.32 |
| 108 | 3.5 ± 0.03 | — | 3.76 |
| 109 | 74 ± 4.5 | — | 4.04 |
| 110 | 18 ± 0.7 | — | 3.93 |
| 111 | 865 ± 89 | — | 3.44 |
| 112 | >5,000 | — | 3.52 |
| 113 | >5,000 | — | 3.42 |
| 114 | 2,900 | — | 4.36 |
| 115 | 2,200 | — | 5.35 |
| 116 | >5,000 | — | 4.85 |
| 117 | >5,000 | — | 4.69 |
| 118 | >5,000 | — | 2.74 |
| 119 | 426 ± 24.5 | — | 1.54 |
| 120 | 256 ± 10 | — | 0.5 |
| 121 | 1,360 | — | 0.03 |
| 122 | 814 ± 21 | — | −0.27 |
| 123 | 380 ± 3.5 | — | 2.45 |
| 124 | 1,900 | — | 3.06 |
| 125 | >5,000 | — | 5.71 |
| 126 | 426 ± 42 | — | 0.75 |
| 127 | 84 ± 7.6 | — | 0.87 |
| 128 | 231 ± 4.4 | — | 3.8 |
| 129 | 958 | — | 0.43 |

^{a}IC₅₀ values represent the half maximal (50%) inhibitory concentration as determined in the CellTiter-Glo ® (Promega) assay. Error represents SD (n = 3). All compounds were inactive when counter-screened against the HCT116 cell line (IC₅₀ > 5 μM).
^{b}T_{1/2} values represent the half life for compound phase I metabolic stability using female ICR/CD-1 mouse liver S9 fractions. NA = Not Assayed. (compounds 71-80 were also not assayed for microsomal stability)
^{c}Calculated using MarvinSketch (version 6.3.0).
^{d}Error represents SD (n = 2). All compounds were inactive when counter-screened against the HCT116 cell line (IC₅₀ > 5 μM).

SAR of Monocyclic Functionalized Arylsulfonamides. Our SAR studies initiated with an evaluation of the arylsulfonamide moiety. As shown in Table 4, compared to the parent 4-methoxyphenyl-substituted comparator TASIN-1 (compound 6), replacement with an unsubstituted phenyl ring (compound 5) led to about a 5-fold reduction in antiproliferative activity. A survey of various para-substituents indicated that strongly electron-withdrawing (—NO₂, compound 7; —CO₂Me, compound 8; or —CN, compound 9) or polar hydrophilic substituents (—NH₂, compound 10) were not tolerated. Increasing the size of the 4-alkoxy substituent from methoxy (compound 6) to a propoxy (compound 18), butoxy (compound 19) or benzyloxy (compound 20) also led to a significant drop in activity. That steric hindrance in the para-position might be the culprit was in agreement with the observation that the 4-methyl substituted compound 11 yielded single-digit nanomolar activity (IC₅₀=9.1 nM), whereas increasing the size of the 4-alkyl substituent (ethyl, compound 12; isopropyl, compound 13, or t-butyl, compound 14) abrogated activity in the DLD-1 cell line. Replacement of the 4-methoxy group with fluorinated congeners (—OCF₃, compound 21; —OCHF₂, compound 22) or halides (—Cl, compound 16; —Br, compound 17) improved potency 4- to 28-fold, in agreement with smaller hydrophobic Van Der Waals-interacting substituents being preferred at this position. An exception was noted for the 4-trifluoromethylphenyl compound 15, which was 20-fold less active than the corresponding 4-methylphenyl compound 11. Although less extensively explored, single meta-substituents that improved activity versus the unsubstituted parent compound 5 were restricted to methyl (compound 26, IC₅₀=25 nM) and bromine (compound 29, IC₅₀=3.2 nM), whereas 3-methoxy, 3-nitro, 3-amino, 3-trifluoromethyl, and 3-chloro substitution (compounds 23-25, 27, 28) led to a virtual complete loss of antiproliferative activity. Ortho-bromo substitution (compound 30) also provided high potency (IC₅₀=3 nM). Trends for a series of disubstituted arylsulfonamides were less clear. Addition of a 2-methoxy substituent to the 4-methylphenyl compound 11 (IC₅₀=9.1 nM) yielded an inactive compound 34, whereas 2-methoxy substitution did not diminish activity in combination with 3-bromo (compound 33 versus compound 29), and dramatically increased potency of an otherwise inactive monosubstituted meta-methoxyphenyl compounds (compound 32, IC₅₀=4.5 nM versus compound 23). The 2,4-dimethoxyphenyl analog (compound 31) lost about 2-fold in potency versus the 4-methoxyphenyl comparator (compound 6). Interestingly, a 3-trifluoromethyl group enhanced significantly the activity of the otherwise inactive para-nitrophenyl analog (compound 35, IC₅₀=56 nM versus compound 7, IC₅₀=981 nM), but diminished the activity of the para-chlorophenyl parent (compound 39, IC₅₀=17 nM versus compound 16, IC₅₀=2.2 nM). Additional introduction of an ortho-ethyl or -chloride substituent slightly enhanced activity (compound 38, IC₅₀=0.6 nM and compound 41, IC₅₀=1 nM versus compound 17, IC₅₀=3.1 nM and compound 16, IC₅₀=2.2 nM respectively), whereas a 2-trifluoromethoxy group had minimal effect (compound 45, IC₅₀=5 nM versus compound 17, IC₅₀=3.1 nM). When attaching an additional methyl, trifluoromethyl, or chlorine to the meta-position, the potency of the corresponding monosubstituted para-methyl, -trifluoromethyl, -chloro, or -bromo analogs was diminished 1.3- to 7.7-fold (compound 37, IC₅₀=24 nM; compound 39, IC₅₀=17 nM; compound 40, IC₅₀=2.9 nM; compound 43, IC₅₀=12 nM versus compound 11, IC₅₀=9.1 nM; compound 16, IC₅₀=2.2 nM; compound 17, IC₅₀=3.1 nM). The 2-cyano-5-methylphenyl analog compound 36 was 11-fold less active than ortho-methylphenyl comparator compound 26. The 3,5-dichlorophenyl analog compound 42 displayed very weak activity, unlike other dihalo substitution patterns (compounds 40, 41, 43). Based on the moderate activity of compounds 44 and 50, fluorine substitution did not appear beneficial.

SAR of Monocycic Functionalized Arylsulfonamides. Although not perfect and with some exceptions (e.g. compounds 16, 17, 37, 42), the SAR-patterns of substituted arylphenylsulfonamide analogs correlated best with a Hansch-type π-σ parameter dependency,^{28,29} in addition to steric restrictions at the 4-position. Specifically, potent analogs in this series are characterized by substitution with relatively apolar substituents with low electron-withdrawing ability (Br, Cl, alkyl, MeO, CF₃O, CHF₂O), particular beneficial at the ortho- and para-positions, but restricted in size at the 4-position. Combining these features in a set of trisubstituted arylsulfonamides resulted in a series of very potent compounds (compounds 46-49, IC₅₀=0.03-3.0 nM). Overall, fifteen analogs displayed IC₅₀-values below 5 nM against the DLD-1 colon cancer cell line, with two breaking the picomolar barrier (compound 47, IC₅₀=30 μM and compound 49, IC₅₀=600 μM). None of the compounds tested registered any activity in the corresponding colon cancer cell line with wild-type APC status (HCT116), attesting to their highly specific genotype-selective mode-of-action. Unfortunately, eleven were rapidly metabolized with half-lives between 5 and 30 minutes when subjected to murine S9 microsomal fractions, and another 3 with half-lives between 32 and 41 minutes. Only the 4-difluoromethoxyphenyl analog compound 22 retained very potent cellular activity (IC₅₀=4.8 nM) while exhibiting excellent microsomal stability (T_{1/2}>240 min). The 3-chloro-4-bromophenyl analog compound 43 was slightly less potent (IC₅₀=12 nM) but also exhibited acceptable microsomal stability (T_{1/2}=43 min). Not surprisingly given the bipiperidinyl moiety, the C log D7.4 value for all potent analogs was below 2.93 (range 0.41-2.93; C log P range 3.21-4.91). Other parameters such as rotatable bonds, hydrogen bond donors and acceptors, total polar surface area (tPSA, range 40.62-59.08) and MW (range 336-485) are also within the range of drug-like properties for orally available small molecules.

SAR of Biaryl Sulfonamides. Next, we decided to briefly explore ortho-, meta-, and para-aryl substituted phenylsulfonamides (biaryl analogs, Table 4). In the ortho-series, only the unsubstituted biphenyl analog compound 51 displayed potent selective cytotoxicity against the DLD-1 cell line with truncating APC-mutations. Ortho-biphenyl analogs with additional substituents at the 4′-position (compounds 52-55) were >120-fold less active, indicating a potential size restriction along the 4′-vector. For para-biphenyls, the unsubstituted biphenyl analog compound 56 and those with additional 4′-substitution (compounds 57, 58) where significantly less potent than those with additional 2′-substituents (compounds 59, 60). We had previously observed that bulkier substituents in the para-position of the arylsulfonamide was dendrimental to activity (Table 4, compounds 12-14 and 18-20). However, given the potent activity of compounds 59 and 60, both containing a large aryl group in the para-position, one might speculate that this size-restriction is limited to 3-dimensional substituents, and space is allowed for a flat properly oriented aryl ring. For the biaryl series of analogs, the ortho-position appears to be the sweet spot for connecting the additional aromatic ring, and all ortho-biaryl analog compounds 61-70 displayed potent activity with IC₅₀'s between 2 and 41 nM. The biaryl series of analogs also appeared to provide opportunities to improve metabolism. Indeed, with the exception of compounds 51, 66, and 67 all other potent biaryl analogs had acceptable half-lives between 31 and 289 min in the in vitro murine S9 microsomal stability assay. Despite these initial promising results within the biaryl series, we decided not to further pursue them in light of the significant price to be paid in terms of increased molecular weight and lipophilicity (C log D_{7.4} range 2.19-3.37; C log P range 4.8-5.7).

SAR of Heterocyclic and Fused Bicyclic Sulfonamides. In our search for heterocyclic and fused bicyclic replacements for the arylsulfonamide ring, it became quickly apparent that the more desirable (drug-like properties) heterocyclic ring systems such as pyridines, imidazoles, thiazoles, and isoxazoles were not a fruitful avenue of pursuit. With the exception of thiazole compound 81, all such heterocyclic replacements led to inactive compounds (compounds 71-80, 82). Interestingly, whereas the methyl-substituted chlorothiazole compound 81 was very potent (IC₅₀=1.6 nM), removing the methyl-substituent (compound 80), or replacement with an isopropyl (compound 82) largely abolished activity of these chlorothiazoles. Despite the potency and excellent C log D_{7.4} of 0.63, the chlorothiazole analog compound 81 was rapidly metabolized in murine S9 microsomal fractions (T_{1/2}=10 min). On the other hand, activity results for the relatively apolar fused benzodioxoles and dihydrobenzofurans were more in alignment with results displayed in Table 2. Whereas benzodioxole compound 83 displayed mediocre activity (IC₅₀=853 nM), the corresponding isomeric benzodioxole compound 84 exhibited a 14-fold improvement (IC₅₀=62 nM). Bromo-dihydrobenzopyran compound 86 was of intermediate potency (IC₅₀=96 nM), while introduction of additional methyl-groups led to the very potent and metabolically stable compound 85 (IC₅₀=1.2 nM; T1/2 (S9)=67 min) but increased lipophilicity (C log D_{7.4=2.78}; C log P=5.4). Finally, lipophilic naphtyl analog compounds 87 and 88 were very potent (0.1-0.65 nM) but metabolically labile (T_{1/2}<5-14 min) while the more polar acetamidonaphtyl and isoquinoline analog compound 89 and 90 largely lost activity.

SAR of the Terminal Piperidine Ring. With a rather extensive survey of the arylsulfonamide moiety completed, the next phase entailed evaluation of the terminal piperidine ring. As shown in Table 4, moving the terminal methyl group from the 4- to the 3-position as in compound 91 reduced activity ˜20-fold versus the comparator compound 22 (105 vs 4.8 nM). Replacement of the 4-methyl with a propargyl (compound 92) or propargyloxyethyl (compound 94) increased activity significantly versus comparator compounds 17 and 5 (0.2 and 85 nM vs 3.1 and 294 nM). Moving those two substituents to the 2-position as in compounds 93 and 95 on the other hand was not tolerated. Other groups that were tolerated in the 4-position in decreasing order of potency are isopropyl (compound 98, 0.3 nM), benzyl (compound 108, 3.5 nM), 2-oxohex-5-yn-1-yl (compound 96, 3 nM), (4-fluorophenyl)methyl (compound 110, 18 nM), 2-hydroxyethyl (compound 100, 19 nM), (2-fluorophenyl)methyl (compound 109, 74 nM), and hydroxy (compound 101, 106 nM). A phenyl (compound 111) or 2-cyanoethyl (compound 99) in that position greatly diminished activity (865 to 7,400 nM). The unsubstituted piperidine compound 102 lost almost all activity, whereas the 3,5-Me₂-substituted compound 97 was active at 17 nM. When other nitrogen-containing ring systems were evaluated, it was revealed that pyrrolidine compound 104, morpholine compound 107, and piperazine compound 106 all lost activity, whereas 1,3-oxazinane compound 105 was of intermediate potency (IC₅₀=92 nM). Only azepane compound 103 retained single-digit nanomolar potency. Removing the ring-nitrogen altogether, such as in 1,3-dioxane compound 112 and phenyl or tolyl compounds 115 and 116 led to a complete loss of activity. Introducing a carbonyl between the two piperidine ring systems (compound 113), or replacing the terminal piperidine ring with acyclic substituents such as aniline compound 114, or amide compounds 117 and 118 were also unproductive. To conclude this section, the above results indicate that a basic nitrogen within a six to seven-membered ring (piperidine or azepine) is crucial for retaining cellular activity. A number of substituents, mostly hydrophobic in nature that can include aliphatic, hydroxylated alkyl, propargylic, ether, ketone, or benzylic substitution are well tolerated. Introduction of an additional polar nitrogen or oxygen in the terminal azacycle is contraindicated for bioactivity. Of the single-digit nanomolar compounds, azepane compound 103 had the lowest C log D7.4 (2.09).

Miscellaneous SAR. The final structural attributes to be explored are the role of the sulfonamide functionality and the central piperidine ring. As shown in Table 4, replacement of the sulfonamide linker with an amide (compound 119, IC₅₀=426 nM), urea (compound 121, IC₅₀=1,360 nM), or sulfamamide linker (compound 122, IC₅₀=814 nM ) led to a significant 13 to 90 fold reduction in activity when compared to their sulfonamide congener compounds 22 and 6 (IC₅₀=4.8 and 63 nM). The carbamate replacement compound 120 fared better with a more marginal 4-fold drop versus sulfonamide compound 6—a modification that further lowered the C log D_{7.4} to 0.5. Substitution of the central piperidine ring with an aminocyclohexyl (123), aminoethyl (compound 124), or phenyl linker (compound 125) led to a substantial or complete loss of activity. Finally, replacement of the bipiperidine with an amino-bipiperidine (compound 129), N-(N-methylpiperidin-4 yl)piperazine (compound 126), or adamantanylpiperazine (compound 128) led to compounds with mediocre potency. Of interest for future analog design, a quinuclidine compound 127 with altered position of the tertiary nitrogen retained significant activity (IC₅₀=83 nM), was metabolically stable (S9 T_{1/2}=193 min) and decreased C log D_{7.4} significantly to 0.87.

TABLE 5
Antiproliferative Activity of Selected Analogs
in Cell Lines with Truncated APC.

| | IC₅₀ (nM)^{a} | | |
|---|---|---|---|
| Compound No. | DLD-1 | HT-29 | CTRPA A1309^{b} |
| 6 | 63 ± 5.6 | 53 ± 2.3 | 122 ± 6.5 |
| 22 | 4.8 ± 0.5 | 2 ± 0.1 | 3.8 ± 0.7 |
| 29 | 3.2 ± 0.06 | 1.2 ± 0.07 | 6.9 ± 0.8 |
| 30 | 3 ± 0.25 | 3 ± 0.05 | 2.8 ± 0.03 |
| 32 | 4.5 ± 0.2 | 4 ± 0.1 | 10.5 ± 0.3 |
| 33 | 2 ± 0.006 | 2 ± 0.004 | 0.7 ± 0.02 |
| 38 | 0.6 ± 0.02 | 0.5 ± 0.1 | 0.8 ± 0.03 |
| 40 | 2.9 ± 0.34 | 2.2 ± 0.06 | 3.4 ± 0.7 |
| 45 | 5 ± 0.07 | 4 ± 0.02 | 9.6 ± 0.3 |
| 46 | 3 ± 0.23 | 2.2 ± 0.3 | 3.4 ± 0.7 |
| 47 | 0.03 ± 0.0001 | 0.9 ± 0.08 | 0.04 ± 0.002 |
| 48 | 2 ± 0.05 | 1.1 ± 0.04 | 3.2 ± 0.4 |
| 49 | 0.6 ± 0.01 | 0.45 ± 0.01 | 0.7 ± 0.05 |
| 51 | 0.96 ± 0.002 | 0.74 ± 0.03 | 1.2 ± 0.06 |
| 60 | 5 ± 0.06 | 6 ± 0.07 | 10.2 ± 0.8 |
| 61 | 2 ± 0.3 | 2 ± 0.07 | 1.6 ± 0.3 |
| 81 | 1.6 ± 0.03 | 1.5 ± 0.03 | 3.4 ± 0.3 |
| 85 | 1.2 ± 0.03 | 0.8 ± 0.03 | 1.5 ± 0.08 |
| 87 | 0.65 ± 0.002 | 0.34 ± 0.02 | 0.94 ± 0.01 |
| 92 | 0.2 ± 0.03 | 0.12 ± 0.05 | 0.9 ± 0.07 |
| 96 | 3 ± 0.04 | 2.1 ± 0.07 | 3.4 ± 0.8 |
| 98 | 0.3 ± 0.002 | 0.2 ± 0.001 | 0.6 ± 0.003 |
| 108 | 3.5 ± 0.03 | 3 ± 0.2 | 4.3 ± 0.1 |

^{a}IC₅₀ values represent the half maximal (50%) inhibitory concentration as determined in the CellTiter-Glo ® (Promega) assay. Error represents SD (n = 3). All compounds were inactive when counter-screened against the HCT116 cell line with wild-type APC status (IC₅₀ > 5 μM).
^{b}All compounds were inactive when counter-screened against the isogenic CTRPA cell line with wild-type APC status (IC₅₀ > 5 μM).

Activity in Other Cell Lines. As disclosed previously, TASIN-1 (compound 6) was identified as a selective cytotoxin that specifically kills colon cancer cell lines with truncating mutations in the APC tumor suppressor gene. Above we described an extensive medicinal chemistry effort to identify analogs of TASIN-1 with improved potency and physicochemical properties. To ensure that these analogs remained on target, we have additionally counter-screened them for activity against the HCT-116 cell line with wild-type APC. In Table 5 above, we represent additional cytotoxicity data for a selection of 23 compounds that displayed single-digit nanomolar activity against the DLD-1 cell line. All 23 compounds were found to be equally effective against another human colon cancer cell line with truncating APC-mutations (HT29). Given the heterogenous genetic background between all these cultured human colon cancer cell lines (DLD-1, HT29, HCT116), we further evaluated these analogs against an isogenic cell line pair derived from primary human colonic epithelial (HCEC) cells. As disclosed previously, introduction of cyclin-dependent kinase 4 (CDK4), telomerase (T) into primary HCEC cells was sufficient to produce an immortalized, nontransformed diploid cell line (CT) with multipotent stem-like characteristics that can differentiate in three dimensional culture conditions. Additional introduction of oncogenic KRAS^{V12}, mutant TP53 (key-alterations in CRC), and knockdown of APC established the CTRPA cell line. Additional ectopic expression of mutant APC truncated at amino acid 1309 led to the isogenic APC^{mut} cell line (CTRPA A1309). As can be seen from data in Table 5, all compounds retained exquisite selectivity for cells with truncating APC mutations with low nanomolar IC₅₀'s in CTRPA A1309 and no apparent effect on the isogenic cell line CTRPA (IC₅₀>5 μM).

TABLE 6
Pharmacokinetics of Compounds 6, 16, 22, and 92 in Mouse.^{a}

| | | Compound | | | | | | |
|---|---|---|---|---|---|---|---|---|
| | | 6 | 22 | 22 | 22 | 16 | 92 | 92 |
| Route | | ip | iv | ip | po | iv | iv | ip |
| Dose (mg/kg) | | 10 | 5 | 10 | 20 | 10 | 10 | 10 |
| Plasma | T_{1/2} (min) | 48 | 162 | 168 | 182 | 81 | 135 | 171 |
| pK | Cₘₐₓ (ng/mL) | 2,390 | 742 | 1,117 | 691 | 191 | 303 | 145 |
| | Tₘₐₓ (min) | 10 | 10 | 10 | 10 | 30 | 10 | 10 |
| | AUCₗₐₛₜ | 104,103 | 197,571 | 205,914 | 328,296 | 21,581 | 39,146 | 27,904 |
| | (ng · min/mL) | | | | | | | |
| | Vz (mL) | 162 | 135 | 156 | 153 | 1243 | 1152 | 1390 |
| | CL (mL/min) | 2.32 | 0.58 | 0.64 | 0.58 | 10.6 | 5.92 | 5.64 |
| Large | T_{1/2} (min) | 570 | 912 | 903 | 506 | 13,861 | 504 | 678 |
| Intestinal | Cₘₐₓ (ng/g) | 10,678 | 1,816 | 10,146 | 3,252 | 1,414 | 2,974 | 5,754 |
| pK | Tₘₐₓ (min) | 10 | 10 | 10 | 10 | 10 | 10 | 10 |
| | AUClast | 737,709 | 511,878 | 790,425 | 890,969 | 262,874 | 559,037 | 633,289 |
| | (ng · min/mL) | | | | | | | |

^{a}Elimination half-life (T_{1/2}), maximum observed concentration (Cₘₐₓ), time to Cₘₐₓ (Tₘₐₓ), apparent volume of distribution during terminal phase (Vz), area under the concentration-time curve from time zero to the last measured concentration (AUCₗₐₛₜ), clearance (CL), intravenous (i.v.), intraperitoneal (i.p.), per os (p.o.).

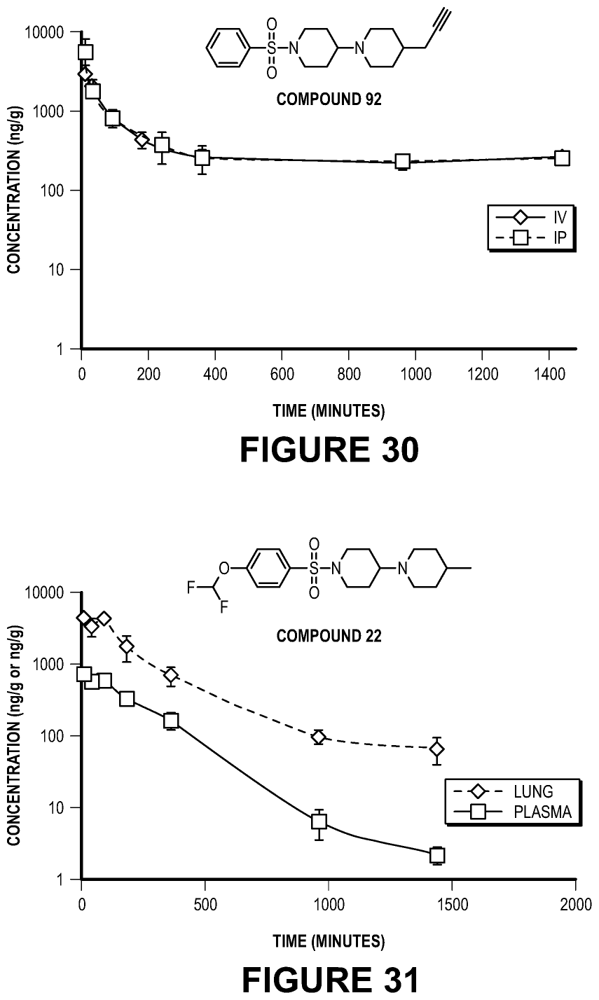

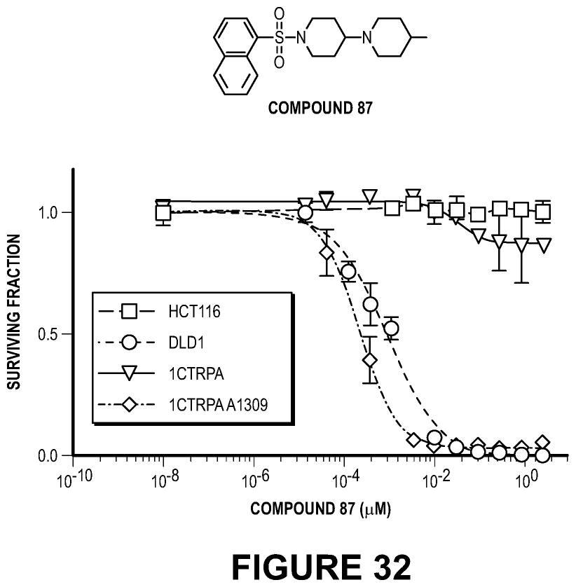

PK Properties of Select Compounds. As a prelude for future in vivo efficacy studies in xenografts and genetic models of CRC, we selected compounds 16, 22, and 92 for in vivo pharmacokinetic (PK) analysis because of their cellular potency (IC₅₀<5 nM) and low microsomal clearance (murine S9 T_{1/2} 41-240 min; CLᵢₙₜ 2.9-16.9 μL/min/mg protein). The administration routes can include, but not limited to, intraperitoneal injection (ip), intravenous (IV) injection or infusion, and Per os (PO, taken orally). As can be seen from the data compiled in Table 6, the PK characteristics of compounds 16, 22, and 92 mirrored those of the previously characterized TASIN-1 (compound 6). When dosed intravenously (i.v.), compounds 16 (5 mg/kg), 22 (10 mg/kg), and 92 (10 mg/kg) had low to moderate plasma clearance between 0.58 and 10.6 mL/min, a half-life between 1.35 and 2.8 h, and a Cₘₐₓ between 191 and 742 ng/mL 10 to 30 min after dosing. Plasma exposure for compounds 16 and 92, while good, was significantly lower than for compound 22 and TASIN-1 (compound 6). Intraperitoneal (i.p.) dosing led to similar plasma clearance, Cₘₐₓ, terminal half-life and exposure as for the i.v. route. The % plasma protein binding was determined using ultrafiltration (compound 22, 69% bound) or rapid equilibrium dialysis (compound 92, 83% bound; compound 6, 8% bound). We selected compound 22 for oral bioavailablity, which was determined to be excellent (52%, 20 mg/kg) and leading to higher plasma and intestinal exposures than when dosed i.v. at 5 mg/kg. Future efficacy studies will include evaluation of select compounds in a genetically engineered mouse apc inactivation model of colonic adenoma-carcinoma progression (CPC;APC mice). Therefore, we assessed the PK of these compounds in the large intestine, the intended target organ. Gratifyingly, large intestinal exposure was excellent, irrespective of the delivery method and between 2.6-23 fold higher than the plasma exposure. The Tₘₐₓ was achieved 10 min after dosing with a the terminal half-life between 8-15 h except compound 16 with an extraordinary long half-life of 231 h. Examination of the concentration-time curves indicated that elimination reached a plateau phase after an initial seemingly normally decaying half-life, an example which is provide in FIG. 30. This behavior results in the apparent high AUC numbers for this class of compounds. The observed accumulation in the large intestine is likely not limited to this organ. For example, a PK analysis of compound 22 indicated similar levels of accumulation in the lung (5.7-fold higher than plasma), and probably other highly perfused organs (FIG. 31). As a result, the volume of distribution for compounds 22, 16, and 92 was large, ranging from 5.87 to 63 L/kg. This phenomenon is not unexpected as many lipohilic amine drugs (e.g.) are known to be deposited in highly perfused, lysosome-rich organs via lysosomal trapping. Together with their ability to bind phospholipids, this lysosomal trapping contributes to presystemic extraction and the large volume of distribution of many cationic amphiphilic drugs including imipramine, tamoxifen, propranolol, and others. Although we have not yet experimentally assessed lysosomal trapping for the analogs described herein, a future evaluation is warranted as lysosomal accumulation has in some instances been implicated as a cause for phospholipidosis. FIG. 32 shows the pharmacological data of compound 87 in different cell lines.

In conclusion, there are currently no small molecule therapeutics that target specifically oncogenotypes that drive the development and progression of colorectal cancers. Germline truncating mutations in the APC tumor suppressor gene lead to Familial Adenomatous Polyposis (FAP) and early development of CRC, whereas somatic truncating APC mutations are observed in the vast majority of sporadic CRC patients. We previously disclosed the discovery of TASIN-1, a small molecule that selectively targets human colorectal cancer cell lines expressing mutant-APC with high specificity through inhibition of endogenous cholesterol biosynthesis. Here, we reported an extensive Structure-Activity Relationship study through the design of analogs of TASIN-1 exploring the structural determinants responsible for cellular activity and selectivity. This study identified several very potent analogs with good drug-like properties that inhibit CRC cells with mutant APC in the single-digit nanomolar to picomolar range, while being innocuous for cells with wildtype APC. Several of these potent analogs exhibited acceptable metabolically stability in murine microsomal fractions, and excellent in vivo exposure whether dosed i.v., i.p. or orally. The high intestinal exposure and half-life of this class bodes well for future efficacy studies in genetic or orthotopic animal models of CRC. However, we note that this significant intestinal accumulation and long half-life could indicate a potential lysosomal trapping of these lipophilic amines, an issue that will be explored in future studies. Lipophilic basic amine drugs are also potentially liable for off-target activity against potassium and sodium channels. Given that our SAR studies indicate that a protonatable amine is absolutely essential for activity, we will have to evaluate our compound collection against these and other potential off-target interactions. The SAR studies described herein indicate that the TASINs represents an excellent scaffold for such SAR-driven optimization, and we are therefore confident that ongoing studies will enable the identification of novel translatable leads for a potential targeted therapy for colorectal cancer.

Although the invention has been described with reference to the above examples, it will be understood that modifications and variations are encompassed within the spirit and scope of the invention. Accordingly, the invention is limited only by the following claims.

1. A compound according to Formula (I): 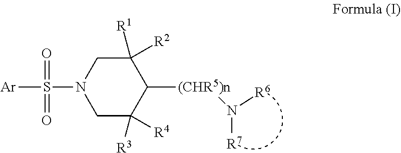
or a pharmaceutically acceptable salt or solvate, a stereoisomer, a diastereoisomer or an enantiomer thereof, wherein
R¹, R², R³, and R⁴ are independently selected from the group consisting of H, F, CF₃, CHF₂, CH₂F, and methyl;
Ar is selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl,
wherein the optional substituent is selected from the group consisting of F, Cl, Br, —OCF₃, —OCHF₂, —OCH₂F, —OCH₂R⁸, —OCHMeR⁸, —OCH(CF₃)R⁸, —OR⁸, —C(O)R⁸, R⁸, C1-4 alkyl, C3-5 cycloalkyl, C2-6 alkenyl, C2-6 alkynyl, —OC1-4 alkyl, —OC3-5 cycloalkyl, —OC2-6 alkenyl, and —OC2-6 alkynyl, in which
C1-4 alkyl or C3-5 cycloalkyl is optionally substituted selected from the group consisting of fluorine, hydroxyl, C1-3 alkoxy group, tetrahydropyranyl optionally substituted with one or more fluorines, hydroxyl, or C1-3 alkoxy group, tetrahydrofuranyl optionally substituted with one or more fluorines, hydroxyl, or C1-3 alkoxy group, and a combination thereof,
C2-6 alkenyl or C2-6 alkynyl is optionally substituted with fluorine, hydroxyl, C1-3 alkoxy group, or a combination thereof,
—OC1-4 alkyl or —OC3-5 cycloalkyl is optionally substituted with fluorine, hydroxyl, C1-3 alkoxy group, or a combination thereof,
—OC2-6 alkenyl or —OC2-6 alkynyl is optionally substituted with fluorine, hydroxyl, C1-3 alkoxy group, or a combination thereof,
n=0 or 1;
when n=1, R⁵ is selected from the group consisting of H, methyl, CF₃, CHF₂, and CH₂F;
R⁶ and R⁷ are independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F or OH; C1-3 alkoxy group; —CH₂CCH; R⁸; CH₂R⁸; OR⁸; OCH₂R⁸; OCHMeR⁸;
or wherein R⁶ and R⁷ are connected to form a nitrogen-containing heterocycle, in such case, R⁶-R⁷ is to be selected from the group consisting of —(CHR¹⁰)CH₂(CHR¹⁰)O(CHR⁹)—, —(CHR⁹)O(CHR¹⁰)₂—, —CH₂(CR¹²R¹³)CH₂—, —(CH₂)₂ (CHR¹¹)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkyldienyl)-, —(CHMe)CH₂O(3,3-oxatenylidenyl)-, —(CH₂)₂(1,1-cycloalkyldienyl)CH₂—, —(CH₂)ₘ— with m=4-6 and the provision that when n=0 then m≠5 and optionally substituted with one or more substituents independently selected from the group consisting of F, OH, and R¹⁰;
R⁸ is phenyl or heteroaryl optionally substituted with one or more substituents independently selected from the group consisting of F, Cl, Br, CF₃, CHF₃, CH₂F, C1-4 alkyl, C3-5 cycloalkyl, —OC1-4 alkyl, and —OC3-5 cycloalkyl, wherein C1-4 alkyl, C3-5 cycloalkyl, —OC1-4 alkyl, or —OC3-5 cycloalkyl is optionally substituted with one or more fluorines;
R⁹ is selected from the group consisting of H, R⁸, C1-4 alkyl, —OC1-3 alkyl, and —OC3-5 cycloalkyl, wherein C1-4 alkyl, —OC1-3 alkyl, or —OC3-5 cycloalkyl is optionally substituted substituents selected from the group consisting of F, OH, R⁸, and a combination thereof;
R¹⁰ is selected from the group consisting of H, R⁸, C1-4 alkyl, C3-6 alkenyl, C3-6 alkynyl, —OC1-3 alkyl, —OC3-5 cycloalkyl, wherein C1-4 alkyl, C3-6 alkenyl, C3-6 alkynyl, —OC1-3 alkyl, or —OC3-5 cycloalkyl is optionally substituted with substituents selected from the group consisting of F, OH, R⁸, OR⁸, OCH₂R⁸, OCHMeR⁸, and a combination thereof;
R¹¹ is selected from the group consisting of H, CO₂H, CO₂R¹⁴, CH₂OH, CH₂OR¹⁴, C1-4 alkyl, C3-6 alkenyl, C3-6 alkynyl, and C3-5 cycloalkyl, wherein C1-4 alkyl, C3-6 alkenyl, C3-6 alkynyl, or C3-5 cycloalkyl is optionally substituted with one or more substituents selected from the group consisting of F, OH, and R⁸;
R¹² and R¹³ are independently selected from the group consisting of H, F, CF₃, CHF₂, CH₂F, CN, OH, OR¹⁴, NHC(O)Me, SO₂Me, OSO₂Me, CO₂H, CO₂R¹⁴, CH₂OH, CH₂OR¹⁴, R⁸, and R¹⁴;
or wherein R¹² and R¹³ are optionally connected to form a cyclic structure, in such a case, R¹²-R¹³ is to be selected from the group consisting of: —CH₂OCH₂—, —(CH₂)₂O—, —(CH₂)₃O—, —(CH₂)₃—, —(CH₂)₄—, —CH₂CF₂CH₂—, —CH₂O(CHCF₃)—, —CH₂SO₂(CHCF₃)—, —CH₂(CHCO₂H)CH₂—, —CH₂(CHCO₂R¹⁴)CH₂—, —CH₂(CHCH₂OH)CH₂—, —CH₂(CHCH₂OR¹⁴)CH₂—, —(CHOH)CH₂O—, —(CHOR¹⁴)CH₂O—, —SO₂(CH₂)₂(CHOH)—, —SO₂(CH₂)₂(CHOR¹⁴)—, —SO₂(CH₂)(CHOH)CH₂—, —SO₂(CH₂)(CHOR¹⁴)CH₂—, —CH₂(CHOH)CH₂O—, —CH₂(CHOR¹⁴)CH₂O—, —(CHOH)(CH₂)₂O—(CHOR¹⁴)(CH₂)₂O—, and —CH₂(3,3-oxetanyl)CH₂-;
and R¹⁴ is selected from the group consisting of C1-4 alkyl, C3-5 cycloalkyl, C2-6 alkenyl, and C2-6 alkynyl, each of which is optionally substituted with one or more substituents selected from F, OH, and Re.

2. The compound according to claim 1, wherein:
R¹, R², R³, and R⁴ are independently selected from the group consisting of H, F, and CF₃;
Ar is selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl, optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted thiazol-5-yl, optionally-substituted oxazol-5-yl;
wherein the optional substituents are one or more substituents independently selected from the group consisting of F, Cl, Br, methyl, CF₃, ethyl, isopropyl, cyclopropyl, —OMe, —OEt, —Oi-Pr, —Ocyclopropyl, —OCF₃, —OCHF₂, —OCH₂F, —OCH₂R⁸, —OR⁸, —C(O)R⁸, and R⁸;
n=0 or 1,
when n=1, R⁵ is H, methyl, or CF₃;
R⁶ and R⁷ are independently selected from the group consisting of H, C1-10 alkyl, C2-10 alkenyl, C2-10 alkynyl, and C3-7 cycloalkyl, wherein C1-10 alkyl, C2-10 alkenyl, C2-10 alkynyl, or C3-7 cycloalkyl is optionally substituted with one or more substituents independently selected from the group consisting of F, OH, and C1-4 alkyl optionally substituted with one or more functional groups selected from F, OH, C1-3 alkoxy, —CH₂CCH, R⁸, CH₂R⁸, OR⁸, and OCH₂R⁸;
or wherein R⁶ and R⁷ can be connected to form a nitrogen-containing heterocycle, in such case, R⁶-R⁷ is selected from the group consisting of —(CHR¹⁰)CH₂(CHR¹⁰)O(CHR⁹)—, —(CHR⁹)O(CHR¹⁰)₂—, —CH₂(CR¹²R¹³)CH₂—, —(CH₂)₂(CHR¹¹)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkyldienyl)-, —(CHMe)CH₂O(3,3-oxatenylidenyl)-, —(CH₂)₂(1,1-cycloalkylidenyl)CH₂—, and —(CH₂)m- with m=4-6 and the proviso that when n=0, m≠5, each of the nitrogen-containing heterocycle is optionally substituted with one or more substituents independently selected from the group consisting of F, OH, and R¹⁰;
R⁸ is phenyl optionally substituted with one or more substituents independently selected from the group consisting of F, Cl, CF₃, CHF₃, CH₂F, cyclopropyl, methyl, ethyl, isopropyl, OMe, OCF₃, OCHF₂, OCH₂F, —OEt, —Oi-Pr, and Ocyclopropyl;
R⁹ is selected from the group consisting of H, R⁸, C1-4 alkyl, and —OC1-3 alkyl, wherein C1-4 alkyl or —OC1-3 alkyl is optionally substituted with one or more substituents selected from the group consisting of F, OH, and R⁸;
R¹⁰ is selected from the group consisting of H, R⁸, C1-4 alkyl, C3-6 alkynyl, and —OC1-3 alkyl, wherein C1-4 alkyl, C3-6 alkynyl, or —OC1-3 alkyl is optionally substituted with one or more substituents selected from the group consisting of F, OH, R⁸, OR⁸, and OCH₂R⁸;
R¹¹ is selected from the group consisting of H, CO₂H, CO₂R¹⁴, CH₂OH, CH₂OR¹⁴, C1-4 alkyl, C3-5 cycloalkyl, and C3-6 alkynyl, wherein C1-4 alkyl, C3-5 cycloalkyl, or C3-6 alkynyl is optionally substituted with one or more substituents selected from the group consisting of F, OH, and R⁸;
R¹² and R¹³ are independently selected from the group consisting of H, F, CF₃, CHF₂, CH₂F, CN, OH, OR¹⁴, NHC(O)Me, SO₂Me, OSO2Me, CO₂H, CO₂R¹⁴, CH₂OH, CH₂OR¹⁴, R⁸, and R⁴;
or wherein R¹² and R¹³ are optionally connected to form a cyclic structure, in such a case, R¹²-R¹³ is selected from the group consisting of —CH₂OCH₂—, —(CH₂)₂O—, —(CH₂)₃O—, —(CH₂)₃—, —(CH₂)₄—, —CH₂CF₂CH₂—, —CH₂O(CHCF₃)—, —CH₂SO₂(CHCF₃)—, —CH₂(CHCO₂H)CH₂—, —CH₂(CHCO₂R¹⁴)CH₂—, —CH₂(CHCH₂OH)CH₂—, —CH₂(CHCH₂OR¹⁴)CH₂—, —(CHOH)CH₂O—, —(CHOR¹⁴)CH₂O—, —SO₂(CH₂)₂(CHOH)—, —SO₂(CH₂)₂(CHOR¹⁴)—, —SO₂(CH₂)(CHOH)CH₂—, —SO₂(CH₂)(CHOR¹⁴)CH₂—, —CH₂(CHOH)CH₂O—, —CH₂(CHOR¹⁴)CH₂O—, —(CHOH)(CH₂)₂O—, —(CHOR⁴)(CH₂)₂O—, and —CH₂(3,3-oxetanyl)CH₂-;
and R⁴ is selected from the group consisting of C1-4 alkyl, C3-5 cycloalkyl, C2-6 alkenyl, and C2-6 alkynyl, each of which is optionally substituted with one or more substituents selected from F, OH, and R⁸.

3. The compound according to claim 1, wherein Ar is selected from the group consisting of: 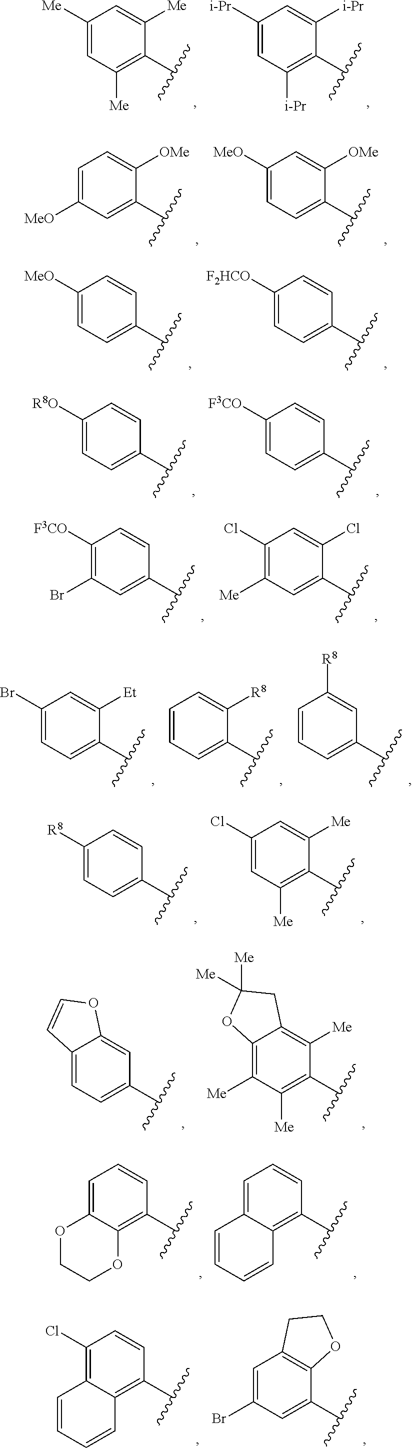 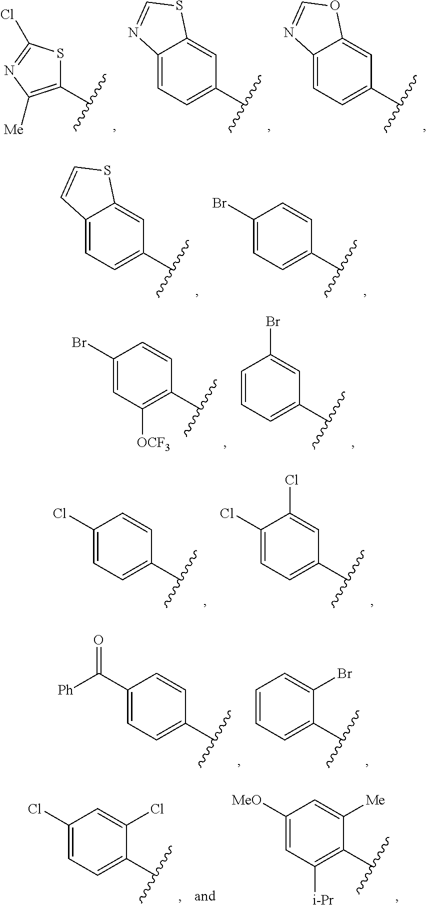
each of which is optionally substituted with one or more substituents independently selected from the group consisting of F, Cl, Br, Me, CF₃, Et, i-Pr, cyclopropyl, OMe, OEt, Oi-Pr, —Ocyclopropyl, —OCF₃, —OCHF₂, —OCH₂F, —OCH₂R⁸, —OR⁸ and R⁸.

4. The compound according to claim 1, wherein when n=0, —NR⁶R⁷ is selected from group consisting of:

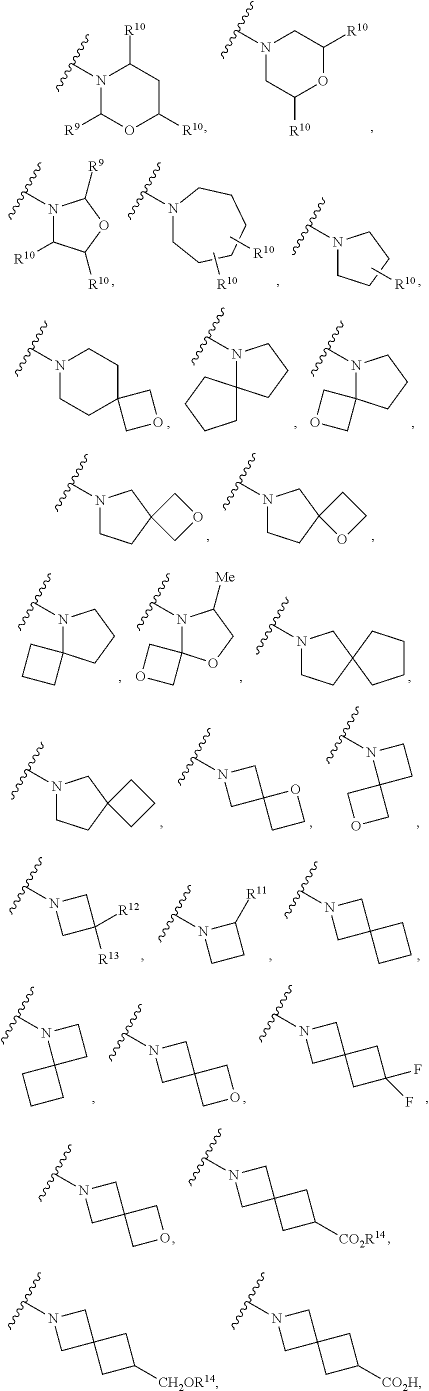

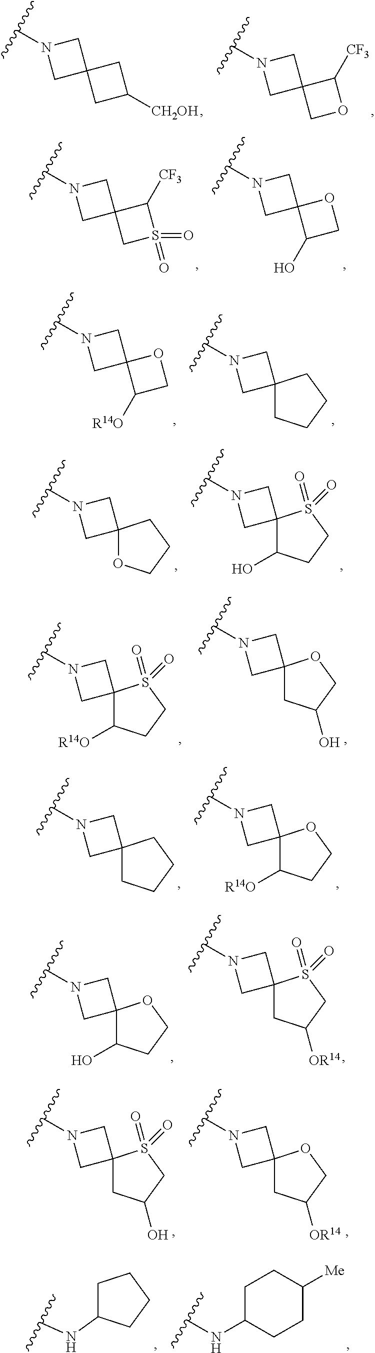

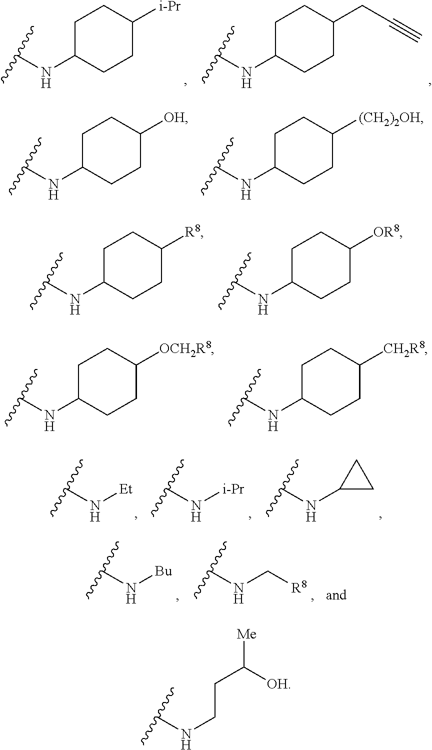

5. The compound according to claim 1, wherein when n=1, —NR⁶R⁷ is selected from group consisting of:

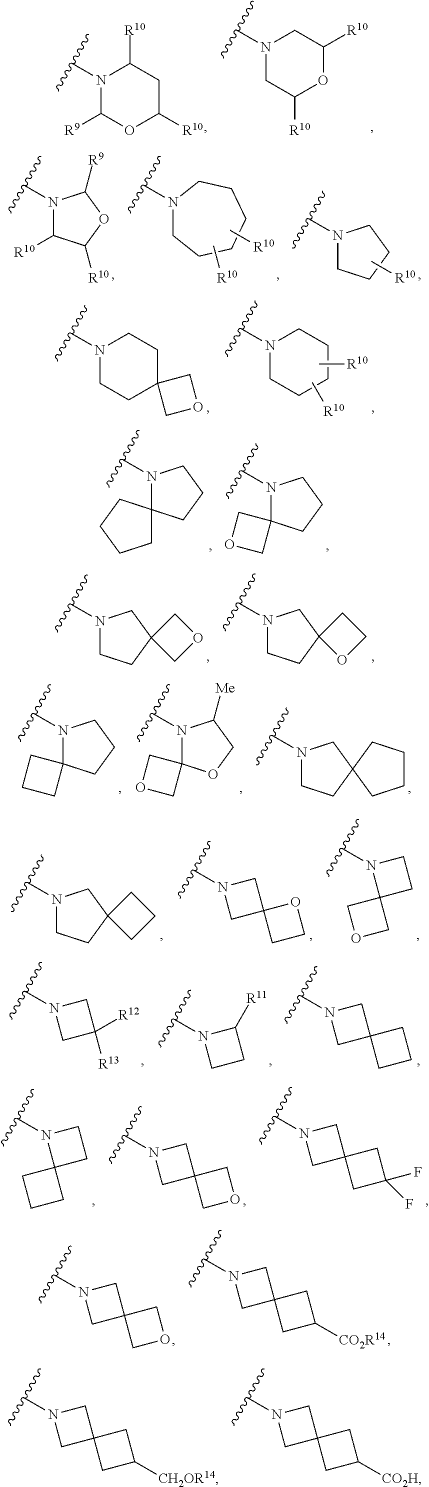

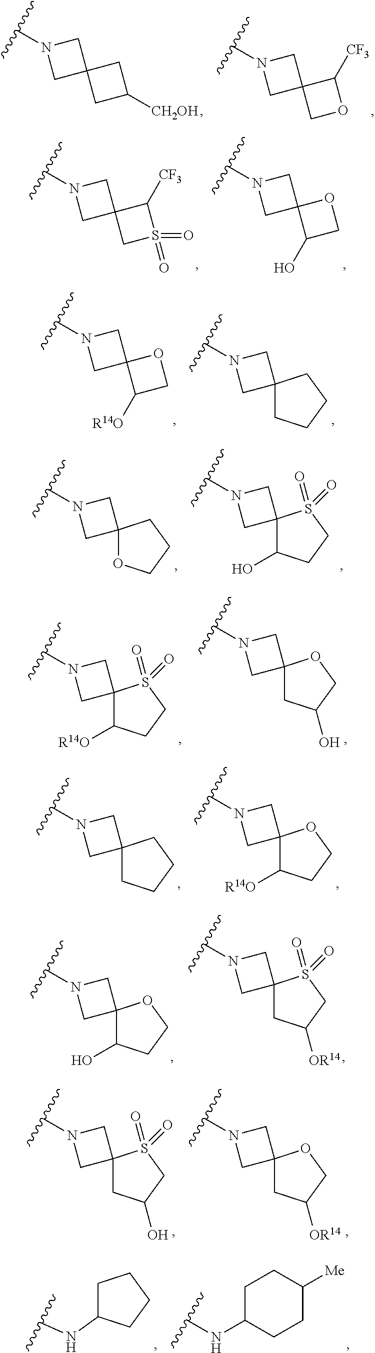

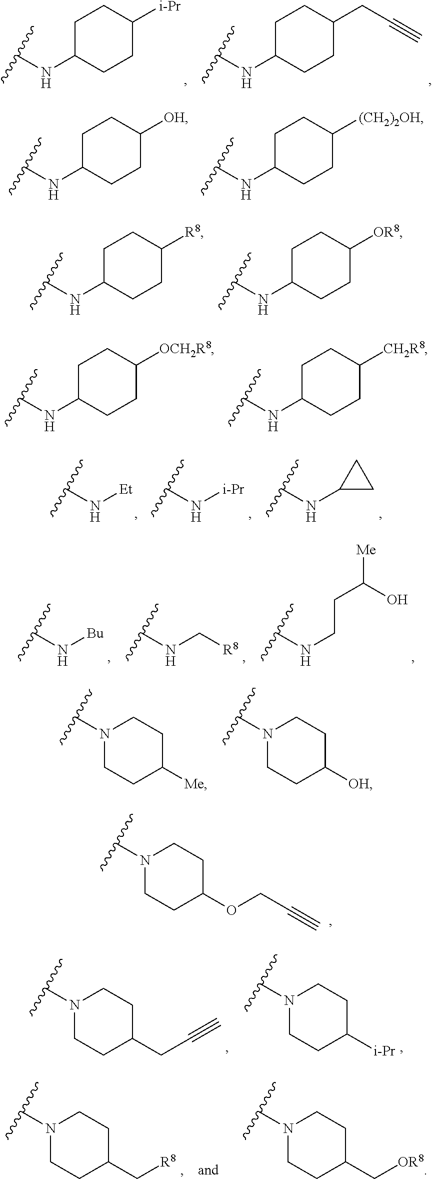

6. The compound according to claim 1, wherein the compound is in form of a pharmaceutical composition comprising a therapeutic amount of the compound and a pharmaceutically acceptable carrier.

7. The compound according to claim 1, wherein the compound is effective to inhibit tumor growth, inhibit tumor proliferation, induce cell death or a combination thereof.

8. A stereoisomer, a diastereoisomer or an enantiomer of the compound according to claim 1.

9. A pharmaceutically acceptable salt or solvate of the compound according to claim 1.

10. The compound according to claim 1, wherein a therapeutic amount of the compound is effective to inhibit Emopamil Binding Protein (EBP) or cholesterol delta8 delta7 somerase.

11-20. (canceled)

21. A compound according to Formula (II): 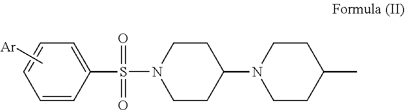
or a pharmaceutically acceptable salt or solvate, a stereoisomer, a diastereoisomer or an enantiomer thereof,
wherein Ar is selected from the group consisting of substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl,
wherein the optional substituents are one or more substituents independently selected from the group consisting of F, Cl, Br, —OCF₃, —OCHF₂, —OCH₂F, —OCH₂R¹, —OCHMeR¹, —OCH(CF₃)R¹, —OR¹, —C(O)R¹, R¹, C1-4 alkyl or C3-5 cycloalkyl optionally substituted with one or more fluorines and/or hydroxy and/or C1-3 alkoxy group, tetrahydropyranyl or tetrahydrofuranyl optionally substituted with one or more fluorines and/or hydroxy and/or C1-3 alkoxy group, C2-6 alkenyl or alkynyl optionally substituted with one or more fluorines and/or hydroxy and/or C1-3 alkoxy group, —OC1-4 alkyl or —OC3-5 cycloalkyl optionally substituted with one or more fluorines and/or hydroxy and/or C1-3 alkoxy group, —OC2-6 alkenyl or alkynyl optionally substituted with one or more fluorines and/or hydroxy and/or C1-3 alkoxy group,
R¹ is phenyl or heteroaryl optionally substituted with one or more substituents independently selected from the group consisting of F, C1, Br, CF₃, CHF₃, CH₂F, C1-4 alkyl or C3-5 cycloalkyl optionally substituted with one or more fluorines, and —OC1-4 alkyl or —OC3-5 cycloalkyl optionally substituted with one or more fluorines,
wherein the compound is not

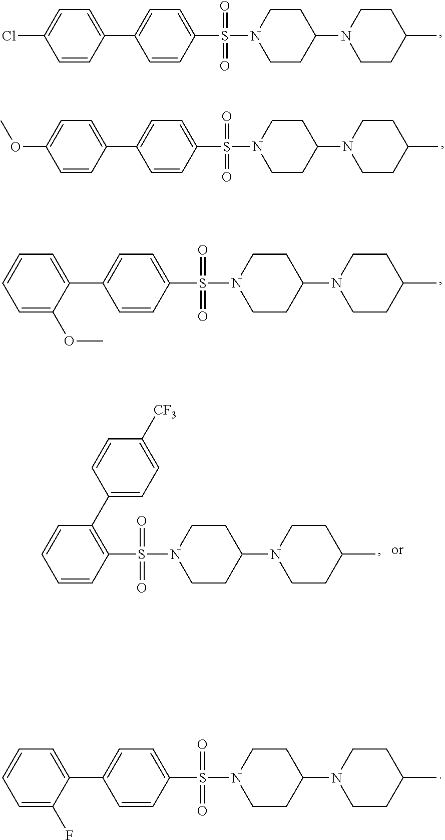

22. The compound according to claim 21, wherein the compound is effective to inhibit tumor growth, inhibit tumor proliferation, induce cell death or a combination thereof.

23. The compound according to claim 21, wherein a therapeutic amount of the compound is effective to inhibit Emopamil Binding Protein (EBP) or cholesterol delta8 delta7 somerase.

24. A pharmaceutical composition comprising a therapeutic amount of the compound according to claim 21 and a pharmaceutically acceptable carrier.

25. A method for treating colorectal cancer in a subject comprising administering the compound according to claim 21.

26. The method of claim 25, comprising administering a chemotherapeutic agent.

27-32. (canceled)

33. A pharmaceutical composition comprising a therapeutic amount of the compound according to claim 21 and a pharmaceutically acceptable carrier. 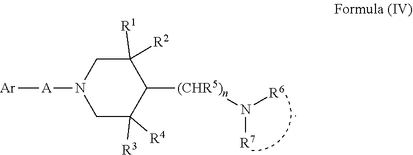
or a pharmaceutically acceptable salt or solvate, a stereoisomer, a diastereoisomer or an enantiomer thereof, wherein
A is —NR⁸—SO₂— or —NR⁸—CO—;
R¹, R², R³, and R⁴ is independently selected from the group consisting of H, F, CF₃, CHF₂, CH₂F, and methyl;
R⁸ is selected from the group consisting of H and optionally-substituted C1-C4 alkyl;
Ar is selected from the group consisting of optionally-substituted phenyl, optionally-substituted naphthyl, optionally-substituted benzo[d]thiazol-4-yl, optionally-substituted benzo[d]thiazol-5-yl, optionally-substituted benzo[d]thiazol-6-yl, optionally-substituted benzo[d]thiazol-7-yl, optionally-substituted benzo[d]oxazol-4-yl, optionally-substituted benzo[d]oxazol-5-yl, optionally-substituted benzo[d]oxazol-6-yl, optionally-substituted benzo[d]oxazol-7-yl, optionally-substituted 2,3-dihydrobenzofuran-4-yl, optionally-substituted 2,3-dihydrobenzofuran-5-yl, optionally-substituted 2,3-dihydrobenzofuran-6-yl, optionally-substituted 2,3-dihydrobenzofuran-7-yl, optionally-substituted benzofuran-4-yl, optionally-substituted benzofuran-5-yl, optionally-substituted benzofuran-6-yl, optionally-substituted benzofuran-7-yl, optionally-substituted benzo[b]thiophen-4-yl; optionally-substituted benzo[b]thiophen-5-yl, optionally-substituted benzo[b]thiophen-6-yl, optionally-substituted benzo[b]thiophen-7-yl, optionally-substituted 2,3-dihydrobenzo[b]thiophen-4-yl, optionally-substituted 2,3-dihydrobenzo[b]thiophen-5-yl, optionally-substituted 2,3-dihydrobenzo[b]thiophen-6-yl, optionally-substituted 2,3-dihydrobenzo[b]thiophen-7-yl, optionally-substituted 2,3-dihydrobenzo[b]thiophen-4-yl 1-oxide, optionally-substituted 2,3-dihydrobenzo[b]thiophen-5-yl 1-oxide, optionally-substituted 2,3-dihydrobenzo[b]thiophen-6-yl 1-oxide, optionally-substituted 2,3-dihydrobenzo[b]thiophen-7-yl 1-oxide, optionally-substituted thiazol-2-yl, optionally-substituted thiazol-5-yl, optionally-substituted thiazol-4-yl, optionally-substituted oxazol-2-yl, optionally-substituted oxazol-4-yl, and optionally-substituted oxazol-5-yl;
the optional substituent for Ar is selected from the group consisting of F, Cl, Br, —OCF₃, —OCHF₂, —OCH₂F, —OCH₂R⁹, —OCHMeR⁹, —OCH(CF₃)R⁹, —OR⁹, —C(O)R⁹, R⁹, C1-4 alkyl, C3-5 cycloalkyl, C2-6 alkenyl, C2-6 alkynyl, —OC1-4 alkyl, —OC3-5 cycloalkyl, —OC2-6 alkenyl, and —OC2-6 alkynyl;
C1-4 alkyl or C3-5 cycloalkyl is optionally substituted selected from the group consisting of fluorine, hydroxyl, C1-3 alkoxy group, tetrahydropyranyl optionally substituted with one or more fluorines, hydroxyl, or C1-3 alkoxy group, tetrahydrofuranyl optionally substituted with one or more fluorines, hydroxyl, or C1-3 alkoxy group, and a combination thereof,
C2-6 alkenyl or C2-6 alkynyl is optionally substituted with fluorine, hydroxyl, C1-3 alkoxy group, or a combination thereof,
—OC1-4 alkyl or —OC3-5 cycloalkyl is optionally substituted with fluorine, hydroxyl, C1-3 alkoxy group, or a combination thereof,
—OC2-6 alkenyl or —OC2-6 alkynyl is optionally substituted with fluorine, hydroxyl, C1-3 alkoxy group, or a combination thereof,
n is 0 or 1;
when n=1, R⁵ is selected from the group consisting of H, methyl, CF₃, CHF₂, and CH₂F;
R⁶ and R⁷ are independently selected from the group consisting of H, C1-10 alkyl, C2-10 alkenyl, C2-10 alkynyl, and C3-7 cycloalkyl. The alkyl/alkenyl/alkynyl/cycloalkyl groups are optionally further functionalized with one or more substituents independently selected from the group consisting of F, OH, C1-4 alkyl optionally substituted with one or more F or OH; C1-3 alkoxy group; —CH₂CCH; R⁹; CH₂R⁹; OR⁹; OCH₂R⁹; OCHMeR⁹;
or R⁶ and R⁷ are connected to form a nitrogen-containing heterocycle, in such case, R⁶-R⁷ is to be selected from the group consisting of —(CHR¹¹)CH₂(CHR¹¹)O(CHR¹⁰)—, —(CHR¹⁰)O(CHR¹¹)₂—, —CH₂(CR¹³R¹⁴)CH₂—, —(CH₂)₂ (CHR¹²)—, —(CH₂)₂(2,2-oxetanylidenyl)CH₂—, —(CH₂)₂(3,3-oxetanylidenyl)CH₂—, —(CH₂)₃(3,3-oxetanylidenyl)-, —(CH₂)₂(3,3-oxetanylidenyl)(CH₂)₂—, —(CH₂)₂(3,3-oxetanylidenyl)-, —(CH₂)₃(1,1-cycloalkyldienyl)-, —(CHMe)CH₂O(3,3-oxetanylidenyl)-, —(CH₂)₂(1,1-cycloalkyldienyl)CH₂—, —(CH₂)ₘ— with m=4-6 and optionally substituted with one or more substituents independently selected from the group consisting of F, OH, and R¹¹;
R⁹ is phenyl or heteroaryl optionally substituted with one or more substituents independently selected from the group consisting of F, Cl, Br, CF₃, CHF₃, CH₂F, C1-4 alkyl, C3-5 cycloalkyl, —OC1-4 alkyl, and —OC3-5 cycloalkyl, wherein C1-4 alkyl, C3-5 cycloalkyl, —OC1-4 alkyl, or —OC3-5 cycloalkyl is optionally substituted with one or more fluorines;
R¹⁰ is selected from the group consisting of H, R⁹, C1-4 alkyl, —OC1-3 alkyl, and —OC3-5 cycloalkyl, wherein C1-4 alkyl, —OC1-3 alkyl, or —OC3-5 cycloalkyl can be optionally substituted substituents selected from the group consisting of F, OH, R⁹, and a combination thereof;
R¹¹ is selected from the group consisting of H, R⁹, C1-4 alkyl, C3-6 alkenyl, C3-6 alkynyl, —OC1-3 alkyl, —OC3-5 cycloalkyl, wherein C1-4 alkyl, C3-6 alkenyl, C3-6 alkynyl, —OC1-3 alkyl, or —OC3-5 cycloalkyl is optionally substituted with substituents selected from the group consisting of F, OH, R⁹, OR⁹, OCH₂R⁹, OCHMeR⁹, and a combination thereof,
R¹² is selected from the group consisting of H, CO₂H, CO₂R¹⁵, CH₂OH, CH₂OR¹⁵, C1-4 alkyl, C3-6 alkenyl, C3-6 alkynyl, and C3-5 cycloalkyl, wherein C1-4 alkyl, C3-6 alkenyl, C3-6 alkynyl, or C3-5 cycloalkyl is optionally substituted with one or more substituents selected from the group consisting of F, OH, and R⁹;
each R¹³ and R¹⁴ is independently selected from the group consisting of H, F, CF₃, CHF₂, CH₂F, CN, OH, OR¹⁵, NHC(O)Me, SO₂Me, OSO₂Me, CO₂H, CO₂R¹⁵, CH₂OH, CH₂OR¹⁵, R⁹, and R¹⁵, or R¹³ and R¹⁴ are optionally connected to form a cyclic structure, in such a case, R¹³-R¹⁴ is to be selected from the group consisting of: —CH₂OCH₂—, —(CH₂)₂O—, —(CH₂)₃O—, —(CH₂)₃—, —(CH₂)₄—, —CH₂CF₂CH₂—, —CH₂O(CHCF₃)—, —CH₂SO₂(CHCF₃)—, —CH₂(CHCO₂H)CH₂—, —CH₂(CHCO₂R¹⁵)CH₂—, —CH₂(CHCH₂OH)CH₂—, —CH₂(CHCH₂OR¹³)CH₂—, —(CHOH)CH₂O—, —(CHOR¹³)CH₂O—, —SO₂(CH₂)₂(CHOH)—, —SO₂(CH₂)₂(CHOR¹⁵)—, —SO₂(CH₂)(CHOH)CH₂—, —SO₂(CH₂)(CHOR¹³)CH₂—, —CH₂(CHOH)CH₂O—, —CH₂(CHOR¹³)CH₂O—, —(CHOH)(CH₂)₂O—(CHOR¹⁵)(CH₂)₂O—, and —CH₂(3,3-oxetanyl)CH₂-; and
R¹⁵ is selected from the group consisting of C1-4 alkyl, C3-5 cycloalkyl, C2-6 alkenyl, and C2-6 alkynyl, each of which is optionally substituted with one or more substituents selected from F, OH, and R⁹.

34. The compound according to claim 33, wherein Ar is selected from the group consisting of: 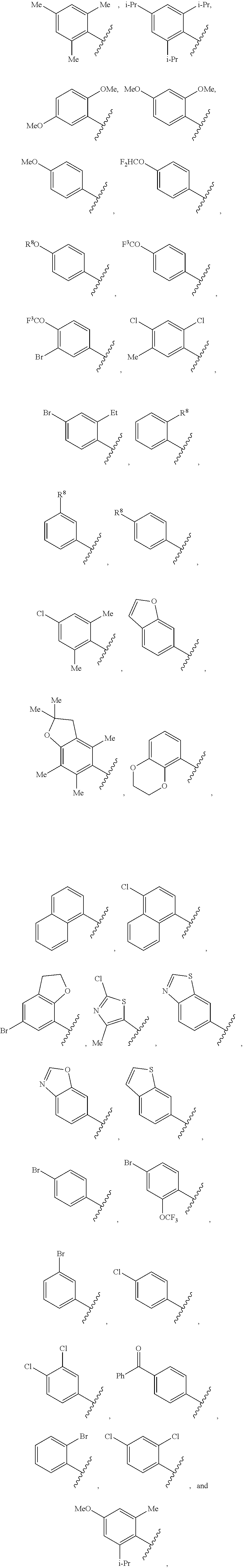
each of which is optionally further substituted with one or more substituents independently selected from the group consisting of F, Cl, Br, Me, CF₃, Et, i-Pr, cyclopropyl, OMe, OEt, Oi-Pr, —Ocyclopropyl, —OCF₃, —OCHF₂, —OCH₂F, —OCH₂R⁹, —OR⁹ and R⁹.

35. The compound according to claim 33, wherein when n=0, —NR⁶R⁷ is selected from group consisting of:

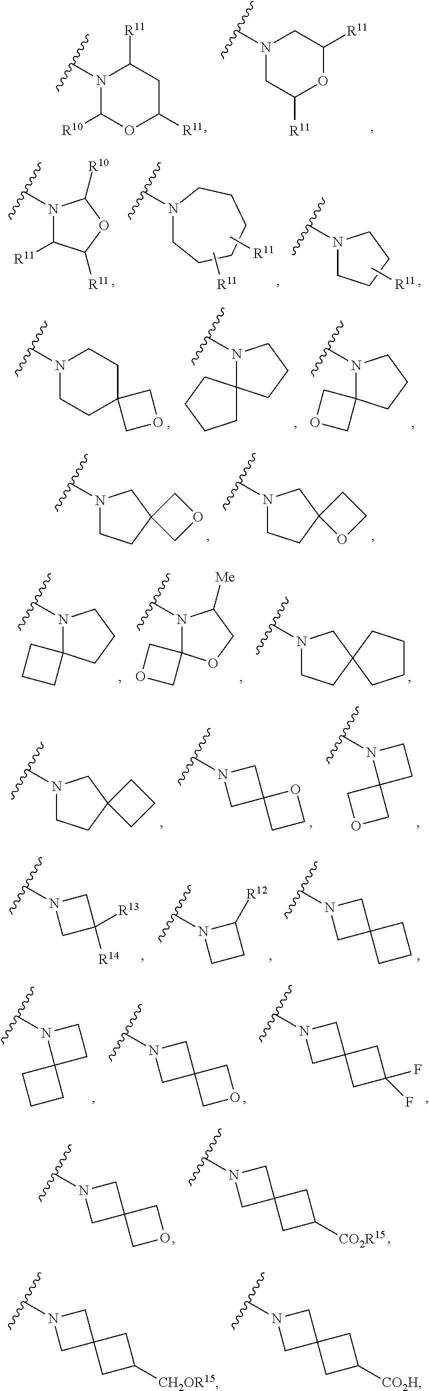

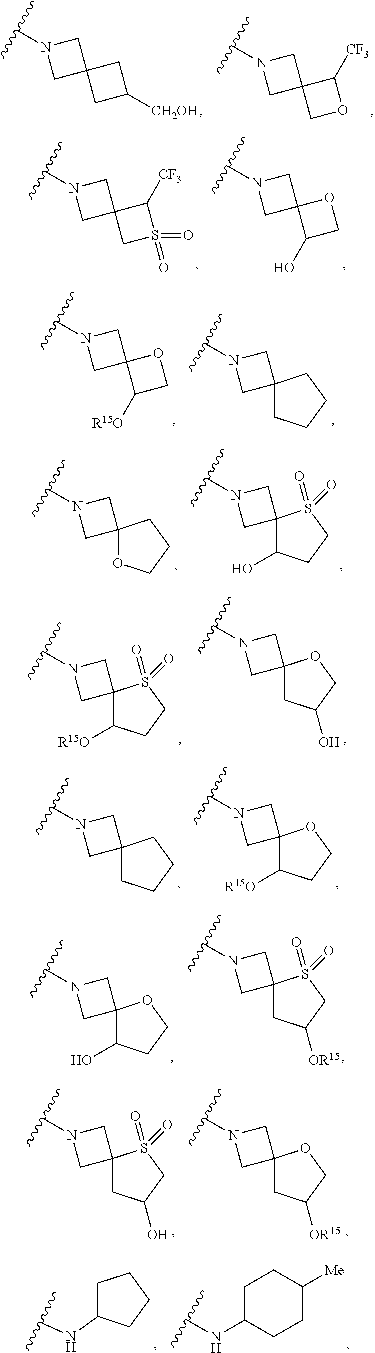

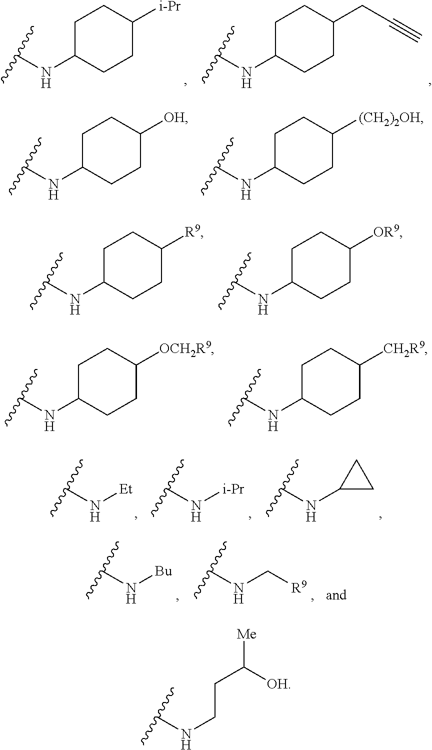

36. The compound according to claim 33, wherein when n=1, —NR⁶R⁷ is selected from group consisting of: 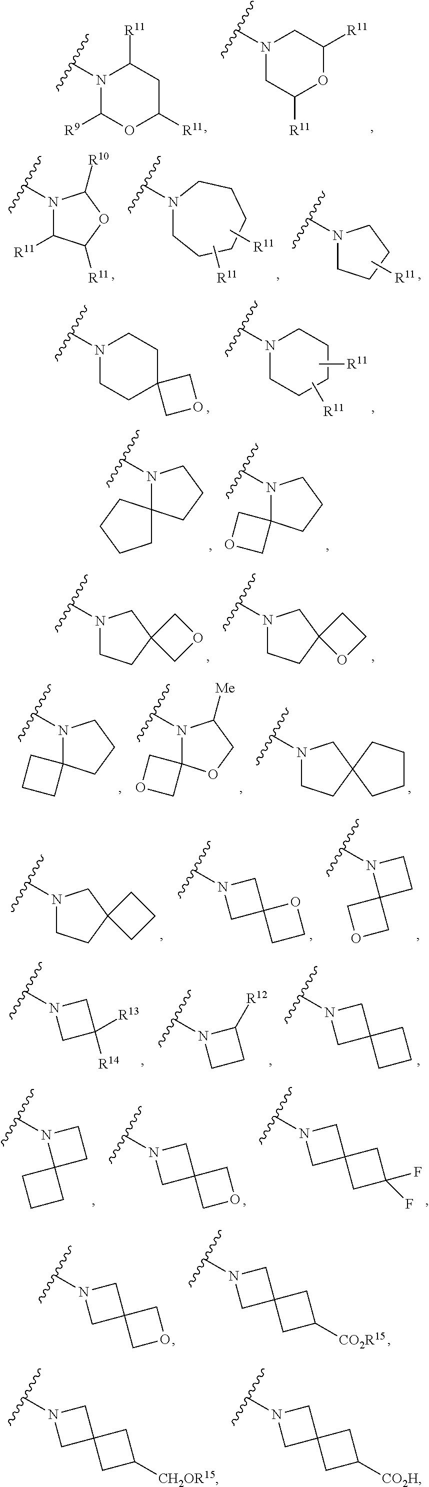 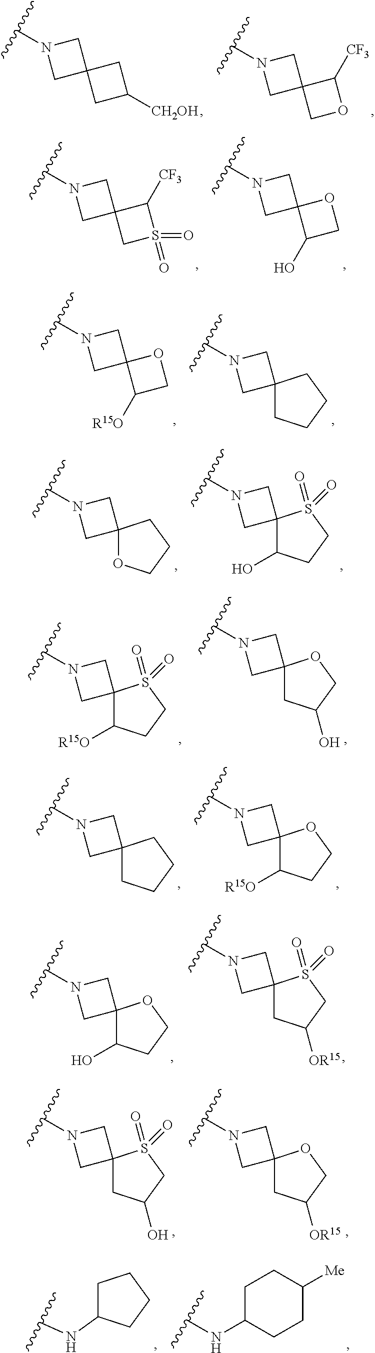 [chemical structure]
